(19) United States
(12) Patent Application Publication
(10) Pub. No.: US 2024/0392273 A1
(43) Pub. Date: Nov. 28, 2024

(54) FACILITATED DELIVERY OF CONCENTRATED ANTIBODY FORMULATIONS USING HYALURONIDASE

(71) Applicant: Takeda Pharmaceutical Company Limited, Osaka (JP)

(72) Inventors: Joris HOEFINGHOFF, Vienna (AT); Peter LEIDENMUEHLER, Vienna (AT); Bagirath GANGADHARAN, Vienna (AT); Norbert HAIDER, Vienna (AT); Andras NAGY, Vienna (AT); Zhaoyang LI, Newton, MA (US)

(21) Appl. No.: 18/691,573

(22) PCT Filed: Sep. 14, 2022
(86) PCT No.: PCT/IB22/58670

§ 371 Date: Mar. 13, 2024

Related U.S. Application Data
(60) Provisional application No. 63/243,832, filed on Sep. 14, 2021.

(51) Int. Cl. C12N 9/26; A61K 9/00; C07K 16/00

(52) U.S. Cl. CPC C12N 9/2474; A61K 9/0019; C07K 16/00; C07K 2317/90; C12Y 302/01035

(57) ABSTRACT

Provided is a concentrated pharmaceutical formulation of an immune globulin (IG), and convenient methods for the subcutaneous administration of this pharmaceutical formulation in a warmed state. Such products can be used in methods of treating IG-treatable diseases or conditions. Also provided are combinations, compositions and kits containing an immune globulin (IG) composition and a soluble hyaluronidase composition formulated for subcutaneous administration.

### CROSS REFERENCES TO APPLICATIONS

This application claims priority to U.S. Provisional Patent Application Ser. No. 63/243,832, filed Sep. 14, 2021, the disclosure of which is hereby incorporated herein by reference in its entirety for all purposes.

### FIELD OF THE INVENTION

The invention resides in the field of antibody therapeutics and in the hyaluronidase facilitated subcutaneous delivery of viscous formulations of therapeutic antibodies.

### BACKGROUND OF THE INVENTION

Immune globulin products from human plasma were first used in 1952 to treat immune deficiency. Initially, intramuscular or subcutaneous (SC) administration of IgG were the methods of choice. For injecting larger amounts of IgG necessary for effective treatment of various diseases, however, intravenous administrable products with lower concentrated IgG (50 mg/mL) were developed. Usually, intravenous immunoglobulin (IVIG) contains the pooled immunoglobulin G (IgG) immunoglobulins from the plasma of more than a thousand blood donors. Typically containing more than 95% unmodified IgG, which has intact Fc-dependent effector functions, and only trace amounts of immunoglobulin A (IgA) or immunoglobulin M (IgM), IVIGs are sterile, purified IgG products are primarily used in treating three main categories of medical conditions: 1. immune deficiencies such as X-linked agammaglobulinemia, hypogammaglobulinemia (primary immune deficiencies), and acquired compromised immunity conditions (secondary immune deficiencies), featuring low antibody levels: 2. inflammatory and autoimmune diseases; and 3. acute infections.

A number of IVIG commercial suppliers provide a variety of IVIG products. More than a dozen IgG products are available in North America and Europe, which vary with respect to IG concentration, infusion frequency, route of administration, and other considerations. Perez, et al., J Allergy Clin Immunol. (2017), 139: S1-S46. Compared to the older lyophilized IVIG products containing only 50 mg/mL protein in the solution after re-dissolving, current formulations provide a 100 mg/mL and 200 mg/mL ready-for-use sterile, liquid preparation of highly purified and concentrated human IgG antibodies.

More recently entering the IgG therapeutic market are IgG formulations formatted for subcutaneous administration. These formulations represent a significant advance in the overall patient experience with IgG formulations. For example, a patient or caregiver trained in subcutaneously infusing an IgG formulation, can infuse the formulation in practically any setting. This innovation frees the patient from visits to an infusion center, allows them to infuse, e.g., self-infuse, in the comfort of their own home, or anywhere of their choosing. Exemplary subcutaneously infused IgG formulations include HyQvia® [Immune Globulin Infusion 10% (Human) with Recombinant Human Hyaluronidase], Hizentra® [Immune Globulin Subcutaneous Human 20% Liquid].

The European experience with rapid infusions of SCIG demonstrated that rates up to 40 mL/hour using two to four sites to administer 40 mL (6.4 g) per infusion were well-tolerated in primary immune deficiency disease (“PIDD”) patients. In the US, the clinical trials for the first FDA approved SCIG product (16%) used a maximum of 15 mL per site and a maximal rate of 20 mL/site/hour. Subsequent studies increased the dose per site to 30 mL and the rate of infusion to 30 mL/hour/site. The package inserts for the 10% and 20% SCIG products recommend limiting the volume of the IgG to 20 mL per site for PIDD patients with a body weight less than 40 kg and 30 mL per site in patients weighing more than 40 kg. The suggested initial infusion rates are 15 mL/site/hour (<40 kg) or 20 mL/site/hour (>40 kg), increasing to 20 mL/site/hour and 30 mL/site/hour, respectively. Multiple sites can be infused simultaneously using sites on the abdomen, thighs, upper arms, or lower back, with most infusions able to be completed in less than 90 minutes. Patients may choose to use more infusion sites, thus shortening the overall infusion time as less volume is infused per site, or they may prefer to infuse the product more slowly in order to tolerate larger volumes per site and use fewer sites. Although the recommended dosing interval is weekly, more frequent dosing (daily or 2 to 3 times per week) may improve serum IgG levels further and result in fewer infections. Shapiro reported a retrospective analysis of 104 patients with PIDD receiving SCIG using either rapid push administration or an infusion pump. 71% of patients chose to use the rapid push method and received an average dose of 32.11 g/month given in doses approximately three times per week. The volume of SCIG per site ranged from 3 to 20 mL and was given over 5-20 minutes (1 mL/minute) using a 25-gauge butterfly needle and a 12 mL syringe. The serum IgG levels and rate of systemic adverse events were similar between the two methods. Kobrynski L, Biologics (2012), 6:277-287.

Concentrated formulations of IgG (e.g., 20% IgG) are of interest as a means for infusing lower dosage volumes while achieving delivery of a full pre-determined dose, over potentially shortened infusion times for the full pre-determined dose, both of which are attractive to patients and enhance compliance with a prescribed dosing regimen. The design of such formulations and dosage regimens incorporating them is not a trivial task, and the promise of a broadly tolerated concentrated IgG formulations in a rapidly infusible format has not yet been borne out.

For example, a recent clinical study with a 20% IgG formulation demonstrates the difficulties with incorporating IgG formulations with IgG concentrations higher than 10% in dosage regimens incorporating high infusion rates (e.g., >200 mL/hr). Anderson et al., J. Clin. Immunol. (2021) 41:458-469. When the researchers increased the infusion rate of the 20% IgG formulation from 25 mL/hr/site to 75 mL/hr/site, approximately 30% of the cohort ceased the infusion or lowered its rate, because infusing the formulation at the selected rate was poorly tolerated (i.e., pain, discomfort). Further, when the infusion rate was increased to 100 mL/min/site, approximately 40% of the cohort interrupted the infusion. Thus, while this 20% IgG formulation provides increased delivery of IgG per unit volume, it does not appear to be as broadly tolerated as an analogous 10% IgG formulation when infused at higher rates, such as 100 mL/hr/site.

Despite the difficulties to date in designing an IgG dosing regimen incorporating a concentrated IgG pharmaceutical formulation administered at a high subcutaneous infusion rate, such a dosing regimen would provide subjects receiving this therapy the significant advantages of decreased infusion duration, leading to greater convenience concomitant with tolerability at the higher infusion rates providing this enhanced convenience. Such a regimen, and a combination of administration components effecting this regimen, is expected to lead to improved patient compliance due to the increased convenience, briefer infusion duration, and an overall more satisfying patient infusion experience. The present invention provides such a regimen, including a combination of administration components, a kit containing the combination of administration components, and methods and systems for using the combination of administration components.

### BRIEF SUMMARY OF THE INVENTION

In various embodiments, the present invention provides a dosing regimen for subcutaneous infusion of a pharmaceutical formulation of IgG incorporating a concentrated IgG formulation and methods of infusing these formulations that solve the problems of customary dosing regimens for subcutaneous infusion of IgG. In selected embodiments, the invention provides a kit for subcutaneous infusion of a pharmaceutical formulation of IgG at high infusion rates. An exemplary kit includes a stable 20% (w/v) IgG pharmaceutical formulation, a pharmaceutical formulation of hyaluronidase and instructions for using the formulations to infuse IgG at a high dose/volume ratio to a first infusion site at an unexpectedly high rate with excellent patient tolerability. The prior art neither discloses nor suggests methods of infusing a concentrated (e.g., 20%) IgG formulation into a first infusion site of a subject at a high rate.

Limitations with subcutaneous infusion of IgG formulations include the frequency and duration of infusion. Two approaches can be pursued (1) increasing the concentration of the IgG in the formulation or (2) increasing the volume infused per site. There are concerns remaining regarding discomfort and inconvenience related to high volume infusions per site, its potential effects on the body, local tolerability and local site reactions. Increasing the concentration of IgG in a formulation from 10% to 20% reduces the administered volume by about 50% A major challenge of increased concentration, however, is the higher viscosity of the concentrated solution, limiting the feasible infusion speed and leading to longer infusion times partly offsetting the advantage of the more concentrated formulation.

One approach to facilitating subcutaneous infusion involves administering a pharmaceutical formulation of hyaluronidase (e.g., rHuPH20) to a first infusion site prior to infusing the IgG formulation. Despite the promise of this approach studies designed to assess the feasibility of high flow rate (e.g., 3-5 mL/min) administration of the IgG formulation with rHuPH20 indicated, based on the magnitude of tissue back pressure against the IgG infusion, that rHuPH20 itself was not sufficient to support IgG flow rates of 3 mL/min or greater, which are desired for reducing infusion times of a standard dose of IgG. FIG. 12. Thus, until the present invention, it was not apparent how to obtain high infusion rates, e.g., 3-5 mL/min of an IgG subcutaneous infusion while maintaining the current quality and tolerability of an analogous 10% IgG formulation after administering hyaluronidase to the infusion site.

A recent clinical trial involving subcutaneously infusing a 20% IgG formulation illustrates the challenges in subcutaneously infusing a concentrated IgG formulation at high infusion rates. Anderson et al., J. Clin. Immunol. (2021) 41:458-469. The authors of this study reported that when the infusion rate of a 20% IgG formulation was 100 mL/hr/site, approximately 40% of the cohort ceased the infusion. Thus, it can be concluded that a dosing regimen for subcutaneous infusion of a concentrated (e.g., 20%) IgG formulation such that the infusion is broadly tolerable at even high flow rates (e.g., 120, 150, . . . even up to about 300 mL/hr/site) is neither straightforward nor trivial.

Though infusing a concentrated IgG formulation is an attractive option for a number of reasons, a concern to be addressed, is that the final formulation is preferably easily loaded into the infusion device by the patient (or caregiver), and equally easily expelled from this device through a hypodermic needle once the needle is placed in the infusion site. The complex dynamics of concentrated IgG solutions must be addressed during the manufacture of such formulations, as well as during finish and fill of vials containing the formulation. Given the complexity of the concentrated IgG solution system, achieving a formulation with fluid characteristics supporting rapid infusion (e.g., acceptable viscosity and tolerability) is neither a pre-determined, nor an apparent outcome of any particular course of investigation.

Antibody properties such as self-association and aggregation, solubility and viscosity pose significant challenges to developing high concentration antibody formulations that are easily infused and well-tolerated by patients and both pharmaceutically and economically acceptable. Antibody properties at high concentration can negatively impact solution stability, the viscosity of such formulations makes them difficult to administer to a patient, to manufacture the formulation at large scale, and negatively impacts the yields of these two processes. The researcher must take these properties into account when designing a new IgG dosing regimen using a concentrated IgG formulation and combinations of administration components and, given the experience in the art, would not begin such research expecting that the process would be straightforward and/or trivial.

The present invention addresses these and other issues by providing a pharmaceutical formulation of at least about 20% (w/v) IgG in a pharmaceutically acceptable carrier, methods of facilitating the infusion of the formulation at unexpectedly high infusion rates, a kit of components facilitating the infusion at a high rate, and a system useful in infusing the formulation at such a rate.

In an exemplary embodiment, the 20% (w/v) IgG pharmaceutical formulation is formatted for subcutaneous infusion and is a component of a kit. The kit also includes a pharmaceutical formulation of hyaluronidase. The kit further includes instructions for infusing the IgG formulation at a first infusion site following infusing the hyaluronidase formulation at this site. In an exemplary embodiment, the instructions direct the person infusing the IgG how to infuse the IgG at a high rate at a first infusion site. In an exemplary embodiment, the 20% (w/v) IgG is infused at the first infusion site at ambient temperature (about 25° C.).

A recent clinical study involving subcutaneously infusing the 20% (w/v) IgG formulation to a first infusion site subsequent to administering hyaluronidase to this site was accompanied by surprising results. The researchers found that infusion of 80 U/g IgG of hyaluronidase to the first infusion prior to infusing the 20% (w/v) IgG formulation allowed the IgG to be infused to the first infusion site at a rate similar to that generally achieved with an analogous 10% IgG formulation facilitated with the same dosage of hyaluronidase. This result was unexpected in view of the fact that the 20% (w/v) IgG formulation is approximately 4-times as viscous as the corresponding 10% (w/v) IgG formulation. See, Example 5. The infusion of the 20% IgG formulation was surprisingly well- and broadly-tolerated across the cohort at infusion rates of up to 300 mL/hr. In pre-clinical dosing with 20% (w/v) IgG in pigs facilitated with hyaluronidase, it was found that even a 5- to 10-fold increase in administered hyaluronidase did not significantly decrease the tissue back pressure to levels equivalent to those seen when administering a 10% (w/v) IgG formulation facilitated with hyaluronidase. Given the number of subjects interrupting the 75 mL/hr/site and 100 mL/hr/site infusion in the Anderson et al. study, supra, the person of ordinary skill in the art, unaware of the pre-clinical studies, would have been justified in concluding that difficulties accompanying the 4-fold greater viscosity of 20% (w/v) IgG might require at least partial offsetting by infusing a correspondingly increased dosage of hyaluronidase per gram of IgG compared to that used in HyQvia® [Immune Globulin Infusion 10% (Human) with Recombinant Human Hyaluronidase], however, this did not prove to be the case. The person aware of the pre-clinical studies would have concluded that high infusion rates of 120-300 mL/hr/site when administering the viscous 20% (w/v) IgG formulation would either not be possible, would not be well-tolerated by the subjects, or both; however, surprisingly, neither of these assumptions proved true during the clinical study. It should be noted that the inventors themselves initially believed that the 20% (w/v) IgG formulation would have to be warmed to overcome its viscosity to reach infusion rates of at least about 300 mL/hr, if that rate was even achievable in human subjects, and such high infusion rates could not be reached with 20% (w/v) IgG at room temperature.

According to various embodiments of the present invention, facilitated and warmed or unwarmed 20% (w/v) IgG allows for administration of a standard dose of IgG in 50% of the current standard volume for such a dose infusing a 10% IgG formulation, with reduced infusion time, the infusion rate and tolerability surprisingly unhampered by the increased viscosity of the unwarmed 20% (w/v) IgG formulation.

In some embodiments, the invention provides a method of subcutaneously infusing the 20% (w/v) IgG formulation in a warmed state to a first infusion site subsequent to subcutaneously infusing hyaluronidase at this site. In these embodiments, the 20% (w/v) IgG formulation is warmed to a temperature appropriate to reduce the formulation viscosity to a desired value prior to it being infused, during its infusion or both, leading to acceptable patient tolerability. Surprisingly, a modest increase in temperature above room temperature produced a significant decrease in viscosity. FIG. 1. Though it is widely understood that warming antibody and other protein solutions can degrade the proteins, and cause their aggregation, the 20% (w/v) IgG formulations of the invention were not negatively impacted by warming to as high as about 40° C.

In various embodiments, the invention provides a method of rapidly infusing a warmed 20% (w/v) IgG formulation to at least a first infusion site following administration of a pharmaceutical formulation of hyaluronidase to the infusion site.

Pre-clinical studies on infusion of a 20% (w/v) IgG formulation demonstrated that infusion rates as high as 7.5 mL/min can be achieved using an in-line warming device to overcome inherent issues with infusing a highly viscous protein solution. Pharmacokinetic results from these pre-clinical studies indicate that warmed and facilitated 20% (w/v) IgG formulation infused at 5 mL/min is readily dispersed from the subcutaneous space and taken up by the systemic compartment. The invention provides a significant advantage over prior art IgG infusion formulations, methods and systems without the need for any significant redesign of the formulation other than increasing the concentration of the IgG found therein

The kits, methods and formulations of the invention bring unexpected and significant improvements to the patient experience of those patients requiring subcutaneous infusion of an IgG formulation: the concentration of IgG in the formulation provides a subject with an infusion experience of shorter duration than prior 10% (w/v) IgG formulations and 20% (w/v) formulations. Further, in the embodiment in which the formulation is warmed, the infusion of the 20% (w/v) IgG formulation is accompanied by less back pressure attributable to the reduced viscosity of the antibody solution, and there is greater ease of infusion, compatibility of the infusion with a large number of infusion pump and infusion set combinations, and less discomfort to the patient. Simple, quick, and broadly tolerated infusion procedures favor patient compliance with a recommended treatment regimen, bringing advantages to the patient and the overall healthcare economy.

In an exemplary embodiment, the invention provides a pharmaceutical formulation contained within a system for delivery of the pharmaceutical formulation by infusion to a subject in need thereof. The pharmaceutical formulation comprises at least about 20% (w/v) of an immune globulin in an aqueous pharmaceutically acceptable carrier in which the immune globulin is dissolved. The system includes a first vessel containing the pharmaceutical formulation: a first hypodermic needle comprising a first terminus configured to penetrate a first infusion site of the subject, and a terminal opening disposed therein through which the pharmaceutical formulation is delivered to the first infusion site: a first connecting member in fluidic connection with the first vessel and the hypodermic needle; and a first warming device in thermal contact with a system component selected from the first vessel, the first connecting member, and a combination thereof, the first warming device configured to heat the pharmaceutical formulation to at least about 30° C., maintain the pharmaceutical formulation at a temperature of at least about 30° C. and a combination thereof.

In an exemplary embodiment, the pharmaceutical formulation is at a temperature of at least about 30° C., preferably from about 30° C. to about 40° C., e.g., from about 35° C. to about 40° C.

In various embodiments, the invention provides a pharmaceutical formulation of an immune globulin (e.g., IgG). The formulation comprises at least about 20% (w/v) of an immune globulin; and an aqueous pharmaceutically acceptable carrier dissolving the immune globulin. The pharmaceutical formulation has a viscosity allowing infusion of the pharmaceutical formulation into a first subcutaneous infusion site of a subject in need of such infusion at a rate of greater than about 3 mL/min, the pharmaceutical formulation under a first pressure from about 7000 Pa to about 47000 Pa. In an exemplary embodiment the pressure in tissue proximate the infusion site is from about 25 to about 200 mm Hg, e.g., from about 25 to about 150 mm Hg. In an exemplary embodiment, the pressure in the tissue proximate the first infusion site is of a magnitude at the desired infusion rate insufficient to cause the subject discomfort sufficient for the subject to discontinue the infusion.

An exemplary formulation does not include a small molecule agent incorporated expressly to reduce the viscosity of the formulation. In various embodiments, the formulation is not a suspension of the antibody in a mixture of a water and an organic solvent, e.g., an alcohol, e.g., ethanol.

IgG-based therapeutics are generally administered alone at monthly doses within a range of about 100 mg to about 2 g of protein agent per kg/patient/dose, e.g., about 1 g of protein agent per kg/patient/dose. In an exemplary embodiment, the therapeutic of the invention is infused to treat a neuroimmunological indication, and the dose is from about 1 to about 2 grams of protein agent per kg/patient/dose. In an exemplary embodiment, the indication is selected from Primary Immunodeficiency (PID) and Secondary Immunodeficiency (SID). In an exemplary embodiment, when used to treat PID or SID, the therapeutic of the invention is administered in an amount of from about 400 to about 800 mg/kg/patient/dose.

In various embodiments, in addition to a full dose being administered in a single infusion period, the dose can also be split and administered step-wise across a selected time period. Thus, for example, a dose can be split into bi-weekly doses (½ dose) or weekly doses (¼ dose)

The present disclosure recognizes the source of a problem associated with highly concentrated IgG therapeutic formulations, which can present administration challenges (e.g., difficulty in administration, patient discomfort) that diminish patient compliance due to high viscosity of the therapeutic formulation and/or due to aggregation of the IgG in the formulation. Among other things, the present disclosure provides pharmaceutical formulations of IgG containing at least about 20% IgG, which are transiently of low-viscosity, i.e., of lower viscosity than such formulations are at room temperature (a “reference formulation”). Accordingly, in various embodiments, the formulations of the invention provide a subject being treated with the therapeutic agent with a more agreeable, more tolerable infusion experience than that experienced with current analogous products. In various embodiments, this experience is contrasted with current regimens by, for example, a quicker infusion time for the required dose, and increased or similar levels of tolerability despite administration of a standard dose in reduced time.

In an exemplary embodiment, the therapeutic formulation of the invention provides for infusion of a standard IgG dose for a given indication in a time frame that is at least about 1.2-fold, at least about 1.4-fold, at least about 1.6-fold, at least about 1.8-fold or at least about 2-fold, or higher, than the time required to administer an analogous 10% (w/v) formulation of IgG.

In some embodiments, despite the 20% (w/v) concentration of IgG in the formulation, the present disclosure provides low-aggregation pharmaceutical formulations of the IgG. In some embodiments, without being bound to a particular theory, the present disclosure encompasses the recognition that reducing surface adsorption and/or interfacial interaction can have beneficial effects for certain protein formulations. Among other things, in some embodiments, the present disclosure provides formulations of therapeutic protein agents with relatively low surface adsorption and/or interfacial interaction (as compared with that observed for an appropriate reference formulation, e.g., a 20% formulation of a different protein, e.g., a different antibody, or a 10% formulation of IgG). In some embodiments, provided formulations can be injected either subcutaneously (SC) or intramuscularly (IM). The present disclosure also provides methods of making and/or using such formulations.

In an exemplary embodiment, the invention provides a method of infusing a pharmaceutical formulation of an immune globulin into a first infusion site of a subject in need thereof. The formulation infused according to the method comprises at least about 20% (w/v) of an immune globulin fraction in about 80% (w/v) of an aqueous pharmaceutically acceptable carrier dissolving the immune globulin fraction. The method includes delivering the pharmaceutical formulation from a first vessel through a first hypodermic needle and into the first infusion site, wherein the first vessel, and the first hypodermic needle are maintained in fluidic communication through a first connecting member, and wherein the pharmaceutical formulation is at an infusion temperature of from about 30° C. to about 40° C. as it enters into the first infusion site. In an exemplary embodiment, the infusion temperature is about 30° C., about 31, about 32, about 33, about 34, about 35, about 36, about 37, about 38, about 39 or about 40° C.

In an exemplary embodiment, the invention provides a method of infusing a concentrated IgG formulation such that the infusion of the pharmaceutical formulation at the infusion temperature is not accompanied by greater subject discomfort than that experienced by the subject upon infusion, under identical infusion parameters, of an otherwise identical pharmaceutical formulation comprising about 10% (w/v) of an immune globulin in an aqueous pharmaceutical carrier. In various embodiments, the infusion is accompanied by less patient discomfort than the administration of the 10% (w/w) IgG formulation, supra.

In an exemplary embodiment, the invention provides a method of infusing a concentrated IgG formulation (e.g., greater than 20% (w/v)) such that the infusion of the pharmaceutical formulation at the infusion temperature is not accompanied by greater subject discomfort than that experienced by the subject upon infusion, under identical infusion parameters, of an otherwise identical pharmaceutical formulation comprising about 20% (w/v) of an immune globulin in an aqueous pharmaceutical carrier. In various embodiments, the infusion is accompanied by less patient discomfort that the infusion of a similar or the same 20% (w/v) IgG formulation at a temperature less than 30° C.

In various embodiments, any of the formulations and methods set forth above is augmented (facilitated) by infusion into the IgG infusion site of a predetermined dosage of a pharmaceutical formulation of hyaluronidase prior to or in conjunction with the infusion at the site of the IgG formulation of the invention. The hyaluronidase is administered at the same temperature as the IgG or at a different temperature.

Additional embodiments, objects and advantages of the invention will be apparent from the following detailed description.

### BRIEF DESCRIPTION OF THE DRAWINGS

FIG. 1A and FIG. 1B are displays of dynamic viscosity varying with temperature of an exemplary 20% (w/v) IgG formulation of the invention.

FIG. 2 displays an exemplary experimental set up for an infusion warmer investigation directed to determining the effects of warming on 20% (w/v) IgG.

FIG. 3 depicts an in vivo proof of concept study in pigs, in which an experimental set up is provided with a purpose of comparing infusion pressures and local reaction of IgG 20% (w/v) versus warmed IgG 20% (w/v) versus warmed and facilitated IgG 20% (w/v), in which the experimental set up is based on experience with HyQvia® [Immune Globulin Infusion 10% (Human) with Recombinant Human Hyaluronidase] and uses pigs due to a high relevancy for humans based on a similarity in skin anatomy between pigs and humans, whereby the experimental set up utilized a first set up of 5 mL of rHuPH20 (recombinant human hyaluronidase) or buffer at a flow rate of 2 mL/min and a second set up of 50 mL of the IgG 20% (w/v) solution at a flow rate of 3 and 5 mL/min. In the experimental set up, intra-animal control is provided at lateral side in comparison of two treatment approaches within an individual animal. In the experimental set up, monitoring of in-line pressure and temperature is provided (about 34-35° C. shortly before needle through physiological temperature).

FIG. 4 shows graphs of mean in line pressure vs. time across pooled data sets with a 5 mL/min flow rate. Red: buffer+warmed IGSC, 20% (n=8); Green: PH20 (hyaluronidase)+warmed IGSC, 20% (n=6); Buffer+IGSC, 20% (n=8).

FIG. 5 shows graphs of mean in line pressure vs. time, comparison of treatment approaches with a 5 mL/min flow rate, and pooled data sets for pairs of infusion conditions.

FIG. 6 is a table displaying a summary of the data from FIG. 4 and FIG. 5.

FIG. 7 shows graphs of mean in line pressure vs. time across pooled data sets from multiple infusion experiments in pigs, infused with a 3 mL/min flow rate.

FIG. 8 shows graphs of mean in line pressure vs. time, comparison of treatment approaches with a 3 mL/min flow rate.

FIG. 9 is a table displaying individual data sets and statistical comparison for a 3 mL/min flow rate.

FIG. 10 is a graph of mean in-line pressure vs time for a variety of 20% IgG infusion regimens with variable amounts of rHuPH20.

FIG. 11 displays results from infusion experiments using a 19G needle at infusion rates from 3 to 7.5 mL/min.

FIG. 12 includes graphs showing in-line pressure vs. time for IgG formulations in buffer, with rHuPH20, and with rHuPH20 and warmed, demonstrating that facilitation without warming showed marginal reduction in infusion pressure. In contrast, facilitation with warming provides a distinct reduction in subcutaneous infusion pressure. Green-Formulation A (rHuPH20+warmed IG, 20%); Red-Buffer+IG (20%). Dosage, 50 mL IG (20%), 3 pigs per group.

FIG. 13 is a table displaying pharmacokinetic parameters from a pig infusion study. Treatment Arm 1: In-line Warmed and Facilitated IGSC 20% (n=3): 5 ml rHuPH20 at 2 ml/min and 50 ml* (400 mg/kg) warmed IgG 20% at 5 ml/min (→infusion time approx. 10 minutes). Treatment Arm 2: IGSC 20% SC (n=3): 5 ml Buffer at 2 ml/min and 50 ml* (400 mg/kg) lg 20% at 1 ml/min (→infusion time approx. 50 minutes). Sampling times: pre-dose, 5 minutes to 28 days post-dose. Bioanalytics: ELISA assay for Human IgG in pig serum.

FIG. 14 is a schematic of the overall study design of the Phase I, single-dose, single-center, open-label, three-arm study to assess the tolerability and safety of Immune Globulin Subcutaneous (Human), 20% Solution with Recombinant Human Hyaluronidase (TAK-881) at various infusion rates in healthy adult subjects. All subjects were admitted to Clinical Research Center (CRC) on Day-1 prior to dosing and discharged on Day 4. Abbreviations: ADA=anti-drug antibody; EOS=end of study; ET=early termination; IgG=immunoglobulin G.

### DETAILED DESCRIPTION OF THE INVENTION

### I. Introduction

Though the initial IG formulations were intended for intravenous administration, subcutaneous administration of IgG has become widely accepted with the development of formulations allowing the subcutaneous out-patient infusion, e.g., by an IgG recipient, care giver, or home health worker of an acceptable dosage of IgG. The convenience of self-administration makes subcutaneous IgG therapy the preferred option for many patients. Weekly subcutaneous administration provides relatively stable serum IgG levels between administrations and reduces the disparate peak and trough levels associated with intravenous administration every 3-4 weeks.

Since the first subcutaneous IgG (SCIG) product (VIVAGLOBIN® [Immune Globulin Subcutaneous (Human)], 16%) was introduced to the US in 2006, the only major change has been the concentration of IgG. At least two 10% IgG products previously licensed for IV administration have received FDA approval for SC administration (GAMMAGARD LIQUID® [Immune Globulin Infusion (Human)], 10%), (GAMUNEX®-C [Immune Globulin Injection (Human) 10%]) and another 10% IgG product (GAMMAKED™ [Immune globulin injection (human), 10% caprylate/chromatography purified) has been introduced for both IV and SC administration. 20% IgG products (CUVITRU® [Immune Globulin Subcutaneous (Human), 20% Solution]), (HIZENTRA® [Immune Globulin Subcutaneous (Human) 20% Liquid]) solely for SC administration are also now available in the US. The 20% IgG products are advantageous in that the infusion volume is smaller, potentially decreasing the number of sites required to administer the SCIG. Alternate methods of administration, such as rapid SC push, giving SCIG daily, biweekly, and bimonthly, have also been reported.

An approach to reducing infusion times, thereby increasing overall patient satisfaction with the infusion experience and, consequently, patient compliance with an infusion regimen would be based on a more rapid subcutaneous infusion of a predetermined dosage of IgG. This goal might be reached by the infusion of a concentrated subcutaneous IgG pharmaceutical formulation at high infusion rates. This approach, however, is not trivial. Amongst the difficulties arising in concentrated antibody formulation is the increase in solution viscosity, which significantly complicates product handling (e.g., loading a syringe) and infusion. The viscosity of protein solutions is highly sensitive to the protein amino acid sequence, to the buffer composition and also the presence of protein aggregates. Nicoud et al., Soft Matter, 11 (2015): 5513. It is not a trivial matter to control aggregation and viscosity in high-concentration antibody solutions (EP 2538973). This is evidenced by the few antibody products currently on the market as high-concentration formulations (>100 mg/mL) (EP 2538973).

Concentrated IgG formulations have high viscosity, which can make them difficult to load into and expel from an infusion device, difficult to administer by infusion, particularly for subcutaneous delivery, where delivery of a useful dosage of a high viscosity solution within a reasonable time frame requires the use of larger bore needles, which can result in more painful subcutaneous infusions.

The U.S. FDA does not permit subcutaneous infusions of volumes over approximately 1.5 mL of a formulation with a viscosity exceeding approximately 50 centipoise (cP). Shire et al., J Pharm Sci (2004), 93:1390-1402. Viscous concentrated antibody formulations, having a strong resistance to flow, are challenging to handle and administer to patients. Lowering the viscosity of concentrated antibody formulations is seen to be critical to fully deploying their benefits to patients.

The concentration dependence of the viscosity of aqueous solutions of gamma globulin is exponential, not linear. Thus, small increases in antibody concentration in an aqueous formulation leads to a significant increase in viscosity and the drawbacks associated with highly viscous aqueous antibody formulations. Srinivasan et al., Pharm Res (2013) 30″ 1749-1757.

Given the exponential dependence between viscosity and concentration, controlling the viscosity of an antibody solution requires balancing of a complex network of solution components and characteristics. The viscosity of antibody solutions is also dependent on shear rate. Salts of different composition, and pH also have an effect on antibody solution viscosity. The viscosity of antibody solutions is highly dependent on the protein concentration and increases non-linearly with increasing antibody concentration. Under high concentration conditions, the antibody can undergo self-association, the degree of which is a function of concentration. Reversible self-association has a major impact on the physical properties of a protein formulation. In fact, these multi-valent, low affinity interactions can result in unusually high viscosity of the concentrated antibody formulation. Reduction of the reversible protein-protein interactions results in a reduction in viscosity. Liu et al., J Pharm Sci, 94:9 (2005): 1928-1940; Shire et al., J Pharm Sci. 93:6 (2004): 1390-1402.

The art is unsettled on the role antibody aggregates play in the viscosity of antibody solutions. Contrary to the results above, other workers have found that the viscosity of a solution including an aggregate of a protein, such as an antibody, is lower than the viscosity of a monomeric sample of a similar occupied volume fraction due to the polydispersity of the aggregate distribution. See, Nicoud et al., supra, noting that the art generally recognized that the formation of aggregates and the reversible self-association of proteins increased solution viscosity. Accordingly, there remains uncertainty regarding the relevance of aggregate formation to solution viscosity.

In various embodiments, the present invention addresses the shortcomings of current concentrated IgG pharmaceutical formulations arising due to the viscosity of such formulations. In one embodiment, there is provided a pharmaceutical formulation having a viscosity reduced from that at room temperature, i.e., a warmed formulation. Also provided is a system in which the pharmaceutical formulation is contained and with which the viscosity can be reduced from its room temperature value and can, in fact, be “tuned” to a desired value to minimize infusion time while maximizing the comfort of the patient to whom the formulation is being administered. Also provided is a method for administering a concentrated IgG formulation of the invention to a patient in need thereof. An exemplary IgG formulation of the invention includes at least about 20% IgG (w/v) in a pharmaceutically acceptable carrier. Various methods of delivery are augmented by the infusion of a pre-determined dosage of a hyaluronidase formulation at or near the IgG infusion site prior to and approximately contemporaneous with infusing the IgG at the site.

Reference will now be made in detail to implementation of exemplary embodiments of the present disclosure as illustrated in the accompanying drawings. The same reference indicators will be used throughout the drawings and the following detailed description to refer to the same or like parts. Those of ordinary skill in the art will understand that the following detailed description is illustrative only and is not intended to be in any way limiting. Other embodiments of the present disclosure will readily suggest themselves to such skilled persons having benefit of this disclosure.

In the interest of clarity, not all the routine features of the implementations described herein are shown and described. It will be appreciated that, in the development of any such actual implementation, numerous implementation-specific decisions are made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be within the abilities of those of ordinary skill in the art having the benefit of this disclosure.

Many modifications and variations of the exemplary embodiments set forth in this disclosure can be made without departing from the spirit and scope of the exemplary embodiments, as will be apparent to those skilled in the art. The specific exemplary embodiments described herein are offered by way of example only, and the disclosure is to be limited only by the terms of the appended claims, along with the full scope of equivalents to which such claims are entitled.

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4.sup.th Ed, John Wiley & Sons, Inc. which are incorporated herein by reference) and chemical methods. In addition, Harlow & Lane, A Laboratory Manual Cold Spring Harbor, N.Y., is referred to for standard Immunological Techniques.

### II. Definitions

Unless defined otherwise, all technical and scientific terms used herein generally have the same meaning as commonly understood by one of ordinary skill in the art to which this invention belongs. Generally, the nomenclature used herein and the laboratory procedures in organic chemistry, pharmaceutical formulation, and medical imaging are those well-known and commonly employed in the art.

The articles “a” and “an” are used herein to refer to one or to more than one (i.e. to at least one) of the grammatical object of the article. By way of example, “an element” means one element or more than one element.

A “disease” is a state of health of an animal wherein the animal cannot maintain homeostasis, and wherein if the disease is not ameliorated then the animal's health continues to deteriorate.

As used herein, “pharmaceutically acceptable carrier” includes any material, which when combined with the conjugate retains the activity of the conjugate activity and is non-reactive with the subject's immune system. Examples include, but are not limited to, any of the standard pharmaceutical carriers such as a phosphate buffered saline solution, water, emulsions such as oil/water emulsion, and various types of wetting agents. Other carriers may also include sterile solutions. Typically, such carriers contain excipients.

Excipients can be used in the invention for a wide variety of purposes, such as adjusting physical, chemical, or biological properties of formulations, such as adjustment of viscosity, and or processes of the invention to further improve effectiveness and or to further stabilize such formulations and processes against degradation and spoilage due to, for instance, stresses that occur during manufacturing, shipping, storage, pre-use preparation, administration, and thereafter. The term “excipient” generally includes fillers, binders, disintegrants, coatings, sorbents, anti-adherents, glidants, preservatives, antioxidants, solvents, co-solvents, buffering agents, chelating agents, viscosity imparting agents, surface active agents, diluents, humectants, carriers, diluents, preservatives, emulsifiers, stabilizers and tonicity modifiers.

Acceptable excipients are preferably pharmaceutically acceptable, i.e. nontoxic to recipients at the dosages and concentrations employed.

Exemplary excipients include, without limitation: amino acids such as glycine, alanine, glutamine, asparagine, threonine, proline, 2-phenylalanine, including charged amino acids, preferably lysine, lysine acetate, arginine, glutamate and/or histidine preservatives, including antimicrobials such as antibacterial and antifungal agents antioxidants such as ascorbic acid, methionine, sodium sulfite or sodium hydrogen-sulfite: buffers, buffer systems and buffering agents which are used to maintain the composition at physiological pH or at a slightly lower pH, typically within a pH range of from about 5 to about 8 or 9; examples of buffers are borate, bicarbonate, Tris-HCl, citrates, phosphates or other organic acids, succinate, phosphate, histidine and acetate; for example Tris buffer of about pH 7.0-8.5, or acetate buffer of about pH 4.0-5.5; non-aqueous solvents such as propylene glycol, polyethylene glycol, vegetable oils such as olive oil, and injectable organic esters such as ethyl oleate; aqueous carriers including water, alcoholic/aqueous solutions, emulsions or suspensions, including saline and buffered media; biodegradable polymers such as polyesters; bulking agents such as mannitol or glycine; chelating agents such as ethylenediamine tetraacetic acid (EDTA); isotonic and absorption delaying agents; complexing agents such as caffeine, polyvinylpyrrolidone, beta-cyclodextrin or hydroxypropyl-beta-cyclodextrin) fillers; monosaccharides: disaccharides; and other carbohydrates (such as glucose, mannose or dextrins): carbohydrates may be non-reducing sugars, preferably trehalose, sucrose, octasulfate, sorbitol or xylitol: (low molecular weight) proteins, polypeptides or proteinaceous carriers such as human or bovine serum albumin, gelatin or immunoglobulins, preferably of human origin: coloring and flavouring agents: sulfur containing reducing agents, such as glutathione, thioctic acid, sodium thioglycolate, thioglycerol, [alpha]-monothioglycerol, and sodium thio sulfate diluting agents; emulsifying agents; hydrophilic polymers such as polyvinylpyrrolidone) salt-forming counter-ions such as sodium; preservatives such as antimicrobials, anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide); metal complexes such as Zn-protein complexes; solvents and co-solvents (such as glycerin, propylene glycol or polyethylene glycol); sugars and sugar alcohols, including polyols, trehalose, sucrose, octasulfate, mannitol, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; and polyhydric sugar alcohols: suspending agents; surfactants or wetting agents such as pluronics, PEG, sorbitan esters, polysorbates such as polysorbate 20, polysorbate, triton, tromethamine, lecithin, cholesterol, tyloxapal; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000; stability enhancing agents such as sucrose or sorbitol; tonicity enhancing agents such as alkali metal halides, preferably sodium or potassium chloride, mannitol sorbitol; parenteral delivery vehicles including sodium chloride solution, Ringer's dextrose, dextrose and sodium chloride, lactated Ringer's, or fixed oils; intravenous delivery vehicles including fluid and nutrient replenishers, electrolyte replenishers (such as those based on Ringer's dextrose).

It is evident to those skilled in the art that the different excipients of the pharmaceutical composition (e.g., those listed above) can have different effects, for example, and amino acid can act as a buffer, a stabilizer and/or an antioxidant: mannitol can act as a bulking agent and/or a tonicity enhancing agent: sodium chloride can act as delivery vehicle and/or tonicity enhancing agent: etc.

Polyols are useful stabilizing agents in both liquid and lyophilized formulations to protect proteins from physical and chemical degradation processes, and are also useful for adjusting the tonicity of formulations. Polyols include sugars, e.g., mannitol, sucrose, and sorbitol and polyhydric alcohols such as, for instance, glycerol and propylene glycol, and, for purposes of discussion herein, polyethylene glycol (PEG) and related substances. Mannitol is commonly used to ensure structural stability of the cake in lyophilized formulations. It ensures structural stability to the cake. It is generally used with a lyoprotectant, e.g., sucrose. Sorbitol and sucrose are commonly used agents for adjusting tonicity and as stabilizers to protect against freeze-thaw stresses during transport or the preparation of bulks during the manufacturing process. PEG is useful to stabilize proteins and as a cryoprotectant.

Surfactants routinely are used to prevent, minimize, or reduce surface adsorption. Protein molecules may be susceptible to adsorption on surfaces and to denaturation and consequent aggregation at air-liquid, solid-liquid, and liquid-liquid interfaces. These effects generally scale inversely with protein concentration. These deleterious interactions generally scale inversely with protein concentration and typically are exacerbated by physical agitation, such as that generated during the shipping and handling of a product. Commonly used surfactants include polysorbate 20, polysorbate 80, other fatty acid esters of sorbitan polyethoxylates, and poloxamer 188. Surfactants also are commonly used to control protein conformational stability. The use of surfactants in this regard is protein-specific since, any given surfactant typically will stabilize some proteins and destabilize others.

Antioxidants can—to some extent—prevent deleterious oxidation of proteins in pharmaceutical formulations by maintaining proper levels of ambient oxygen and temperature and by avoiding exposure to light. Antioxidant excipients can be used as well to prevent oxidative degradation of proteins. Among useful antioxidants in this regard are reducing agents, oxygen/free-radical scavengers, and chelating agents. Antioxidants for use in therapeutic protein formulations are preferably water-soluble and maintain their activity throughout the shelf life of a product. EDTA is a useful example.

Metal ions can act as protein co-factors and enable the formation of protein coordination complexes. Metal ions also can inhibit some processes that degrade proteins.

Salts may be used in accordance with the invention to, for example, adjust the ionic strength and/or the isotonicity of the pharmaceutical formulation and/or to further improve the solubility and/or physical stability of the antibody construct or other ingredient. As is well known, ions can stabilize the native state of proteins by binding to charged residues on the protein's surface and by shielding charged and polar groups in the protein and reducing the strength of their electrostatic interactions, attractive, and repulsive interactions. Furthermore, ionic interaction with charged and polar groups in a protein also can reduce intermolecular electrostatic interactions and, thereby, prevent or reduce protein aggregation and insolubility. Ionic species differ in their effects on proteins. A number of categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic.” Destabilizing solutes are referred to as “chaotropic.” Kosmotropes commonly are used at high concentrations (e.g., >1 molar ammonium sulfate) to precipitate proteins from solution (“salting-out”). Chaotropes commonly are used to denture and/or to solubilize proteins (“salting-in”). The relative effectiveness of ions to “salt-in” and “salt-out” defines their position in the Hofmeister series.

Free amino acids can be used in the pharmaceutical composition as stabilizers, and antioxidants, as well as other standard uses. Lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant.

Exemplary useful excipients for formulating the pharmaceutical composition include sucrose, trehalose, mannitol, sorbitol, arginine, lysine, polysorbate 20, polysorbate 80, poloxamer 188, pluronic and combinations thereof. Said excipients may be present in the pharmaceutical composition in different concentrations, as long as the composition exhibits the desirable properties as exemplified herein, and in particular promotes stabilization of the contained bispecific single chain antibody constructs. For instance, sucrose may be present in the pharmaceutical composition in a concentration between 2% (w/v) and 12% (w/v), i.e. in a concentration of 12% (w/v), 11% (w/v), 10% (w/v), 9% (w/v), 8% (w/v), 7% (w/v), 6% (w/v), 5% (w/v), 4% (w/v), 3% (w/v) or 2% (w/v). Preferred sucrose concentrations range between 4% (w/v) and 10% (w/v) and more preferably between 6% (w/v) and 10% (w/v). Polysorbate 80 may be present in the pharmaceutical composition in a concentration between 0.001% (w/v) and 0.5% (w/v), i.e. in a concentration of 0.5% (w/v), 0.2% (w/v), 0.1% (w/v), 0.08% (w/v), 0.05% (w/v), 0.02% (w/v), 0.01% (w/v), 0.008% (w/v), 0.005% (w/v), 0.002% (w/v) or 0.001% (w/v). Preferred Polysorbate 80 concentrations range between 0.002% (w/v) and 0.5% (w/v), and preferably between 0.005% (w/v) and 0.02% (w/v).

The pharmaceutical composition provided herein may in particular comprise one or more preservatives. Useful preservatives for formulating pharmaceutical compositions generally include antimicrobials (e.g. anti-bacterial or anti-fungal agents), anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide). Antimicrobial preservatives are substances which are used to extend the shelf-life of medicines by reducing microbial proliferation. Preservatives that particularly useful for formulating the pharmaceutical composition of the invention include benzyl alcohol, chlorobutanol, phenol, meta-cresol, methylparaben, phenoxyethanol, propylparaben thiomerosal. The structure and typical concentration for the use of these preservatives are described in Table 1 of Meyer et al. J Pharm Sci. 96 (12), 3155. Compositions comprising such carriers are formulated by well-known conventional methods.

“Infusion system”, as used herein, refers to a system including one or more component(s) that enables an individual (also referred to herein as a user or a patient) to self-administer a dosage of a medicament. An exemplary infusion system includes a reservoir for storing and deploying the IgGSC. An exemplary device includes one or more wearable components to enhance the subject's convenience. An exemplary infusion system includes a warming device capable of bringing the pharmaceutical formulation to the desired infusion temperature, e.g., from about 30° C. to about 40° C. In an exemplary embodiment, the system includes a syringe warmer. In an exemplary system, the warming device is an inline warmer. In an exemplary embodiment, the system includes a component intended to compensate for the viscosity of the pharmaceutical formulation and diminish the injection force needed to administer a dose of the pharmaceutical formulation (e.g., U.S. Pat. Pub. 2020/0268987). In various embodiments, the infusion system includes a structural element for diverting the flow of the pharmaceutical formulation to two or more sites. In various embodiments, the infusion system includes at least one needle (e.g., a hypodermic needle). Exemplary needles are formatted for one or more infusion sites (e.g., bifurcated, etc.).

A “warming device”, as this term is used herein, refers to any device or configuration of devices capable of warming the volume of the pharmaceutical formulation capable of warming a dose (or two or more doses if administered contemporaneously) to the desired administration temperature, e.g., from about 30° C. to about 40° C. prior to the administration. The warming device can warm the pharmaceutical formulation in its static state, while it is flowing, or both. Exemplary devices include syringe warmers and inline warmers. See, e.g., U.S. Pat. Pub. 2014/0207063; 20110166517; 2008/0262409; 2008/0119782; 2008/0269663; 20060153549; 2005/0008354: U.S. Pat. Nos. 7,316,666; 5,250,032; 4,680,445; and 4,532,414. An exemplary warming device is a component of an infusion system utilized to infuse the IgGSC.

When a warmed syringe is used, the warmed syringe can be a standard syringe that is pre-heated using a syringe warmer. The syringe warmer will generally have one or more openings each capable of receiving a syringe containing the pharmaceutical formulation and a means for heating and maintaining the syringe at a specific temperature prior to use. This will be referred to herein as a pre-heated syringe. Suitable heated syringe warmers include those available from Vista Dental Products and Inter-Med. The warmers are capable of accommodating various sized syringes and heating, typically to within 1° C., to any temperature from about 25° C. to about 40° C. In some embodiments the syringe is pre-heated in a heating bath such as a water bath maintained at the desired temperature.

The heated syringe can be a self-heating syringe, i.e. capable of heating and maintaining the liquid formulation inside the syringe at a specific temperature. The self-heating syringe can also be a standard medical syringe having attached thereto a heating device. Suitable heating devices capable of being attached to a syringe include syringe heaters or syringe heater tape available from Watlow Electric Manufacturing Co. of St. Louis, Mo., and syringe heater blocks, stage heaters, and in-line perfusion heaters available from Warner Instruments of Hamden, Conn., such as the SW-61 model syringe warmer. The heater maybe controlled through a central controller, e.g. the TC-324B or TC-344B model heater controllers available from Warner Instruments.

The heated syringe maintains the liquid protein formulation at a specified temperature from about 30° C. to about 40° C. By maintaining the pharmaceutical formulations at an elevated temperature during infusion, the viscosity of the liquid formulation is decreased, the solubility of the antibody in the formulation is increased, or both.

Heat may also be supplied to the pharmaceutical formulation using an inline heater. See, e.g., U.S. Pat. No. 10,933,200; U.S. Pat. Pub. 2014/0091083; 2011/0184501.

Infusion systems of use in the invention include those equipped with a pump to drive the pharmaceutical formulation from a reservoir of the system into the hypodermic needle or through a connection means intermediate between the reservoir and the hypodermic needle. See, for instance, U.S. Pat. Pub. 2004/0073161: U.S. Pat. No. 6,554,791:5,782,805.

As used herein, the term “infusion” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. Subcutaneous infusion is an exemplary mode of administration. In some embodiments, infusion may involve only a single dose. In some embodiments, infusion may involve administration of a fixed number of doses. In some embodiments, infusion may involve dosing that is intermittent (e.g., a plurality of doses separated in time) and/or periodic (e.g., individual doses separated by a common period of time) dosing. In some embodiments, infusion may involve continuous dosing (e.g., perfusion) for at least a selected period of time. In some embodiments, infusion includes administration of more than one dose at more than one site. In some embodiments, infusion is of a pre-determined dosage at one, two or more sites with the pre-determined dosage divided across the plurality of sites. The use of one, two, or more sites for an infusion is suitable for subcutaneous infusions.

Injectability or syringeability: As generally used herein, the term “injectability” or “syringeability” refers to the injection (infusion) performance of a pharmaceutical formulation through a syringe equipped with a needle of a selected gauge, e.g., an 18-32 gauge needle, optionally a thin walled needle, wherein the needle is a hypodermic needle. Injectability generally depends upon factors such as pressure or force required for infusion, evenness of flow, aspiration qualities, and freedom from clogging the needle. Injectability of the liquid pharmaceutical formulations may be assessed by comparing the infusion force of a reduced-viscosity formulation to a standard formulation without added viscosity-reducing agents. The reduction in the infusion force of the formulation of the invention reflects improved injectability of that formulation. The formulations of the invention have improved injectability. For various formulations, the infusion force is reduced by about 10%, 20%, 30%, 50%, 75% or more when compared to a formulation with the same concentration of protein under otherwise the identical conditions while obtaining the same injectability. In some embodiments, the amount the infusion force is reduced is within a range bounded by a lower limit and an upper limit, the upper limit being larger than the lower limit. In some embodiments, the lower limit may be about 5%, about 10%, or about 15%. In some embodiments, the upper limit may be about 50%, or about 75%. In some embodiments, the range may be about 10% to about 30%. In some embodiments, the range may be about 10% to about 50%. In some embodiments, the range may be about 10% to about 75%. Alternatively, injectability of liquid pharmaceutical formulations may be assessed by comparing the time required to inject the same volume, such as 0.5 mL to about 1 mL, of the liquid protein formulations when the syringe is depressed with the same force.

As generally used herein, the term “infusion force” refers to the force required to push a given liquid formulation through a given syringe equipped with a given needle gauge at a given infusion rate. The infusion force is typically reported in Newtons. For example, the infusion force may be measured as the force required to push a liquid formulation through a 1 mL plastic syringe (e.g., plastic, glass, metal) with a 0.25 inch inside diameter that is equipped with a 0.50 inch, 27 gauge needle at a 250 mm/min infusion rate. Testing equipment can be used to measure the infusion force. When measured under the same conditions, a formulation with lower viscosity, such as that of the invention, will generally require an overall lower infusion force.

An exemplary “tissue back pressure,” as this term is used herein, refers to the force exerted by the tissue of the subcutaneous compartment opposing the force exerted by the IgGSC as it enters the subcutaneous compartment, injected by the infusion system, providing resistance to the infusion into and distribution within the subcutaneous compartment of the IgGSC. In various embodiments, the infusion methods of the invention are not accompanied by tissue back pressure sufficient to induce the subject to whom the IgG formulation is being administered to terminate the infusion or to reduce its rate due to a perception of discomfort or pain.

IgG-based therapeutics are generally administered alone at doses within a range of about 100 mg to about 2 g/kg/patient/dose of protein agent per infusion. The present disclosure recognizes the source of a problem associated with highly concentrated IgG therapeutic formulations, which can present administration challenges due to high viscosity and/or due to aggregation. Among other things, the present disclosure provides pharmaceutical formulations of IgG containing at least about 20% IgG, which are transiently of low-viscosity, i.e., of lower viscosity than such formulation are at room temperature (a “reference formulation”). The invention further provides for “facilitated” formulations, “facilitated” infusion of these formulations, and systems containing, and used for infusion of “facilitated” formulations. As used herein, “facilitated” refers to the co-administration or contemporaneous administration of a formulation of hyaluronidase (e.g., rHuPH20) and the 20% IgG formulation.

“Facilitated IGSC (20%)” refers to infusing IGSC (20%) and hyaluronidase, which facilitates the infusion of the antibody formulation. In an exemplary embodiment, the facilitated IGSC (20%) is infused at a first infusion site at a rate of at least about 100 mL/hr, at least about 120 mL/hr, at least about 140 mL/hr, at least about 160 mL/hr, at least about 180 mL/hr, at least about 200 mL/hr, at least about 220 mL/hr, at least about 240 mL/hr, at least about 260 mL/hr, at least about 280 mL/hr, or at least about 300 mL/hr.

Kinematic viscosity: As used herein, the term “kinematic viscosity” refers to a measure of the rate at which momentum is transferred through a fluid. It is measured in Stokes (St). The kinematic viscosity is a measure of the resistive flow of a fluid under the influence of gravity. When two fluids of equal volume and differing viscosity are placed in identical capillary viscometers and allowed to flow by gravity, the more viscous fluid typically takes longer than the less viscous fluid to flow through the capillary. The dimension of kinematic viscosity is length/time. Commonly, kinematic viscosity is expressed in centiStokes (cSt). The SI unit of kinematic viscosity is mm²/s, which is equal to 1 cSt.

As used herein, the terms “improve”, “increase”, “inhibit”, “decrease”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable systems known or expected to respond in a particular way, in presence of the relevant agent or treatment.

As generally used herein, the term “reduced-viscosity formulation” refers to a liquid formulation with a high concentration of a high-molecular-weight protein, such as IgG that is modified by its infusion using a system described herein, thereby lowering the viscosity of the formulation infused, as compared to a corresponding formulation infused at a lower temperature.

As used herein, “membrane anchored HASEGP”, refers to a family of membrane anchored Hyaluronidases that share common structural features as described herein. As described and illustrated herein, hyaluronidases (i.e. glycosaminoglycanases capable of breaking down hyaluronan, preferably those exhibiting at least some activity in the ranges of neutral pH) that are normally membrane anchored can be converted to soluble HASEGPs or sHASEGPs by removing or otherwise modifying one or more of the regions that are associated with anchoring the hyaluronidase in the membrane.

As used herein, “soluble hyaluronidase” refers to a polypeptide characterized by its solubility under physiologic conditions. Soluble HASEGP can be distinguished for example by its partitioning into the aqueous phase of a Triton X-114 solution warmed to 37° C. (Bordier et al J Biol Chem. 1981 Feb. 25; 256 (4): 1604-7). Lipid anchored HASEGP on the other hand will partition into the detergent rich phase, but will partition into the detergent poor or aqueous phase following treatment with Phospholipase-C.

As used herein, a “sHASEGP”, whenever referenced herein, refers to the soluble PH20 polypeptides set forth in U.S. Pat. No. 10,588,983, the contents of which are incorporated by reference herein in their entirety for all purposes. In particular, the HASEGP polypeptide is provided. The polypeptide is a single or two chain polypeptide. Smaller portions thereof that retain Hyaluronidase activity are also provided. The Hyaluronidase domains from sHASEGPs vary in size and constitution, including insertions and deletions in surface loops. Thus, for purposes herein, the catalytic domain is a portion of a sHASEGP, as defined herein, and is homologous to a domain of other hyaluronidase like sequences, such as HYAL1, HYAL2, HYAL3, which have been previously identified: it was not recognized, however, that an isolated single chain form of the human Hyaluronidase domain could function in in vitro assays. The Aspartate and Glutamate residues necessary for activity are present in conserved motifs.

In particular, the sHASEGP polypeptide is provided. The polypeptide is a single or two chain polypeptide. Smaller portions thereof that retain Hyaluronidase activity are also provided. The Hyaluronidase domains from sHASEGPs vary in size and constitution, including insertions and deletions in surface loops. Thus, for purposes herein, the catalytic domain is a portion of a sHASEGP, as defined herein, and is homologous to a domain of other hyaluronidase like sequences, such as HYAL1, HYAL2, HYAL3, which have been previously identified: it was not recognized, however, that an isolated single chain form of the human Hyaluronidase domain could function in in vitro assays. The Aspartate and Glutamate residues necessary for activity are present in conserved motifs.

As used herein, a “neutral hyaluronidase domain of a soluble sHASEGP” refers to a beta-1,4 endoglucosaminidase domain of a sHASEGP that exhibits Hyaluronidase activity at neutral pH, is soluble under conditions as described and shares homology and structural features with the hyaluronidase glycosyl-hydrolase family domains but contains additional sequences in the carboxy terminus that are required for neutral activity. Hence it is at least the minimal portion of the domain that exhibits Hyaluronidase activity as assessed by standard in vitro assays and remains soluble. Contemplated herein are such Hyaluronidase domains and catalytically active portions thereof. Also provided are truncated forms of the Hyaluronidase domain that include the smallest fragment thereof that acts catalytically as a single chain form. As used herein and in the art, neutral or neutral-active refers to a protein exhibiting activity at neutral pH (e.g. exhibiting activity at about pH 7) and which is therefore active at a pH range characteristic of many physiological tissues. As will also be appreciated by those of skill in the art, a protein generally exhibits a range of activity around its pH optimum. The pH optimum of a neutral active protein will typically be within one to several pH units above or below pH 7, but its range of activity may extend over many pH units.

Thus, for exemplary purposes herein, the Hyaluronidase domain is a portion of a sHASEGP, as defined herein, and is homologous to a domain of other sHASEGPs. As with the larger class of enzymes of the hyaluronidase family, the sHASEGP catalytic domains share a high degree of amino acid sequence identity. The Asp and Glu residues necessary for activity are present in conserved motifs.

As used herein, the “catalytically active domain of a sHASEGP” refers to the neutral active endoglucosaminidase domain as defined by activity in vitro towards a glycosaminoglycan substrate.

sHASEGPs of interest include those that are active against chondroitin sulfates and chondroitin sulfate proteoglycans (CSPG's) in vivo and in vitro; and those that are active against hyaluronan. As used herein, a human sHASEGP is one encoded by nucleic acid, such as DNA, present in the genome of a human, including all allelic variants and conservative variations as long as they are not variants found in other mammals.

As used herein, “nucleic acid encoding a Hyaluronidase domain or catalytically active portion of a sHASEGP” shall be construed as referring to a nucleic acid encoding only the recited single chain Hyaluronidase domain or active portion thereof, and not the other contiguous portions of the sHASEGP as a continuous sequence.

As used herein, recitation that a glycoprotein consists essentially of the “Hyaluronidase domain” means that the only sHASEGP portion of the polypeptide is a Hyaluronidase domain or a catalytically active portion thereof. The polypeptide can optionally, and generally will, include additional non-sHASEGP-derived sequences of amino acids.

As used herein, “domain” refers to a portion of a molecule, e.g., glycoproteins or the encoding nucleic acids that is structurally and/or functionally distinct from other portions of the molecule.

As used herein, “Hyaluronidase” refers to an enzyme catalyzing hydrolysis of glycosaminoglycans including hyaluronans. Included in this definition are naturally occurring hyaluronidases, and recombinant hyaluronidases, both human and from other sources.

For clarity reference to Hyaluronidase refers to all forms, and particular forms will be specifically designated. For purposes herein, the Hyaluronidase domain includes the membrane bound and soluble forms of a sHASEGP protein.

“HuPH20” refers to human hyaluronidase, e.g., human recombinant hyaluronidase (rHuPH20).

As used herein, a “conventional infusion rate” is less than or equal to about 60 mL/hr/site, label values typical of currently approved subcutaneous IgG formulations.

As used herein, a “higher infusion rate” is from about 60 to about 100 mL/hr/site.

“As used herein, a “high infusion rate” is from about 100 to about 300 mL/hr/site, e.g., at least about 120, at least about 140, at least about 160, at least about 180, at least about 200, at least about 220, at least about 240, at least about 260, at least about 280, at least about 300 mL/hr/site, or higher.

### III. The Embodiments

### A. Kits

As a means to improve and facilitate the overall patient experience of subjects infusing a concentrated IgG formulation, in various embodiments, the invention provides a kit including a first container comprising a pharmaceutical formulation of hyaluronidase, e.g., human hyaluronidase, e.g., recombinant human hyaluronidase in a pharmaceutically acceptable carrier, a second container comprising a pharmaceutical formulation of 20% (w/v) IgG in a pharmaceutically acceptable carrier, and instructions providing guidance for sequentially subcutaneously infusing into a first infusion site, (i), a first aliquot of a pre-determined dosage of the pharmaceutical formulation of recombinant human hyaluronidase and, (ii), following (i), a first aliquot of a pre-determined dosage of the pharmaceutical formulation of 20% (w/v) IgG.

In various embodiments, the pharmaceutical formulation of recombinant human hyaluronidase contains 160 U/mL hyaluronidase, e.g., recombinant human hyaluronidase. In an exemplary embodiment, the recombinant human hyaluronidase is rHuPH20.

In various embodiments, the kit further includes an infusion apparatus for sequentially or simultaneously subcutaneously infusing (i), the pharmaceutical formulation of recombinant human hyaluronidase and, (ii), following (i), the pharmaceutical formulation of 20% (w/v) IgG. In various embodiments, the kit further includes a subcutaneous needle set.

In various embodiments, the instructions are a component of a Dosage and Administration section of Complete Prescribing Information. In an exemplary embodiment, the instructions provide guidance for subcutaneously infusing the pharmaceutical formulation of rHuPH20 to the first infusion site. In one embodiment, the instructions provide guidance for subcutaneously infusing from about 50 U/g to about 100 U/g IgG of rHuPH20 to the first infusion site.

In some embodiments, the instructions provide guidance for subcutaneously infusing at up to at least about 100 mL, at up to at least about 150 mL, up to at least about 200 mL, up to at least about 250 mL, or up to at least about 300 mL of the pharmaceutical formulation of 20% (w/v) IgG to the first infusion site. In an exemplary embodiment, the instructions provide guidance for subcutaneously infusing the first pre-determined dosage of the pharmaceutical formulation of 20% (w/v) IgG to the first infusion site at a rate of at least about 120 mL/hr, at least about 150 mL/hr, at least about 200 mL/hr, at least about 250 mL/hr, or at least about 300 mL/hr. In various embodiments, the instructions provide guidance for subcutaneously infusing at least about 120 mL of the pharmaceutical formulation of 20% (w/v) IgG to the first infusion site at a rate of at least about 120 mL/hr, at least about 150 mL/hr, at least about 200 mL/hr, at least about 250 mL/hr, or at least about 300 mL/hr. In various embodiments, the instructions provide, (b) guidance for subcutaneously infusing at least about 300 mL of the pharmaceutical formulation of 20% (w/v) IgG to the first infusion site. In an exemplary embodiment, the instructions provide (a) guidance for subcutaneously infusing at least about 300 mL of the pharmaceutical formulation of 20% (w/v) IgG to the first infusion site at a rate of at least about 300 mL/hr.

In various embodiments, the instructions provide guidance for subcutaneously infusing the pharmaceutical formulation of 20% (w/v) IgG warmed to a temperature of from about 30° C. to about 41° C., said pharmaceutical formulation warmed to the temperature prior to the infusing, during the infusing, and a combination thereof.

In various embodiments, the instructions further provide guidance on simultaneously or sequentially subcutaneously infusing at a second infusion site, (i), a second aliquot of the pre-determined dosage of the pharmaceutical formulation of recombinant human hyaluronidase and, (ii), following (i), subcutaneously infusing a second aliquot of the pre-determined dosage of the pharmaceutical formulation of 20% (w/v) IgG at the second infusion site.

In various embodiments, the instructions provide guidance on subcutaneously infusing the pharmaceutical formulation of rHuPH20 to the first infusion site, followed by infusing the pharmaceutical formulation of 20% (w/v) IgG to the first infusion site using a member selected from: (i) a subcutaneous needle set; (ii) a pooling bag; (iii) a gravity fill set with vented spike; (iv) a syringe; (v) a pump; (vi) a warming device; (vii) tubing; and a combination thereof.

### B. Formulations

Highly concentrated formulations of macromolecules, such as therapeutic protein agents, including whole antibodies, or fragments thereof, with low-viscosity, are of great value for their ease of storage and delivery in vivo. However, very few techniques exist for the preparation of high concentration, low-viscosity protein agent formulations above 200 mg protein agent per mL solution that are also stable, and do not form aggregates in any appreciable or interfering amount. The present disclosure, among other things, identifies the source of a problem relating to high concentration protein agent compositions. Among other things, the present disclosure appreciates that such compositions can pose a number of challenges such as high viscosity, lower stability, and difficulty in handling and manufacturing. In addition, the present disclosure appreciates that certain viscosity-reducing agents sometimes proposed for use in the art may be required in large amounts in order to reduce viscosity sufficiently, and sometimes these agents can be toxic or not pharmaceutically acceptable.

The present disclosure appreciates that high concentrations of protein agents often must be handled with considerable care, since they can be extremely prone to aggregation and high degrees of protein-protein interactions. Solutions with high protein agent concentrations have a tendency to aggregate and form particulates during processing and/or storage, which makes manipulation during further processing and/or delivery difficult. Concentration-dependent degradation and/or aggregation can present significant challenges for development of high concentration protein agent formulations.

The present disclosure provides, among other things, high concentration formulations (e.g., at concentrations greater than 200 mg/mL) of protein agents with reduced viscosity, including therapeutic agents. In general, the provided formulations are suitable for parenteral administration (e.g., by infusion), and in many embodiments by parenteral administration that does not involve infusion and/or that is other than intravenous administration. In particular, in many embodiments, the present disclosure provides formulations suitable for administration by subcutaneous (SC) and/or intramuscular (IM) infusion. In many embodiments, provided formulations are suitable for administration via 18-32 gauge needles.

In an exemplary embodiment, the invention provides a pharmaceutical formulation contained within a system for delivery of the pharmaceutical formulation by infusion to a subject in need thereof. The pharmaceutical formulation comprises at least about 20% (w/v) of an immune globulin in an aqueous pharmaceutically acceptable carrier in which the immune globulin is dissolved. The system includes a first vessel containing the pharmaceutical formulation: a first hypodermic needle comprising a first terminus configured to penetrate a first infusion site of the subject, and a terminal opening disposed therein through which the pharmaceutical formulation is delivered to the first infusion site: a first connecting member in fluidic connection with the first vessel and the hypodermic needle; and a first warming device in thermal contact with a system component selected from the first vessel, the first connecting member, and a combination thereof, the first warming device configured to heat the pharmaceutical formulation to at least about 30° C., maintain the pharmaceutical formulation at a temperature of at least about 30° C. and a combination thereof.

In an exemplary embodiment, the pharmaceutical formulation is at a temperature of at least about 30° C., at least about 32, at least about 34, at least about 36, at least about 38 or at least about 40° C. when infused. In various embodiments, the formulation is at at least one of these temperatures before, after or as it enters the infusion site.

In some embodiments, the first infusion site is a first subcutaneous infusion site.

In various embodiments, the invention provides a pharmaceutical formulation of an immune globulin. The formulation comprises at least about 20% (wt/v) of an immune globulin; and an aqueous pharmaceutically acceptable carrier dissolving the immune globulin. The pharmaceutical formulation has a viscosity allowing infusion of the pharmaceutical formulation into a first subcutaneous infusion site of a subject in need of such infusion at a rate of greater than about 3 mL/min (e.g., from about 3 to about 7.5 mL/min, e.g. from about 3 to about 45 mL/min), the pharmaceutical formulation under a first pressure from about 7000 Pa to about 47000 Pa. An exemplary formulation does not include a small molecule agent incorporated expressly to reduce the viscosity of the formulation. In various embodiments, the formulation is not a suspension of the antibody in a mixture of a water and an organic solvent, e.g., an alcohol, e.g., ethanol. An exemplary formulation has a viscosity which is less than or about 10 mPa/sec at from about 30 to about 40° C.

In some embodiments, the present disclosure provides low-aggregation pharmaceutical formulations of an antibody. In some embodiments, the present disclosure encompasses the recognition that reducing surface adsorption and/or interfacial interaction can have beneficial effects for certain protein formulations. Among other things, in some embodiments, the present disclosure provides formulations of therapeutic protein agents with relatively low surface adsorption and/or interfacial interaction (as compared with that observed for an appropriate reference formulation). The provided formulations can be injected subcutaneously (SC) or intramuscularly (IM).

In an exemplary embodiment, the invention provides an infusion system in which the first vessel is selected from an infusion bag and a syringe.

In various embodiments, the first connecting member of the system is a length of hollow tubing attached to both the first vessel and the hypodermic needle.

In various embodiments, the system further comprises a means for sufficiently pressurizing the first vessel to drive the pharmaceutical formulation from the first vessel through the first connecting member and into the first hypodermic needle, from which the pharmaceutical formulation exits the system via the terminal opening thereof. When the pressure is not supplied in the course of manually operating the syringe, an exemplary pressurizing means is a pump.

In an exemplary embodiment, the warming means of the system is configured to warm the IgG formulation during its residence in the first vessel, during its transit through the system, e.g., while it is resident in the first connecting member or both. An exemplary system is configured to provide the pharmaceutical formulation exiting the terminal opening of the first hypodermic needle at a first flow rate, and the first flow rate is selected to allow the pharmaceutical formulation to be heated to, or maintained at, at least about 30° C. during its passage through the first connecting member at the first flow rate. In some embodiments, the formulation is heated to, or maintained at from about 30° C. to about 40° C.

In various embodiments, the system is configured to provide the pharmaceutical formulation transiting the first connecting member and/or exiting the terminal opening of the first hypodermic needle at a first flow rate, the first flow rate is selected to allow the pharmaceutical formulation to be heated to, or maintained at, from about 28° C. to at least about 40° C., e.g., about 30, 31, 32, 33, 34, 35, 36, 37, 38, 39 or 40° C. during its passage through the first connecting member at the first flow rate.

In an exemplary embodiment, the connecting member is a length of tubing. In some embodiments, the length of tubing is maintained within the warming means, and the IgG formulation is warmed as it transits the connecting means.

In an exemplary embodiment, the pharmaceutical formulation contained in the first vessel is formatted as a single unit dosage formulation.

It is within the scope of the present invention for a subject to administer more than one, e.g., 2, 3, 4, 5 or more unit dosages at 2, 3, 4, or more sites to achieve the desired dosage. In an exemplary embodiment, the unit dosage is administered to 2 or more sites, and a branched connecting member in which each branch terminates with the hypodermic needle and each hypodermic needle is inserted into a unique administration site. Thus, in some embodiments, the single unit dosage formulation is formatted for delivering the pharmaceutical formulation to the first infusion site. In various embodiments, the single unit dosage formulation is formatted for delivering the pharmaceutical formulation to the first infusion site and a second infusion site.

The pharmaceutical formulation can include components other than or in addition to the IgG and the pharmaceutically acceptable carrier, or it can consist essentially of these two elements, thereby providing a pharmaceutical formulation essentially free of a protein other than the immune globulin. In an exemplary embodiment, the pharmaceutical formulation includes albumin.

According to the present invention, the pharmaceutical formulation achieves a viscosity and syringeability appropriate for subcutaneous administration without the need for adding to the formulation any small organic molecule incorporated into the formulation expressly to reduce the viscosity thereof. The invention contemplates exemplary formulations in which small organic or inorganic molecules are included in the formulation, however, these additives will be incorporated for a purpose other than reducing the formulation viscosity.

In an exemplary embodiment the viscosity of the IgG formulation at a temperature of about 30° C., 31, 32, 33, 34, 35, 36, 37, 38, 39, or 40° C. is from about 7 mPa-s to about 10 mPa-s.

The invention provides pharmaceutical formulations of pre-determined and controlled viscosity and, therefore, of pre-determined and controlled flow rates through the system containing the pharmaceutical formulation. In an exemplary embodiment, the system containing the pharmaceutical formulation is configured such that the pharmaceutical formulation, at about 30° C., has flow rate of from about 3 mL/min to about 7.5 mL/min as it exits the terminal opening of the first hypodermic needle, which is a 21-24 gauge hypodermic needle.

In various embodiments, the present invention provides a pharmaceutical formulation according to any previous embodiment and further includes a system, whereby an exemplary system comprises a pump configured to facilitate dispensing the pharmaceutical formulation of the immune globulin under pressure from the first vessel into the first infusion site of a subject.

In some embodiments, the invention provides a pharmaceutical formulation, wherein the infusion of the pharmaceutical formulation into the first infusion site at the infusion temperature is not accompanied by greater discomfort experienced by the subject than that experienced by the subject upon infusion into the first infusion site of an otherwise identical pharmaceutical formulation comprising about 10% (w/v) of an immune globulin; and about 90% (w/v) of an aqueous pharmaceutical carrier under identical infusion parameters. In various embodiments, the subject experiences even less discomfort under this scenario.

In an exemplary embodiment, the formulation does not include a viscosity-reducing agent, e.g., an agent added to the formulation for the express purpose of reducing the viscosity of the formulation and having no other significant purpose in the formulation beyond viscosity reduction. Exemplary viscosity reducing agents absent from the formulation include, without limitation, nicotinic acid (acid form) and/or caffeine, nicotinic acid and/or caffeine citrate, nicotinic acid and/or caffeine nicotinate, or nicotinic acid and/or aspirin: in further combination with one or more of nicotinamide (niacinamide), nicotinic acid sodium salt, benzyl nicotinate, inositol hexanicotinate, nicotinyl alcohol (beta-pyridyl carbinol), xanthine nicotinate, methyl nicotinate, ethyl nicotinate, propyl nicotinate, isopropyl nicotinate, butyl nicotinate, isoamyl nicotinate, hexyl nicotinate, phenyl nicotinate, gauiacyl nicotinate, xanthinol nicotinate, nicametate citrate, nicotinuric acid, nicotinyl hydroxamate, tocopheryl nicotinate, trigonelline, nicotinoyl-dl-alpha-alanine, nicotinoyl-L-alanine, nicotinoyl-dl-valine, nicotinoyl-L-leucine, and nicotinoyl-dl-phenylalanine, ethionamide, niceritrol, nicofuranose, piperocaine, N-ethylpiperidine, caffeine haematin, ethoxycaffeine, methoxy caffeine, 7-Benzyltheophylline, theophylline, paraxanthine, theobromine, 7-[(4-methoxyphenyl)methyl]-1,3-dimethyl-2,3,6,7-tetrahydro-1H-purine-2,6-dione, 1,3-dimethyl-7-[(4-methylphenyl)methyl]-2,3,6,7-tetrahydro-1H-purine-2,6-dione, 7-[(4-chlorophenyl)methyl]-1,3-dimethyl-2,3,6,7-tetrahydro-1H-purine-2,6-dione, 7-[(3,5-dimethylphenyl)methyl]-1,3-dimethyl-2,3,6,7-tetrahydro-1H-purine-2,6-dione, 7-benzyl-1,3-dimethyl-2,3,6,7-tetrahydro-1H-purine-2,6-dione, 1,3-dimethyl-7-{[4-(propan-2-yl)phenyl]methyl}-2,3,6,7-tetrahydro-1H-puri-ne-2,6-dione, 1,3-dimethyl-7-[(2-methylphenyl)methyl]-2,3,6,7-tetrahydro-1H-purine-2,6-dione, 4-[(1,3-dimethyl-2,6-dioxo-2,3,6,7-tetrahydro-1H-purin-7-yl)methyl]benzon-itrile, 7-[(4-bromophenyl)methyl]-1,3-dimethyl-2,3,6,7-tetrahydro-1H-purin-e-2,6-dione, Methyl 4-[(1,3-dimethyl-2,6-dioxo-2,3,6,7-tetra hydro-1H-purin-7-yl)methyl]benzoate, 1,3-dimethyl-7-{[4-(trifluoromethyl)phenyl]methyl}-2,3,6,7-tetrahydro-1H-purine-2,6-dione, 1,3-dimethyl-7-{[4-(methylthio)phenyl]methyl}-2,3,6,7-tetra hydro-1H-purine-2,6-dione, 7-[(3-bromophenyl)methyl]-1,3-dimethyl-2,3,6,7-tetrahydro-1H-purine-2,6-d-ione, 7-(cyclohexylmethyl)-1,3-dimethyl-2,3,6,7-tetrahydro-1H-purine-2,6-d-ione: 1,3-dimethyl-7-[(4-nitrophenyl)methyl]-2,3,6,7-tetrahydro-1H-purine-2,6-dione, 1,3-dimethyl-7-[(3-nitrophenyl)methyl]-2,3,6,7-tetrahydro-1H-purine-2,6-dione, 1,3-dimethyl-7-(1-phenylethyl)-2,3,6,9-tetra hydro-1H-purine-2,6-dione, 8-[(pyrrolidin-1-ylcarbonothioyl) sulfanyl]caffeine, 8-hydrazinocaffeine 8-chlorocaffeine, and 8-(3-butyl-4-phenyl-2,3-dihydro thiazol-2-ylidene) hydrazino-3,7-dihydro-1,3,7-trimethyl-1H-purine-2,6-dione, acetyl salicyclic acid, salicylic acid, phenyl acetic acid, 2-amino-cyclohexane-carboxylic acid, gentisic acid, pthalic acid, anthrallic acid, tetracaine, proxymetacaine, metoclopramide, procaine, chloroprocaine, benzocaine, octisalate, propylparaben, thimerosal, vanillin, cyclomethylcaine, mandelic acid, metoclopramide, L-pantothenic acid hemicalcium salt, L-ascorbic acid, thiamine·HCl, rutin hydrate, riboflavin, folic acid, pyridoxine, biotin, pantoic acid, S-benzoylthiamine, pyridoxal, pyridoxamine, L-histidine, L-lysine, L-arginine, L-2-amino-3-guanidinopropionic acid hydrochloride, 4-guanidinobutyric acid, L-homoarginine·HCl, aspartame, glycine, L-alanine, proline, trans-4-hydroxy-L-proline, L-valine, L-leucine, L-isoleucine, L-methionine, L-serine, tyramine HCl, histamine, imidazole, L-phenyl alanine, tyrosine, tryptophan, threonine, L-glutamic acid, L-aspartic acid, L-valine, 5-fluoro-L-tryptophan, 5-fluro-DL-tryptophan, 5-hydroxy-L-tryptophan, 5-methoxy-DL-tryptophan, tryptamine, argyrin A and B, granisetron, selenomethionine, carnithine, asparagine, glutamine, arginine-HCl, arginine succinate, arginine dipeptide, arginine tripeptide, polyarginine, 2-amino-3-guanidino-propionic acid, guanidine, ornithine, agmatine, guanidobutyric acid, citrulline, N-hydroxy-L-nor-arginine, nitroarginine methyl ester, argininamide, arginine methyl ester, arginine ethyl ester, lysinamide, lysine methyl ester, histidine methyl ester, alaninamide, alanine methyl ester, putrescine, cadaverine, spermidine, spermine, adenine, guanine, cytosine, uracil, thymine, adenosine, guanosine, cytidine, uridine, inosine, thymidine, xanthine, hypoxanthine, 2′-deoxycytidine, 2′-deoxyuridine, orotic acid, ribothymidine, 1-methyl xanthine, 7-methyl xanthine, and 3-methyl xanthine, D-sucrose, D-(+)-trehalose dehydrate, D-(−)-fructose, D-mannitol, L-(+)-arabinose, D-sorbitol, lactose, maltose, D-ribose, D-galactose, glucosamine, hydroxyalkyl starch, hyaluronic acid, pullulane, chitosan, dextran, dextran sulfate, starch, chondroitin sulfate, carboxymethyl dextran, and hydroxylethyl starch, 2-aminopyrimidine, sodium acetate, pyruvate sodium salt, potassium acetate, alpha-ketoglutarate, oxaloacetic acid, fumaric acid, DL-malic Acid, methyl acetoacetate, DL-isocitric acid trisodium salt, succinic acid, procaine HCl, creatinine, thiazole, citric acid, 3-pyridine sulfonic acid, ethylenediaminetetraacetic acid (EDTA), ethanolamine, di-ethanolamine, tri-ethanolamine, dimethylcyclohexylamine HCl, p-hydroxybenzoic acid, sodium benzoate, malonic acid, maleic acid, oxalosuccinate, pyrolline-5-carboxylic acid, ethanol, DMSO, benzyl alcohol, and 1,5-pentanediol, sodium chloride, ammonium chloride, ammonium acetate, ammonium sulphate, calcium chloride, sodium thiocyanate, polysorbate 80, polysorbate 20, n-dodecyl beta-D-maltoside, octyl beta-D-glucopyranoside, aspirin, calcium carrageenan, calcium cyclamate, calcobutrol, caloxetic acid, camphorsulfonic acid, creatinine, dalfampridine, dehydroacetic acid, diazolidinyl urea, dichlorobenzyl alcohol, dimethyl isosorbide, epitetracycline, ethyl maltol, ethyl vanillin, ornidazole, ethanolamide, HEPES (4-(2-hydroxy ethyl)-1-piperazine ethane sulfonic acid), iodoxamic acid, menthol, medronic acid, m-cresol, glutathione, lactobionic acid, maltitol, oxyquinoline, pentetic acid, piparazine, propenyl guaethol, propylene carbonate, protamine sulfate, QUATERNIUM-15, QUATERNIUM-52, satialgine 11, sodium 1,2-ethanedisulfonate, sodium cocoyl sarcosinate, sodium lauroyl sarcosinate, sodium polymetaphosphate, sodium pyrophosphate, pyroglutamic acid, sodium trimetaphosphate, sodium tripolyphosphate, sorbitan, tartaric acid, lactic acid, iofetamine, sucralose, 1-(4-pyridyl)pyridinium chloride, aminobenzoic acid, Sulfacetamide sodium, naphthalene 2-sulfonic acid, tert-butylhydroquinone, trolamine, tromantadine, versetamide, nioxime, methylisothiazolinone, mannose, lidofenin, lactitol, isomalt, imidurea, gluconolactone, methanesulfonic acid, xylenesulfonic acid, sulfobutylether-beta-cyclodextrin, caffeic acid, caffeic acid phenethyl ester, zileuton, inhibitor of leukotrienes, tropane N-heterocycles, atropine, hyoseyamine, scopolamine, tiotropium, ipratropium salts, allithiamine, prosulthiamine, fursulthiamine, benfothiamine, sulbuthiamine, 1-(3-aminopropyl)-2-methyl-1H-imidazole dihydrochloride, cimetidine, piperocaine, cyclomethylcaine, moxifloxacin, chloroquine, mepivacaine, levetriacetam, bupivacaine, cinchocaine, clindamycin, colistin, articane, tetracaine, etidocaine, cyclomethylcaine, piperocaine, phenylephrine, and bupivacaine, Polyethylene glycol, branched PEG, and PolyPEG R, lactobionic acid, glucuronic acid, biotin, brocrinat, cyclopentane propionic acid, hydroxynaphthoic acid, phenylpropionic acid, camphoric acid, mandelic acid, sulfosalicyclic acid, hydroxybenzoyl benzoic acid, cinnamic acid, t-butyl acetic acid, phthalic acid, trimethylacetic acid, N-methylglucamine, morpholine, piperidine, isopropylamine, trimethylamine, diethylamine, triethylamine, tripropylamine, 2-diethylaminoethanol, trimethamine, dicyclohexylamine, lidocaine, hydrabamine, cholines, betaines, ethylenediamine, purines, piperazine, N-methylpiperidinepolyamine, 2-amino-2-hydroxymethyl-propane-1,3-diol (TRIS), 4-aminopyridine, aminocyclohexane carboxylic acid, 1-o-tolybiguanide, urea, benzethonium chloride, 5-amino-1-pentanol, 2-(2-aminoethoxy) ethanol, trans-cyclohexane-1,4-diamine, trans-cyclohexane-1R,2R-diamine, propane-1,3-diamine, butane-1,4-diamine, pentane-1,5-diamine, hexane-1,6-diamine, octane-1,8-diamine, 2-(2-aminoethoxy) ethanamine, 2-(2-(2-aminoethoxy)-ethoxy) ethanamine, 3-(4-(3-aminopropoxy)-butoxy) propan-1-amine, 3-(2-(2-(3-aminopropoxy)-ethoxy)-ethoxy) propan-1-amine, N-(2-(2-aminoethylamino)ethyl) ethane-1,2-diamine, N-(2-aminoethyl) ethane-1,2-diamine, N-1-(2-(2-(2-aminoethylamino)ethylamino)-ethyl) ethane-1,2-diamine, N,N-dimethylhexane-1,6-diamine, N,N,N,N-tetramethylbutane-1,4-diamine, phenyltrimethylammonium salts, choline, 1-(3-aminopropyl)-2-methyl-1H-imidazole, 1-(2-aminoethyl) piperazine, 1-[3-(dimethylamino) propyl]piperazine, 1-(2-aminoethyl) piperidine, 2-(2-aminoethyl-1-methylpyrrolidine and combinations thereof.

### B. Methods

Often, protein agent-based therapeutics are administered through intravenous infusions, which are costly and can require a high level of patient compliance. Some protein agent-based therapeutics may be administered via subcutaneous or intramuscular injection. While these routes can offer clear advantages in ease of administration and cost when compared to intravenous infusions, they can also present challenges that may arise, for example, from limited infusion volume tolerance. Typically, it is preferred that infusion volumes be under about 2 mL for subcutaneous infusions and under about 5 mL for intramuscular injections. Furthermore, it is often preferred that preparations for subcutaneous or intramuscular injections have a viscosity of about 20 centipoise (cP) or lower.

If it is desired to administer a comparable (or identical dose) by a different route, for example by subcutaneous (SC) infusion, then a highly concentrated formulation is useful, given that a permitted volume for such route is so much smaller than that for IV injection. Such high concentration formulations, as discussed herein, can present significant administration challenges, among other things, due to high viscosity. Also, efforts to concentrate protein agents in order achieve smaller volumes for infusion can risk damage to protein agents, for example as a result of chemical and/or physical instability. Still further, subjects sometimes report pain at infusion sites when viscosity is high. Reported antibody concentrations formulated for SC infusions can be up to about 100 mg/mL (Wang et al., J. Pharm. Sci. 96:1-26, 2007) and in some cases, even 150 to 200 mg/mL.

In various embodiments, the invention provides a method of subcutaneously infusing to a first infusion site a pharmaceutical formulation of 20% (w/v) IgG to a subject in need thereof, the method comprising: (a) infusing to the first infusion site, a first aliquot of a pre-determined dosage of hyaluronidase by infusing a pre-determined volume of the pharmaceutical formulation of hyaluronidase to the first infusion site; and (b) following (a), infusing to the first infusion site, a first aliquot of a pre-determined dosage of IgG by infusing a first pre-determined volume of the pharmaceutical formulation of 20% (w/v) IgG to the first infusion site. In an exemplary embodiment, the method further comprises: (c) infusing to a second infusion site, a second aliquot of the pre-determined dosage of hyaluronidase by infusing a second pre-determined volume of the pharmaceutical formulation of hyaluronidase to the second infusion site; and (d) following (c), infusing to the second infusion site, a second aliquot of a pre-determined dosage of IgG by infusing a second pre-determined volume of the pharmaceutical formulation of 20% (w/v) IgG to the second infusion site.

In various embodiments, the first pre-determined volume of the pharmaceutical formulation of 20% (w/v) IgG is at least about 120 mL, at least about 150 mL, at least about 180 mL, at least about 200 mL, at least about 220 mL, at least about 250 mL, at least about 280 mL, or at least about 300 mL.

In various embodiments, the first final pre-determined rate is at least about 120 mL/hr, at least about 150 mL/hr, at least about 180 mL/hr, at least about 200 mL/hr, at least about 220 mL/hr, at least about 250 mL/hr, at least about 280 mL/hr, or at least about 300 mL/hr.

In various embodiments, the first predetermined volume of the pharmaceutical formulation of 20% (w/v) IgG is from about 100 mL to about 300 mL, e.g., from about 150 mL to about 200 mL, from about 200 mL to about 250 mL, from about 250 mL to about 300 mL, and is infused at the first infusion site at a first final rate of from about 100 mL/hr to about 300 mL/hr, e.g., from about 150 mL/hr to about 200 mL/hr, from about 200 mL/hr to about 250 mL/hr, or from about 250 mL/hr to about 300 mL/hr.

In an exemplary embodiment, prior to achieving the first final rate of 300 mL/hr, a first intermediate infusing rate less than 300 mL/hr is maintained for a selected time and increased to the first final pre-determined rate.

In some embodiments, the first pre-determined volume of the pharmaceutical formulation of 20% (w/v) IgG is infused to the first infusion site at a rate of at least about 300 mL/hr without reduction in rate or cessation of infusion due to subject discomfort, pain or a combination thereof.

In one, embodiment, the first pre-determined volume of the pharmaceutical formulation of 20% (w/v) IgG is infused to the first infusion site at a rate encompassing a ramp up phase followed by a terminal phase, wherein the terminal phase rate is about 200 to about 300 mL/hr, e.g., about 220 mL/hr, about 240 mL/hr, about 260 mL/hr, about 280 mL/hr, the terminal phase ending upon infusion of the last of the first pre-determined volume to the first infusion site, the terminal phase proceeding without reduction in rate or cessation of infusion due to subject discomfort, pain or a combination thereof.

An example of infusion rate ramp-up schedules based on body mass is presented in Table 1.

TABLE 1
TAK 881-3001-Immune Globulin Subcutaneous 20% (Human) Infusion Rates.

| | First 2 Infusions | | Subsequent Infusions | |
|---|---|---|---|---|
| | Subjects < 40 kg | Subjects ≥ 40 kg | Subjects < 40 kg | Subjects ≥ 40 kg |
| | (<88 lbs) | (≥88 lbs) | (<88 lbs) | (≥88 lbs) |
| Intervals | Rate per site | Rate per site | Rate per site | Rate per site |
| Minutes | mL per hour | mL per hour | mL per hour | mL per hour |
| 5-15 | 10 | 30 | 20 | 60 |
| 5-15 | 20 | 60 | 40 | 120 |
| 5-15 | 40 | 120 | 80 | 180 |
| 5-15 | 80 | 180 | | |
| Remainder of | 160 | 300 | 160 | 300 |
| infusion | | | | |

In various embodiments, at least about 60% of the first pre-determined volume of the pharmaceutical formulation of 20% (w/v) IgG is infused to the first infusion site during the terminal phase at the first final rate of at least about 200 mL/hr to about 300 mL/hr, e.g., about 220 mL/hr, about 240 mL/hr, about 260 mL/hr, about 280 mL/hr without reduction in rate or cessation of infusion due to subject discomfort, pain or a combination thereof.

In various embodiments, the first predetermined volume is from about 200 mL to about 300 mL, e.g., about 220 mL, about 240 mL, about 260 mL, about 280 mL and the first final rate is from about 200 mL/hr to about 300 mL/hr, e.g., about 220 mL/hr, about 240 mL/hr, about 260 mL/hr, about 280 mL/hr.

In an exemplary embodiment, the second final pre-determined rate is about 300 mL/hr, and prior to achieving the second final pre-determined rate a second intermediate infusing rate is maintained for a selected time and increased to the second final pre-determined rate.

In various embodiments, the pre-determined dosage of the pharmaceutical formulation of hyaluronidase is essentially similar between the method of infusing the pharmaceutical formulation of 20% (w/v) IgG, and a method of infusing an otherwise identical pharmaceutical formulation containing 10% (w/v) IgG.

In exemplary embodiments, the first predetermined dosage of the pharmaceutical formulation of 20% (w/v) IgG is infused to the first infusion site at a rate of from about 2-times to about 3-times greater than that for infusing a pharmaceutical formulation of 20% (w/v) IgG in the absence of the infusing to the first infusion site of the pre-determined dosage of hyaluronidase prior to infusing the pharmaceutical formulation of 20% (w/v) IgG to the first infusion site.

In some embodiments, the method is practiced with a system configured to practice the method. An exemplary system includes: (a) a first container comprising a pharmaceutical formulation of recombinant human hyaluronidase in a pharmaceutically acceptable carrier; (b) a second container comprising a pharmaceutical formulation of 20% w/v IgG in a pharmaceutically acceptable carrier; and (c) means for sequentially subcutaneously infusing into a first infusion site, (i), the first aliquot of a pre-determined dosage of the pharmaceutical formulation of recombinant human hyaluronidase and, (ii), following (i), the first aliquot of a pre-determined dosage of the pharmaceutical formulation of 20% IgG.

In various embodiments, the means for sequentially subcutaneously infusing into a first infusion site, includes: (i) a subcutaneous needle set; (ii) a pooling bag; (iii) a gravity fill set with vented spike; (iv) a syringe; (v) a pump; (vi) a warming device; (vii) tubing; and a combination thereof.

The method of any of the preceding paragraphs can be practiced with the kit according to any of the preceding paragraphs in any combination. The skilled person will find it apparent that each of the elements set forth above can be combined in any useful combination.

In an exemplary embodiment, the invention provides a method of infusing a pharmaceutical formulation of an immune globulin into a first infusion site of a subject in need thereof. The formulation infused according to the method comprises at least about 20% (w/v) of an immune globulin fraction in an aqueous pharmaceutically acceptable carrier dissolving the immune globulin fraction. The method includes delivering the pharmaceutical formulation from a first vessel through a first hypodermic needle and into the first infusion site, wherein the first vessel, and the first hypodermic needle are maintained in fluidic communication through a first connecting member, and wherein the pharmaceutical formulation is at an infusion temperature of from about 30° C. to about 40° C., about 30° C. to about 37° C., about 30° C. to about 35° C., or about 33° C. to about 35° C. as it enters into the first infusion site.

In an exemplary embodiment, the invention provides a method of administering a concentrated IgG formulation such that the administration of the pharmaceutical formulation at the infusion temperature is not accompanied by greater subject discomfort than that experienced by the subject upon administration, under identical administration parameters, of an otherwise identical pharmaceutical formulation comprising about 10% (w/v) of an immune globulin in an aqueous pharmaceutical carrier. In various embodiments, the administration is accompanied by less patient discomfort than the administration of the 10% (w/v) formulation.

In an exemplary embodiment, the invention provides a method of administering a concentrated IgG formulation such that the administration of the pharmaceutical formulation at the infusion temperature is not accompanied by greater subject discomfort than that experienced by the subject upon administration, under identical administration parameters, of an otherwise identical pharmaceutical formulation comprising about 20% (w/v) of an immune globulin in an aqueous pharmaceutical carrier. In various embodiments, the administration is accompanied by less patient discomfort than the administration of a similar or the same 20% (w/v) formulation at a temperature less than 30° C.

In an exemplary embodiment, the invention provides a method of administering a concentrated IgG formulation such that the administration of the pharmaceutical formulation at the infusion temperature is not accompanied by greater subject discomfort than that experienced by the subject upon administration of an otherwise identical pharmaceutical formulation comprising about 20% (w/v) of an immune globulin in an aqueous pharmaceutical carrier at 25° C. In various embodiments, the administration is accompanied by less patient discomfort than the administration of the 20% (wt/v) formulation at 25° C.

In various embodiments, the bleb resulting from the infusion is regularly shaped, indicating increased dispersion of the infused formulation, and is essentially completely resolved between about 8 and about 24 hours post infusion.

It is to be recognized that, in some embodiments, the IgG formulation flow rate will slow as the formulation comes into contact with tissue within the administration site. According to this embodiment, the pharmaceutical formulation is infused into the subject at the first infusion site at a second flow rate, which is different from the flow rate at which it exits the distal end of the needle, and this flow rate may alter as the formulation occupies the subcutaneous space.

In an exemplary embodiment, the second flow rate is at least about 3 mL/min, e.g., at least about 5 mL/min, and flow rates about as high as about 7.5 mL/min are achievable using the formulation, method and system of the invention.

### C. Systems

In various embodiments, the invention provides a system for subcutaneously infusing a pharmaceutical formulation of 20% (w/v) IgG. The system is configured for subcutaneously infusing the pharmaceutical formulation to a first infusion site of a subject in need thereof. The pharmaceutical formulation comprises at least about 20% (w/v) of IgG and an aqueous pharmaceutically acceptable carrier in which the IgG is dissolved. An exemplary system includes: a first vessel containing the pharmaceutical formulation of 20% (w/v) IgG: a second vessel containing a pharmaceutical formulation of hyaluronidase: a first hypodermic needle comprising a first terminus configured to penetrate a first infusion site of the subject, and a terminal opening disposed therein through which the pharmaceutical formulation of 20% (w/v) IgG is delivered to the first infusion site: an optional first connecting member configured for fluidic connection with the first vessel and the hypodermic needle; and a first warming device configured for thermal contact with a system component selected from the first vessel, the first connecting member, and a combination thereof, the first warming device configured to heat the pharmaceutical formulation of 20% (w/v) IgG to at least about 30° C., maintain the pharmaceutical formulation of 20% (w/v) IgG at a temperature of at least about 30° C., and a combination thereof.

In exemplary embodiments, at least one component of the system is configured to heat the pharmaceutical formulation of 20% (w/v) IgG to a temperature of from about 30° C. to about 41° C., to maintain the pharmaceutical formulation of 20% (w/v) IgG at a temperature of from about 30° C. to about 41° C., and a combination thereof. An exemplary warming device is configured to maintain the pharmaceutical formulation of 20% (w/v) IgG essentially constant through the duration of the infusion to the first infusion site.

In an exemplary embodiment, the invention provides an infusion system wherein the system is configured such that the pharmaceutical formulation, at about 30° C., is delivered into the first infusion site via the first hypodermic needle at a flow rate of from about 3 to about 7.5 mL/min with a tissue backpressure of not more than about 47000 Pa (350 mmHg).

In various embodiments, the system further includes a means for driving the pharmaceutical formulation of 20% (w/v) IgG from the first vessel through the first connecting member and into the first hypodermic needle, from which the pharmaceutical formulation exits the system via the terminal opening thereof. In an exemplary embodiment, the system further includes a pump for driving the pharmaceutical formulation of 20% (w/v) IgG from the first vessel through the first connecting member and into the first hypodermic needle, from which the pharmaceutical formulation exits the system via the terminal opening thereof.

In various embodiments, the system is utilized to infuse the pharmaceutical formulation of 20% (w/v) IgG into the first infusion site at a first final flow rate, which is at least about 2 mL/min, at least about 3 mL/min, or at least about 5 mL/min.

In various embodiments, the the pharmaceutical formulation of 20% (w/v) IgG in the system is essentially free of a small organic molecule incorporated into the formulation expressly to reduce the viscosity thereof.

In various embodiments, the first vessel is selected from an infusion bag and a syringe.

As noted above, the pharmaceutical formulation is associated with a system for its administration to the infusion site. An exemplary system includes a first vessel, serving as a reservoir of the pharmaceutical formulation, a means to expel the formulation from the reservoir, a hypodermic needle directly or indirectly fluidically communicating with the first vessel, and a means to heat the pharmaceutical formulation to the infusion temperature (about 30° C. to about 40° C.). The system optionally further contains other components of a known injector apparatus. In one embodiment, the system comprises a pump connected to the reservoir and configured to apply an amount of pressure to the IgG formulation sufficient to drive it from the reservoir, through components downstream from the reservoir and into the infusion site. In an exemplary embodiment, the pump is a peristaltic pump. In alternate embodiments the pump is a pressure driven flow control pump or an infusion pump.

In various embodiments, the warming device is a heated syringe. The heated syringe can be a standard syringe that is pre-heated using a syringe warmer. The syringe warmer will generally have one or more openings each capable of receiving a syringe containing the protein formulation and a means for heating and maintaining the syringe at a specific (typically above the ambient) temperature prior to use. This will be referred to herein as a pre-heated syringe. Suitable heated syringe warmers include those available from Vista Dental Products and Inter-Med. The warmers are capable of accommodating various sized syringes and heating, typically to within about 1° C., to any temperature up to about 130° C. In some embodiments the syringe is pre-heated in a heating bath such as a water bath maintained at the desired temperature.

The heated syringe can be a self-heating syringe, i.e. capable of heating and maintaining the liquid formulation inside the syringe at a specific temperature. The self-heating syringe can also be a standard medical syringe having attached thereto a heating device. Suitable heating devices capable of being attached to a syringe include syringe heaters or syringe heater tape available from Watlow Electric Manufacturing Co. of St. Louis, Mo., and syringe heater blocks, stage heaters, and in-line perfusion heaters available from Warner Instruments of Hamden, Conn., such as the SW-61 model syringe warmer. The heater maybe controlled through a central controller, e.g. the TC-324B or TC-344B model heater controllers available from Warner Instruments.

The heated syringe maintains the liquid protein formulation at a specified temperature from room temperature up to about 60° C. as long as the IgG formulation is sufficiently stable at that temperature. By heating the IgG formulations to an elevated temperature before and/or during infusion, the viscosity of the liquid formulation is decreased, the solubility of the IgG in the formulation is increased, or both.

In various embodiments, the IgG formulation is infused using a needle set designed for SC infusions. Exemplary needles are mounted at a 90° angle to plastic wings or a clear plastic disk to facilitate proper insertion of the needle into the subcutaneous fat, and to help keep the needles in place during the infusion. Numerous infusion sets are available, with needle sizes from 19-27 gauge, and 6, 9, 12 14, 16, and 19 mm lengths. In an exemplary embodiment, the system of the invention includes a 19G needle.

In some embodiments, the first vessel and the hypodermic needle are attached by and in fluidic communication through a first connecting member. An exemplary connecting member is a length of tubing or tubing set. The type of tubing set selected will depend on the number of sites and infusion pumps used. Tubing sets are available with single, bifurcated, trifurcated, quadfurcated, and five leg branches attached to a single trunk, which can be connected to the syringe or infusion pump.

The IgG formulation is heated to from about 30° C. to about 40° C. prior to and/or during infusion. The product is drawn up into one or more syringes depending on the amount to be infused, and the number and the type of infusion pumps being used. The infusion needle set tubing is connected to the syringe and the tubing is primed prior to insertion of the needle. Some infusion pumps use an IV bag or “cassette,” which is filled with the IgG formulation and connected to the pump. A number of different infusion pumps have been used for administration of IgG formulation. Most of these are syringe pumps which will accept a 50 mL syringe and some can be programmed to set different infusion rates. A useful pump will have sufficient power to infuse into the SC space which generates a much higher resistance to flow compared to IV infusions. The pharmaceutical formulation can be heated while in any of the components of the infusion system.

In various embodiments, the system includes one or more components of the system set forth in WO2016/205687, and/or WO2020/072230.

### D. Hyaluronidase Formulations and Methods

### 1. Hyaluronidases

Hyalruonidases are included in the formulations, methods and combinations provides provided herein. Soluble hyalurondases include any, that, upon expression and secretion from a cell, exist in soluble form. Such soluble hyaluronidases include, but are not limited to, non-human soluble hyaluronidases, including those referred to as sHASEPGs), bacterial soluble hyaluronidases, bovine PH20, ovine PH20, and variants thereof. Included among the soluble hyaluronidases are human PH20 polypeptides that have been been modified, generally by C-terminal truncation, so that they are secreted when expressed and are soluble. For example, hyaluronidases, such as human PH20, that contain a glycophophatidylinositol (GPI) anchor can be made soluble by truncation of and removal of all or a portion of the GPI anchor. In one example, the human hyaluronidase PH20, which is normally membrane anchored via a GPI anchor, is made soluble by truncation of and removal of all or a portion of the GPI anchor at the C-terminus.

### (a) Soluble Human PH20

Exemplary of a soluble hyaluronidase is soluble human PH20. Soluble forms of recombinant human PH20 have been produced and can be used in the compositions, combinations and methods described herein. The description of and production of such soluble forms of PH20 is described, for example, in U.S. Pat. Nos. 7,767,429, 8,202,517, 8,431,380, 8,431,124, 8,450,470 8,765,685, 8,772,246, 7,871,607, 7,846,431, 7,829,081, 8,105,586, 8,187,855, 8,257,699, 8,580,252, 9,677,061, and 9,677,062 which are incorporated by reference herein.

Soluble hyaluronidases include neutral active hyaluronidases, such as the soluble human PH20 polypeptides. In a particular example, the hyaluronidase for use in the compositions, combinations and methods herein is a soluble neutral active hyaluronidase.

Exemplary of hyaluronidases include a soluble form of a PH20 from any species, such as a soluble form of a PH20 of any of SEQ ID NOs: 3 and 32-66 [first set of sequences attached], and such as the soluble PH20 polypeptides set forth in SEQ ID NOs. 3 and 44-49. Such soluble forms include truncated forms thereof lacking all or a portion of the C-terminal GPI anchor, so long as the hyaluronidase is soluble (secreted upon expression) and retains hyaluronidase activity. Such forms also typically are mature forms that, when expressed in a cell, lack the signal peptide. Also included among soluble hyaluronidases are soluble forms of variants of any of the PH20s from any species set forth in SEQ ID NOs: 3 and 32-66 that exhibit hyaluronidase activity. Variants include polypeptides having at least 60%, 70%, 80%, 85%, 90%, 91%, 92%, 93%, 94%, 95%, 96%, 97%, 98%, 99% or more sequence identity to any of SEQ ID NOs: 3 and 32-66. Amino acid variants include conservative and non-conservative mutations. It is understood that residues that are important or otherwise required for the activity of a hyaluronidase, such as any described above or known to skill in the art, are generally invariant and cannot be changed. These include, for example, active site residues. Thus, for example, amino acid residues 111, 113 and 176 (corresponding to residues in the mature PH20 polypeptide set forth in SEQ ID NO: 3) of a human PH20 polypeptide, or soluble form thereof, are generally invariant and are not altered. Other residues that confer glycosylation and formation of disulfide bonds required for proper folding also can be invariant.

In some instances, the soluble hyaluronidase is normally GPI-anchored (such as, for example, human PH20) and is rendered soluble by truncation at the C-terminus. Such truncation can remove all of the GPI anchor attachment signal sequence, or can remove only some of the GPI anchor attachment signal sequence. The resulting polypeptide, however, is soluble. In instances where the soluble hyaluronidase retains a portion of the GPI anchor attachment signal sequence, 1, 2, 3, 4, 5, 6, 7 or more amino acid residues in the GPI anchor attachment signal sequence can be retained, provided the polypeptide is soluble. Polypeptides containing one or more amino acids of the GPI anchor are termed extended soluble hyaluronidases. One of skill in the art can determine whether a polypeptide is GPI-anchored using methods well known in the art. Such methods include, but are not limited to, using known algorithms to predict the presence and location of the GPI anchor attachment signal sequence and w-site, and performing solubility analyses before and after digestion with phosphatidylinositol-specific phospholipase C (PI-PLC) or D (PI-PLD).

Extended soluble hyaluronidases, such as those set forth in SEQ ID NOs: 61-66, can be produced by making C-terminal truncations to any naturally GPI-anchored hyaluronidase such that the resulting polypeptide is soluble and contains one or more amino acid residues from the GPI anchor attachment signal sequence (see, e.g., U.S. Pat. No. 8,927,249). These include hyaluronidases that are neutral active, soluble, contain amino acid substitutions, and have at least 60%, 70%, 80%, 85%, 90%, 91%, 92%, 93%, 94%, 95% or more sequence identity to any of SEQ ID NOs: 61-66.

Typically, for use in the compositions, combinations and methods herein, a soluble human hyaluronidase, such as a soluble human PH20, is used, such as a PH20 polypeptide of any of SEQ ID NOs: 3 and 45-49 and variants having, for example, at least 98% sequence identity thereto. Hyaluronidases used in the methods herein can be recombinantly produced or can be purified or partially-purified from natural sources, such as, for example, from testes extracts. Methods for production of recombinant proteins, including recombinant hyaluronidases, are well known in the art.

Recombinant soluble forms of human PH20 have been generated and can be used in the compositions, combinations and methods provided herein. For example, with reference to SEQ ID NO: 1, which sets forth the sequence of full length precursor PH20, which includes a signal sequence (residues 1-35), soluble forms include, but are not limited to, C-terminal truncated polypeptides of human PH20 set forth in SEQ ID NO: 1 having a C-terminal amino acid residue 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO: 1, or polypeptides that exhibit at least 85%, 90%, 91%, 92%, 93%, 94%, 95%, 96%, 97%, 98%, 99%, or more sequence identity thereto, have activity at neutral pH, and are soluble (secreted into the medium when expressed in a mammalian cell). Soluble forms of human PH20 generally include those that contain amino acids 36-464 set forth in SEQ ID NO: 1. For example, when expressed in mammalian cells, the 35 amino acid N-terminal signal sequence is cleaved during processing, and the mature form of the protein is secreted. Thus, the mature soluble polypeptides include those that contain amino acids 36 to 467, 468, 469, 470, 471, 472, 473, 474, 475, 476, 477, 478, 479, 480, 481, 482 and 483 of SEQ ID NO: 1. Exemplary of soluble hyaluronidases are soluble human PH20 polypeptides that are 442, 443, 444, 445, 446 or 447 amino acids in length, such as set forth in any of SEQ ID NOs: 3 and 44-49 and variants thereof that have, for example, at least 85%, 86%, 87%, 88%, 89%, 90%, 91%, 92%, 93%, 94%, 95%, 96%, 97%, 98% or 99% sequence identity to a sequence of amino acids set forth in any of SEQ ID NOs: 3 and 44-49 and retains hyaluronidase activity. The generation of such soluble forms of recombinant human PH20 are described, for example, in U.S. Pat. Nos. 7,767,429, 8,202,517, 8,431,380, 8,431, 124, 8,450,470 8,765,685, 8,772,246, 7,871,607, 7,846,431, 7,829,081, 8,105,586, 8,187,855, 8,257,699, 8,580,252, 9,677,061, and 9,677,062.

Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

(b) rHuPH20

rHuPH20 refers to the composition produced upon expression in a cell, such as CHO cell, of nucleic acid encoding residues 36-482 of SEQ ID NO: 1, generally linked to the native or a heterologous signal sequence (residues 1-35 of SEQ ID NO: 1). rHuPH20 is produced by expression of a nucleic acid molecule, such as encoding amino acids 1-482 (set forth in SEQ ID NO: 1). Post translational processing removes the 35 amino acid signal sequence, leaving a polypeptide or a mixture of polypeptides, including those set forth in SEQ ID NOs: 3 and 44-49. As produced in the culture medium there is heterogeneity at the C-terminus such that the product, designated rHuPH20, includes a mixture of species that can include any one or more of SEQ ID NOs: 3 and 44-49 in various abundance. Typically, rHuPH20 is produced in cells that facilitate correct N-glycosylation to retain activity, such as CHO cells (e.g. DG44 CHO cells). Generally the most abundant species is the 446 amino acid polypeptide corresponding to residues 36-481 of SEQ ID NO: 1.

### (c) Glycosylation of Hyaluronidases

Glycosylation, including N- and O-linked glycosylation, of some hyaluronidases, including the soluble PH20 hyaluronidases, can be important for their catalytic activity and stability. For some hyaluronidases, removal of N-linked glycosylation can result in near complete inactivation of the hyaluronidase activity. Thus, for such hyaluronidases, the presence of N-linked glycans can be important for generating an active enzyme.

N-linked oligosaccharides fall into several primary types (oligomannose, complex, hybrid, sulfated), all of which have (Man) 3-GlcNAc-GlcNAc-cores attached via the amide nitrogen of Asn residues that fall within-Asn-Xaa-Thr/Ser-sequences (where Xaa is not Pro). Glycosylation at an -Asn-Xaa-Cys- site has been reported for coagulation protein C. In some instances, a hyaluronidase, such as a PH20 hyaluronidase, can contain N-glycosidic and O-glycosidic linkages. For example, PH20 has O-linked oligosaccharides as well as N-linked oligosaccharides. There are six potential N-linked glycosylation sites at N82, N166, N235, N254, N368, N393 of human PH20 exemplified in SEQ ID NO: 1.

### (d) Variants

Variants of the soluble PH20 polypeptides that have altered properties, such as increased stability and/or activity, have been produced. U.S. Pat. Nos. 9,447,401 and 10,865,400, and allowed application Ser. No. 16/824,572, which are incorporated by reference, describe and provide a structure/function map of human PH20 detailing the effects of amino acid replacements at every residue in the catalytic domain of PH20. Theses patents provide about 7000 examples in which the effects of replacing each amino acid with 15 other amino acids on activity and stability were identified and described. By virtue of that publication and earlier publications/patents virtually all variants of soluble PH20 polypeptides, including those with amino acid replacements, deletions, and insertions, are known in the art. A skilled person readily can prepare soluble hyaluronidases and variants thereof and know the properties of the resulting hyaluronidase.

Other variants are known to those of skill in the art. See, International PCT application Nos. WO2020/022791 and WO2020197230A, which are incorporated by reference, and which describe modified PH20 polypeptides. These polypeptides, which are variants of the PH20 polypeptides of SEQ ID NOs: 1, 3 and 32-66 include replacements, insertions, and deletions, including one or more amino acid residues S343E, M345T, K349E, L353A, L3541, N356E, and I361T. Variants that contain such modifications and others are set forth in SEQ ID NOs: 60-115 from International PCT application No WO2020/022791. International PCT application No/WO2021/150079 also provides variant PH20 polypeptides desribed as having increased stablity.

### 2. Administration of IG and Hyaluronidases

In various embodiments, any formulation or method set forth above is augmented by infusion into the IgG administration site of a predetermined dosage of hyaluronidase prior to or in conjunction with the administration at the site of the IgG formulation of the invention. The hyaluronidase is administered at the same temperature as the IgG or at a different temperature.

The diffusion and convective transport of IgG can be enhanced by opening interstitial channels and increasing fluid flow. The size of IgG and the presence of interstitial components such as glycosaminoglycans substantially impairs the diffusion and/or convection of the agents. Several consequences potentially limiting IgG's usefulness can result. For example, the pharmacokinetics of IgG can be effectively impaired by a slowing of absorption and thus distribution of IgG. In addition, trapping of a portion of IgG at or near the site of administration limits its bioavailability and can also cause toxicity as a result of a potentially sustained and high local dose. In the latter regard, local toxicity that may be associated with painful or other side effects is a problem with many large biomolecules that are administered via subcutaneous infusion. As a result, the pharmacokinetic (PK) and/or pharmacodynamic (PD) profile of IgG is enhanced by co-formulating IgG with a sHASEGP (or other glycosaminoglycanase) and/or co-administering IgG with a sHASEGP (or other glycosaminoglycanase), which may be provided before, coincident with or after the IgG, and administered at the same or a different site, which parameters would be the subject of optimization in standard models (such as animal models typically used to assess the pharmacokinetics and pharmacodynamics of IgG).

By way of further illustration and without being limited to a particular type of application, a volume (V) of liquid (L) comprising a sHASEGP or other glycosaminoglycanase (GAG Enzyme) can be introduced into a patient administration site. By means as described and illustrated herein, the IgG formulation can be delivered into and to some extent through the administration site. Without being restricted to a particular mechanism of action, the IgG formulation can be effectively carried into tissue adjacent the administration site by convective transport by the volume of liquid (L). The convective transport can in turn be promoted in part by the hydrostatic pressure associated with L (which can provide a driving pressure). A fluid-driving pressure differential can thus be created.

As will be appreciated by those of skill in the art, V can range from volumes of less than about 0.1 mL to volumes of greater than about 100 mL, for many applications being in the range of about 0.5 mL to about 20 mL, and for many in the range of about 1 mL to about 10 mL, frequently from about 2 to about 5 mL: but can be varied for particular situations as illustrated herein. V can also be specifically optimized within such ranges for a particular application as desired: e.g. by comparing standard pharmacokinetic and/or pharmacodynamic profiles over a range of test volumes.

Subcutaneous infusion of recombinant human hyaluronidase has been shown to facilitate the dispersion of IgG injected SC, allowing patients to infuse larger volumes of IG into a single site on a monthly basis. Knight et al., (2010), 63 (9): 846-7. Clinical trials have demonstrated the safety and efficacy of infusion of a 10% IgG product following infusion of recombinant human hyaluronidase using doses and rates equivalent to monthly IVIG treatment. Injecting IgG 10% every 3-4 weeks at doses comparable to monthly IVIG (320 mg/kg/month-1000 mg/kg/month) using a single SC site and infused at rates up to 300 mL/hr are well tolerated. Schiff et al., Clin Exp Immunol (2008), 154 (Suppl 1): 121. The rate of local reaction was slightly higher than traditional SCIG and the rate of systemic reactions was comparable to SCIG but significantly lower than seen with IVIG. Stein et al., J Allergy Clin Immunol (2012), AB14. In addition, the pharmacokinetics of IgG infused with hyaluronidase SC are comparable to IVIG, with therapeutic IgG trough levels and good bioavailability, and the annual rate of infection for patients in the study was lower than seen for patients on SCIG or IVIG. Stein, supra.

The present invention provides for the use of a pharmaceutical formulation of a hyaluronidase in conjunction with the pharmaceutical formulation of the IgG. The hyaluronidase formulation can be used to improve the subcutaneous delivery of the IgG. In an exemplary embodiment, the IgG formulation (at least about 20% wt/wt) is administered to the administration site at a temperature of from about 22° C. to about 40° C. In an exemplary embodiment, the formulation is administered at about room temperature, e.g., about 22° C. to about 26° C. In various embodiments, the formulation is administered at a temperature approximating the body temperature of the subject, e.g., from about 30° C. to about 40° C.

Many molecules injected percutaneously (e.g. by using a needle or other device that penetrates the skin and is used to deposit the molecules into a sub-dermal layer) reach circulation slowly or with very low efficiency. Several factors regulate the pharmacokinetics and pharmacodynamics of molecules injected subcutaneously (SC) or intramuscularly (IM). Generally, larger molecules reach circulation more slowly and less efficiently without active transport into circulation. Subcutaneous bioavailability is determined by calculating the ratio of area under the curves for SC verses intravenous administration (AUC_{SC}/AUCᵢₙₜᵣₐᵥₑₙₒᵤₛ). A second factor is charge and affinity for matrix molecules that may play a role in sequestration of molecules subcutaneously. If these materials are degraded locally they may never reach their desired targets and thus demonstrate a decreased overall systemic bioavailability to the target organs.

An additional benefit of the invention lies in the ability to deliver equivalent or larger volumes of solutions ID, SC or IM without the pain and morbidity associated with the pressure and volume of the solution at the site of infusion.

Of particular interest is the use of soluble neutral-active hyaluronidases or sHASEGPs of mammalian, including human, origin. Those of skill in this art recognize that, in general, single amino acid substitutions in non-essential regions of a polypeptide do not substantially alter biological activity (see, e.g., Watson et al., (1987) Molecular Biology of the Gene, 4th Edition, The Benjamin/Cummings Pub. co., p. 224).

Parenteral administration of the sHASEGP or a soluble human hyaluronidase domain thereof, generally characterized by injection, either subcutaneously (infusion), intramuscularly or intravenously is also contemplated herein. Injectables can be prepared in conventional forms, either as liquid solutions or suspensions; solid forms suitable for solution or suspension in liquid prior to infusion, or as emulsions. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as, for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of the sHASEGP or a soluble human hyaluronidase domain thereof contained in such parenteral compositions is dependent on the specific nature thereof, as well as the activity of the compound and the needs of the subject.

Parenteral administration of the compositions includes intravenous, subcutaneous and intramuscular administrations. Preparations for parenteral administration include sterile solutions ready for infusion, sterile dry soluble products, such as lyophilized powders, ready to be combined with a solvent or sterile solution just prior to use, including hypodermic tablets, sterile suspensions ready for infusion, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thiomersal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN™80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an infusion provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Injectables are designed for local administration. Typically, a therapeutically effective dosage is formulated to contain a concentration of at least about 0.1% w/w up to about 90% w/w or more, preferably more than 1% w/w of the active compound to the treated tissue(s). The active ingredient, such as a sHASEGP or a soluble human hyaluronidase domain thereof, can be administered at once, or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compounds provided herein can be formulated for parenteral administration by infusion, e.g., by bolus injection or continuous infusion. Formulations for infusion can be presented in unit dosage form, e.g., in ampoules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use. For example, provided herein are parenteral formulations containing an effective amount of sHASEGP or a soluble human hyaluronidase domain thereof, such as 500 to 500,000 Units, in a stabilized solution or a lyophilized from.

The compound can be suspended in micronized or other suitable form or can be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and can be empirically determined.

The sHASEGP polypeptides or soluble human hyaluronidase domains thereof or compositions containing any of the preceding agents can be packaged as articles of manufacture containing packaging material, a compound or suitable derivative thereof provided herein, which is effective for treatment of a diseases or disorders contemplated herein, within the packaging material, and a label that indicates that the compound or a suitable derivative thereof is for treating the diseases or disorders contemplated herein. The label can optionally include the disorders for which the therapy is warranted.

The articles of manufacture provided herein contain packaging materials. Packaging materials for use in packaging pharmaceutical products are well known to those of skill in the art (see, e.g., U.S. Pat. Nos. 5,323,907, 5,052,558 and 5,033,352). Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated, as are a variety of treatments for any disorder in which HCV infection is implicated as a mediator or contributor to the symptoms or cause.

Kits containing the compositions and/or the combinations with instructions for administration thereof are also provided herein. The kit can further include a needle or syringe, typically packaged in sterile form, for injecting the composition, and/or a packaged alcohol pad. Instructions are optionally included for administration of the active agent by a clinician or by the patient. For example, provided herein is a kit containing a small volume syringe with an effective amount of sHASEGP or a soluble human hyaluronidase domain thereof, such as 1 to 5000 Units of the soluble glycoprotein, in a 5 to 50 μL volume, optionally containing a second syringe containing a viscoelastic. Also provided herein is a kit containing a small volume syringe containing an effective amount of sHASEGP or a soluble human hyaluronidase domain thereof, such as 1 to 500 Units of the soluble glycoprotein, and a therapeutic amount of a second active ingredient, such as a drug, a small molecule, a protein or a nucleic acid.

### E. Exemplary Embodiments

In various embodiments, the invention provides a kit comprising:
(a) a first container comprising a pharmaceutical formulation of recombinant human hyaluronidase in a pharmaceutically acceptable carrier;
(b) a second container comprising a pharmaceutical formulation of 20% (w/v) IgG in a pharmaceutically acceptable carrier; and
(c) instructions providing guidance for sequentially subcutaneously infusing into a first infusion site, (i), a first aliquot of a pre-determined dosage of the pharmaceutical formulation of recombinant human hyaluronidase and, (ii), following (i), a first aliquot of a pre-determined dosage of the pharmaceutical formulation of 20% (w/v) IgG.

In various embodiments, the invention provides a kit according to the paragraph above, wherein the pharmaceutical formulation of recombinant human hyaluronidase contains 160 U/mL recombinant human hyaluronidase.

In various embodiments, the invention provides a kit according to any paragraph above, wherein the recombinant human hyaluronidase is rHuPH20.

In various embodiments, the invention provides a kit according to any paragraph above, further comprising an infusion apparatus for sequentially or simultaneously subcutaneously infusing (i), the pharmaceutical formulation of recombinant human hyaluronidase and, (ii), following (i), the pharmaceutical formulation of 20% (w/v) IgG.

In various embodiments, the invention provides a kit according to any paragraph above, further comprising a subcutaneous needle set.

In various embodiments, the invention provides a kit according to any paragraph above, wherein the instructions are a component of a Dosage and Administration section of Complete Prescribing Information.

In various embodiments, the invention provides a kit according to any paragraph above, wherein the instructions provide guidance for subcutaneously infusing the pharmaceutical formulation of rHuPH20 to the first infusion site.

In various embodiments, the invention provides a kit according to any paragraph above, wherein the instructions provide guidance for subcutaneously infusing from about 50 U/g to about 100 U/g IgG of rHuPH20 to the first infusion site.

In various embodiments, the invention provides a kit according to any paragraph above, wherein the instructions provide guidance for subcutaneously infusing at up to at least about 100 mL, at up to at least about 150 mL, up to at least about 200 mL, up to at least about 250 mL, or up to at least about 300 mL of the pharmaceutical formulation of 20% (w/v) IgG to the first infusion site.

In various embodiments, the invention provides a kit according to any paragraph above, wherein the instructions provide guidance for subcutaneously infusing the first pre-determined dosage of the pharmaceutical formulation of 20% (w/v) IgG to the first infusion site at a rate of at least about 120 mL/hr, at least about 150 mL/hr, at least about 200 mL/hr, at least about 250 mL/hr, or at least about 300 mL/hr.

In various embodiments, the invention provides a kit according to any paragraph above, wherein the instructions provide guidance for subcutaneously infusing at least about 120 mL of the pharmaceutical formulation of 20% (w/v) IgG to the first infusion site at a rate of at least about 120 mL/hr, at least about 150 mL/hr, at least about 200 mL/hr, at least about 250 mL/hr, or at least about 300 mL/hr.

In various embodiments, the invention provides a kit according to any paragraph above, wherein the instructions provide, (b) guidance for subcutaneously infusing at least about 300 mL of the pharmaceutical formulation of 20% (w/v) IgG to the first infusion site.

In various embodiments, the invention provides a kit according to any paragraph above, wherein the instructions provide (a) guidance for subcutaneously infusing at least about 300 mL of the pharmaceutical formulation of 20% (w/v) IgG to the first infusion site at a rate of at least about 300 mL/hr.

In various embodiments, the invention provides a kit according to any paragraph above, wherein the instructions provide guidance for subcutaneously infusing the pharmaceutical formulation of 20% (w/v) IgG warmed to a temperature of from about 30° C. to about 41° C., said pharmaceutical formulation warmed to the temperature prior to the infusing, during the infusing, and a combination thereof.

In various embodiments, the invention provides a kit according to any paragraph above, wherein the instructions further provide guidance on simultaneously or sequentially subcutaneously infusing at a second infusion site, (i), a second aliquot of the pre-determined dosage of the pharmaceutical formulation of recombinant human hyaluronidase and, (ii), following (i), subcutaneously infusing a second aliquot of the pre-determined dosage of the pharmaceutical formulation of 20% (w/v) IgG at the second infusion site.

In various embodiments, the invention provides a kit according to any paragraph above, wherein the instructions provide guidance on subcutaneously infusing the pharmaceutical formulation of rHuPH20 to the first infusion site, followed by infusing the pharmaceutical formulation of 20% (w/v) IgG to the first infusion site using a member selected from:
(i) a subcutaneous needle set;
(ii) a pooling bag;
(iii) a gravity fill set with vented spike;
(iv) a syringe;
(v) a pump;
(vi) a warming device;
(vii) tubing; and a combination thereof.

In various embodiments, the invention provides a method of subcutaneously infusing to a first infusion site a pharmaceutical formulation of 20% (w/v) IgG to a subject in need thereof, the method comprising:
(a) infusing to the first infusion site, a first aliquot of a pre-determined dosage of hyaluronidase by infusing a pre-determined volume of the pharmaceutical formulation of hyaluronidase to the first infusion site; and
(b) following (a), infusing to the first infusion site, a first aliquot of a pre-determined dosage of IgG by infusing a first pre-determined volume of the pharmaceutical formulation of 20% (w/v) IgG to the first infusion site.

In various embodiments, the invention provides a method according to paragraph above, further comprising:
(c) infusing to a second infusion site, a second aliquot of the pre-determined dosage of hyaluronidase by infusing a second pre-determined volume of the pharmaceutical formulation of hyaluronidase to the second infusion site; and
(d) following (c), infusing to the second infusion site, a second aliquot of a pre-determined dosage of IgG by infusing a second pre-determined volume of the pharmaceutical formulation of 20% (w/v) IgG to the second infusion site.

In various embodiments, the invention provides a kit according to any of paragraphs [00235]-[00236] above, wherein the first pre-determined volume of the pharmaceutical formulation of 20% (w/v) IgG is subcutaneously infused to the first infusion site at a first final pre-determined rate.

In various embodiments, the invention provides a method according to any of paragraphs [00235]-[00236] above, wherein the first pre-determined volume of the pharmaceutical formulation of 20% (w/v) IgG is at least about 120 mL, at least about 150 mL, at least about 180 mL, at least about 200 mL, at least about 220 mL, at least about 250 mL, at least about 280 mL, or at least about 300 mL.

In various embodiments, the invention provides a method according to any of paragraphs [00235]-[00238] above, wherein the first final pre-determined rate is at least about 120 mL/hr, at least about 150 mL/hr, at least about 180 mL/hr, at least about 200 mL/hr, at least about 220 mL/hr, at least about 250 mL/hr, at least about 280 mL/hr, or at least about 300 mL/hr.

In various embodiments, the invention provides a method according to any of paragraphs [00235]-[00239] above, wherein the first predetermined volume of the pharmaceutical formulation of 20% (w/v) IgG is from about 100 mL to about 300 mL, e.g., from about 150 mL to about 200 mL, from about 200 mL to about 250 mL, from about 250 mL to about 300 mL, and is infused at the first infusion site at a first final rate of from about 100 mL/hr to about 300 mL/hr, e.g., from about 150 mL/hr to about 200 mL/hr, from about 200 mL/hr to about 250 mL/hr, or from about 250 mL/hr to about 300 mL/hr.

In various embodiments, the invention provides a method according to any of paragraphs [00235]-[00240] above, wherein prior to achieving the first final rate of 300 mL/hr, a first intermediate infusing rate less than 300 mL/hr is maintained for a selected time and increased to the first final pre-determined rate.

In various embodiments, the invention provides a method according to any of paragraphs [00235]-[00241] above, wherein the first pre-determined volume of the pharmaceutical formulation of 20% (w/v) IgG is infused to the first infusion site at a rate of at least about 300 mL/hr without reduction in rate or cessation of infusion due to subject discomfort, pain or a combination thereof.

In various embodiments, the invention provides a method according to any of paragraphs [00235]-[00242] above, wherein the first pre-determined volume of the pharmaceutical formulation of 20% (w/v) IgG is infused to the first infusion site at a rate encompassing a ramp up phase followed by a terminal phase, wherein the terminal phase rate is about 200 to about 300 mL/hr, e.g., about 220 mL/hr, about 240 mL/hr, about 260 mL/hr, about 280 mL/hr, the terminal phase ending upon infusion of the last of the first pre-determined volume to the first infusion site, the terminal phase proceeding without reduction in rate or cessation of infusion due to subject discomfort, pain or a combination thereof.

In various embodiments, the invention provides a method according to any of paragraphs [00235]-[00243] above, wherein at least about 60% of the first pre-determined volume of the pharmaceutical formulation of 20% (w/v) IgG is infused to the first infusion site during the terminal phase at the first final rate of at least about 200 mL/hr to about 300 mL/hr, e.g., about 220 mL/hr, about 240 mL/hr, about 260 mL/hr, about 280 mL/hr without reduction in rate or cessation of infusion due to subject discomfort, pain or a combination thereof.

In various embodiments, the invention provides a method according to any of paragraphs [00235]-[00244] above, wherein the first predetermined volume is from about 200 mL to about 300 mL, e.g., about 220 mL, about 240 mL, about 260 mL, about 280 mL and the first final rate is from about 200 mL/hr to about 300 mL/hr, e.g., about 220 mL/hr, about 240 mL/hr, about 260 mL/hr, about 280 mL/hr.

In various embodiments, the invention provides a method according to any of paragraphs [00235]-[00245] above, wherein the second final pre-determined rate is about 300 mL/hr, and prior to achieving the second final pre-determined rate a second intermediate infusing rate is maintained for a selected time and increased to the second final pre-determined rate.

In various embodiments, the invention provides a method according to any of paragraphs [00235]-[00246] above, wherein the pre-determined dosage of the pharmaceutical formulation of hyaluronidase is essentially similar between the method of infusing the pharmaceutical formulation of 20% (w/v) IgG, and a method of infusing an otherwise identical pharmaceutical formulation containing 10% (w/v) IgG.

In various embodiments, the invention provides a method according to any of paragraphs [00235]-[00247] above, wherein the first predetermined dosage of the pharmaceutical formulation of 20% (w/v) IgG is infused to the first infusion site at a rate of from about 2-times to about 3-times greater than that for infusing a pharmaceutical formulation of 20% (w/v) IgG in the absence of the infusing to the first infusion site of the pre-determined dosage of hyaluronidase prior to infusing the pharmaceutical formulation of 20% (w/v) IgG to the first infusion site.

In various embodiments, the invention provides a method according to any of paragraphs [00235]-[00248] above, the method practiced with a system configured to practice the method, the system comprising:
(a) a first container comprising a pharmaceutical formulation of recombinant human hyaluronidase in a pharmaceutically acceptable carrier;
(b) a second container comprising a pharmaceutical formulation of 20% w/v IgG in a pharmaceutically acceptable carrier; and
(c) means for sequentially subcutaneously infusing into a first infusion site, (i), the first aliquot of a pre-determined dosage of the pharmaceutical formulation of recombinant human hyaluronidase and, (ii), following (i), the first aliquot of a pre-determined dosage of the pharmaceutical formulation of 20% IgG.

In various embodiments, the invention provides a method according to any of paragraphs [00235]-[00249] above, the means for sequentially subcutaneously infusing into a first infusion site, comprising:
(i) a subcutaneous needle set;
(ii) a pooling bag;
(iii) a gravity fill set with vented spike;
(iv) a syringe;
(v) a pump;
(vi) a warming device;
(vii) tubing; and a combination thereof.

The method of any of paragraphs [00235]-[00250] can be practiced with the kit according to any of paragraphs [00219]-[00234] in any combination. The skilled person will find it apparent that each of the elements set forth above can be combined in any useful combination.

In various embodiments, the invention provides a system for subcutaneously infusing a pharmaceutical formulation of 20% (w/v) IgG, the system configured for subcutaneously infusing the pharmaceutical formulation to a first infusion site of a subject in need thereof, the pharmaceutical formulation comprising:
at least about 20% (w/v) of IgG and an aqueous pharmaceutically acceptable carrier in which the IgG is dissolved;
the system comprising:
a first vessel containing the pharmaceutical formulation of 20% (w/v) IgG;
a second vessel containing a pharmaceutical formulation of hyaluronidase;
a first hypodermic needle comprising a first terminus configured to penetrate a first infusion site of the subject, and a terminal opening disposed therein through which the pharmaceutical formulation of 20% (w/v) IgG is delivered to the first infusion site;
an optional first connecting member configured for fluidic connection with the first vessel and the hypodermic needle; and
a first warming device configured for thermal contact with a system component selected from the first vessel, the first connecting member, and a combination thereof, the first warming device configured to heat the pharmaceutical formulation of 20% (w/v) IgG to at least about 30° C., maintain the pharmaceutical formulation of 20% (w/v) IgG at a temperature of at least about 30° C., and a combination thereof.

In various embodiments, the invention provides a system according to the paragraph above, wherein at least one component of the system is configured to heat the pharmaceutical formulation of 20% (w/v) IgG to a temperature of from about 30° C. to about 41° C., to maintain the pharmaceutical formulation of 20% (w/v) IgG at a temperature of from about 30° C. to about 41° C., and a combination thereof.

In various embodiments, the invention provides a system according to any of paragraphs [00252]-[00253] above, wherein the warming device is configured to maintain the pharmaceutical formulation of 20% (w/v) IgG essentially constant through the duration of the infusion to the first infusion site.

In various embodiments, the invention provides a system according to any of paragraphs [00252]-[00254] above, wherein the system further comprises a means for driving the pharmaceutical formulation of 20% (w/v) IgG from the first vessel through the first connecting member and into the first hypodermic needle, from which the pharmaceutical formulation exits the system via the terminal opening thereof.

In various embodiments, the invention provides a system according to any of paragraphs [00252]-[00255] above, wherein the system further comprises a pump for driving the pharmaceutical formulation of 20% (w/v) IgG from the first vessel through the first connecting member and into the first hypodermic needle, from which the pharmaceutical formulation exits the system via the terminal opening thereof.

In various embodiments, the invention provides a kit according to any of paragraphs [00252]-[00197] above, wherein the pharmaceutical formulation of 20% (w/v) IgG is infused into the first infusion site at a first final flow rate, which is at least about 2 mL/min, at least about 3 mL/min, at least about 4 mL/min, or at least about 5 mL/min.

In various embodiments, the invention provides a system according to any of paragraphs [00252]-[00257] above, wherein the pharmaceutical formulation of 20% (w/v) IgG is essentially free of a small organic molecule incorporated into the formulation expressly to reduce the viscosity thereof.

In various embodiments, the invention provides a system according to any of paragraphs [00252]-[00258] above, wherein the first vessel is selected from an infusion bag and a syringe.

The system of any of paragraphs [00252]-[00259] can be practiced with a method of any of paragraphs [00235]-[00250] and/or can be practiced with the kit according to any of paragraphs [00219]-[00234] in any combination. As will be apparent to the skilled person, any of the elements set forth above can be utilized in any combination.

In various embodiments according to any of the preceding paragraphs, the IgG pharmaceutical formulation includes from about 15% to about 30% (w/v) IgG, for example, from about 15% to about 20%, or from about 20% to about 30%, e.g., about 22%, about 24%, about 26% or about 28% (w/v) IgG. An exemplary formulation includes about 25% (w/v) IgG. In various embodiments, the dose of hyaluronidase needed to facilitate IgG administration at high rates of infusion as this term is defined herein is about the same for these IgG formulations as it is for a 10% (w/v) IgG formulation, or the 20% (w/v) IgG formulation exemplified herein.

The following examples are offered to illustrate exemplary embodiments of the invention and do not define or limit its scope.

### EXAMPLES

### Example 1

TABLE 2
List of Abbreviations.

| Abbreviation | Description |
|---|---|
| AD | Analytical development |
| DDQC | Device Drug Qualification & Compatibility |
| DLS | Dynamic light scattering |
| IGG | Immunoglobulin G |
| IGSC | Immunoglobulin G Subcutaneous |
| IR | Investigational record |
| MFI | Micro-flow imaging |
| MSV/FFF/BPS | Manufacturing Science Vienna/Formulation & Fill/Finish/Biophysical Science |
| MSV/PL&Q/FFF Lab | Manufacturing Science Vienna/Process Labs & Qualification/Formulation, Fill & |
| | Finish Lab |
| n.a. | Not applicable |
| NMT | Not more than |
| PDT | Plasma derived therapies |
| PH20 | Recombinant Hyaluronidase |
| SEC-HPLC | Size Exclusion High performance liquid chromatography |
| SP | Study Plan? |
| UV | Ultraviolet |

Internationally recognized units of measurement (Système International, or SI), standard and widely accepted abbreviations (for example h for hour, L for liter, min for minute and ° C. for degree Celsius) are not included in the list above.

TABLE 3
Disposable materials used for this study.

| Description | Order Number | Manufacturer |
|---|---|---|
| Tubing set 350 cm | FP1002001 | Biegler |
| Intravia Container; 500-ml | 2J8003 | Baxter |
| Infusomat Space Line | 8700036SP | Braun |
| Needle tubing set | EU1M2012 | Baxalta |
| 24 G Needle; 12 mm | EU1M2012 | Baxalta |
| Sterile Square Media | H28600201902503 | Nalgene |
| Bottle; PETG; 250 ml | | |
| Eppendorf Tubes; 1.5-mL | 0030 120.086 | Eppendorf |
| 60-mL Plastic Syringe | 309653 | BD |
| Hypodermic Needle 19 G | 4665317 | B. Braun |
| Aeration Needle 19 G × | Item 3400167 | B. Braun |
| 1 ¼″ bacteria tight | | |

TABLE 4
Equipment used for this study.

| Equipment | Description (optional) |
|---|---|
| Infusion Warmer BW 685 S | Warming system for infusion solutions |
| TubeFlow (AF365F) | Extention of infusion warmer system for tube |
| | between warmer and patient |
| Peristaltic pump | Infusomat Space; Braun; Typ 8713050 |
| Thermometer | Testo 112; CEMM86594 |

### 1. Introduction and Background

The facilitated 20% IGSC-project investigated a potential combination of PH20 with Cuvitru, which would lead to a significant benefit for patients by a reduction of the infusion volume and the infusion time in comparison to Immune Globulin Infusion 10% (Human) with Recombinant Human Hyaluronidase (HyQvia®). A major clinical aspect in that context is the achievable flow rate during infusion. First pre-clinical investigations have shown that due to the higher viscosity of the 20% IGG-product only 2.0 mL/min flow was considered feasible without creating an unacceptably high back pressure. This low flow rate would finally offset the effect of the infusion volume reduction; therefore, methods for decreasing the viscosity of the IgG solution were investigated. One potential approach was to increase the temperature of the infusion liquid, because at higher temperature the viscosity of the 20% IgG was shown to decrease.

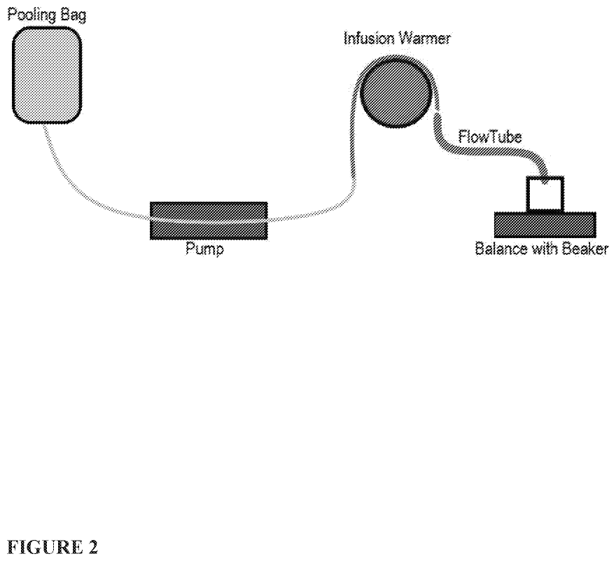

With reference to FIG. 2, the tubing between pooling bag and balance consisted of a tubing set with a spike (gray) connected with a 3.5 m long tube (red, thin), and a tube-24G-needle set (red, thick), the latter mantled with the Flow Tube device for maintaining the temperature after the IGG-solution passed the infusion warmer.

After assembling the infusion warmer setup, the experiments summarized in Table 5 were executed. For preparation of the necessary amount of the 20% IGG-solution for each of these experiments four 50-mL vials of 20% IGG-containers plus 10 mL of a fifth one were transferred with a 60-mL plastic syringe from the glass vials into the pooling bag. For this at first a ventilation needle was injected into the 50-mL container, afterwards the necessary volume (50 mL and 10 mL) was drawn with the 60-mL plastic syringe out of the vial and finally injected in the septum-port of the pooling bag. After pooling was complete, the bag was gently shaken for a short time for homogenization. The 10 mL starting sample was then directly taken via the septum port with a plastic syringe. The required adjustments of peristaltic pump and the infusion warmer were set and the experiment was started by the pump. When approximately 200 mL of the IGG-solution passed the infusion warmer system and was collected in an appropriate container, the run was stopped; from this material 10 mL were drawn, aliquoted according to analytical section Table 6 and immediately analyzed in case of MFI, DLS, visual appearance and turbidity; the aliquots for SEC were frozen≤−60° C.

TABLE 5
Experimental settings for In-Use Study of Infusion
Warmer with 20% IGG

| | | | | | Sampling points | |
|---|---|---|---|---|---|---|
| | | Flow | Temper- | | Pooling | |
| Experi- | Run | Rate | ature | Volume | bag before | Beaker after |
| ment | Order | [mL/min] | [° C.] | [mL]¹ | start [mL] | needle [mL] |
| 1 | 1 | 2.5 | 38 | 210 | 10 | 10 |
| 2 | 6 | 3.5 | 38 | 210 | 10 | 10 |
| 3 | 3 | 4.5 | 38 | 210 | 10 | 10 |
| 4 | 5 | 5.5 | 38 | 210 | 10 | 10 |
| 5 | 4 | 6.5 | 38 | 210 | 10 | 10 |
| 6 | 2 | 7.5 | 38 | 210 | 10 | 10 |

TABLE 6
Analytical tests and total sampling effort for In-Use Study of Infusion Warmer with 20% IGG

| | Sample | | | | Shipment/ |
|---|---|---|---|---|---|
| Test | volume | Matrix or | Test | | Temperature |
| Parameter | [mL] | Equivalent | Requirements | Expected Range | [° C.] |
| Visual | n.a. | n.a. | Information only | Complies (essentially free of | n.a. |
| appearance | | | | visible particles) | |
| MFI | 4.0 | n.a. | Information only | Report result | +2 to 8° C. |
| (Subvisible | | | | | |
| particles) | | | | | |
| SEC-HPLC | 0.4 | n.a. | Information only | IgG monomer + dimer: NLT 95% | ≤−60° C. |
| | | | | IgG polymer: NMT 2% | |
| | | | | IgG fragments: NMT 3% | |
| Turbidity (UV | 0.4 | n.a. | Information only | n.a. | +2 to 8° C. |
| at 350 nm) | | | | | |
| DLS | 0.4 | n.a. | Information only | n.a. | +2 to 8° C. |

The results of testing per Table 6 for the 20% IgG samples from the experiments in Table 5 were within expected ranges, and no increased turbidity or subvisible particles were found.

### Example 2

Example 2 describes an experiment measuring the viscosity of a IgGSC (20%) formulation under various conditions.

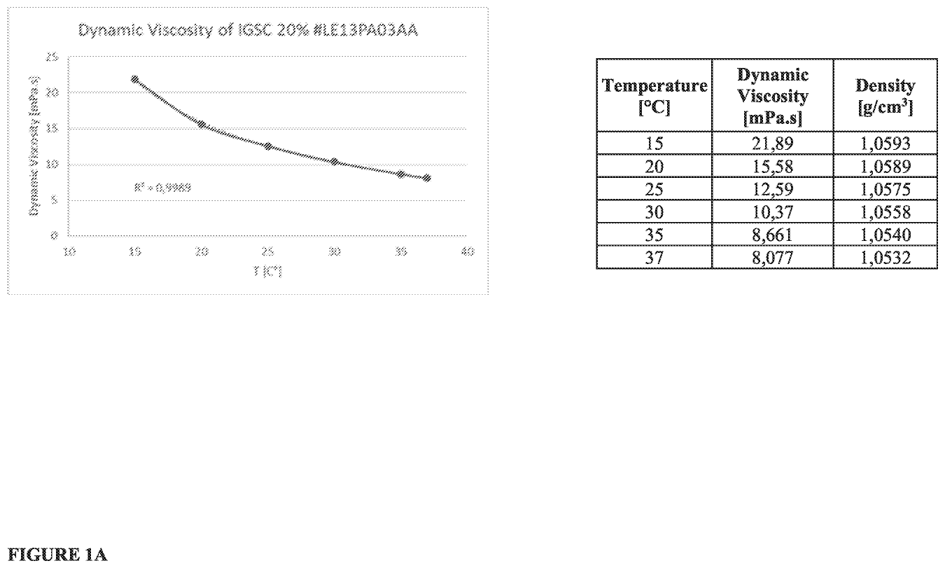

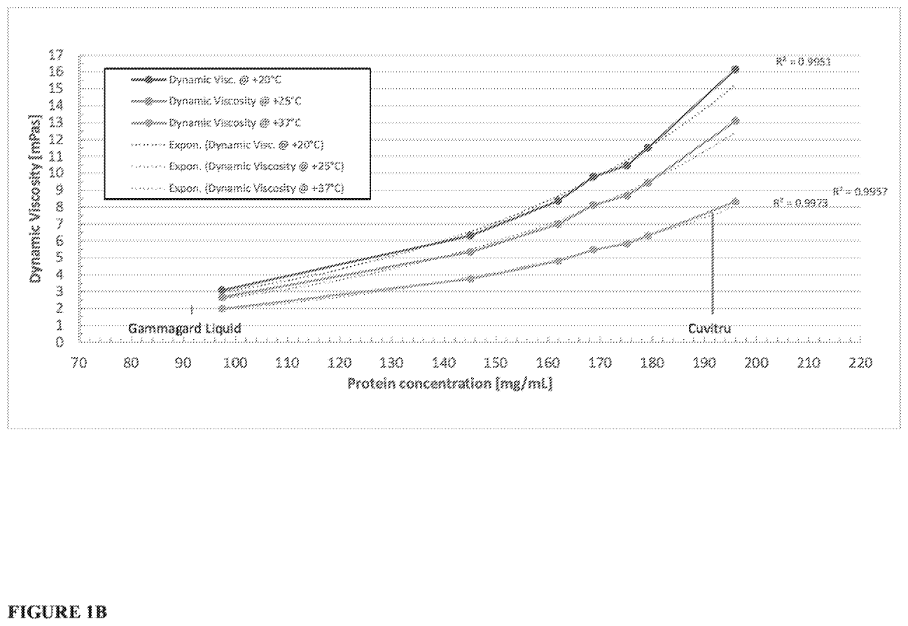

Around 1 mL of 20% IGG solution was transferred with a syringe to the rolling ball viscosimeter Lovis 2000 using a 1.59 mm diameter measurement capillary. A 1.5 mm steel ball was added to the capillary and the instrument was tempered to the required temperature. Afterwards the measurement was started. FIGS. 1A and 1B and Table 7.

TABLE 7
Dynamic Viscosity of IGSC, 20% at varying
temperatures.

| Temperature | Dynamic Viscosity |
|---|---|
| [° C.] | [mPa · s] |
| 15 | 21.89 |
| 20 | 15.58 |
| 25 | 12.59 |
| 30 | 10.37 |
| 35 | 8.661 |
| 37 | 8.077 |

### Example 3

A study was performed in pigs to assess the feasibility and local tolerability of SC infusion of warmed and facilitated IGSC, 20%. The specific objective was to compare the in-line pressure, bleb size, and local reactions at the administration sites after SC administration of facilitated and warmed IGSC, 20% versus conventional IGSC, 20%.

The IG solution was connected to the 3-way stopcock using a tube that passed through the warming device (Biegler GmbH, Model BW685) for the experimental conditions that required warming. The set point for the warming device was 41° C., targeting a physiological temperature at the needle. In one side of the 3-way stopcock, syringes with rHuPH20 or buffer were connected. On the other side of the 3-way stopcock, the pressure transducer for the registration of the in-line pressure was placed before the needle set. For the conditions with warming, a temperature sensor was placed between the pressure transducer and the needle.

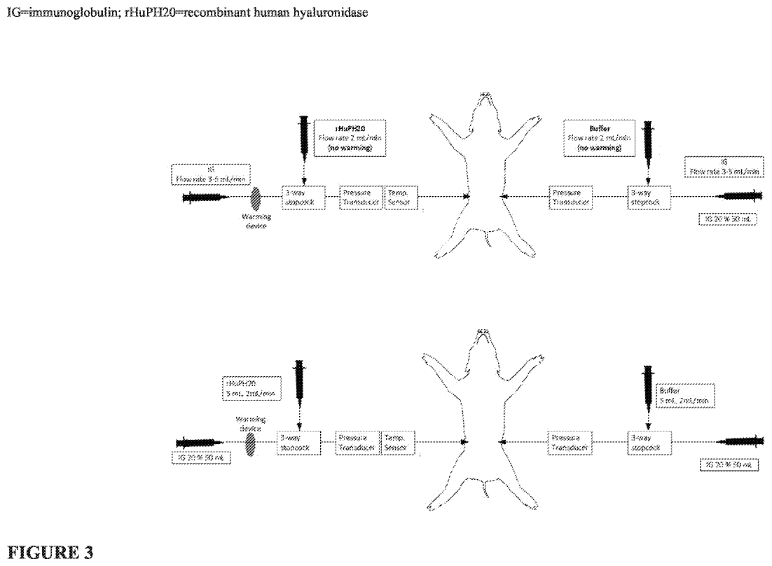

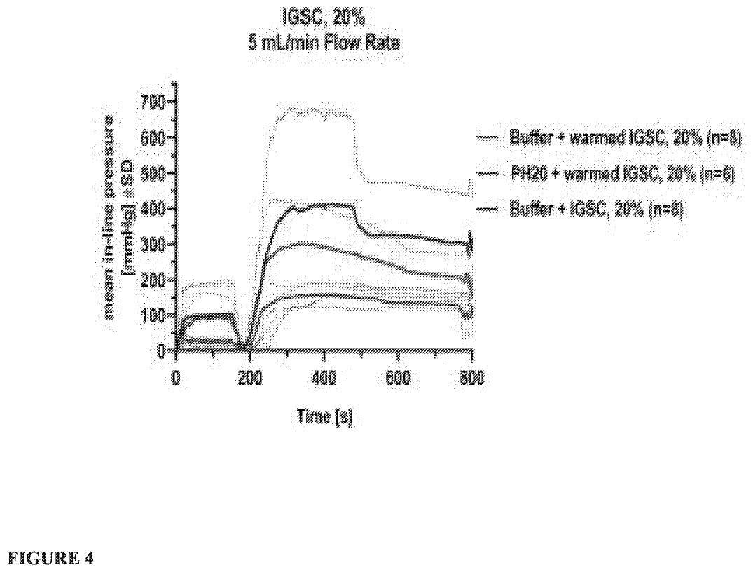

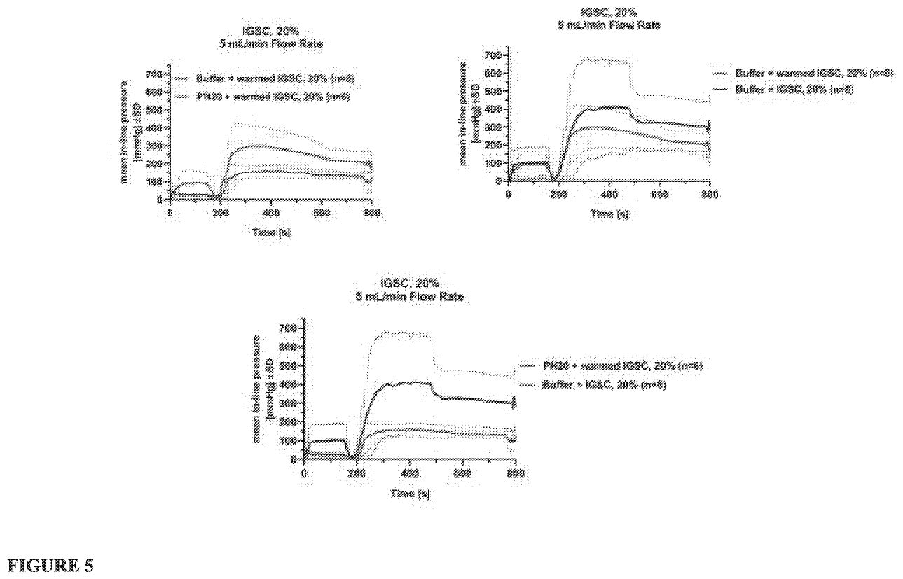

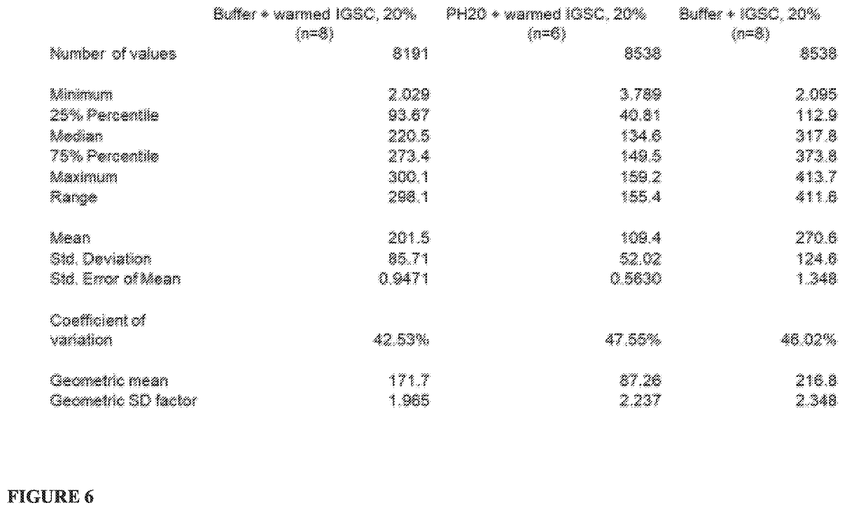

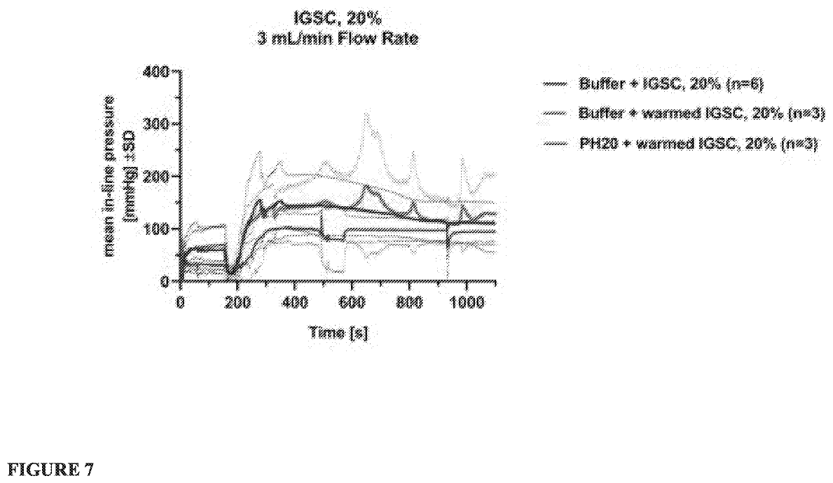

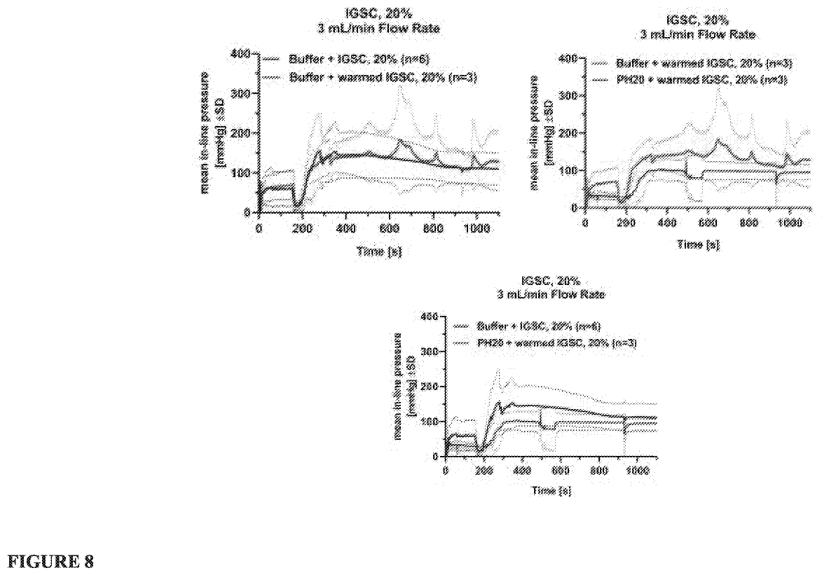

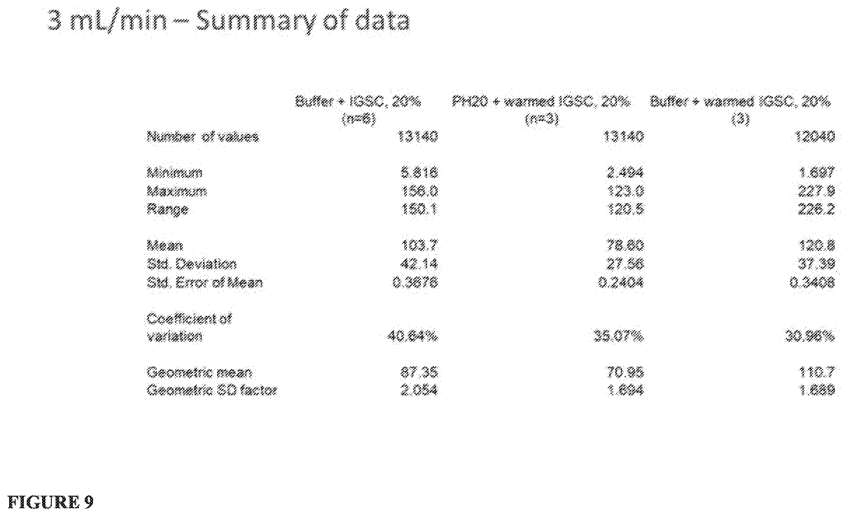

The rHuPH20/buffer filled the system passing by the opened side 3-way stopcock until the needle before animal puncture since this was the first administration. The rHuPH20 was diluted 1:1 with buffer in the case of IGI, 10% conditions to maintain a constant volume and the proper ratio of enzyme to IG (80 U rHuPH20/g IG). After finishing the administration of rHuPH20/buffer, the 3-way stopcock was opened to the other side to perform the administration of the IG solution. The study set-up is presented in FIG. 3.

Three animals per group received two simultaneous SC infusions in the abdominal region at flow rates of 3, 4, or 5 mL/min. For each individual pig, on one side, 5 mL of rHuPH20 followed by 50 mL of warmed IGSC, 20% were sequentially delivered (up to a maximum temperature of 32° C. to 36° C., measured shortly before the needle), and on the contralateral side, 5 mL of buffer followed by 50 mL of non-warmed IGSC, 20% were delivered.

The different treatment approaches are outlined in Table 8.

TABLE 8
Overview of Treatment Approaches and Comparisons.

| | Treatment | | Facilitation | | IG | | | |
|---|---|---|---|---|---|---|---|---|
| | Approach | Number | | Dose (U)/ | | V | | Flow Rate |
| Comparison | (IG Product) | of Pigs | Item | Volume (mL)^{a} | [C] | (mL) | Warm | (mL/min) |
| 1: TI1 vs TI2 | TI1 (CUVITRU) | 9 | Buffer | 0/5 | 20% | 50 | No | 3, 4, 5 |
| | TI2 (warmed | (3/flow | rHuPH20 | 800/5 | 20% | 50 | Yes | |
| | and | rate) | | | | | | |
| | facilitated | | | | | | | |
| | IGSC, 20%) | | | | | | | |
| | Condition B | | rHuPH20 | 800/5 | 20% | 50 | Yes | |
| | (warmed | | | | | | | |
| | and | | | | | | | |
| | facilitated | | | | | | | |
| | IGSC, 20%) | | | | | | | |

IG = immunoglobulin;
rHuPH20 = recombinant human hyaluronidase;
vs = versus
^{a}The ratio of rHuPH20 was kept the same as for the licensed product HYQVIA, which is approximately 80 U rHuPH20 per gram immunoglobulin. rHuPH20/buffer was infused at a flow rate of 2 mL/min.

In-line pressure was assessed during the entire infusion period and curves for in-line pressure revealed from a PowerLab chart. For each treatment, the mean curves for the in-line pressures during infusion were plotted using GraphPad Prism, Version 8.02. Mean and maximum in-line pressure during IG infusion (beginning from the start of the IG infusion until end of infusion) were determined and compared between treatments using an unpaired T-test.

The temperature of the IGSC, 20% component in warmed and facilitated IGSC, 20% was also measured in-line.

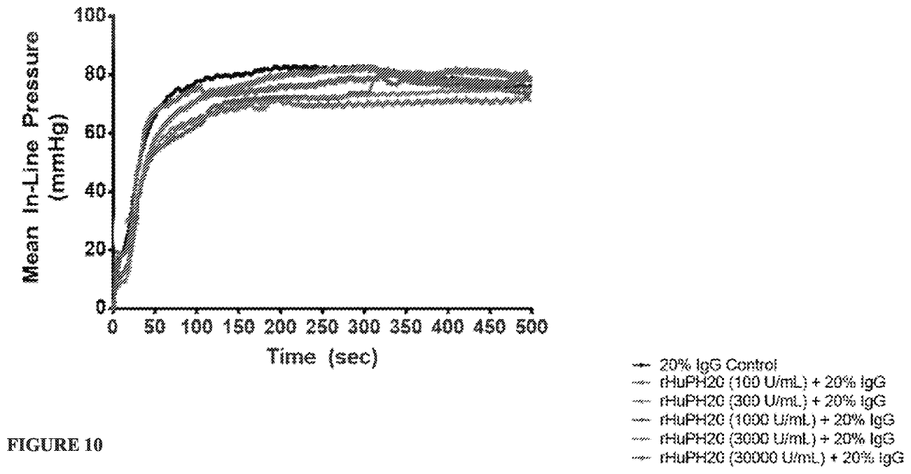

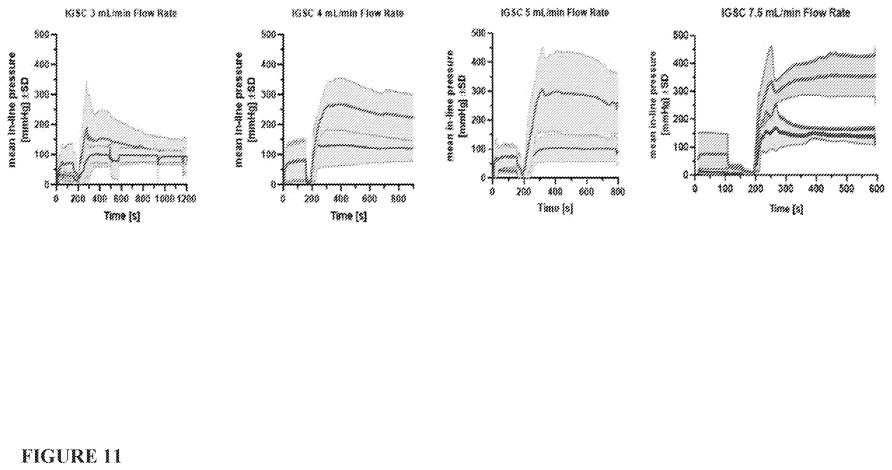

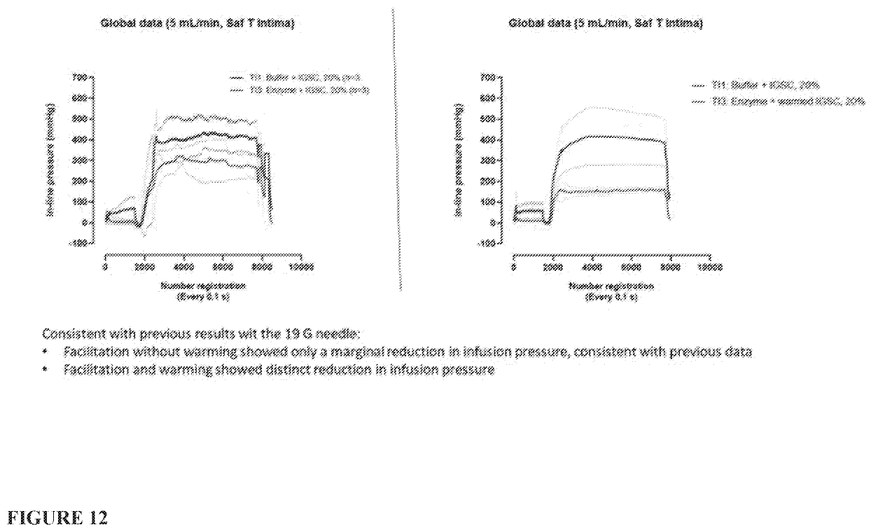

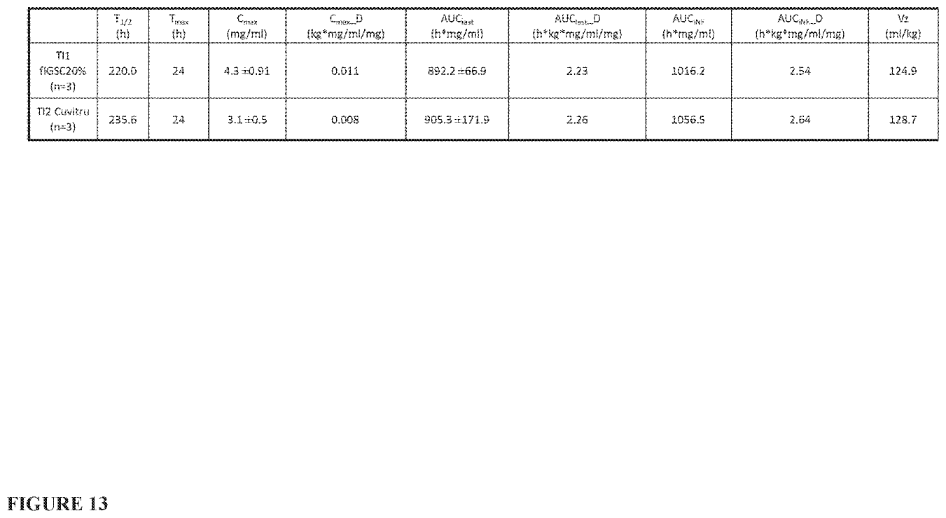

The SC administration of 50 mL of IGSC, 20% at flow rates of 3, 4, or 5 mL/min showed a distinct reduction in the in-line pressures with warmed and facilitated IGSC, 20% compared to conventional IGSC, 20%. In FIG. 10, the results for the in-line pressures during infusion are shown. A summary of the data and the descriptive statistics are presented in Table 9, Table 10, and Table 11 and the statistical comparison in Table 12.

TABLE 9
Descriptive Statistics for In-line Pressure During Immunoglobulin Infusion for 3 mL/min Flow Rate

| Flow Rate | 3 mL/min (immunoglobulin infusion) | | | | | |
|---|---|---|---|---|---|---|
| Treatment | IGSC, 20% | | | warmed and facilitated IGSC, 20% | | |
| Animal ID | S1717 | S1725 | S1728 | S1717 | S1725 | S1728 |
| Temperature | NA | NA | NA | 27.56-35.95 | 26.66-35.62 | 27.90-35.53 |
| Range (° C.) | | | | | | |
| Maximum In-line | 101.0 | 313.0 | 70.4 | 119.0 | 136.0 | 119.2 |
| Pressure (mmHg) | | | | | | |
| In-line Pressure | 89.5 ± 16.8 | 151.5 ± 62.0 | 64.8 ± 9.7 | 110.9 ± 18.7 | 61.2 ± 23.2 | 106.3 ± 15.2 |
| (mmHg) ± SD | | | | | | |
| Mean Maximum | 161.5 ± 76.3 | | | 124.7 ± 5.6 | | |
| In-line Pressure | | | | | | |
| (mmHg) ± SEM | | | | | | |
| Mean In-line | 101.9 ± 25.8 | | | 92.8 ± 15.9 | | |
| Pressure | | | | | | |
| (mmHg) ± SEM | | | | | | |

ID = identification;
IGSC, 20% = Immune Globulin Infusion, 20% (human) subcutaneous;
NA = not applicable;
SD = standard deviation;
SEM = standard error of the mean

TABLE 10
Descriptive Statistics for In-line Pressure During Immunoglobulin Infusion for 4 mL/min Flow Rate.

| Flow Rate | 4 mL/min (immunoglobulin infusion) | | | | | |
|---|---|---|---|---|---|---|
| Treatment | IGSC, 20% | | | warmed and facilitated IGSC, 20% | | |
| Animal ID | S1732 | S1741 | S1749 | S1732 | S1741 | S1749 |
| Temperature | NA | NA | NA | 22.08-31.66 | 23.35-31.91 | 22.21-32.00 |
| Range (° C.) | | | | | | |
| Maximum In-line | 301.7 | 179.7 | 339.5 | 95.84 | 101.2 | 241.3 |
| Pressure (mmHg) | | | | | | |
| In-line Pressure | 255.9 ± 50.2 | 152.3 ± 23.2 | 295.6 ± 45.4 | 87.5 ± 13.8 | 94.2 ± 13.1 | 186.1 ± 29.4 |
| (mmHg) ± SD | | | | | | |
| Mean Maximum | 273.6 ± 48.2 | | | 146.1 ± 47.6 | | |
| In-line Pressure | | | | | | |
| (mmHg) ± SEM | | | | | | |
| Mean In-line | 234.6 ± 42.7 | | | 122.6 ± 31.8 | | |
| Pressure | | | | | | |
| (mmHg) ± SEM | | | | | | |

ID = identification;
IGSC, 20% = Immune Globulin Infusion, 20% (human) subcutaneous;
NA = not applicable;
SD = standard deviation;
SEM = standard error of the mean

TABLE 11
Descriptive Statistics for In-line Pressure During Immunoglobulin Infusion for 5 ml/min Flow Rate

| Flow Rate | 5 mL/min (immunoglobulin infusion) | | | | | |
|---|---|---|---|---|---|---|
| Treatment | IGSC, 20% | | | warmed and facilitated IGSC, 20% | | |
| Animal ID | S1719 | S1722 | S1723 | S1719 | S1722 | S1723 |
| Temperature | NA | NA | NA | 24.65-34.53 | 25.06-34.86 | 25.10-35.24 |
| Range (° C.) | | | | | | |
| Maximum In-line | 304.7 | 264.2 | 472.5 | 152.7 | 115.0 | 149.4 |
| Pressure (mmHg) | | | | | | |
| In-line Pressure | 264.1 ± 53.6 | 219.6 ± 30.8 | 413.1 ± 62.1 | 135.4 ± 20.4 | 107.1 ± 15.2 | 139.5 ± 18.2 |
| (mmHg) ± SD | | | | | | |
| Mean Maximum | 347.1 ± 63.8 | | | 139.0 ± 12.1 | | |
| In-line Pressure | | | | | | |
| (mmHg) ± SEM | | | | | | |
| Mean In-line | 298.9 ± 58.5 | | | 127.3 ± 10.2 | | |
| Pressure | | | | | | |
| (mmHg) ± SEM | | | | | | |

ID = identification;
IGSC, 20% = Immune Globulin Infusion, 20% (human) subcutaneous;
NA = not applicable;
SD = standard deviation;
SEM = standard error of the mean

TABLE 12
Comparison of Mean and Maximum In-line Pressure
Between Treatments for 3, 4, and 5 ml/min Flow Rate.

| | | |
|---|---|---|
| IGSC, 20% vs warmed and facilitated | | |
| IGSC, 20% | Significant | p-value |

Mean Maximum In-line Pressure

| | | |
|---|---|---|
| 3 mL/min | No | 0.656 |
| 4 mL/min | No | 0.133 |
| 5 ml/min | Yes | 0.033 |

Mean In-line Pressure

| | | |
|---|---|---|
| 3 mL/min | No | 0.778 |
| 4 mL/min | No | 0.103 |
| 5 mL/min | Yes | 0.045 |

IGSC, 20% = Immune Globulin Infusion, 20% (human) subcutaneous;
vs = versus

The statistical analysis indicates that these differences are significant for the 5 mL/min flow rates. Generally: a flow rate-dependent increase in mean and maximum in-line pressure was observed with conventional IGSC, 20%, whereas with warmed and facilitated IGSC, 20%, in-line pressure remained at the low level up to the highest flow rate tested.

Additional experiments using the set-up as described above were performed comparing conventional IGSC, 20% and warmed IGSC, 20% without facilitation. Three animals per group received two simultaneous SC infusions in the abdominal region at flow rates of 3, or 5 mL/min. For each individual pig, on one side, 5 mL of buffer followed by 50 mL of warmed IGSC, 20% were sequentially delivered (up to a maximum temperature of 32° C. to 36° C., measured shortly before the needle), and on the contralateral side, 5 mL of buffer followed by 50 mL of non-warmed IGSC, 20% were delivered. In-line pressure during infusion was comparable at 3 ml/min, however at 5 ml/min was reduced with warming as compared to non-warming.

### Example 4

A PK study was conducted in female pigs to compare the PK properties of human IG following 3 different single SC infusion approaches of IGSC, 20%. Pigs in the respective groups (n=3/group) received IGSC, 20% as follows:
1) Group 1: In-line warmed IGSC, 20% facilitated with rHuPH20
2) Group 2: IGSC, 20% without rHuPH20 (IGSC, 20%)
3) Group 3: In-line warmed IGSC, 20% without rHuPH20 (warmed IGSC, 20%)

After SC administration of rHuPH20 (Group 1:79.5 U rHuPH20 per gram IG; infusion speed: 2 mL/min) or buffer for rHuPH20 (Group 2, Group 3), the pigs received 400 mg/kg IGSC, 20% by a single SC infusion of IGSC, 20% in-line warmed (infusion speed: 5 mL/min) (Group 1, Group 3) or at room temperature (infusion speed: 1 mL/min) (Group 2).

Whole blood samples from all animals were collected over 28 days at 0.5, 1, 3, 6, 12, 24, 48, 72, 96,120,168, 216, 288, 336, 408, 480, 576, and 672 hours post-infusion. Serum was prepared from the whole blood samples for analysis of human IgG using an ELISA for total human IgG concentrations. Pharmacokinetic parameters were determined based upon individual pharmacokinetic profiles using a noncompartmental model with WinNonlin® (Certara, Princeton, NJ).

Subcutaneous infusion without in-line warming of the IGSC, 20% solution and without rHuPH20 pretreatment (Group 2), led to mean±standard deviation Cₘₐₓ of 3.8±0.7 mg/mL and was achieved after 40±13.9 hours. The infusion resulted in a mean ty of 192.3±5.6 hours and a mean area under the concentration-time curve from time 0 to the last quantifiable concentration (AUC₀₋ₜ) of 775.1±266.5 hr·mg/mL. Following SC administration of TAK-881 (Group 1) and without rHuPH20 (warmed IGSC, 20%; Group 3), the mean Cₘₐₓ was 4.9±1.5 mg/mL for Group 1 and 3.6±1.5 mg/mL for Group 3 and was achieved after 48±24 hours and 40±13.9 hours, respectively. The in-line warmed IGSC, 20% approach (Group 3) revealed an apparent t_{1/2}, of 218.4±57.2 hours and an AUC₀₋ₜ of 759.7±239.0 hr-mg/mL, whereas the presence of rHuPH20 resulted in ty of 183.7±19.1 hours and an AUC₀₋ₜ of 813.8±109.1 hr·mg/ml (Table 13).

The pooled PK profiles are presented in Table 13, confirming the trend to an increased Cₘₐₓ with warmed and facilitated IGSC, 20%.

TABLE 13
Pharmacokinetics in Pigs after Subcutaneous Administration.

| | Mean (±Standard Deviation) | | |
|---|---|---|---|
| | Group 1 (TI1) | Group 2 | Group 3 (TI3) |
| | Warmed and | (TI2) | Warmed |
| Parameter | facilitated IGSC, 20% | IGSC, 20% | IGSC, 20% |
| Flow rate (mL/min) | 5 | 1 | 5 |
| Number of Animals | 3 | 3 | 3 |
| Cₘₐₓ (mg/mL) | 4.9 ± 1.5 | 3.8 ± 0.7 | 3.6 ± 1.5 |
| Tₘₐₓ (hr) | 48 ± 24.0 | 40 ± 13.9 | 40 ± 13.9 |
| t_{1/2} (hr) | 183.7 ± 19.1 | 192.3 ± 5.6 | 218.4 ± 57.2 |
| AUC₀₋ₜ (mg·hr/mL) | 813.8 ± 109.1 | 775.1 ± 266.5 | 759.7 ± 239.0 |

AUC₀₋ₜ = area under the concentration-time curve from time 0 to the last quantifiable concentration;
Cₘₐₓ = maximum concentration;
IGSC, 20% = Immune Globulin Infusion, 20% (human) subcutaneous;
t½ = half-life;
Tₘₐₓ=time to reach maximum concentration

### Example 5

A Phase I human clinical trial was designed, conducted, and evaluated.

### 1. Background

TAK-881, Immune Globulin Subcutaneous (Human), 20% Solution (IGSC, 20%) with Recombinant Human Hyaluronidase (rHuPH20) is a facilitated subcutaneous immune globulin (IG).

HyQvia®, Immune Globulin Infusion 10% (Human) with Recombinant Human Hyaluronidase, and CUVITRU®, Immune Globulin Subcutaneous (Human), 20% Solution. TAK-881 differs from HyQvia® only by the use of warmed or unwarmed (room temperature) IGSC, 20% (CUVITRU®, Immune Globulin Subcutaneous (Human), 20% Solution) instead of immune globulin infusion (IGI), 10% at room temperature.

TAK-881 is administered by sequential subcutaneous (SC) infusion of rHuPH20 first, followed immediately (within 10 minutes) by warmed or room temperature IGSC, 20%. The ratio of rHuPH20 to IG is the same as for HyQvia®, Immune Globulin Infusion 10% (Human) with Recombinant Human Hyaluronidase, which is 80 U rHuPH20 per gram of IG.

Given the doubled IG concentration delivered with TAK-881, the required infusion volume would be reduced by 50% at an equivalent dose level as compared to HYQVIA. Reduced volumes potentially lead to improved tolerability due to fewer local site reactions with better patient outcomes.

However, the higher IG concentration, as with CUVITRU® [Immune Globulin Subcutaneous (Human), 20% Solution], is associated with higher viscosity, increasing in-line pressure, which only allow lower infusion rates in conventional subcutaneous IgG, 20% therapy.

Dynamic viscosity is inversely proportional to temperature. In liquids, viscous forces are caused by molecules exerting attractive forces on each other and increasing temperature results in a decrease in viscosity as particles gain greater thermal energy and can overcome the attractive forces binding them together.

Therefore, to decrease viscosity, a commercially available in-line warming device is used for TAK-881 (for Treatment Arms 1 and 2 only, in this study) to warm the infusion tubing used to infuse the IGSC, 20% component. In Treatment Arm 3, no in-line warming device was used for TAK-881, and the IGSC, 20% component was administered at room temperature.

By using either decreased viscosity IGSC, 20% with warming and hyaluronidase, or standard viscosity IGSC, 20% at room temperature with hyaluronidase, TAK-881 may allow for faster infusion time as compared to CUVITRU® [Immune Globulin Subcutaneous (Human), 20% Solution] (currently up to 1 mL/min), and lower SC infusion volume with associated shorter infusion time as compared to HYQVIA.

The IGSC, 20% component of TAK-881 is a liquid immunoglobulin G (IgG) product purified from human plasma marketed as CUVITRU® [Immune Globulin Subcutaneous (Human), 20% Solution]. The IgG subclass distribution for the final product is within the normal range for human serum and comprises antibodies to specific bacterial and viral pathogens. The preparation retains all Fab and Fc mediated functions of the IgG molecule.

The rHuPH20 component of TAK-881 is a highly purified, recombinant human hyaluronidase that de-polymerizes the gel-like hyaluronan in local SC tissue where it is infused. This localized effect results in a transient increase in permeability, allowing IGI to disperse and to reach the systemic circulation more ready than without rHuPH20.

Extensive safety data are available for the individual components of TAK-881, rHuPH20 and IGSC, 20%, based on the safety profiles of the approved products, HyQvia®, Immune Globulin Infusion 10% (Human) with Recombinant Human Hyaluronidase and CUVITRU® [Immune Globulin Subcutaneous (Human), 20% Solution]. Additionally, it was demonstrated through analytical and preclinical studies that the warming of IGSC, 20% did not influence critical quality parameters and local tolerability and that warming and facilitation of IGSC, 20% allows higher flow rates than currently used for CUVITRU® [Immune Globulin Subcutaneous (Human), 20% Solution]. Finally, the combination of rHuPH20 and warmed IGSC, 20% was well tolerated in preclinical studies, thereby supporting proof of concept clinical trials.

### 2. Protocol Summary

Protocol number: TAK-881-1001.
Drug: TAK-881-Immune Globulin Subcutaneous (Human), 20% Solution (abbreviated as IGSC, 20%) with Recombinant Human Hyaluronidase (abbreviated as rHuPH20).
Title of the study: A Phase I, Single-Dose, Single-Center, Open-Label, Three-Arm Study to Assess the Tolerability of Safety of Immune Globulin Subcutaneous (Human), 20% Solution with Recombinant Human Hyaluronidase (TAK-881) at Various Infusion Rates in Healthy Adult Subjects.
Number of subjects (total and for each treatment arm): Total sample size for this study is 24 subjects with 8 subjects enrolled/treated in each of the 3 treatment arms.
Site(s) and Region(s): Single site, USA.
Study Subject Population: Healthy male and female subjects aged 19 to 50 years (inclusive) at the time of consent and body mass index (BMI) between 18.0 and 30.0 kg/m² (inclusive) at screening. This study enrolled 8 subjects in each of the 3 treatment arms, and a minimum of 3 subjects in each of the 2 BMI groups (18.0 to <25.0 kg/m², ≥25.0 to 30.0 kg/m²) in each treatment arm.

Inclusion Criteria: Must be considered “healthy.” Healthy as determined by the investigator on the basis of screening evaluations. Healthy status is defined by absence of evidence of any active or chronic disease following a detailed medical and surgical history, a complete physical examination including vital signs, 12-lead ECG, hematology, blood chemistry, and urinalysis. BMI between 18.0 and 30.0 kg/m² inclusive.

### Objectives

1) Primary: To assess the tolerability of TAK-881 at various subcutaneous (SC) infusion rates in healthy adult subjects.
2) Secondary: To assess the safety of TAK-881 at various SC infusion rates and immunogenicity of TAK-881 in healthy adult subjects.
3) Exploratory: To assess serum total immunoglobulin G (IgG) levels.

Rationale: TAK-881 (IGSC, 20% solution with rHuPH20) is a facilitated immune globulin subcutaneous (IGSC) infusion evolved from HyQvia®, Immune Globulin Infusion 10% (Human) with Recombinant Human Hyaluronidase and CUVITRU® [Immune Globulin Subcutaneous (Human), 20% Solution]. Both HyQvia®, Immune Globulin Infusion 10% (Human) with Recombinant Human Hyaluronidase and CUVITRU® [Immune Globulin Subcutaneous (Human), 20% Solution] have very well-established efficacy and safety data. The higher concentration of TAK-881 (IGSC 20%) in comparison with HyQvia®, Immune Globulin Infusion 10% (Human) with Recombinant Human Hyaluronidase has the potential of reducing infusion volumes by 50%, decreasing infusion time, and potentially leading to improved tolerability. This Phase I study was conducted to assess the tolerability, safety, and immunogenicity of TAK-881 at various SC infusion rates in healthy adult subjects with a focus on evaluating key dosing and administration parameters to support further clinical development.

### 3. Study Endpoints

Primary Endpoint: The primary endpoint corresponding to the primary objective of the study was the occurrence of tolerability related to the infusion of TAK-881 per infusion site. A tolerability event is considered to have occurred if an infusion was tolerable. An infusion is considered tolerable if the infusion rate was not reduced or the infusion was not interrupted or stopped, due to any treatment-emergent adverse event (TEAE) related to TAK-881.

### Secondary Endpoints:

1) Safety and immunogenicity endpoints (i.e., occurrence of TEAEs, including but not limited to TAK-881-related and non-related TEAEs; clinical laboratory parameters; vital signs; immunogenicity, e.g., occurrence of binding and neutralizing antibodies to rHuPH20).
2) SC administration endpoints (i.e., supportive tolerability and safety measures: maximum tolerable infusion rate achieved per infusion site; total volume infused per infusion site: time to deliver the total infused volume per infusion site).

Exploratory Endpoint: Serum total IgG levels at predose and postdose of TAK-881 SC administration.

### 4. Study Design

### 4.1 Study Design

This study was a Phase I, single-dose, single-center, open-label, three-arm study to evaluate the tolerability, safety, and immunogenicity of TAK-881 at various infusion rates in healthy adult subjects.

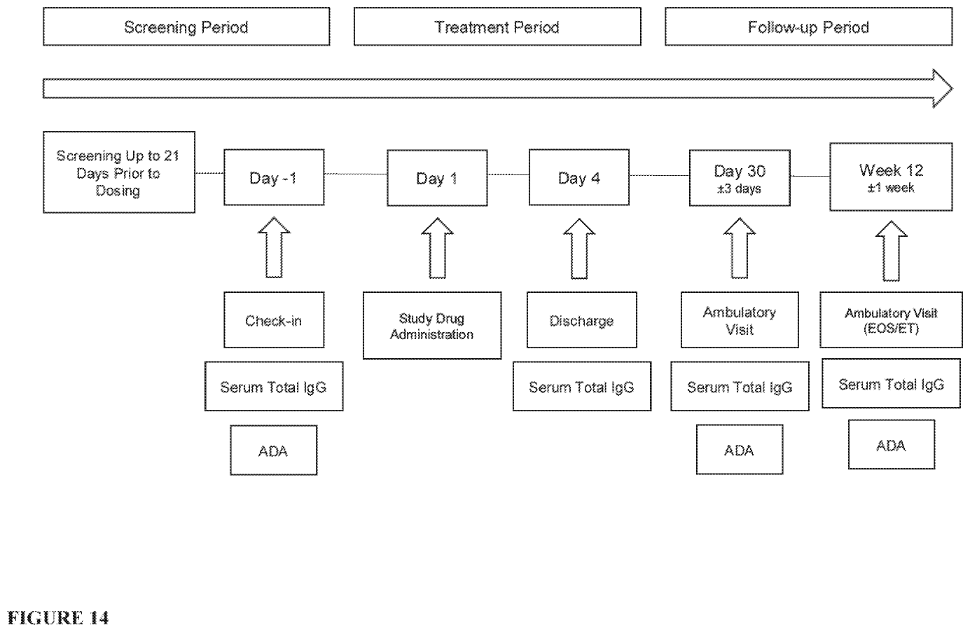

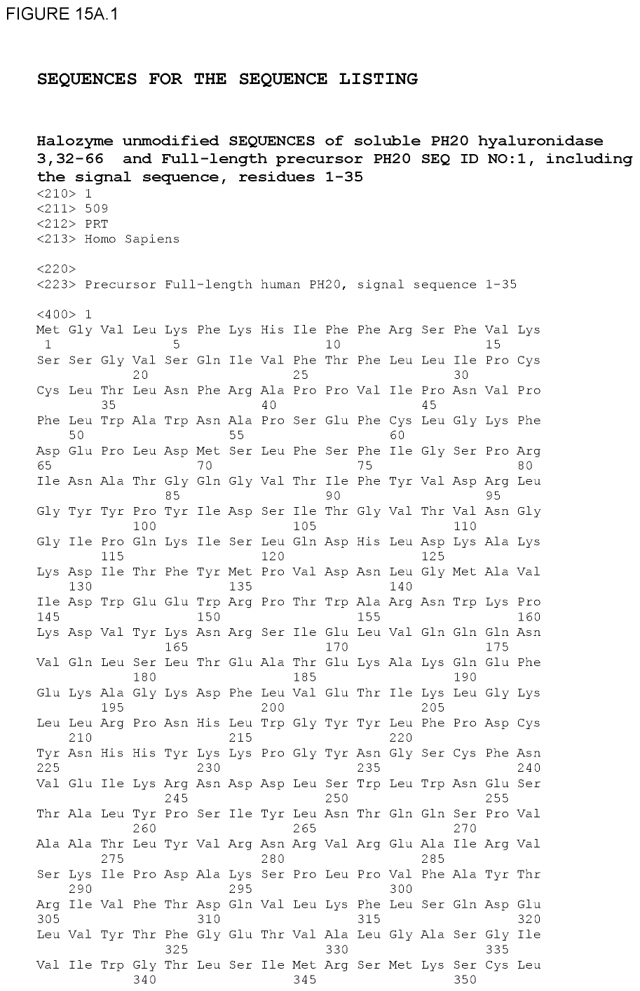

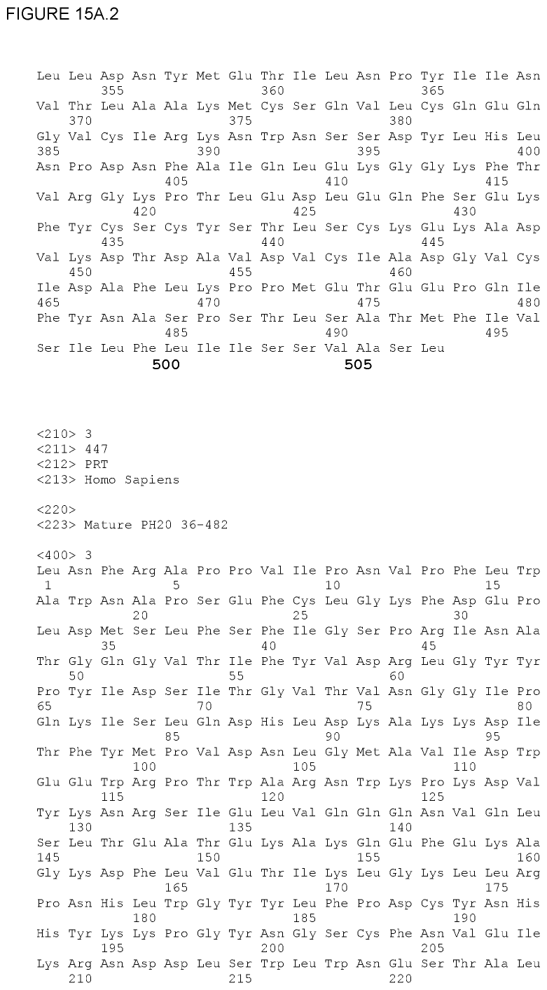

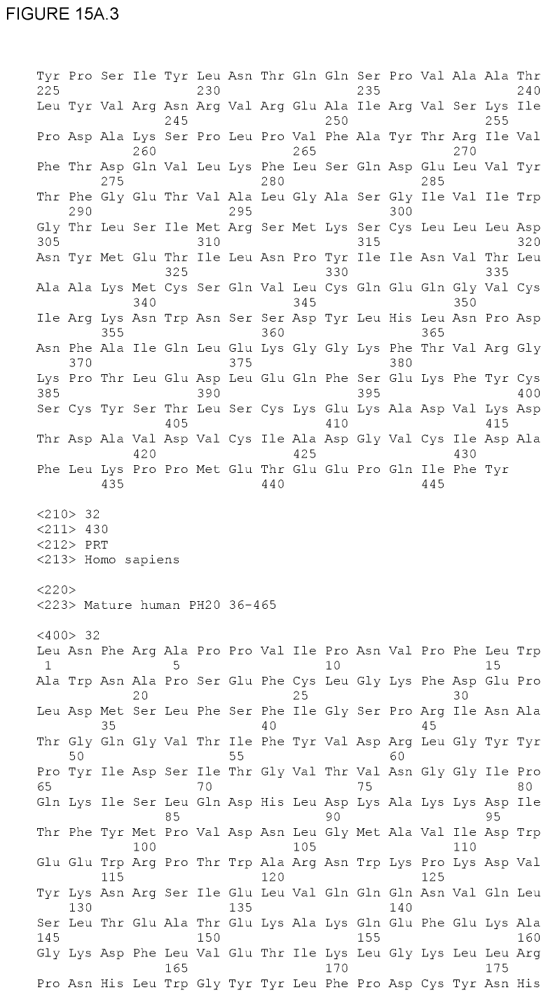

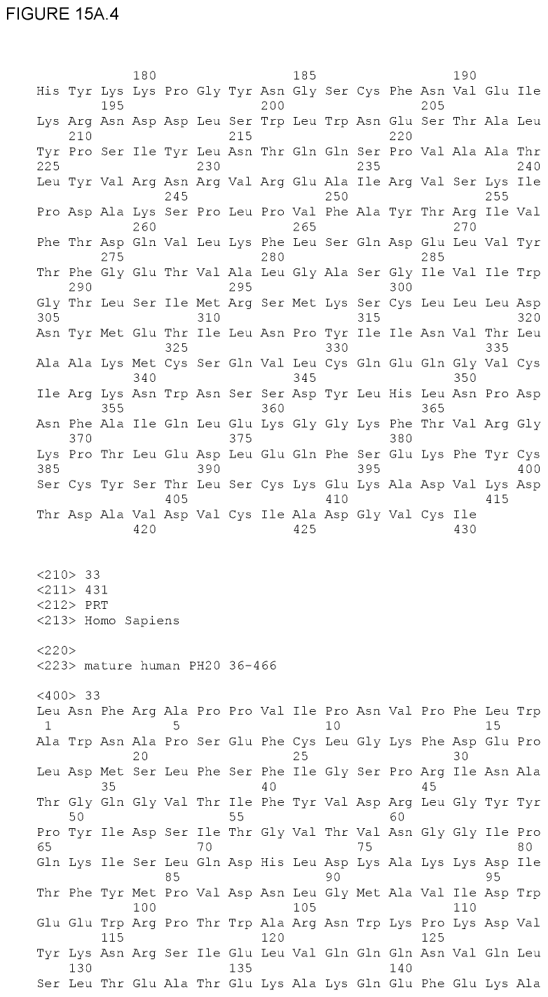

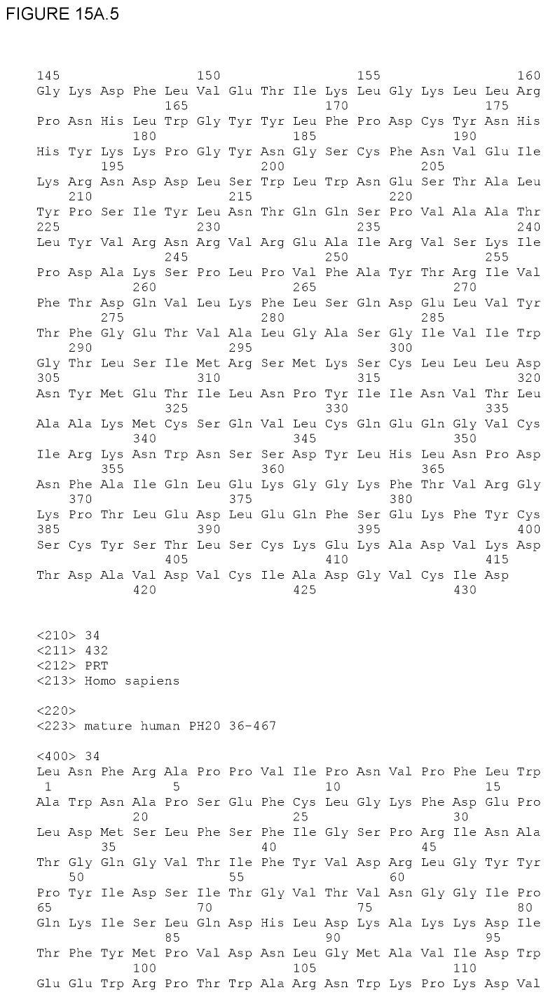

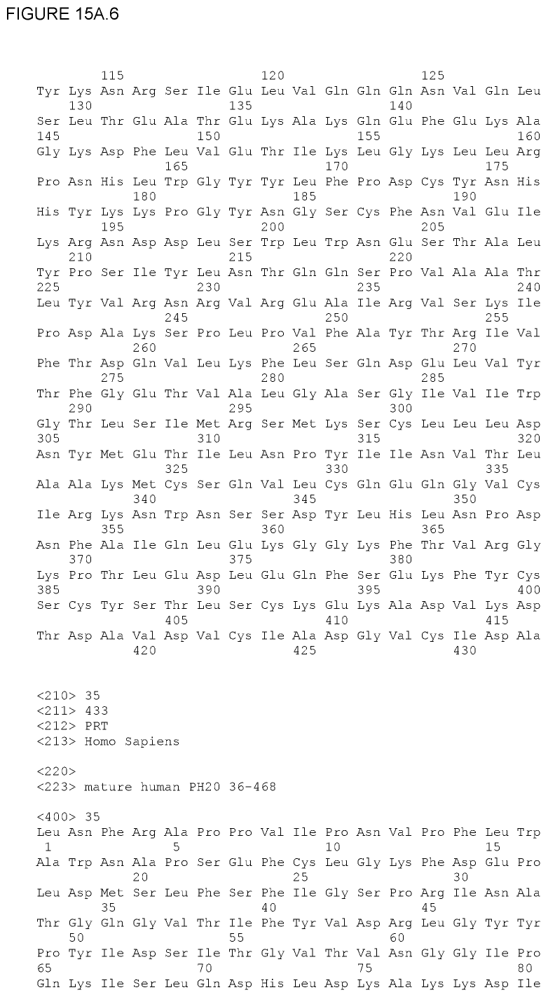

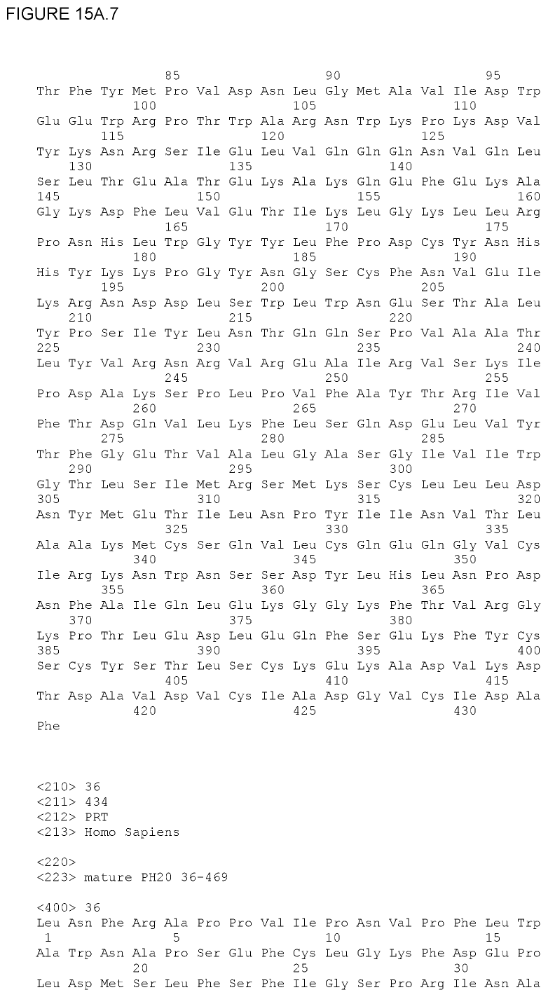

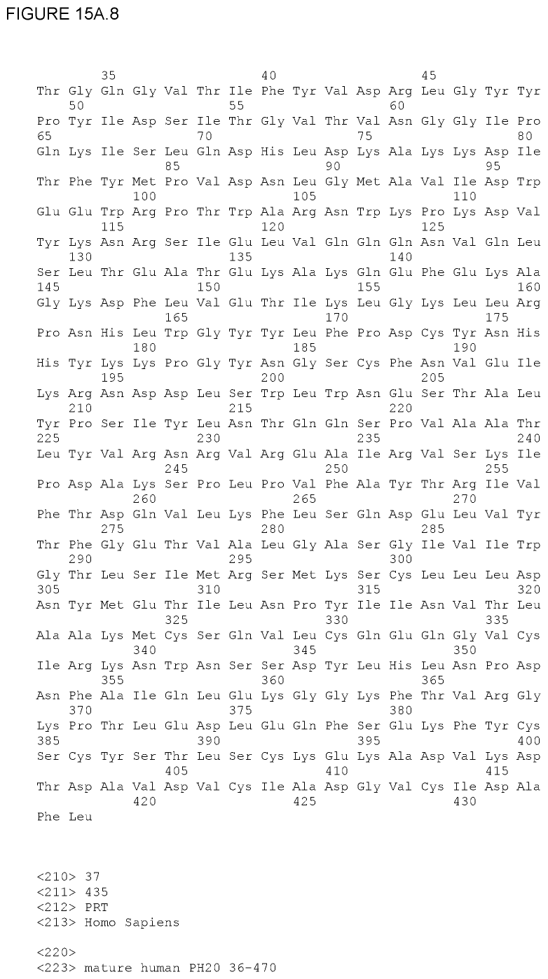

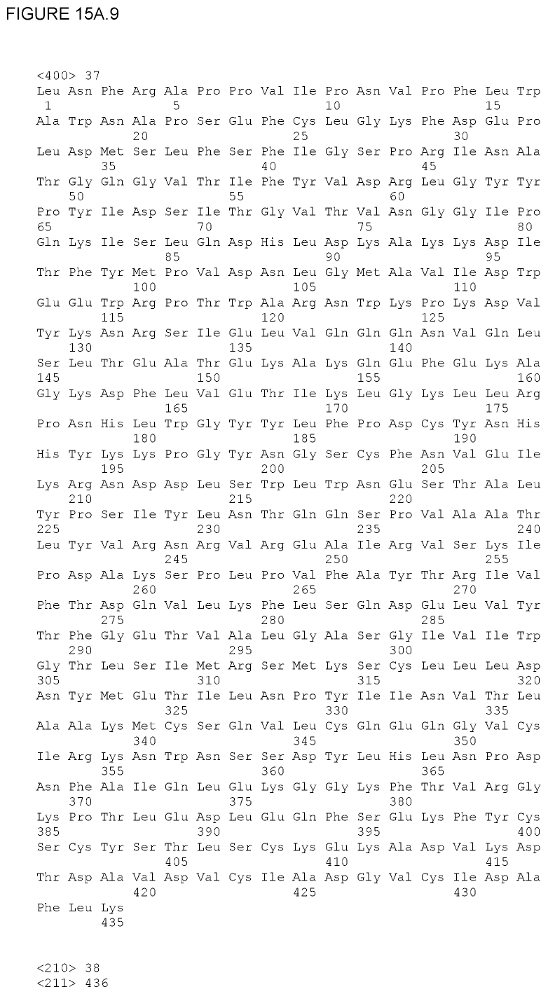

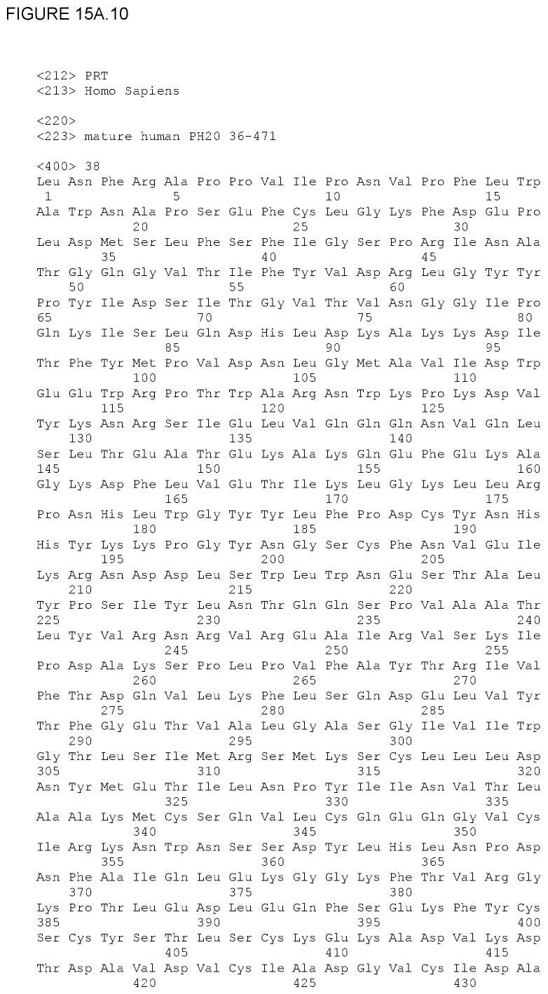

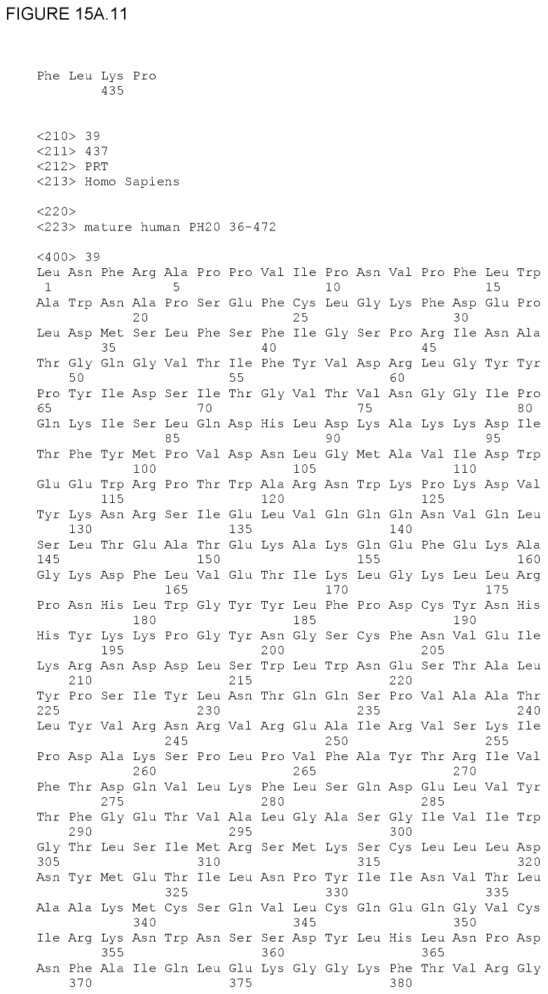

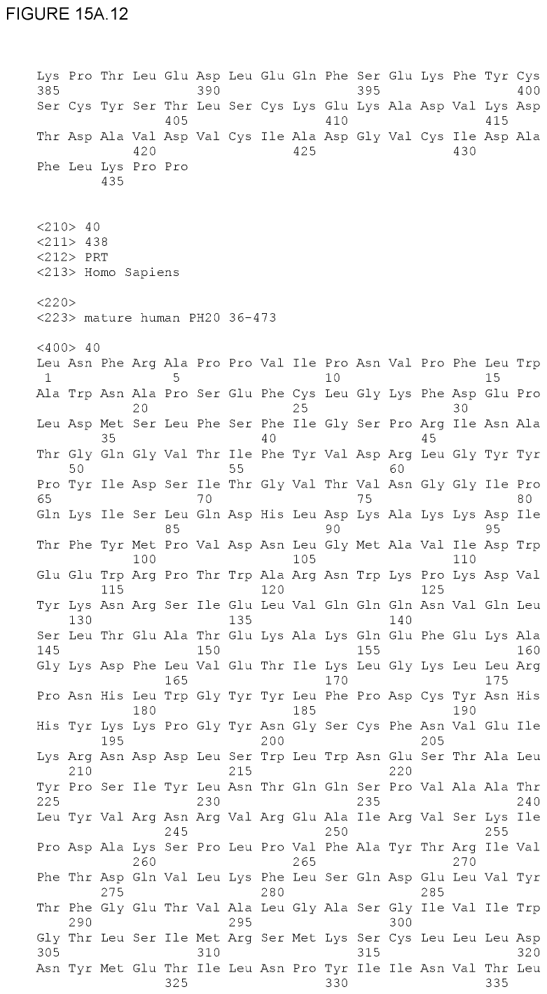

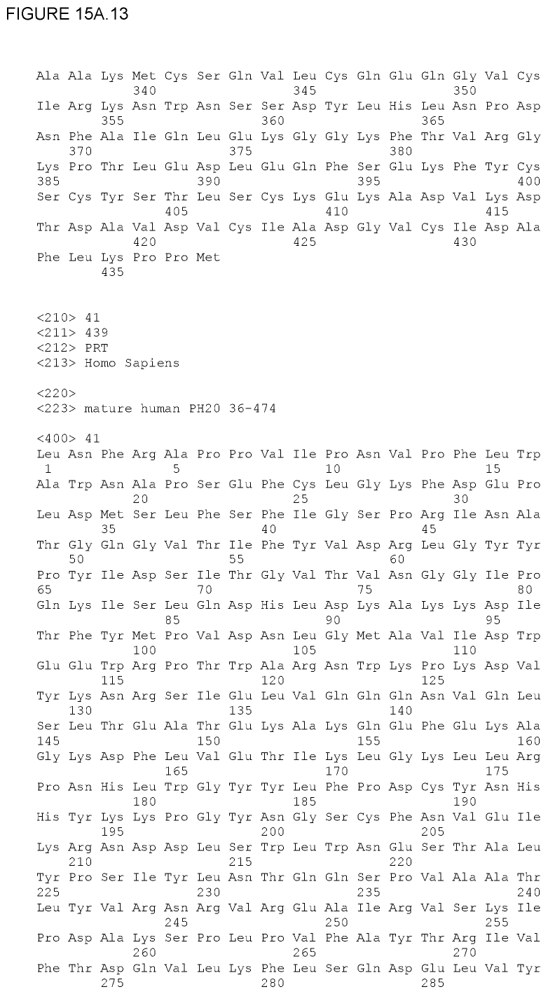

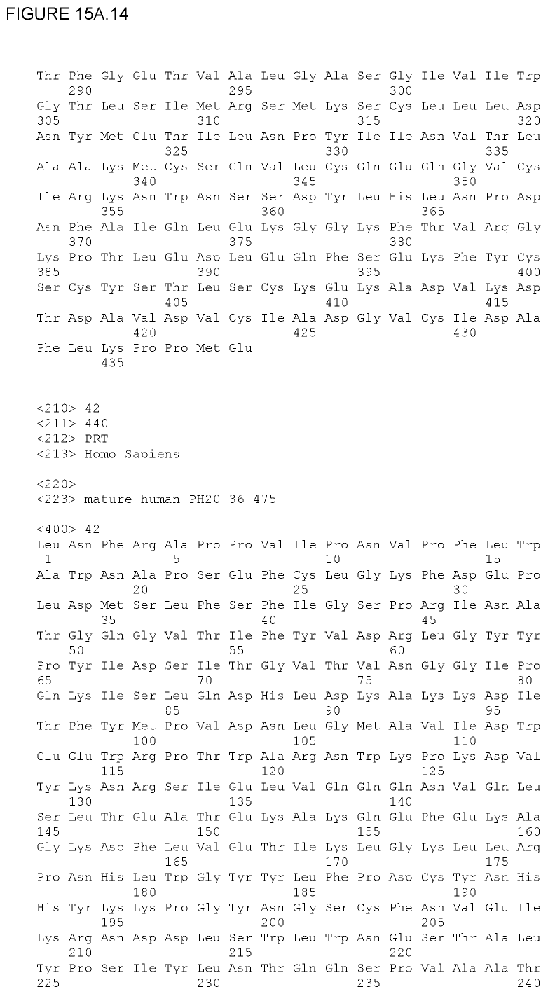

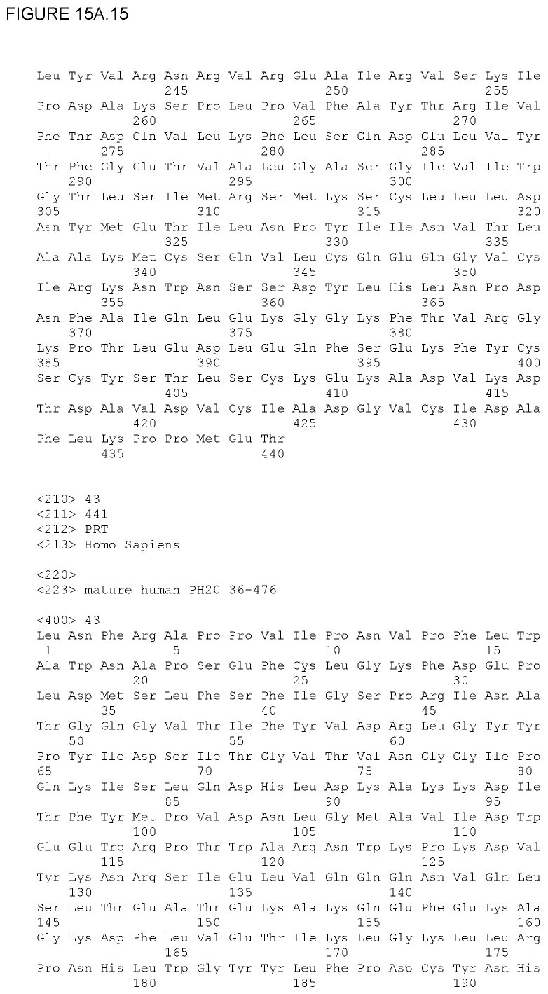

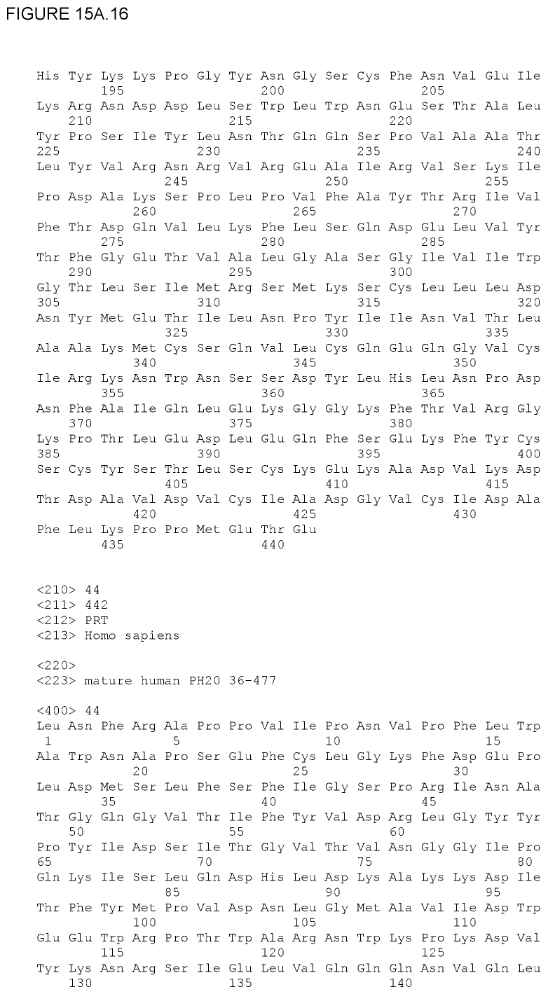

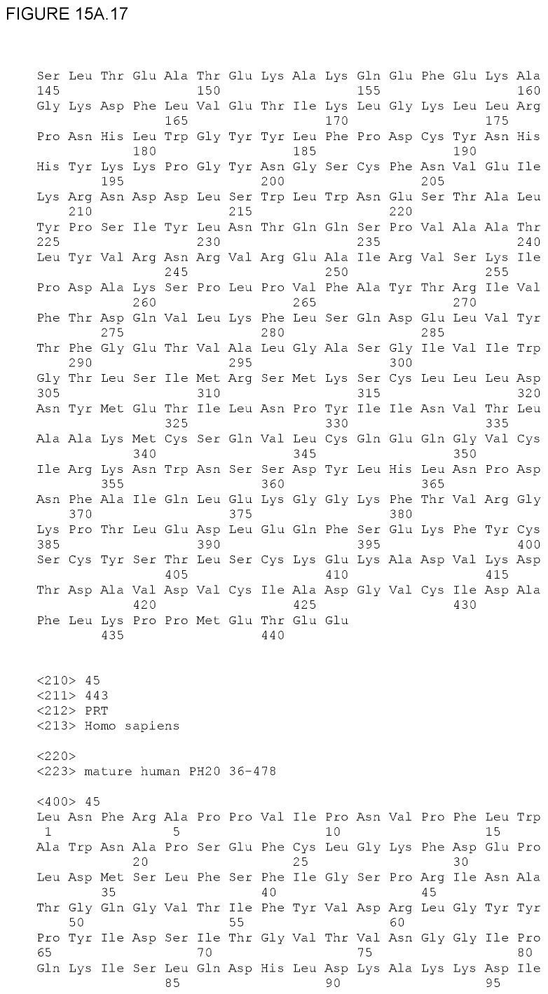

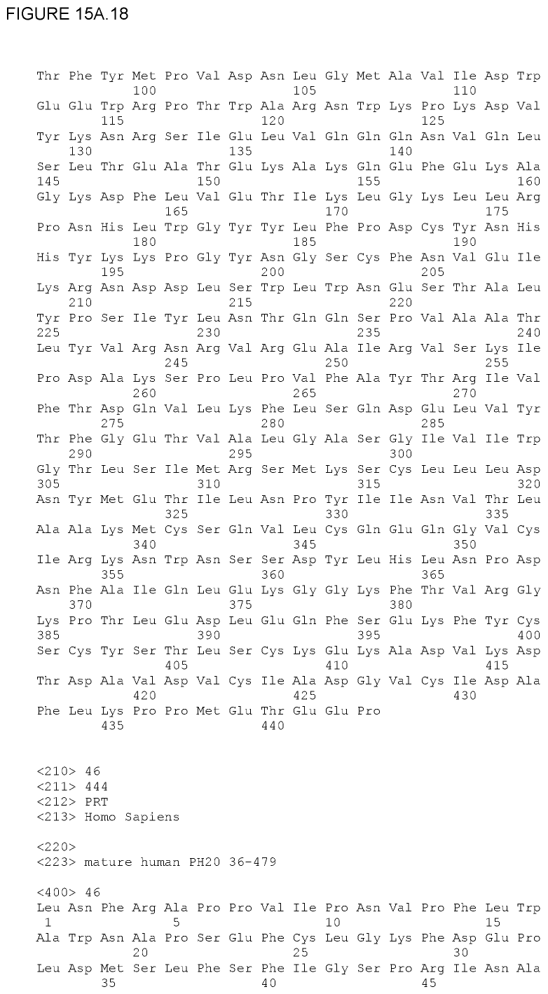

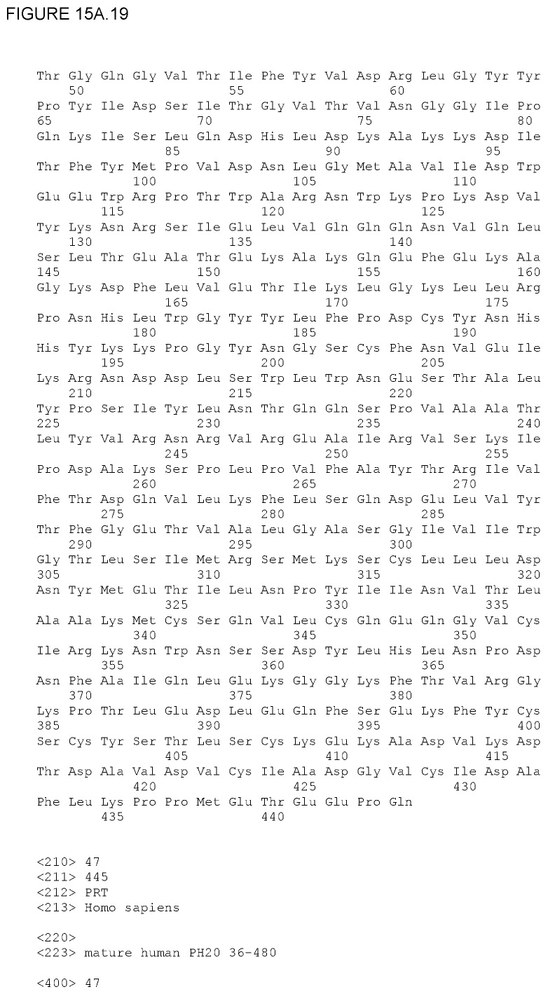

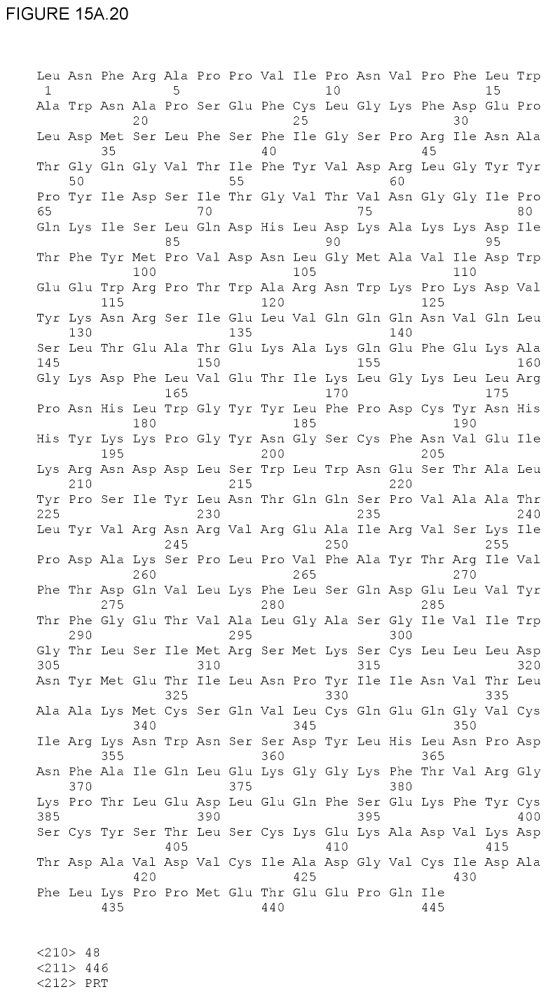

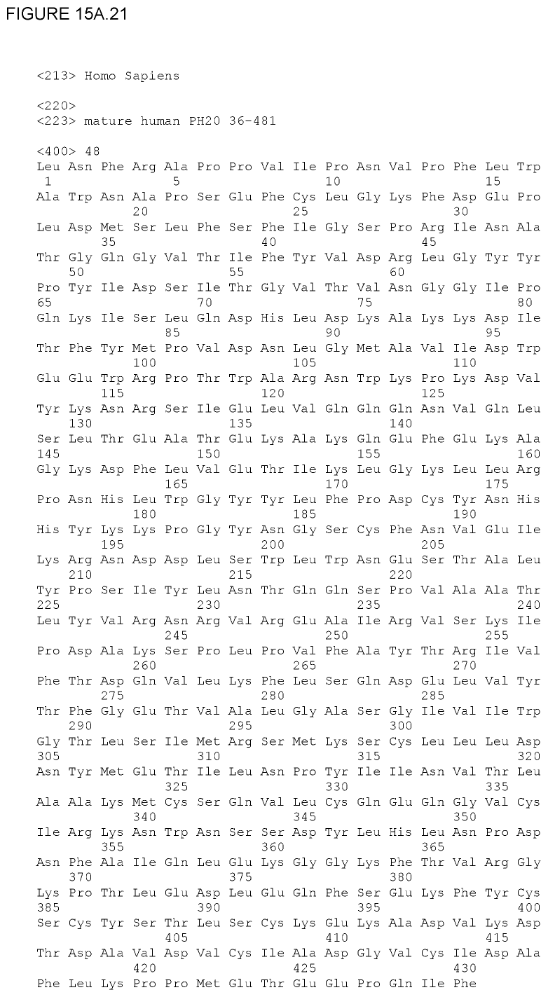

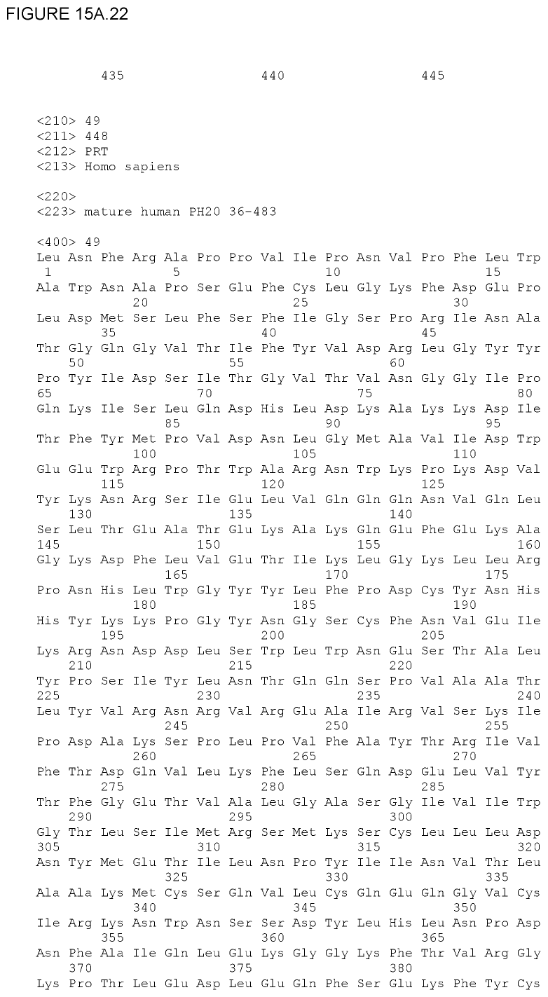

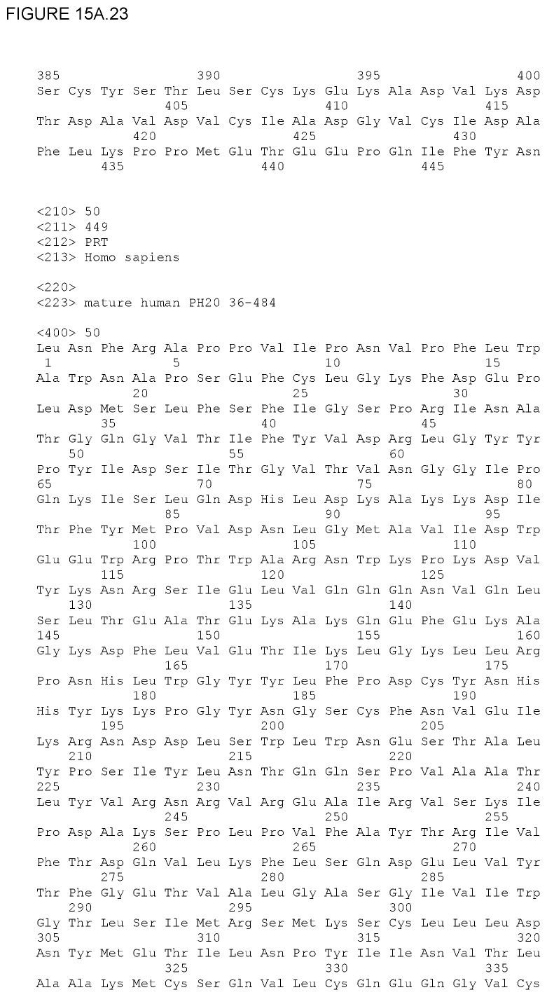

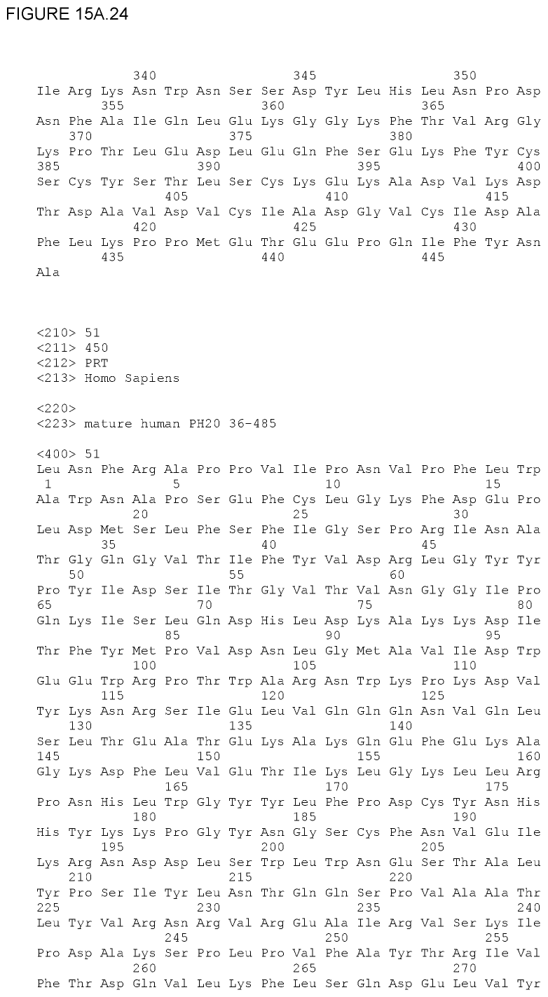

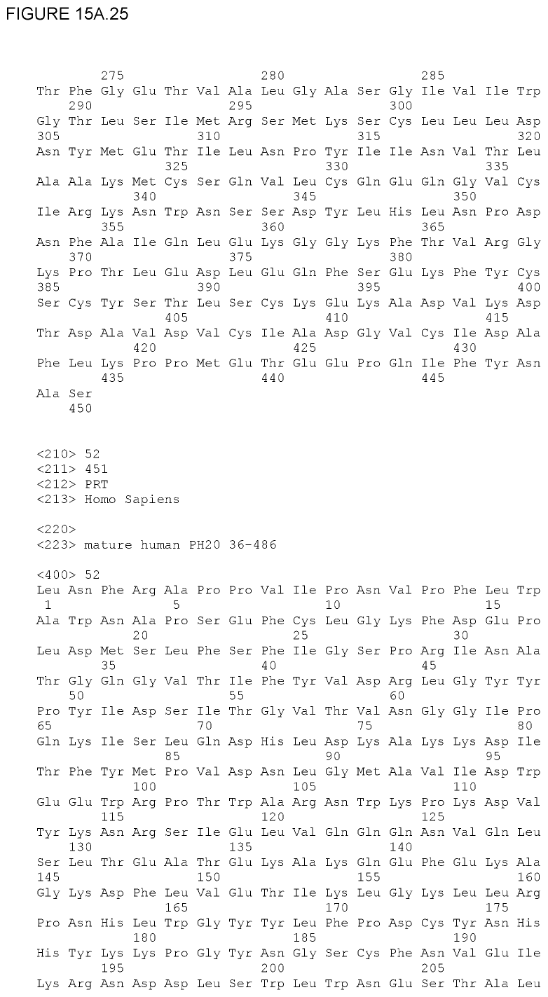

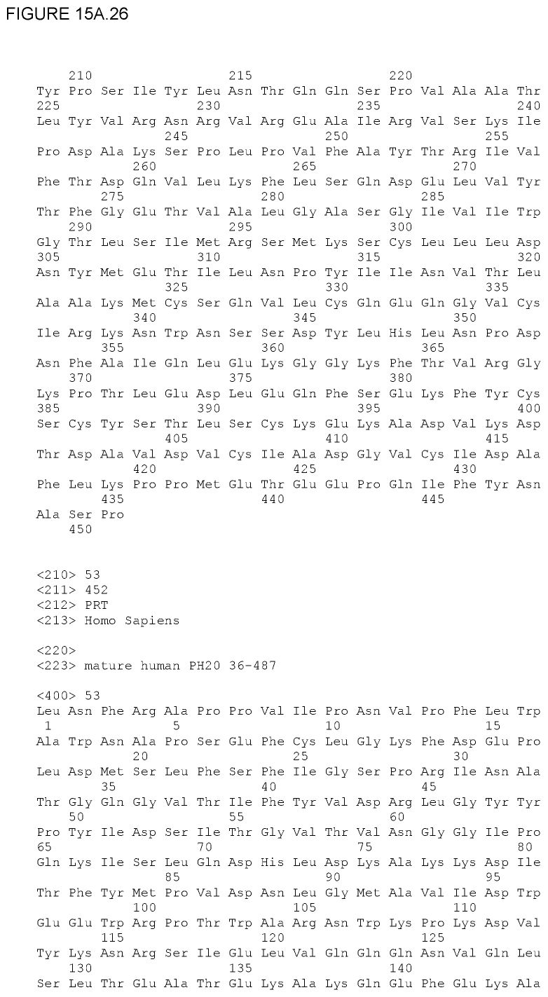

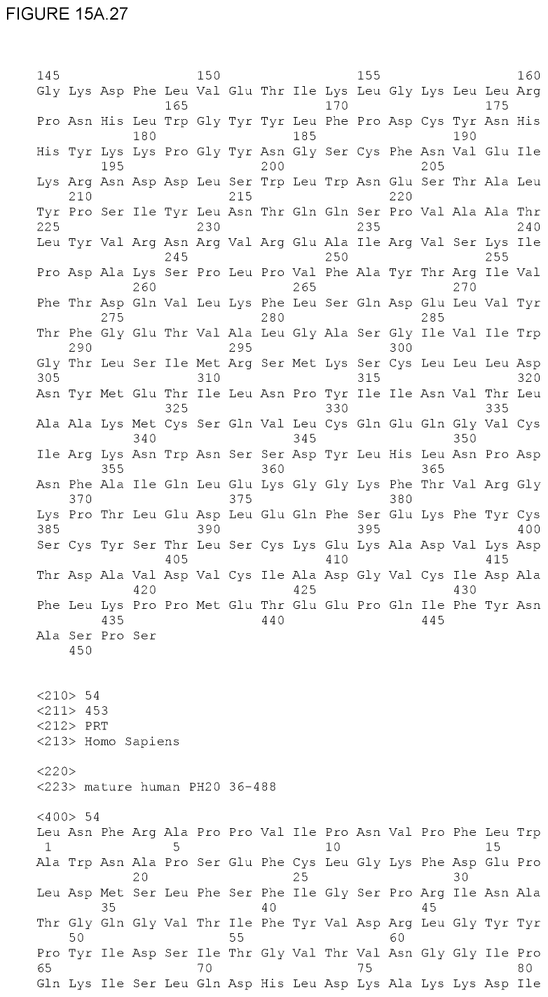

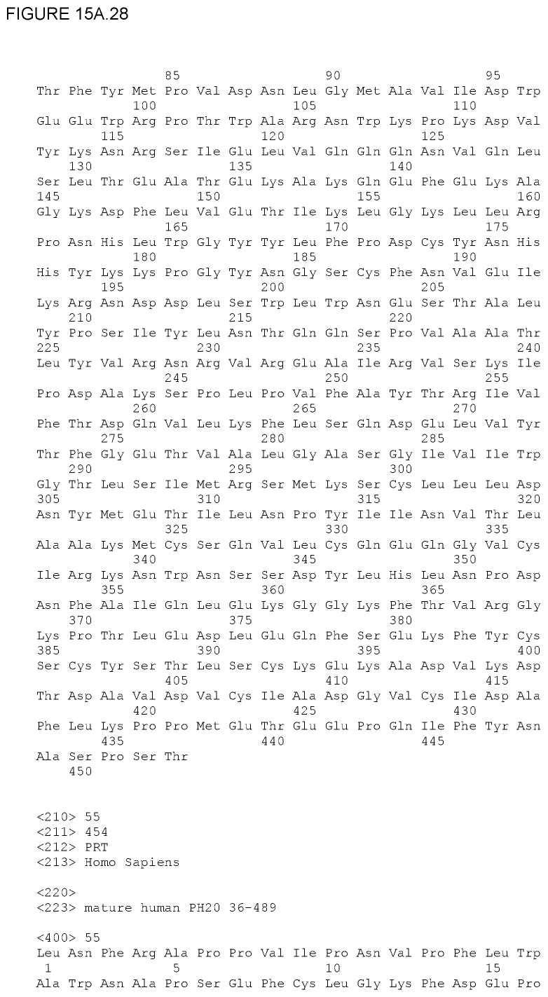

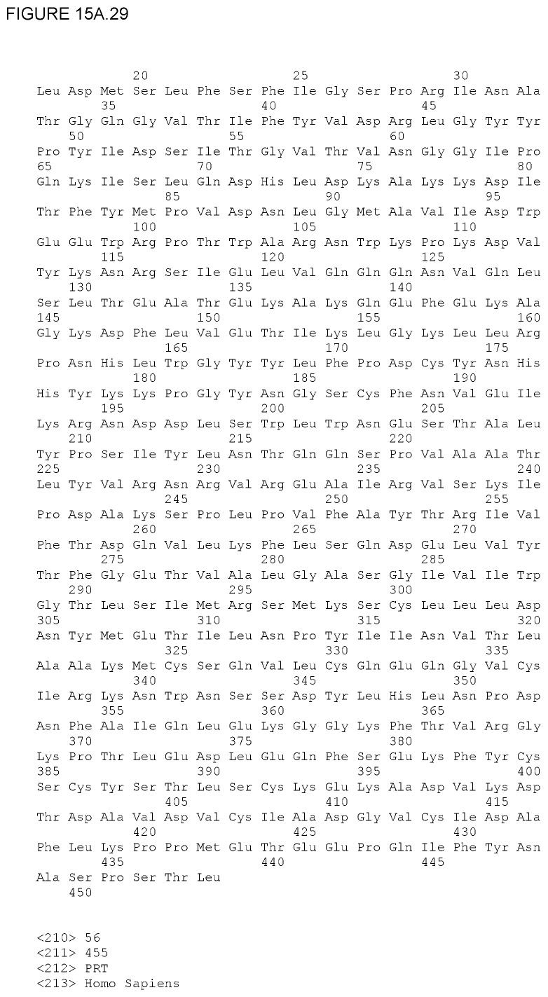

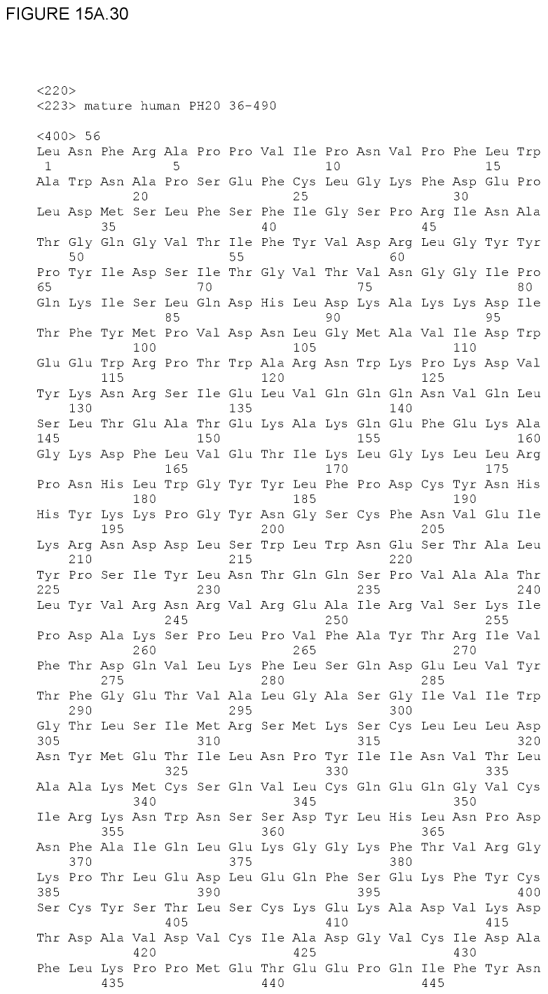

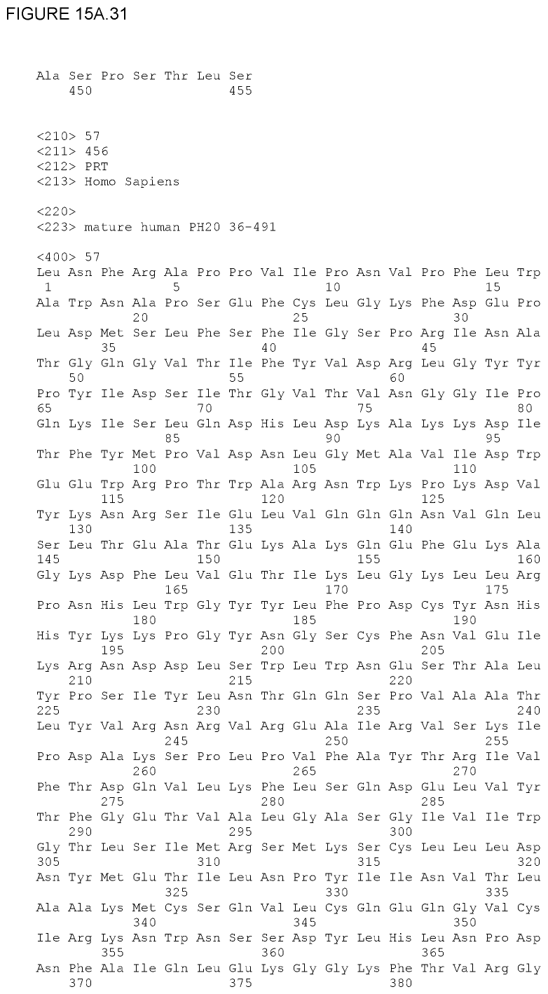

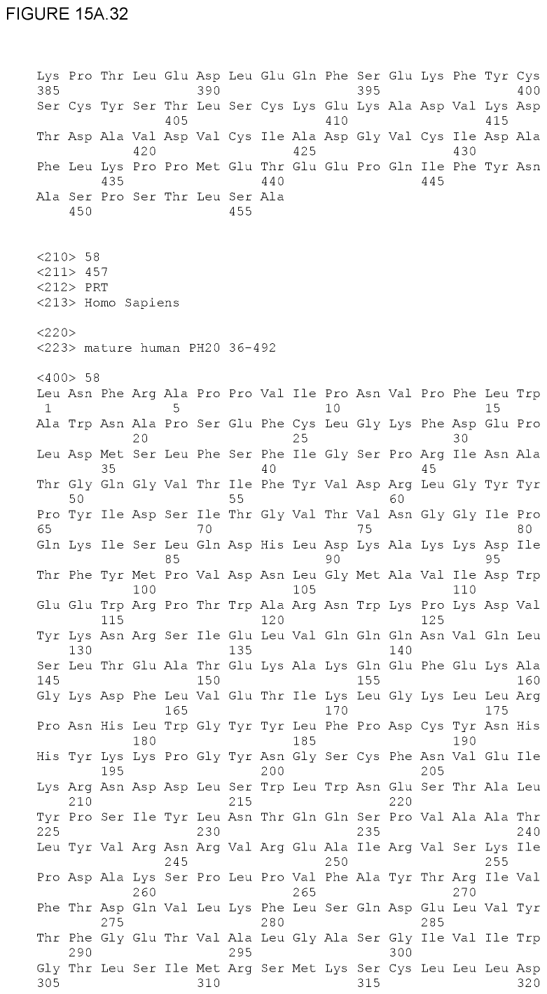

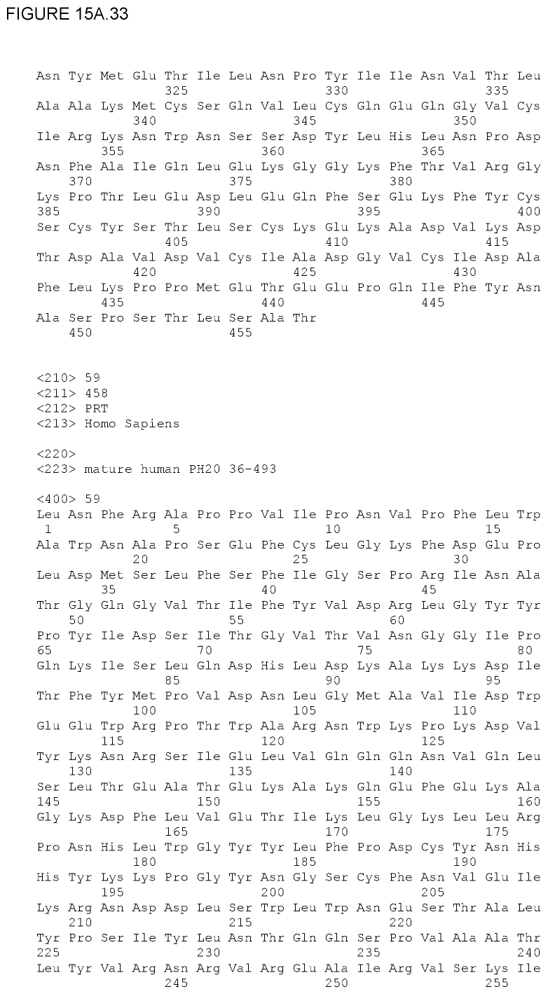

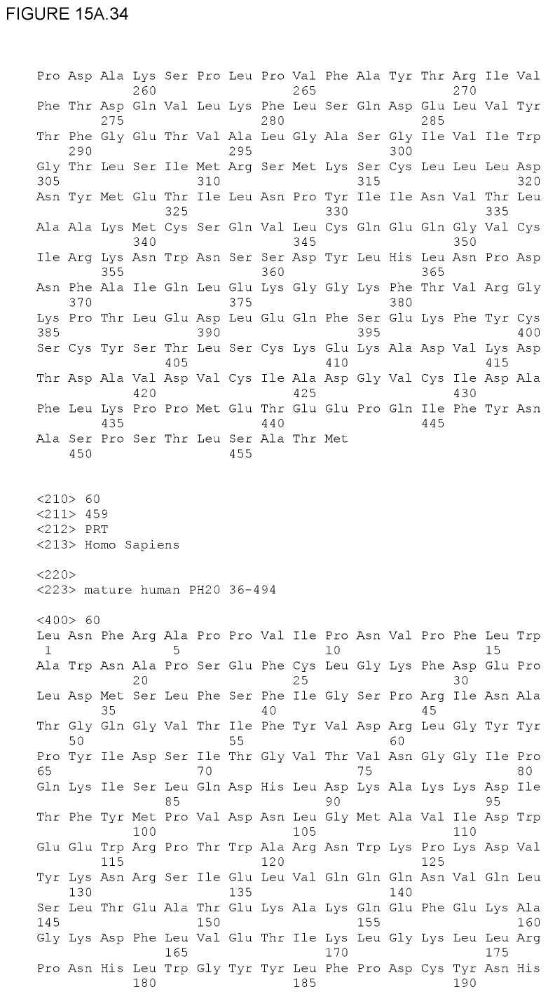

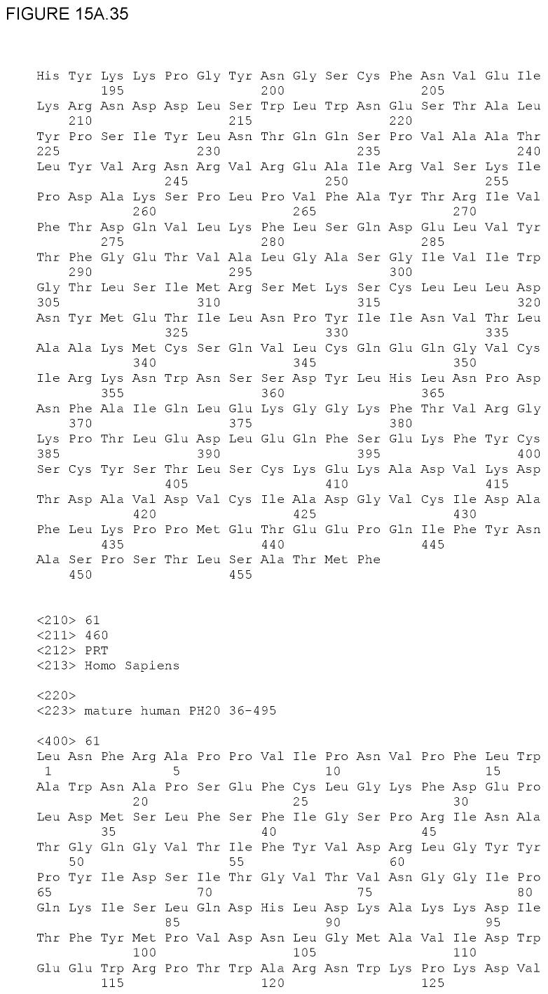

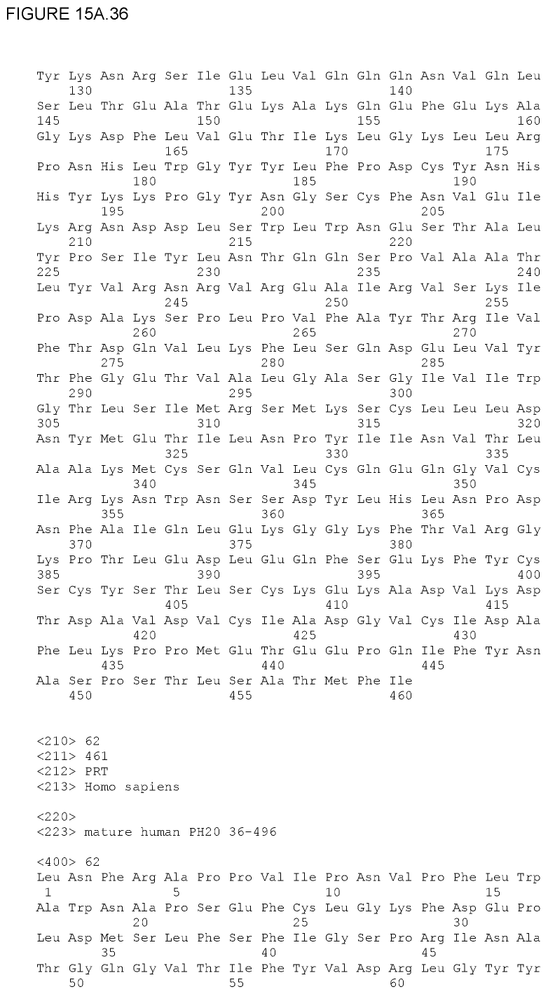

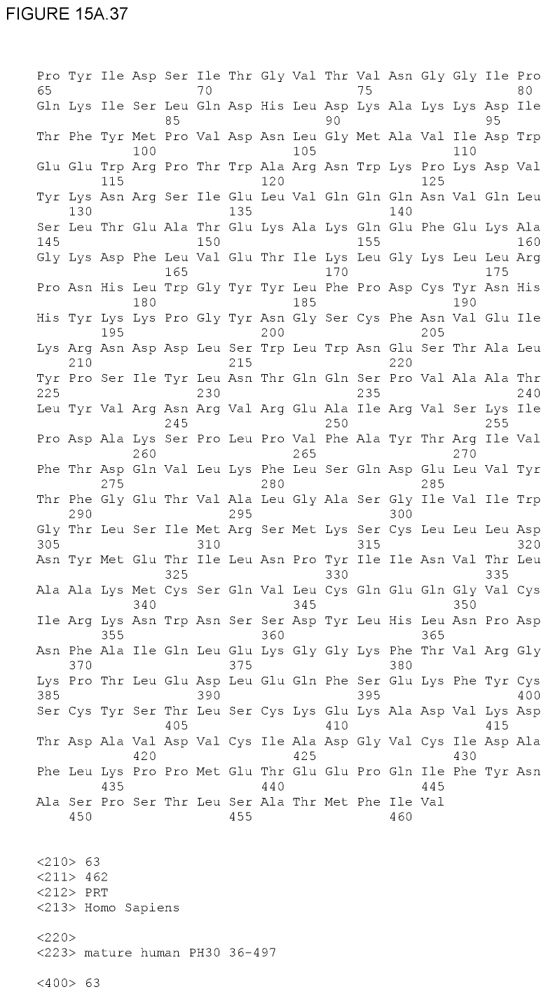

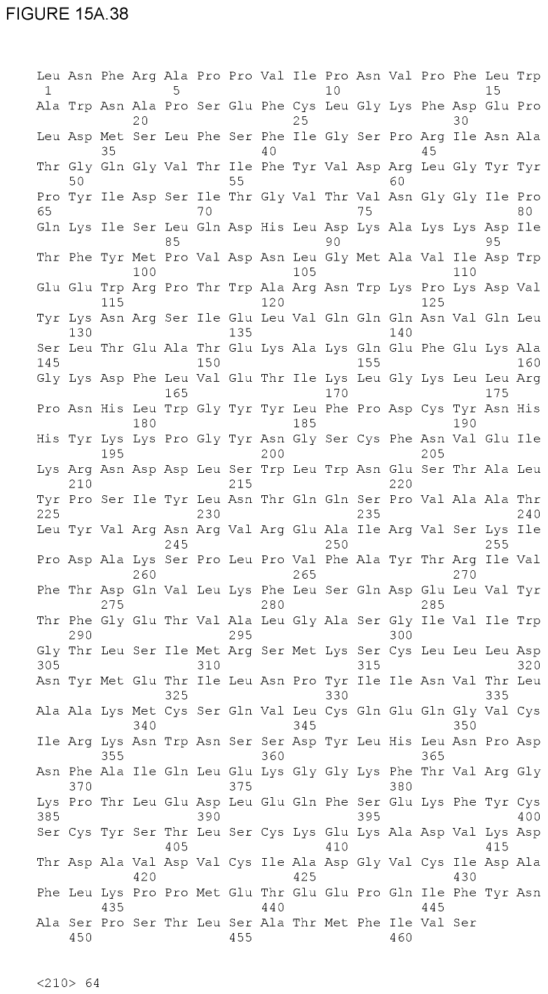

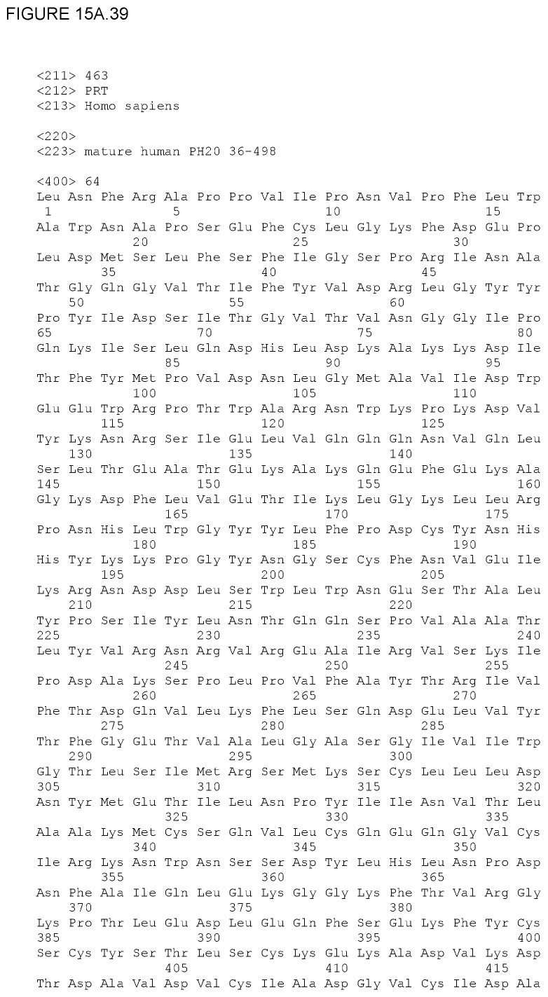

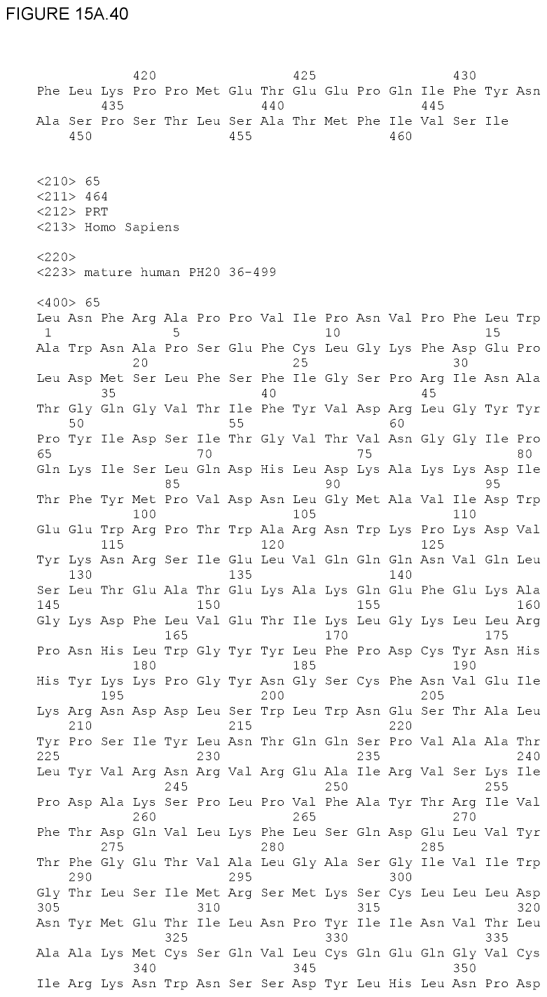

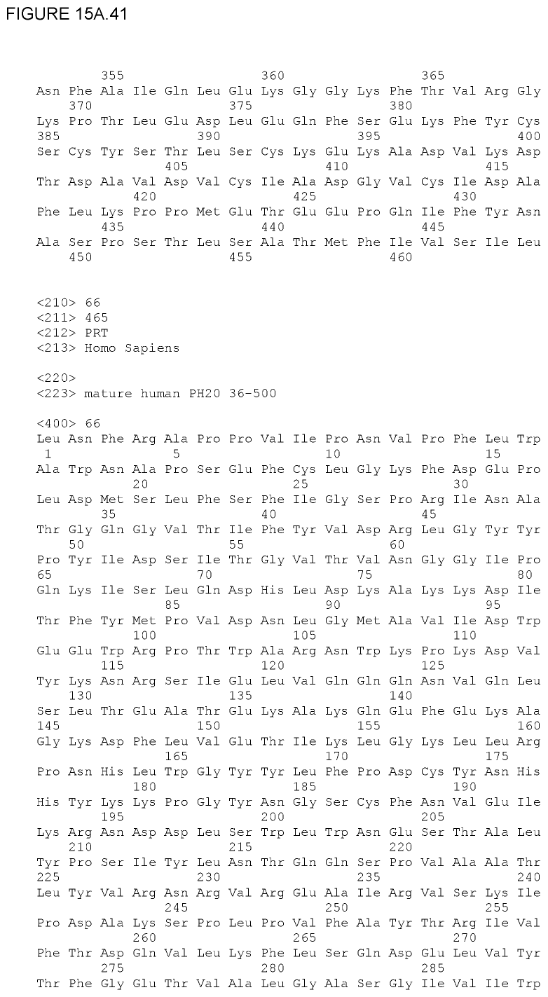

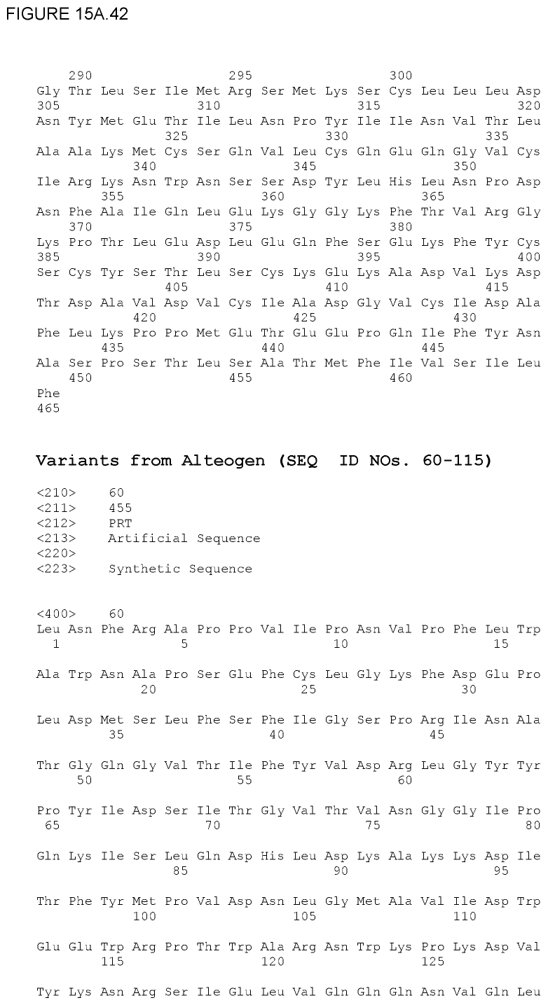

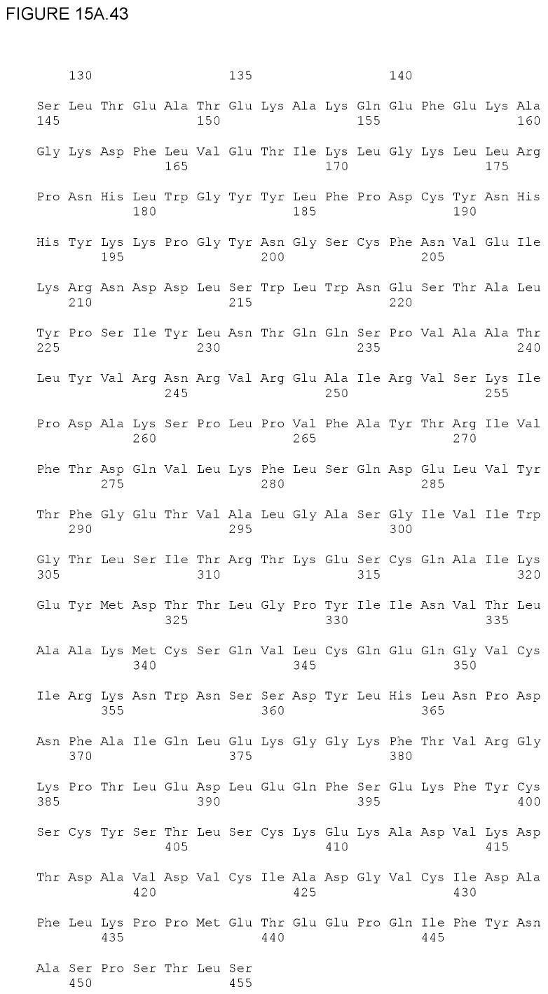

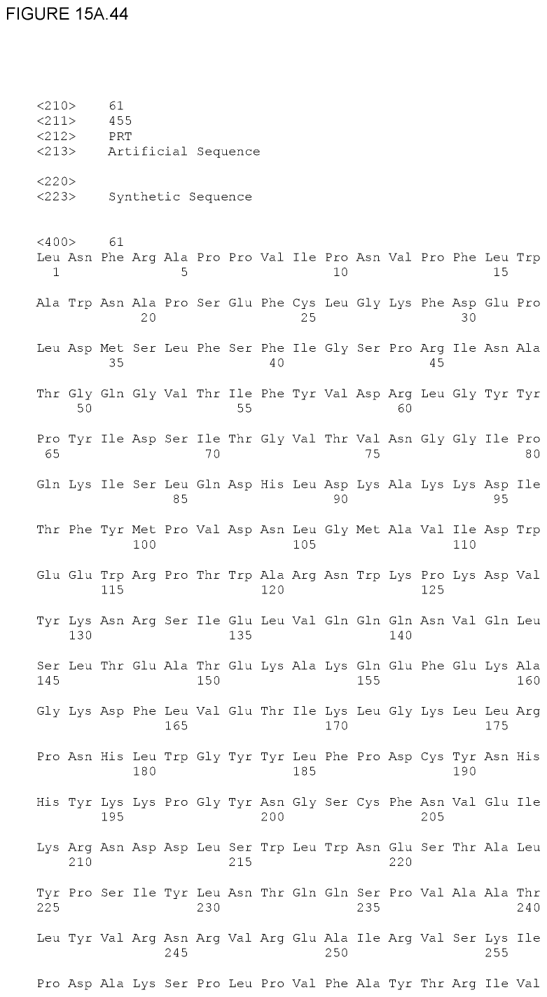

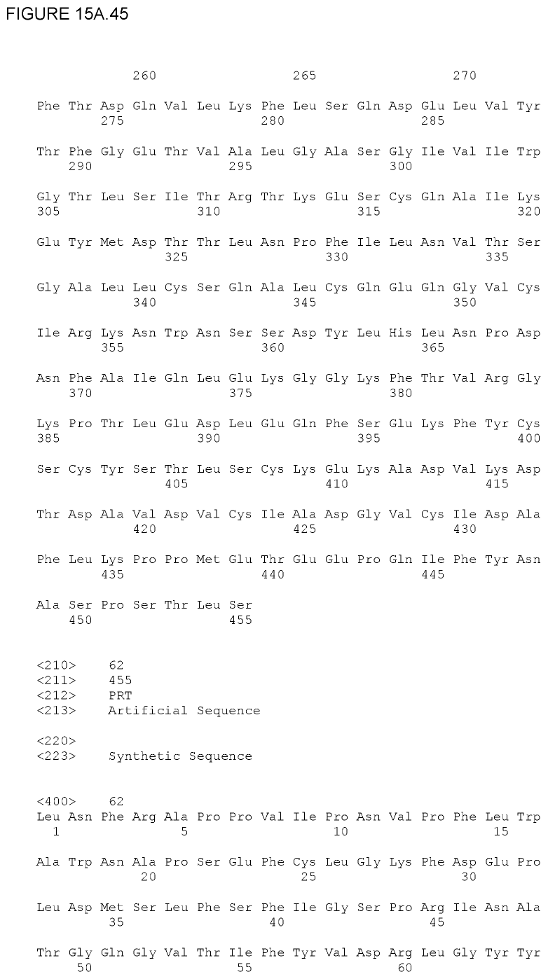

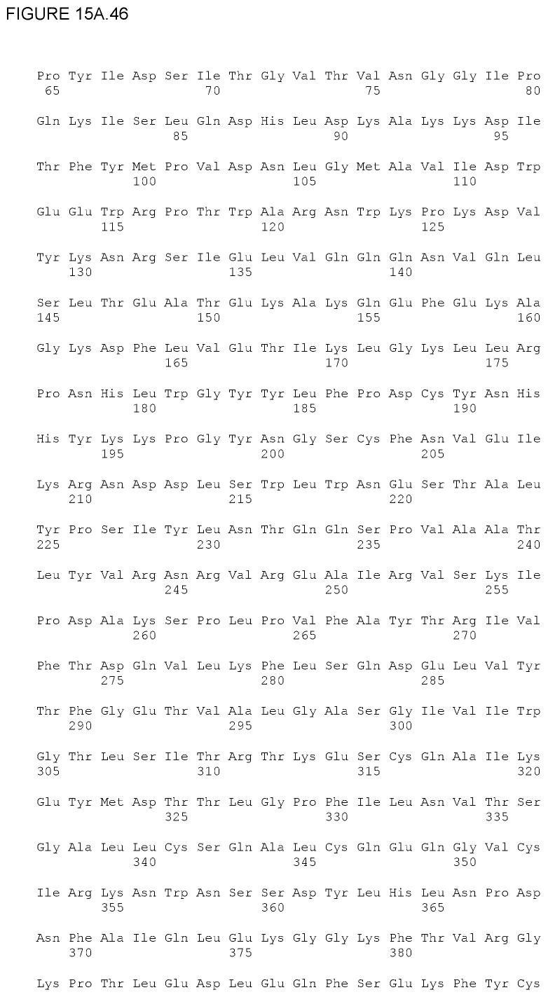

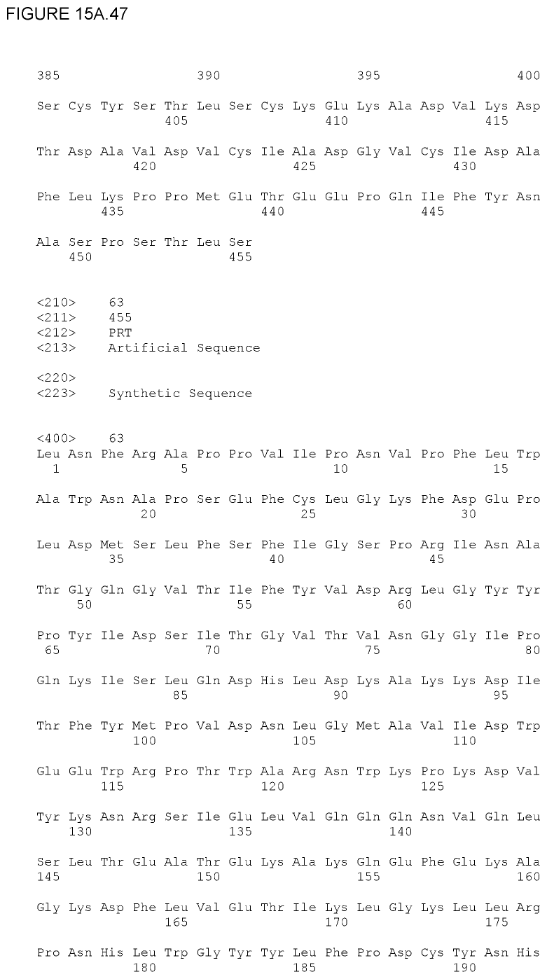

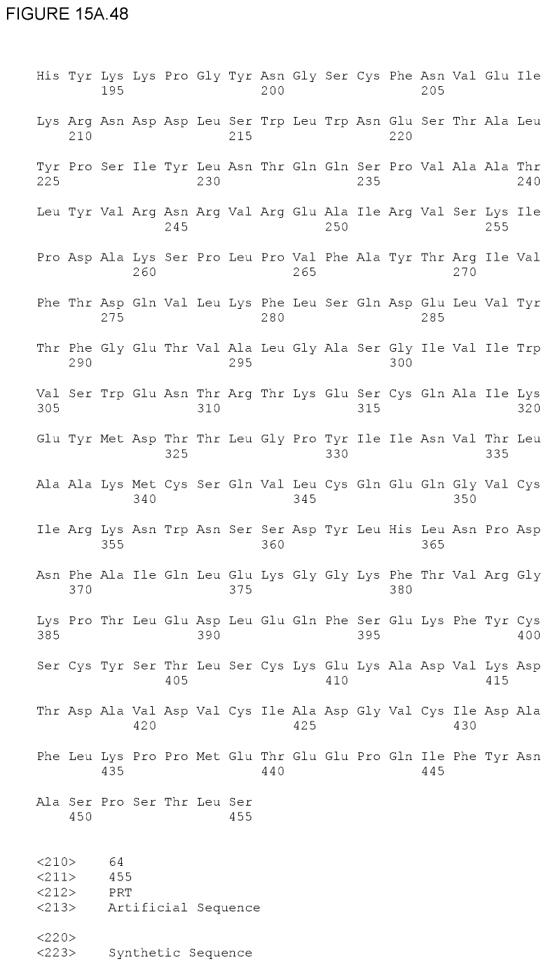

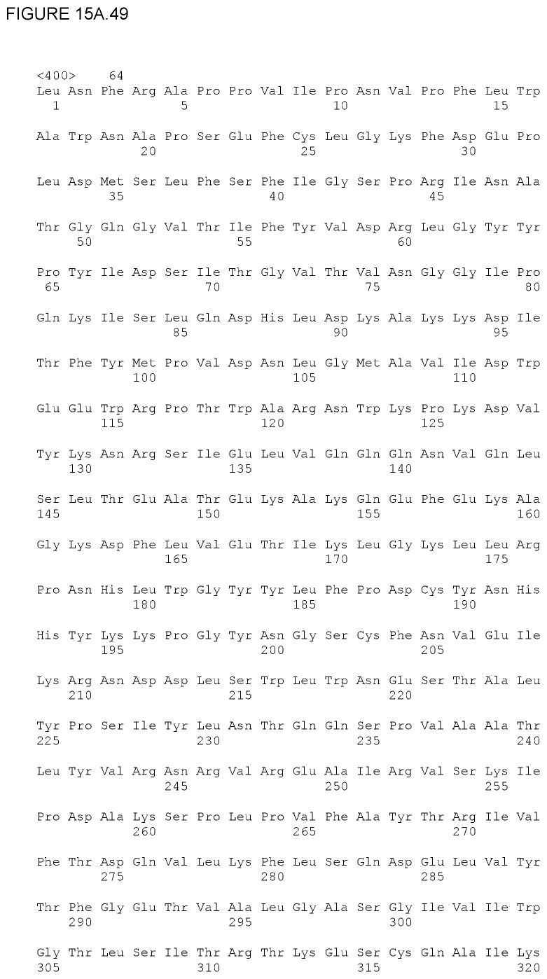

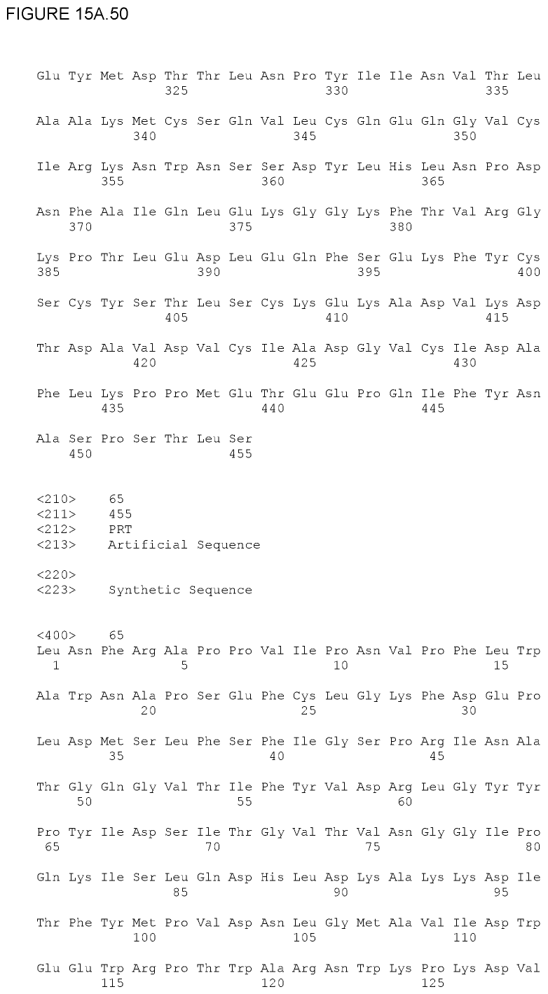

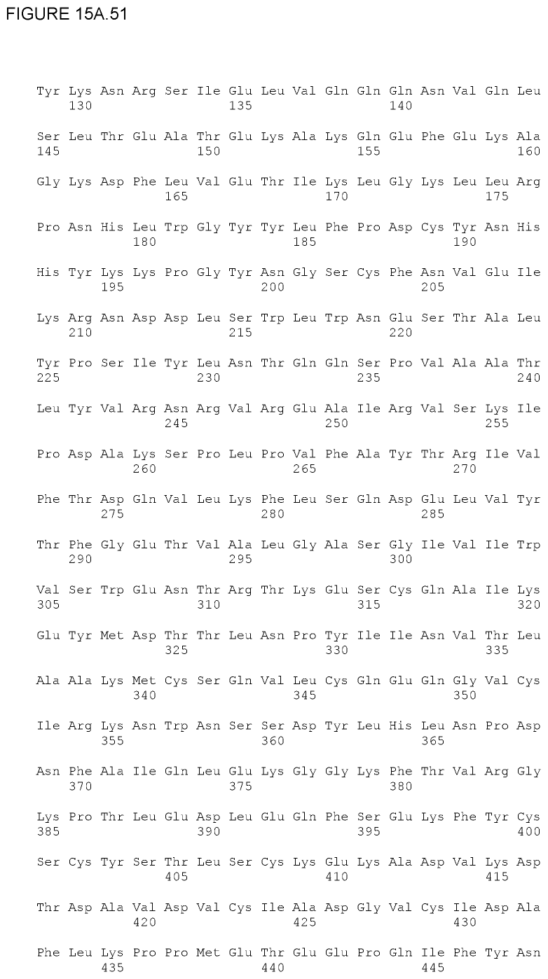

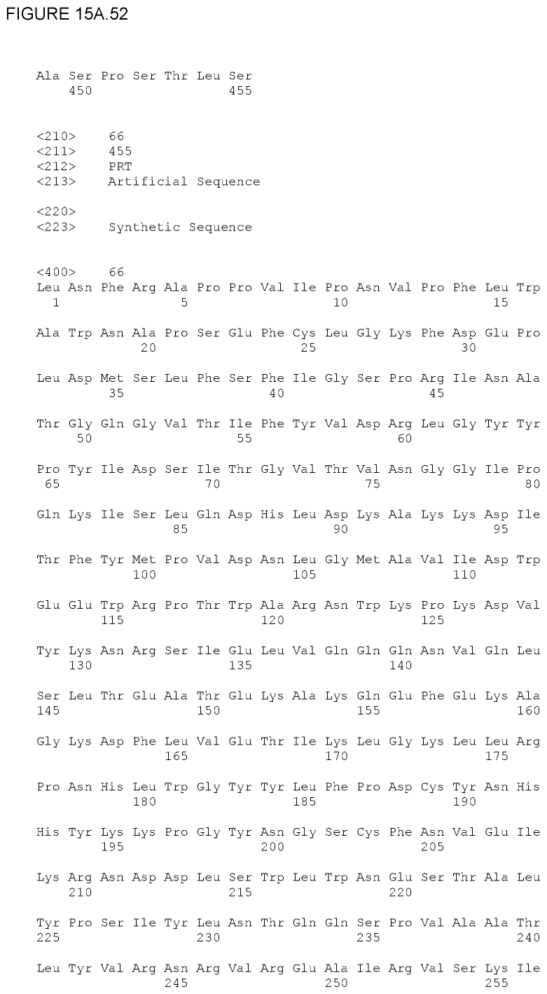

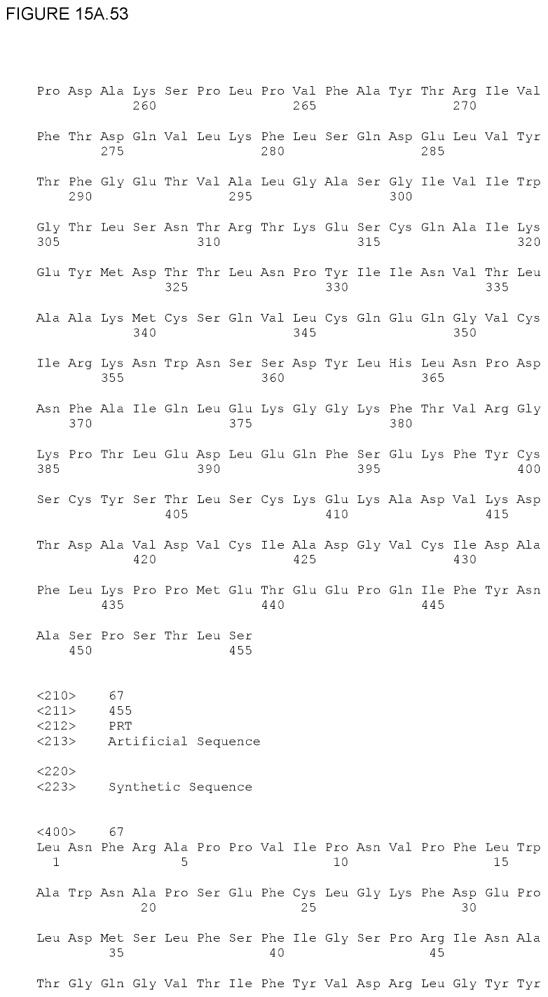

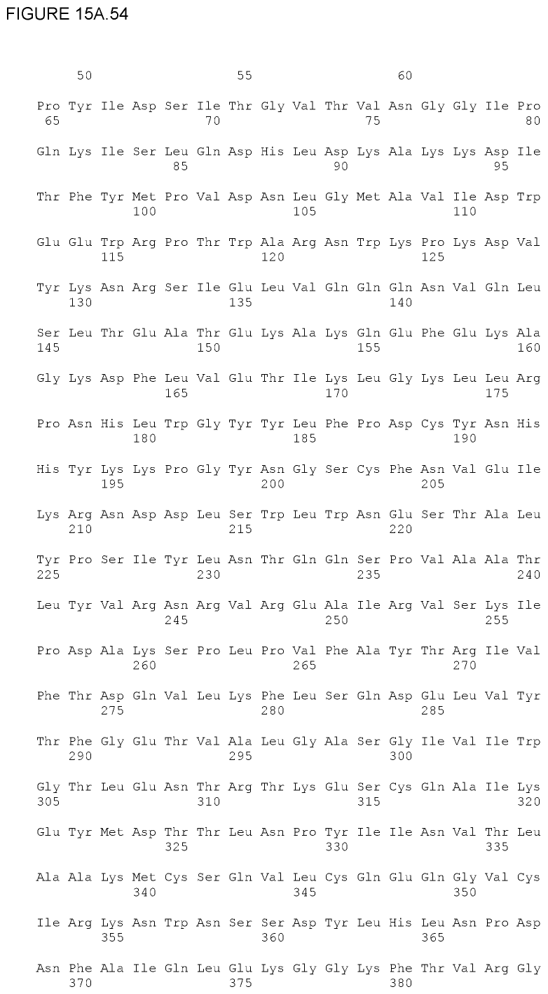

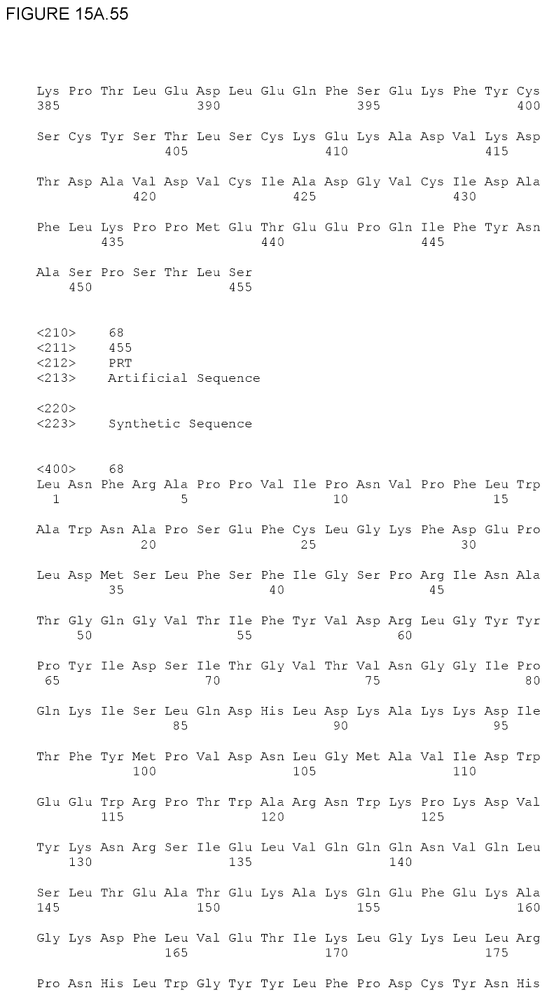

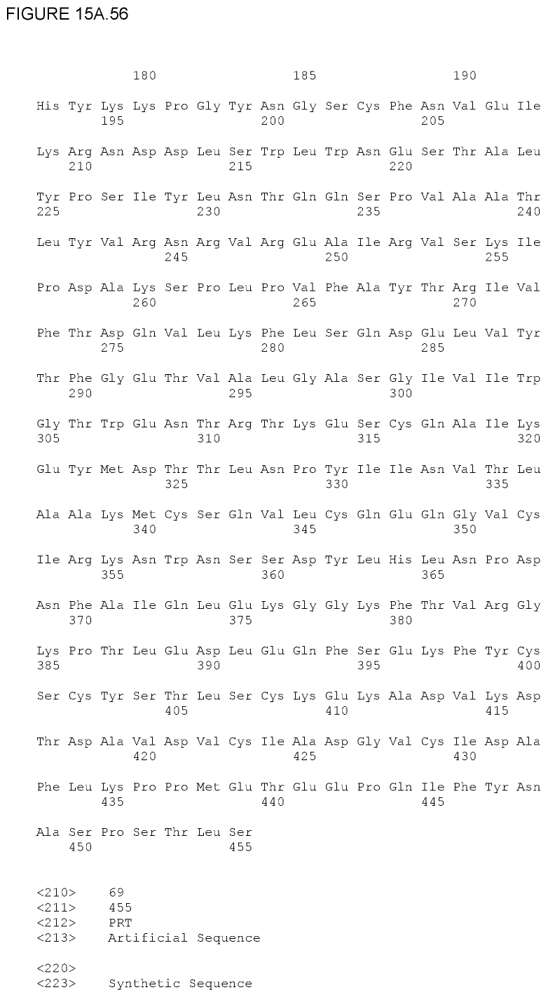

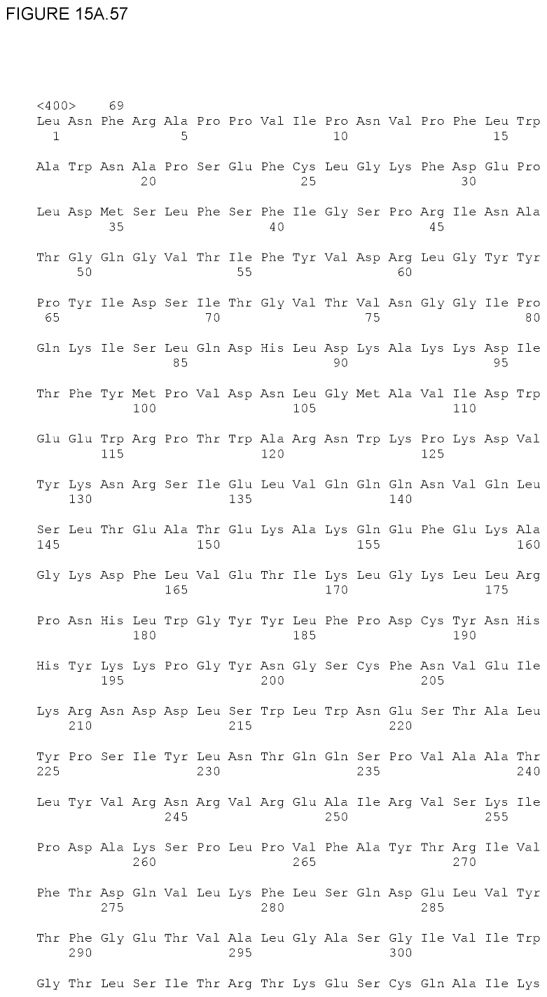

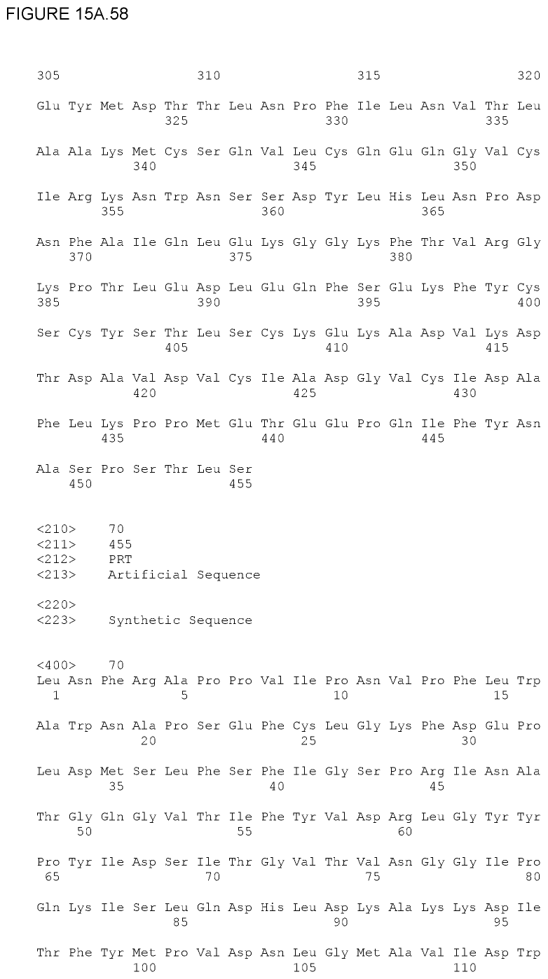

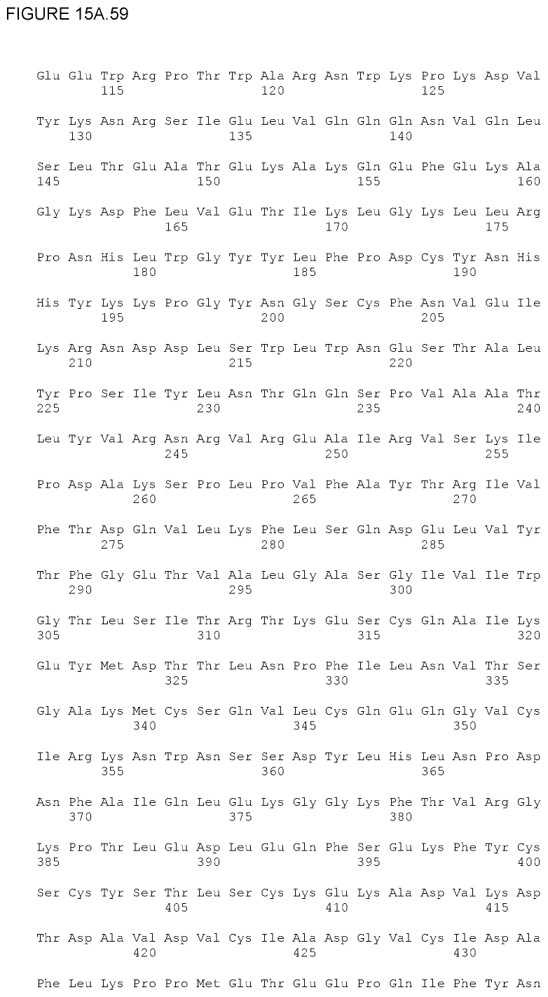

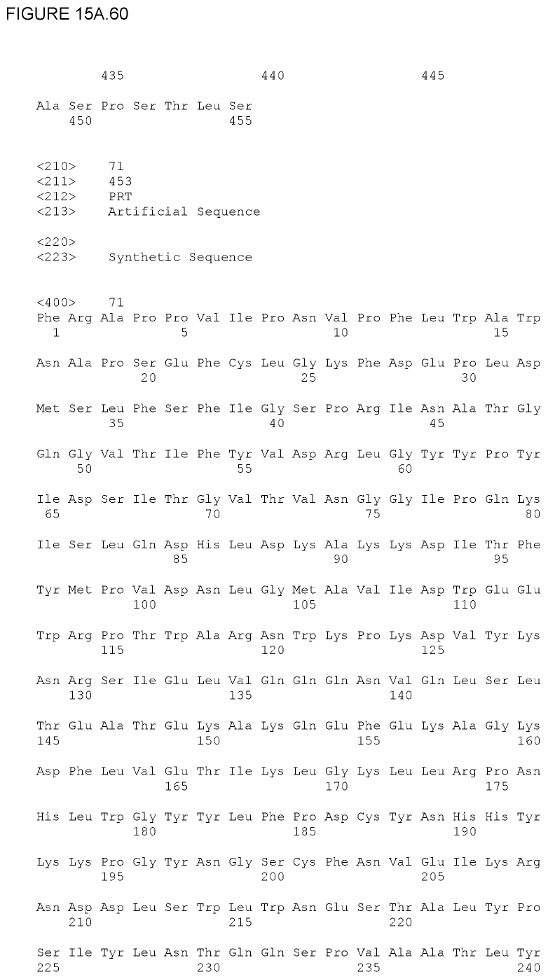

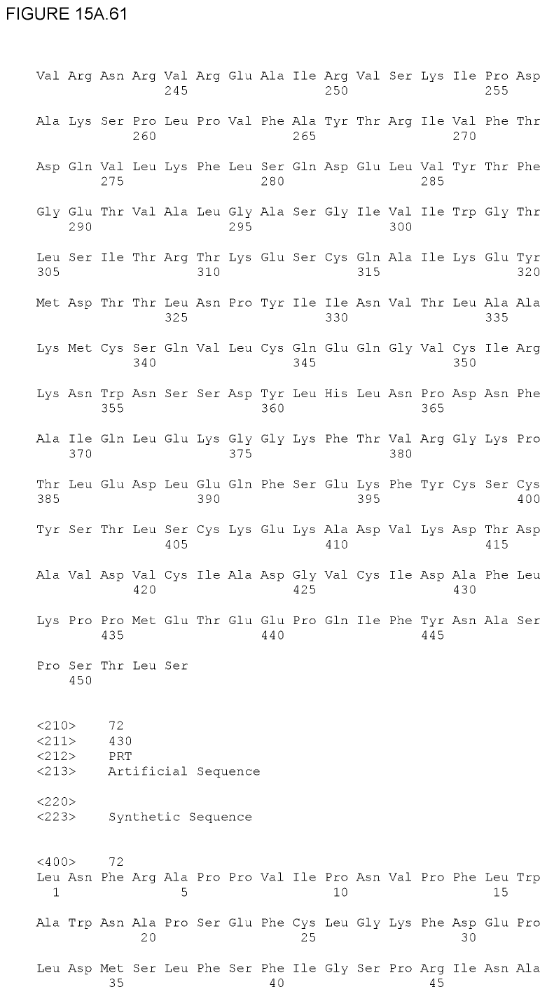

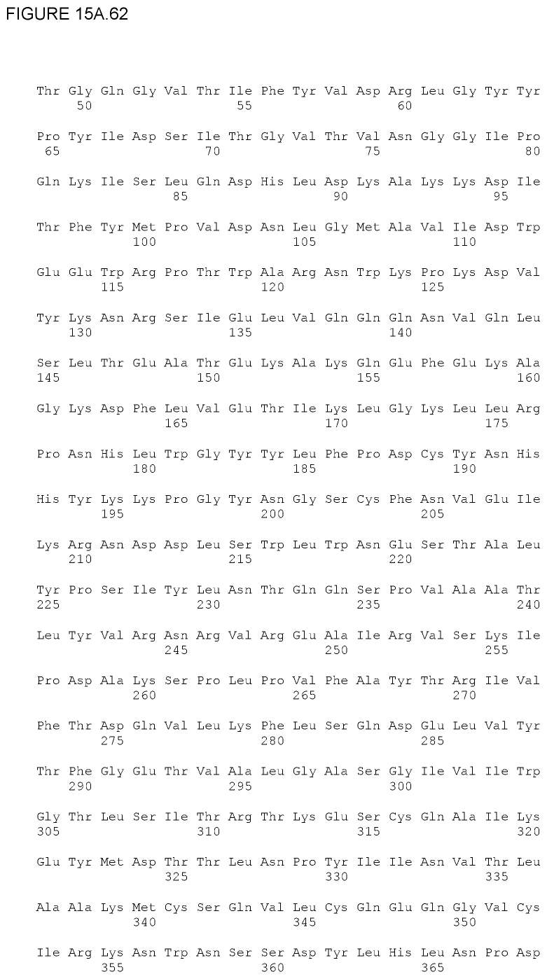

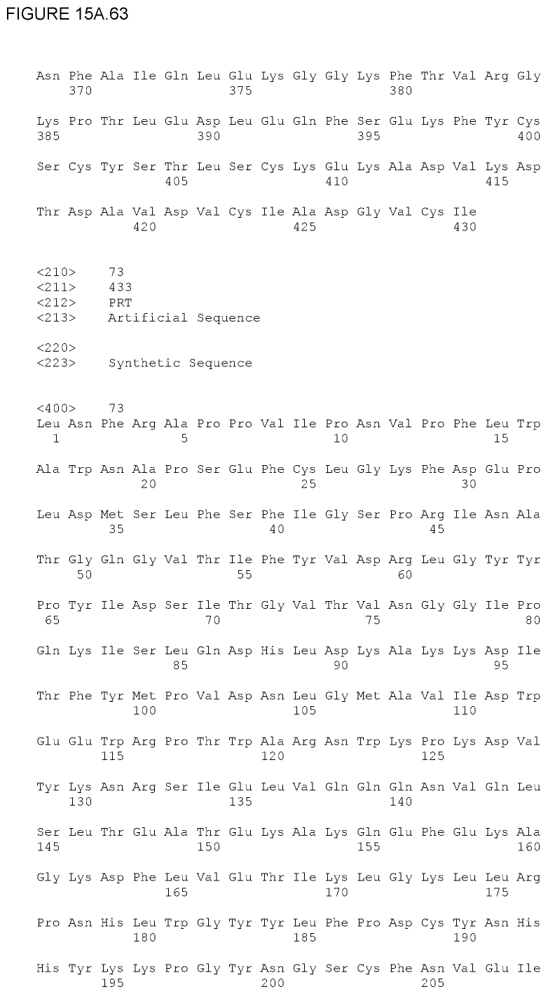

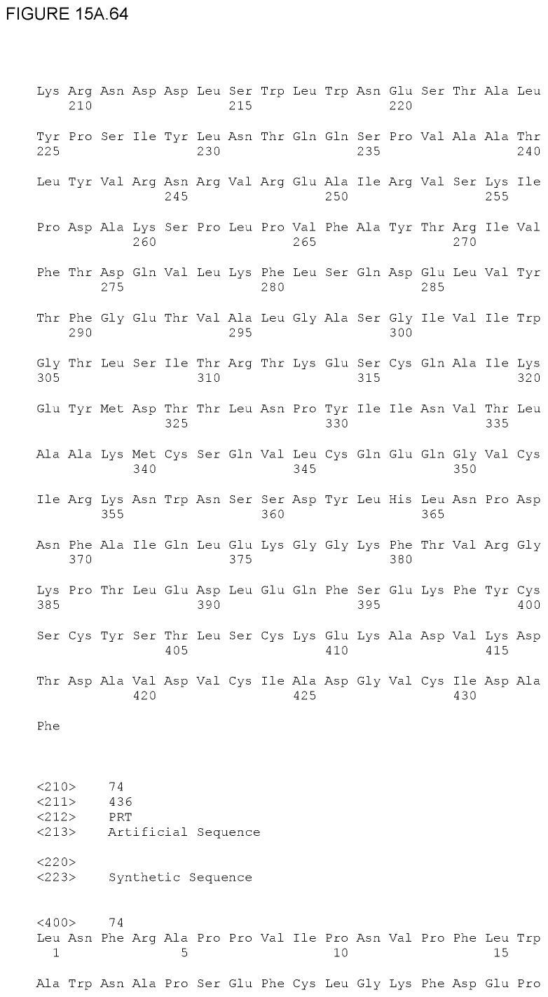

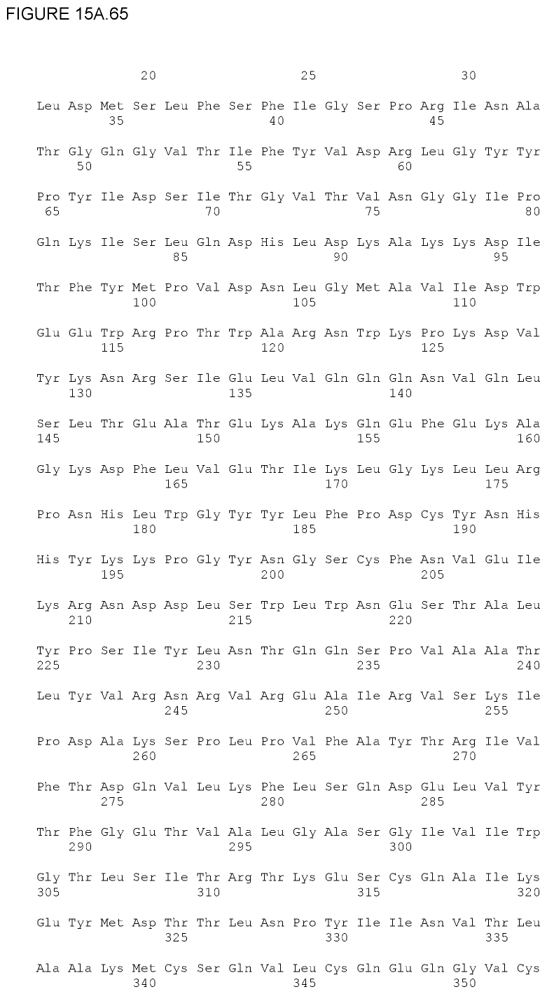

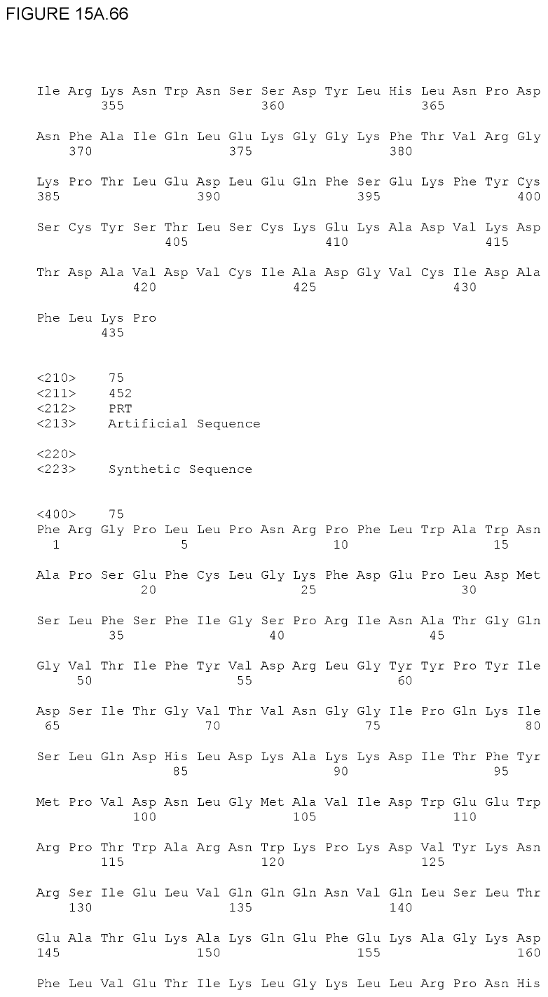

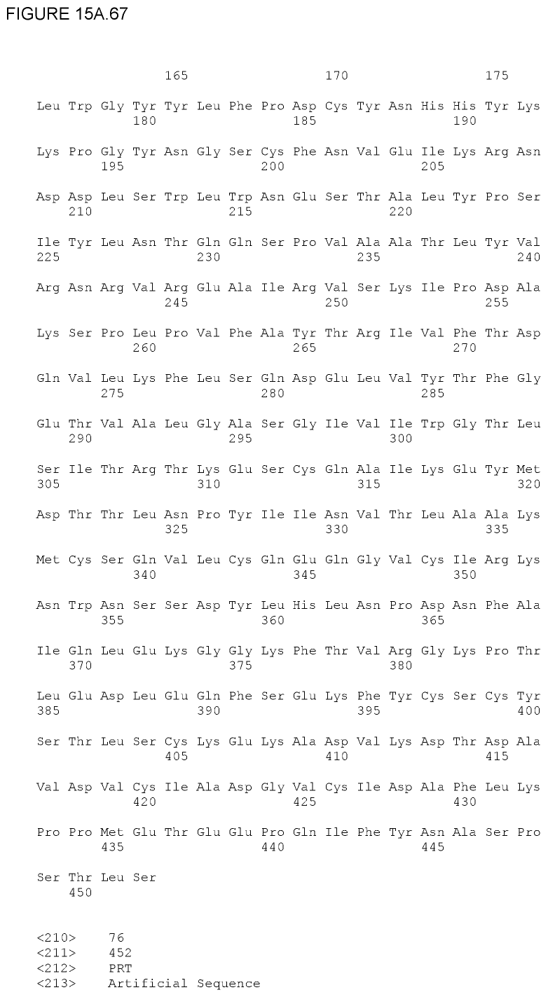

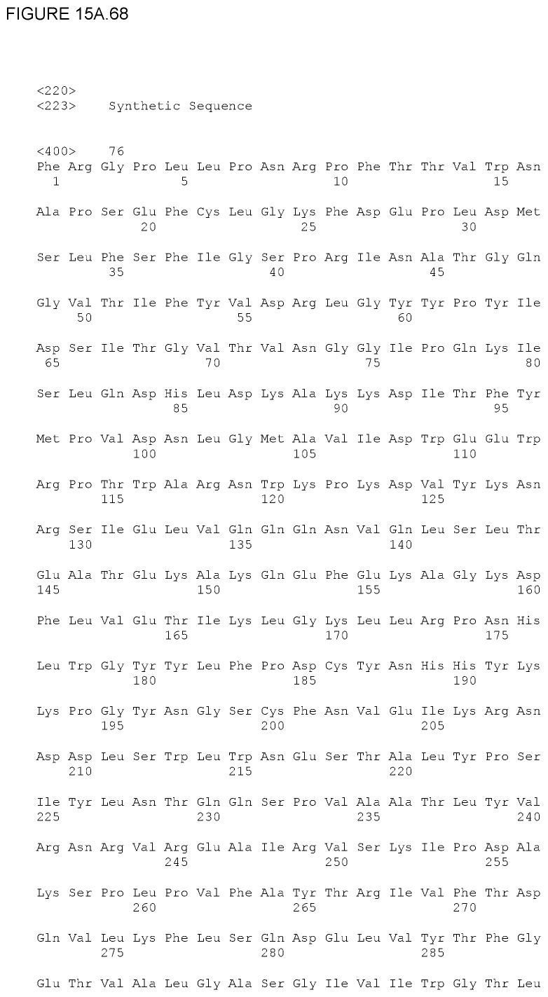

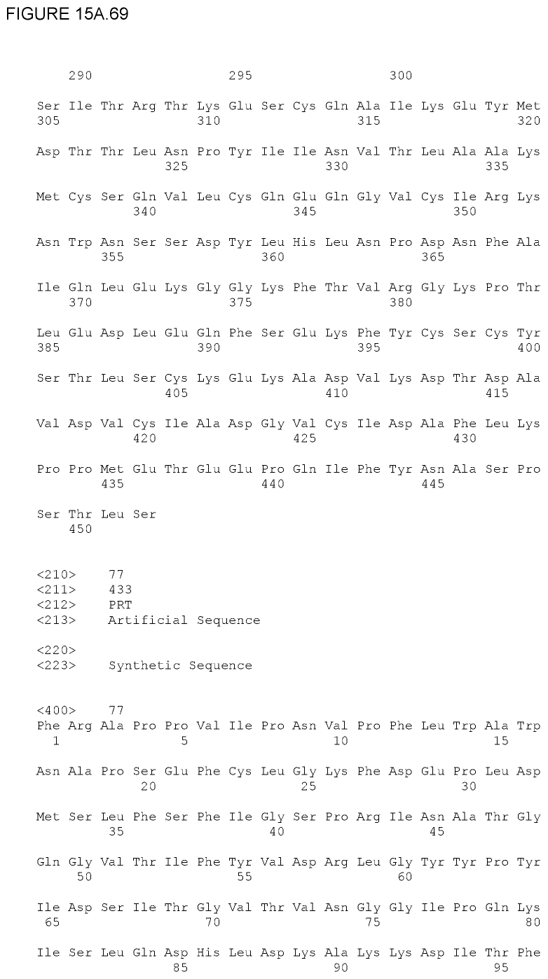

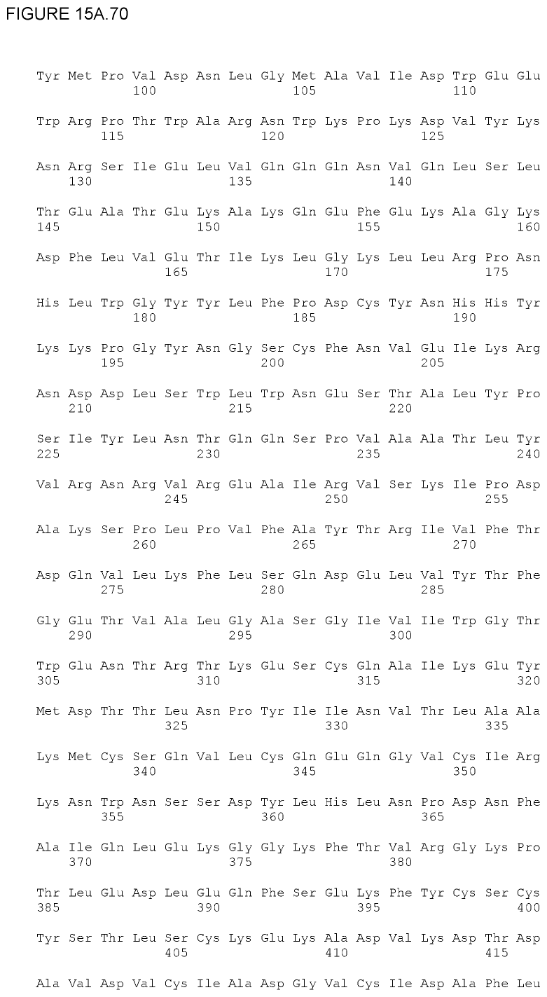

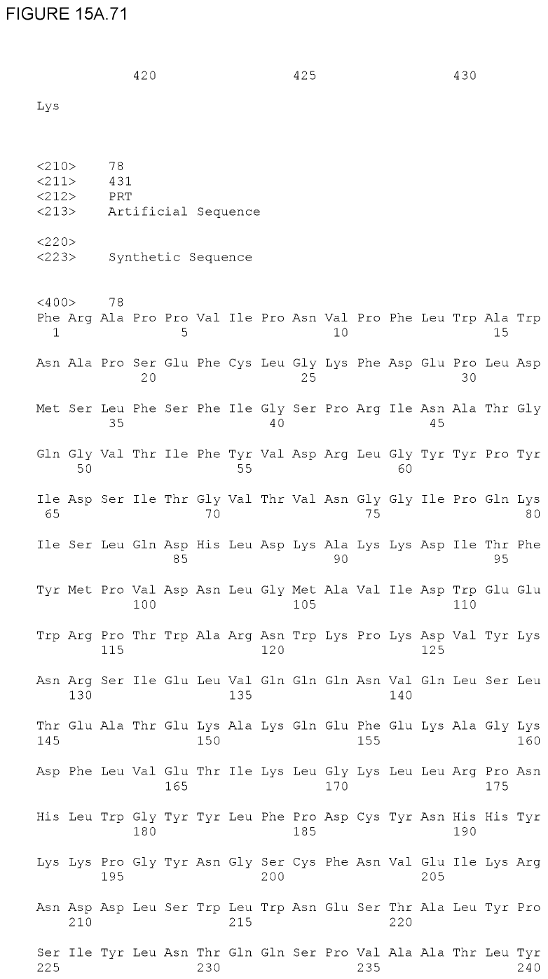

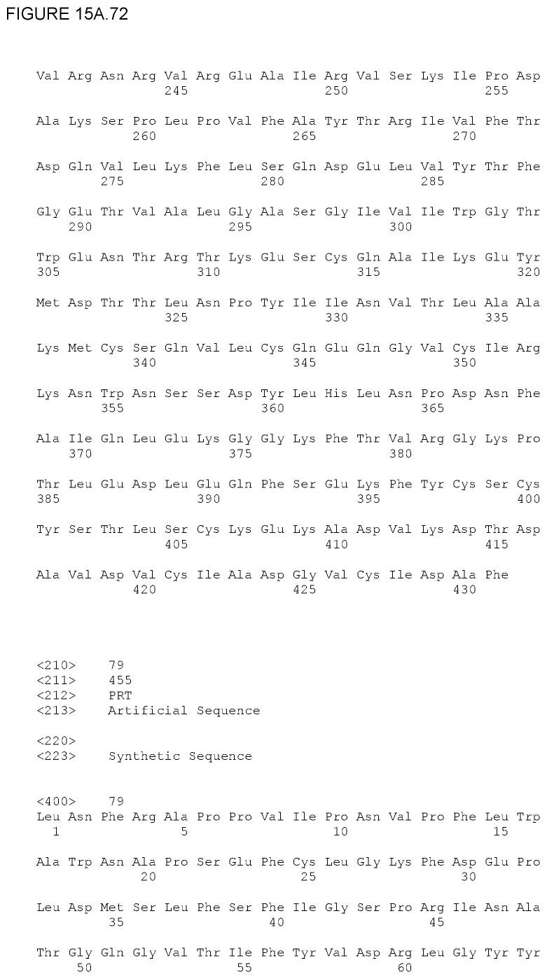

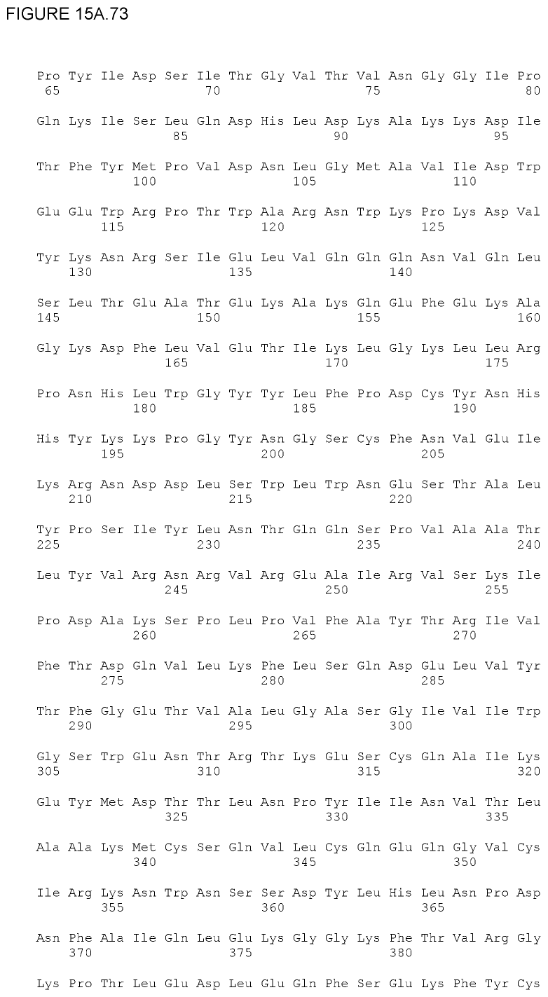

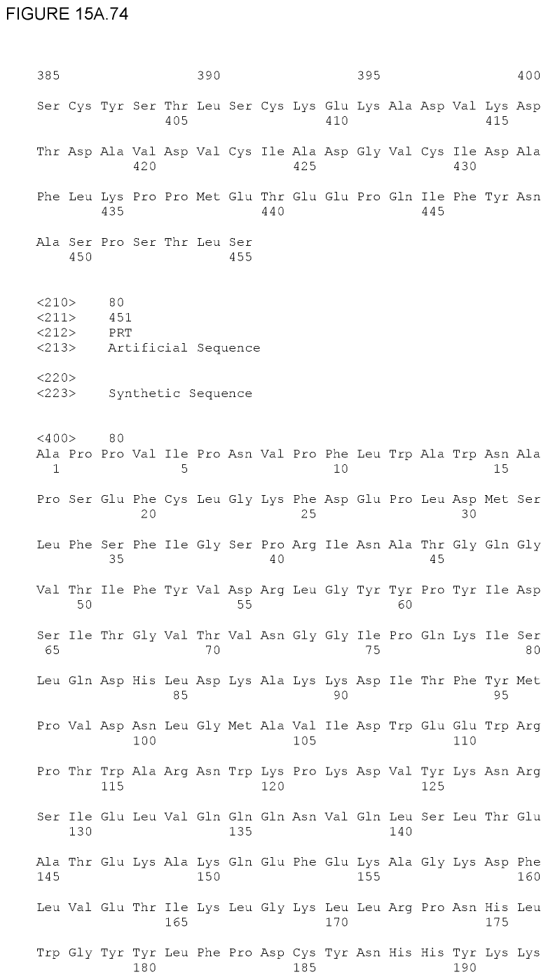

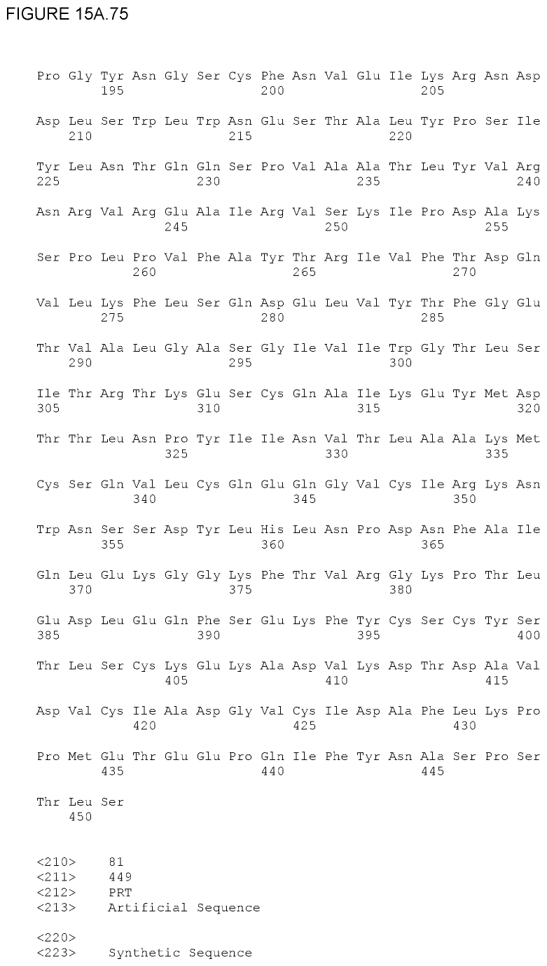

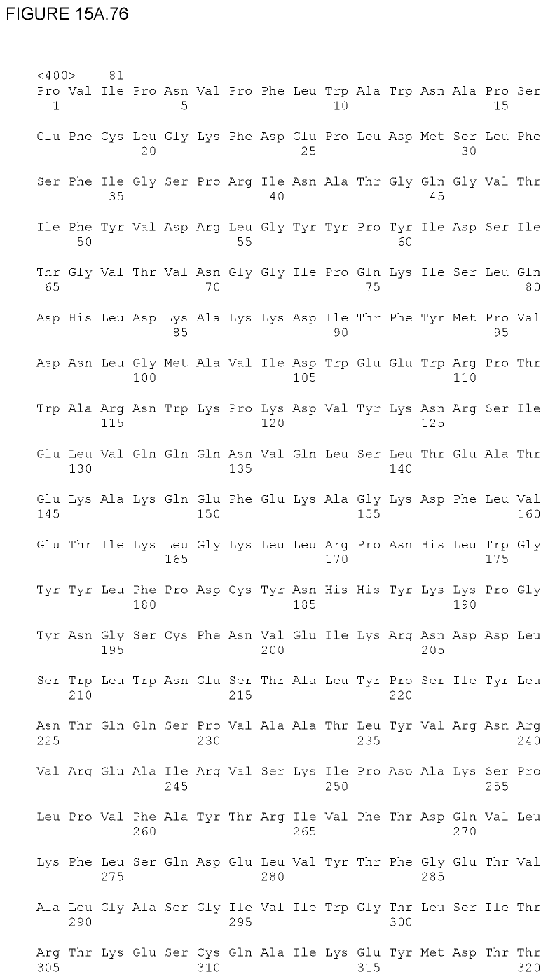

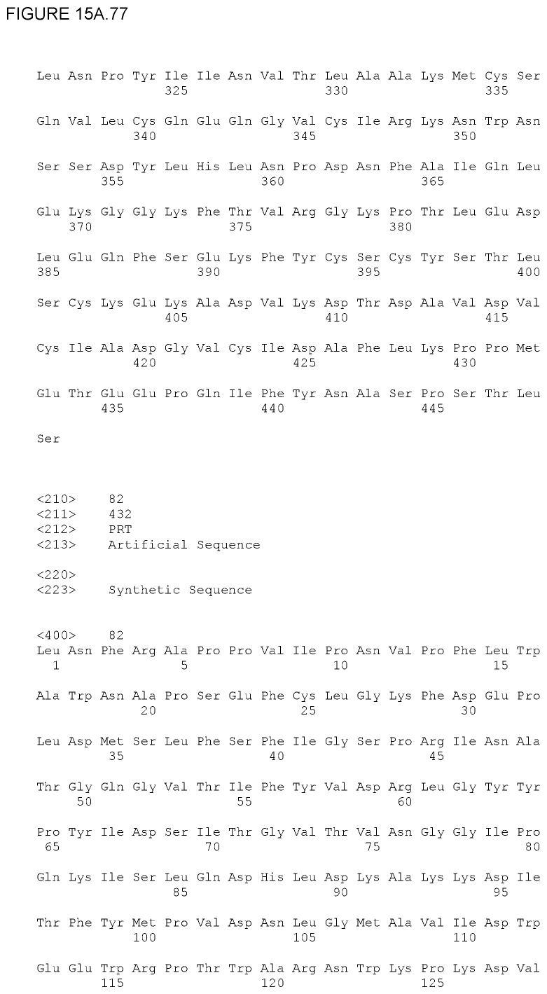

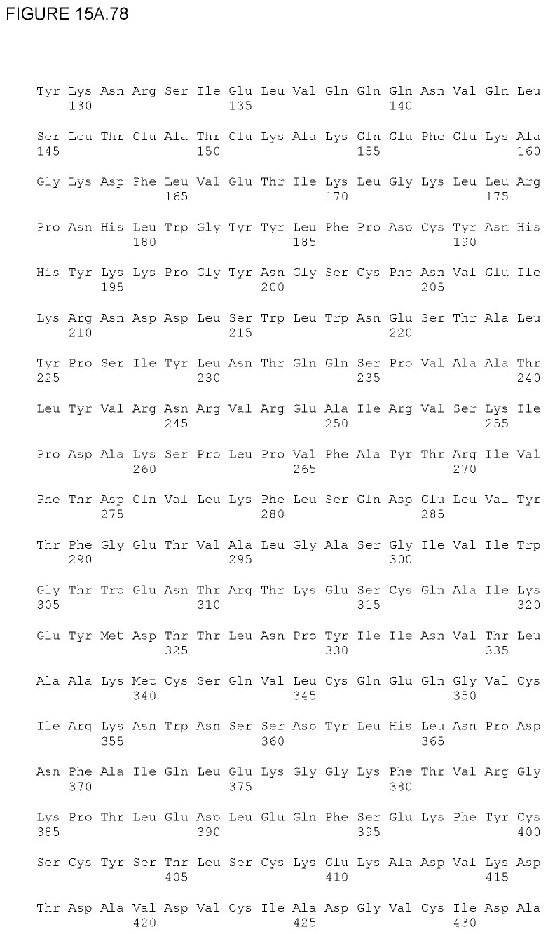

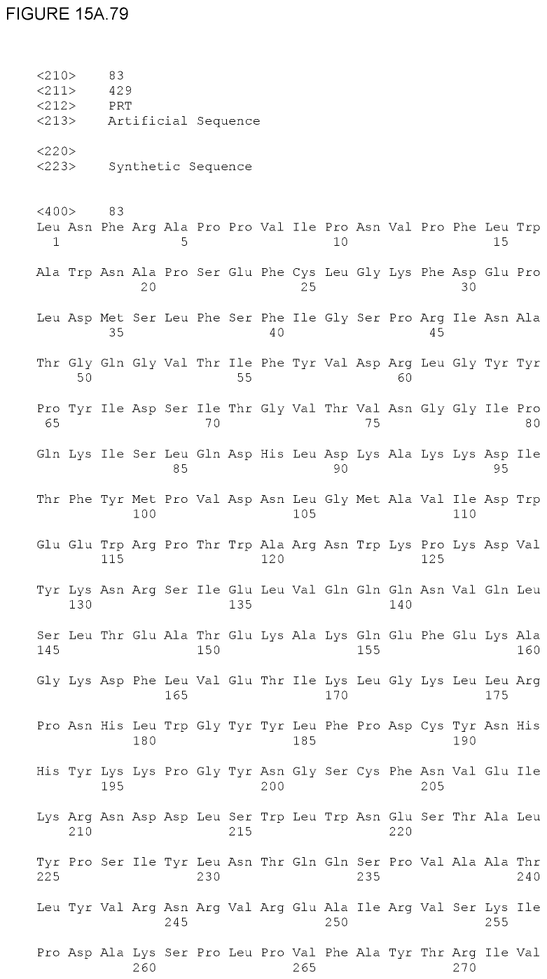

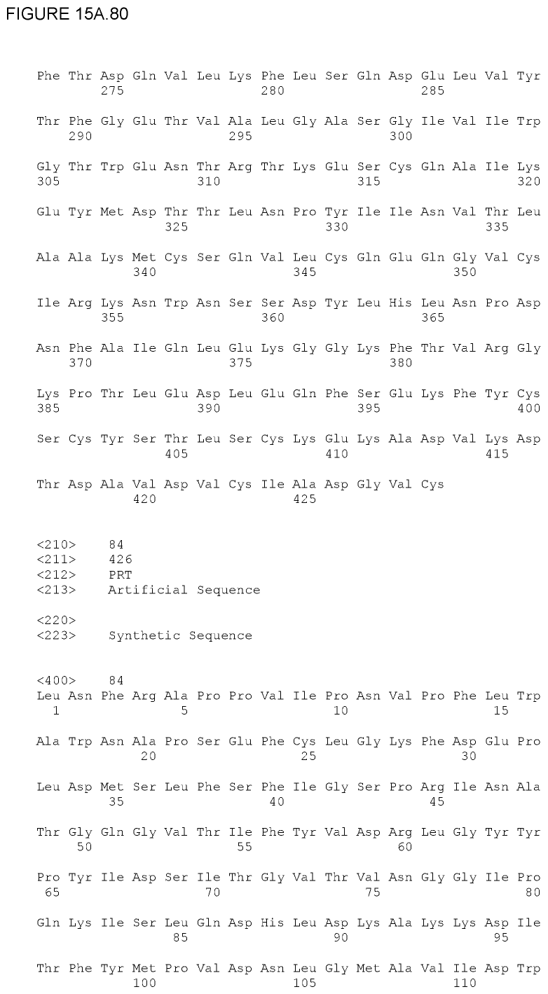

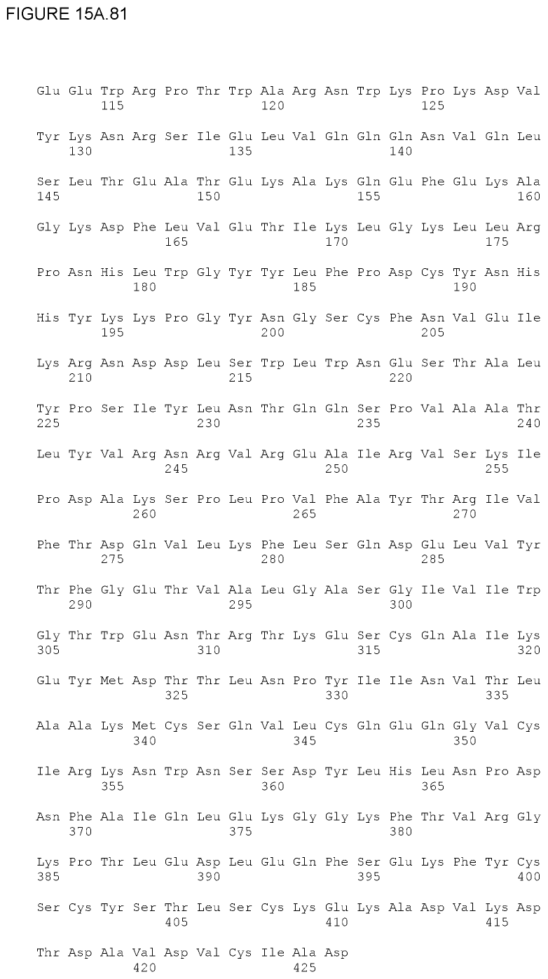

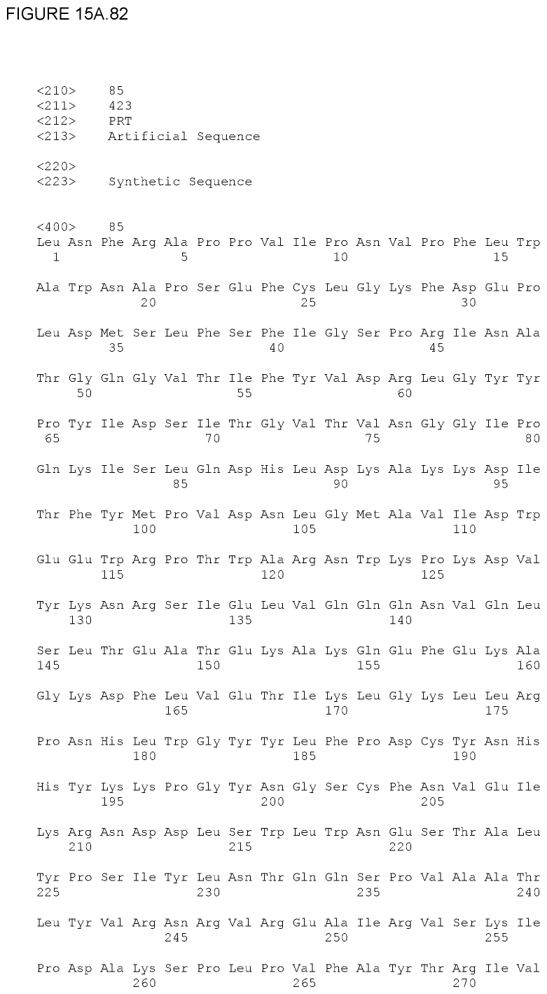

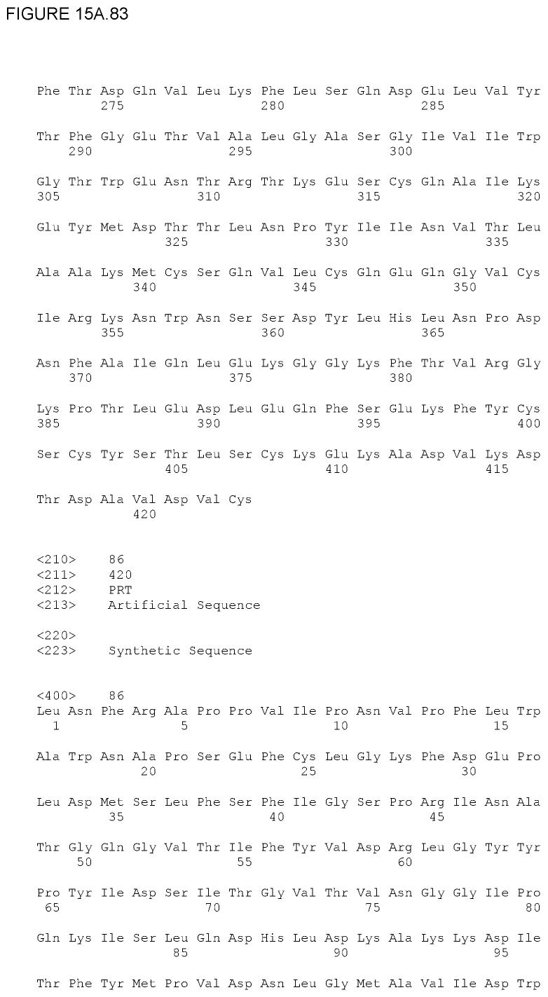

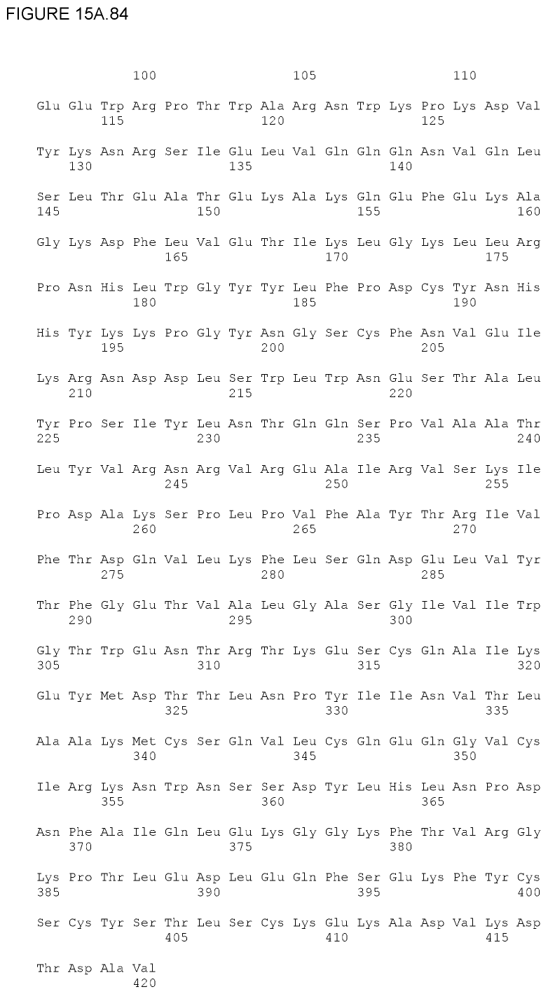

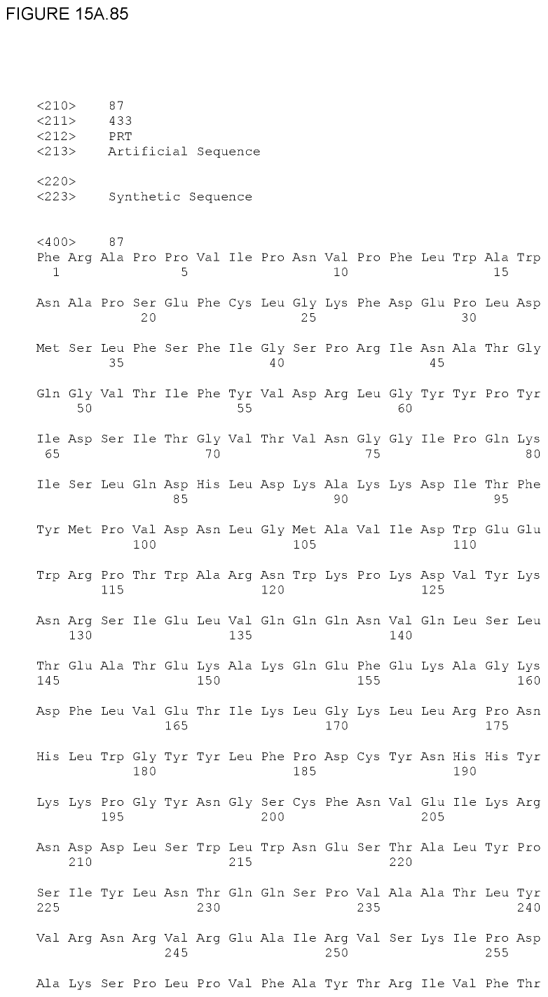

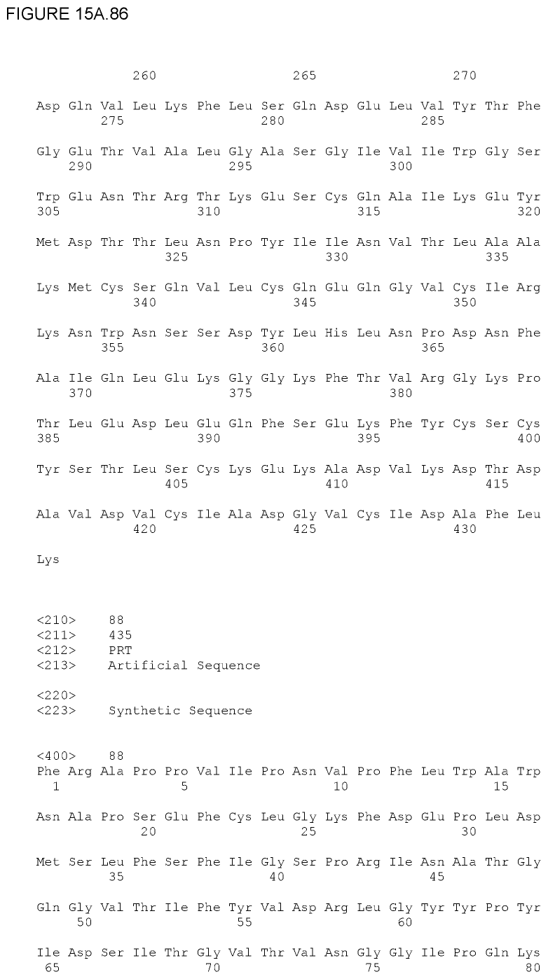

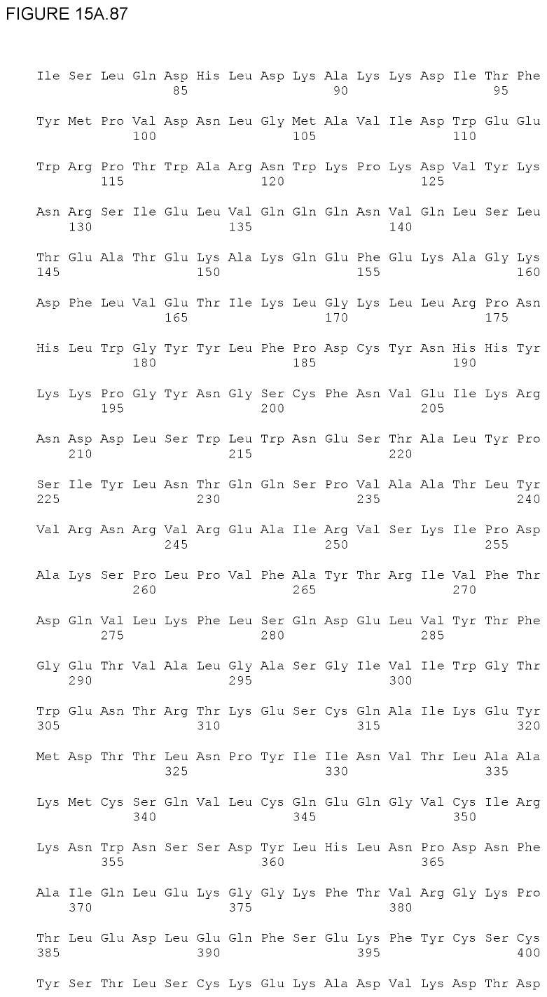

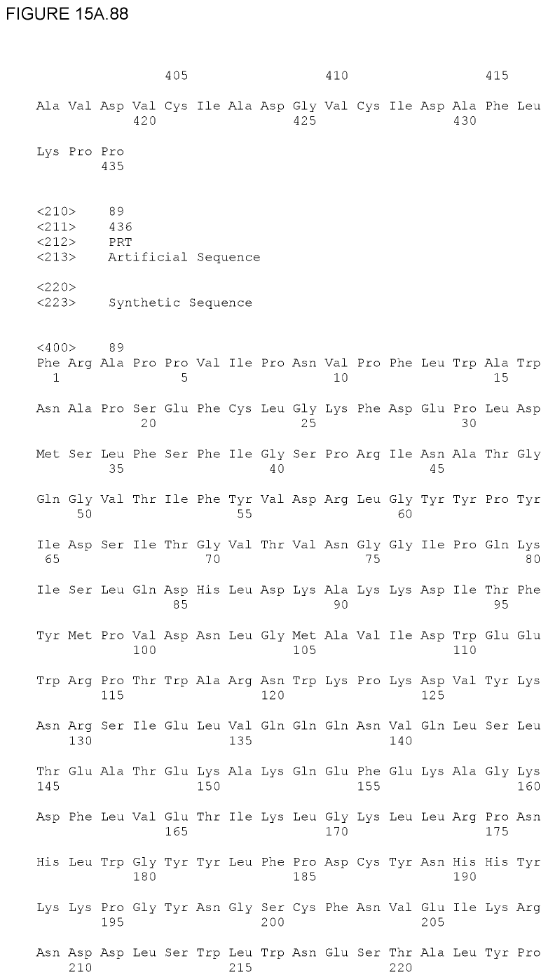

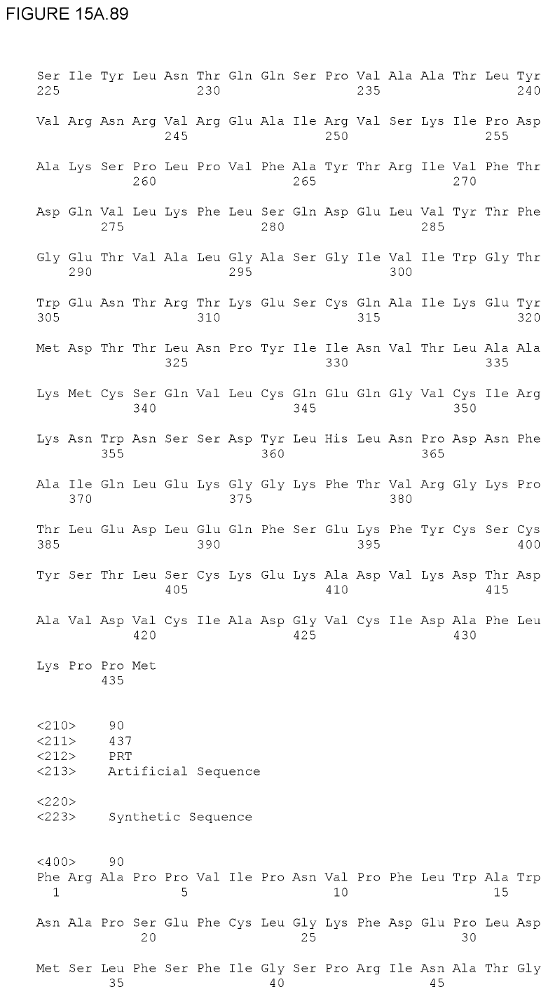

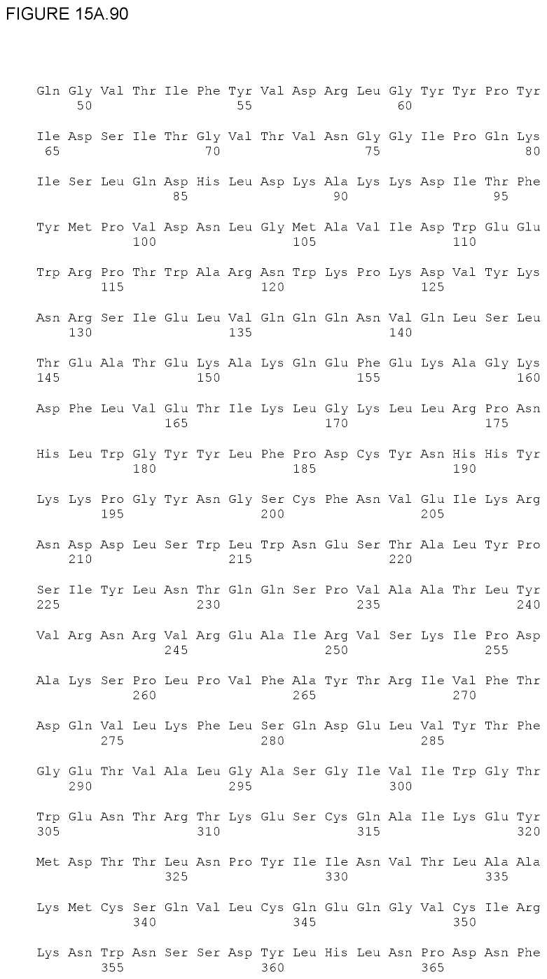

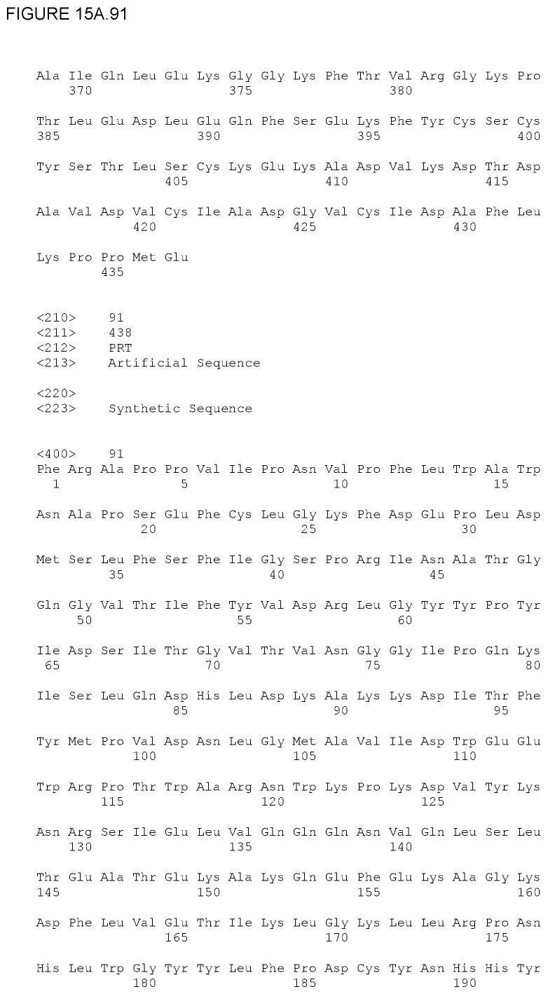

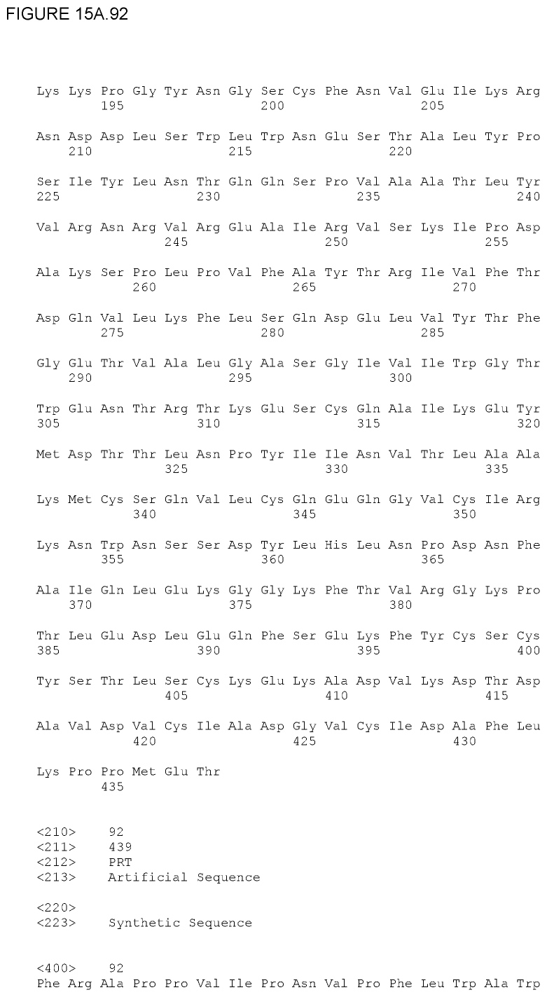

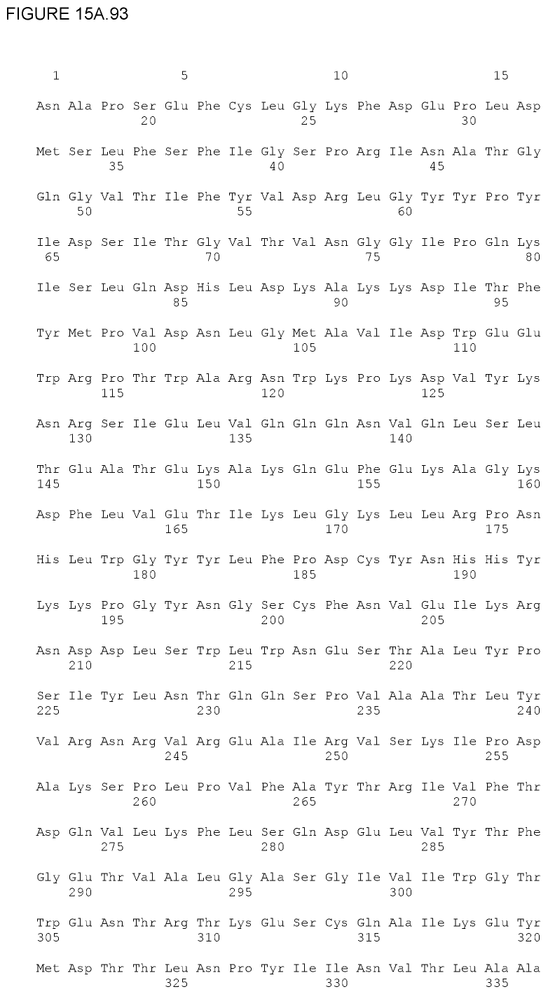

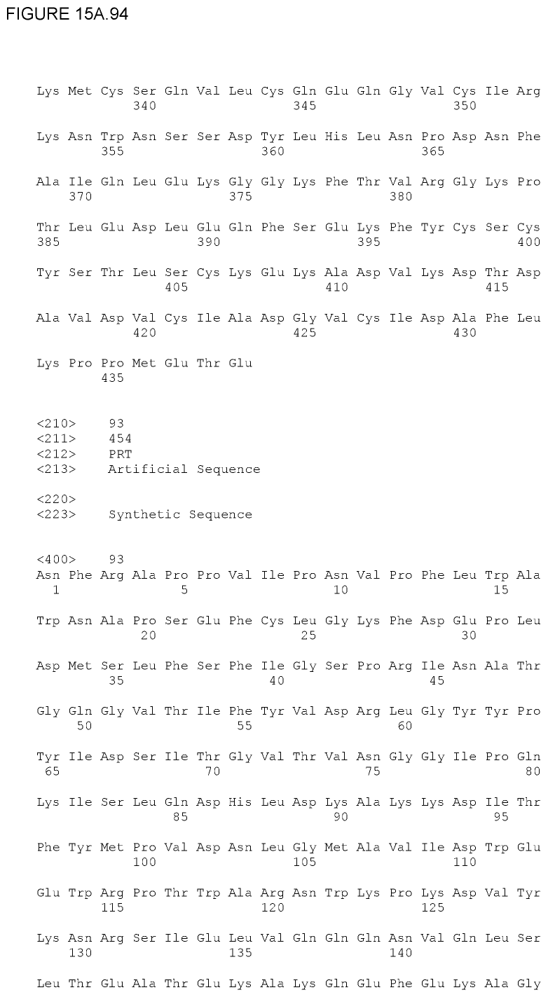

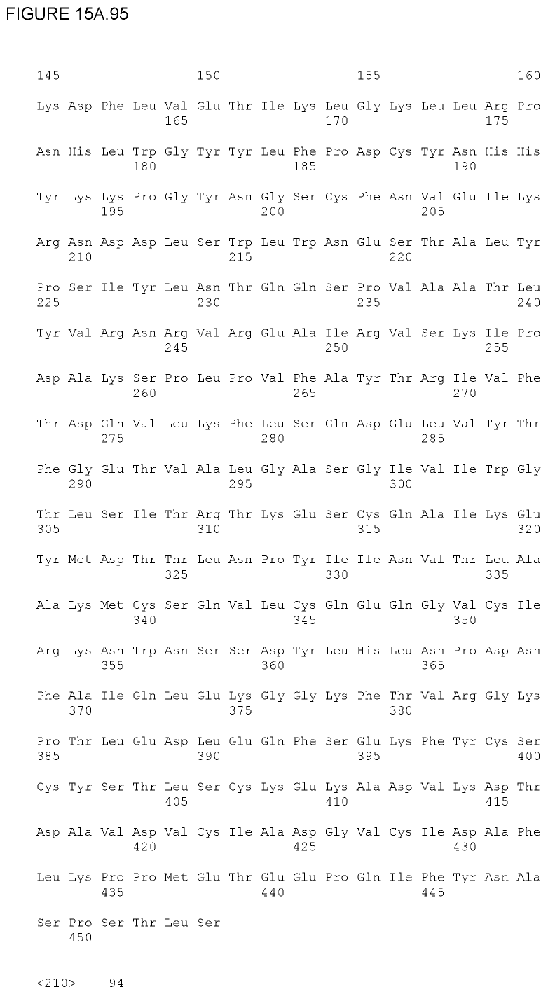

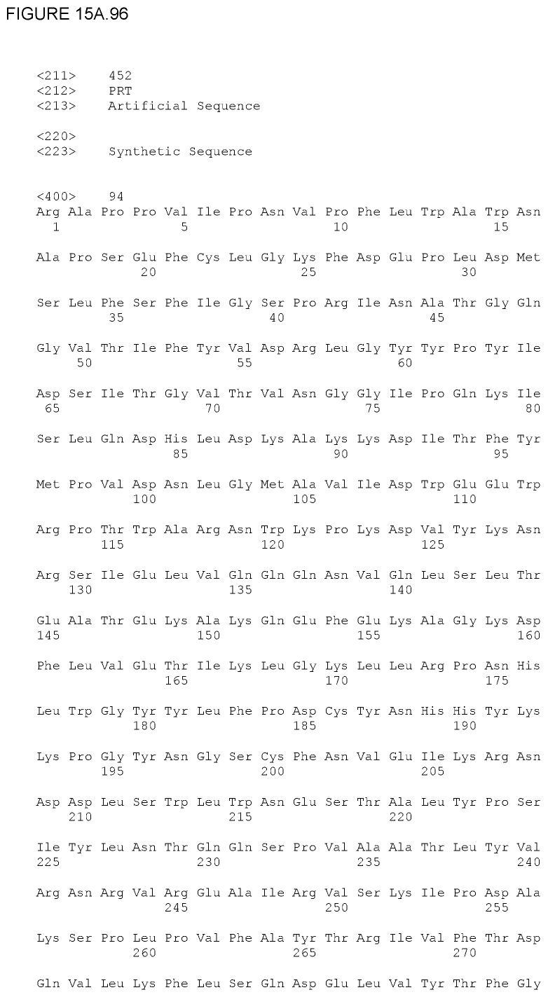

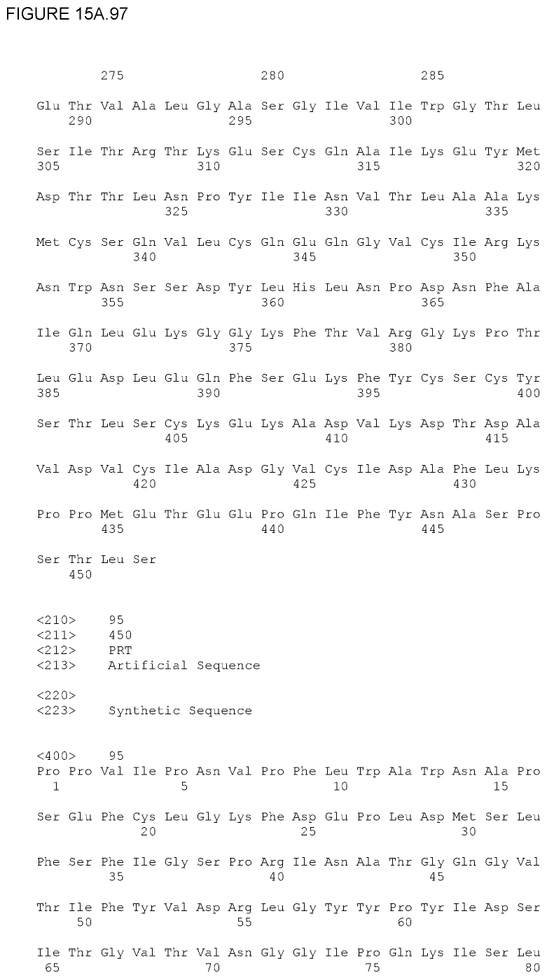

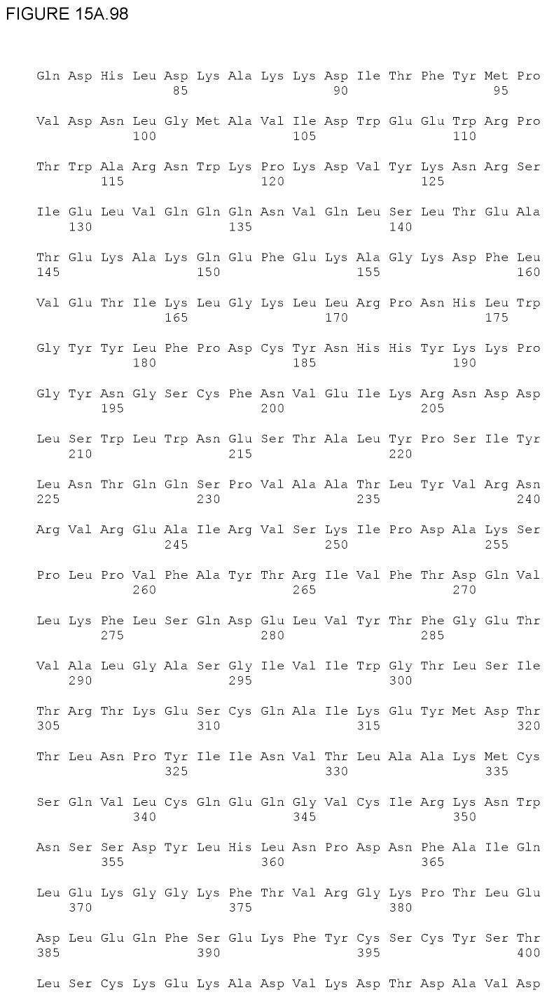

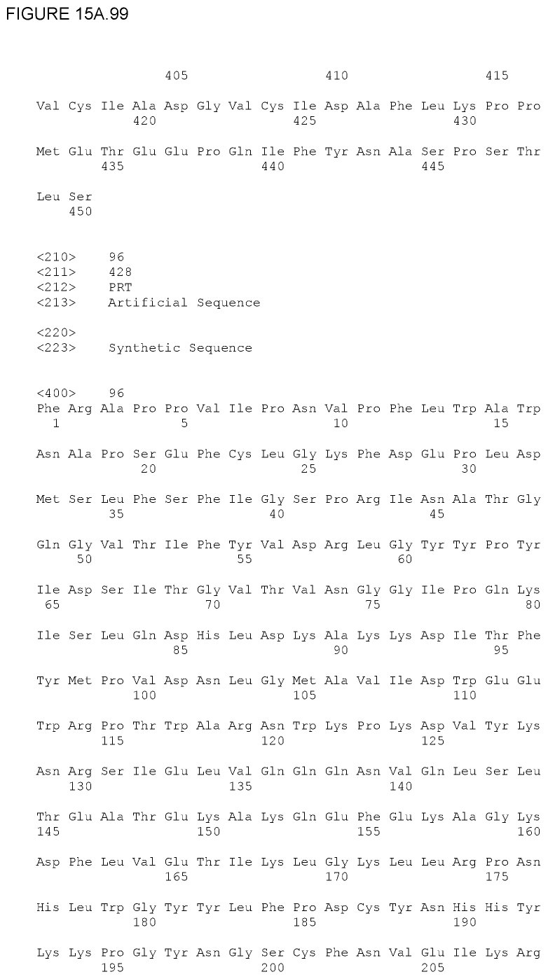

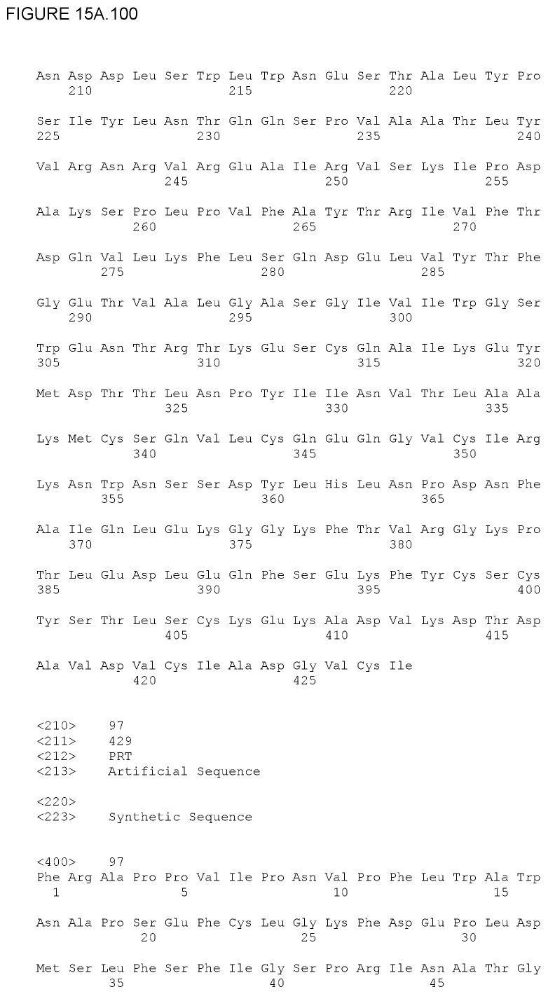

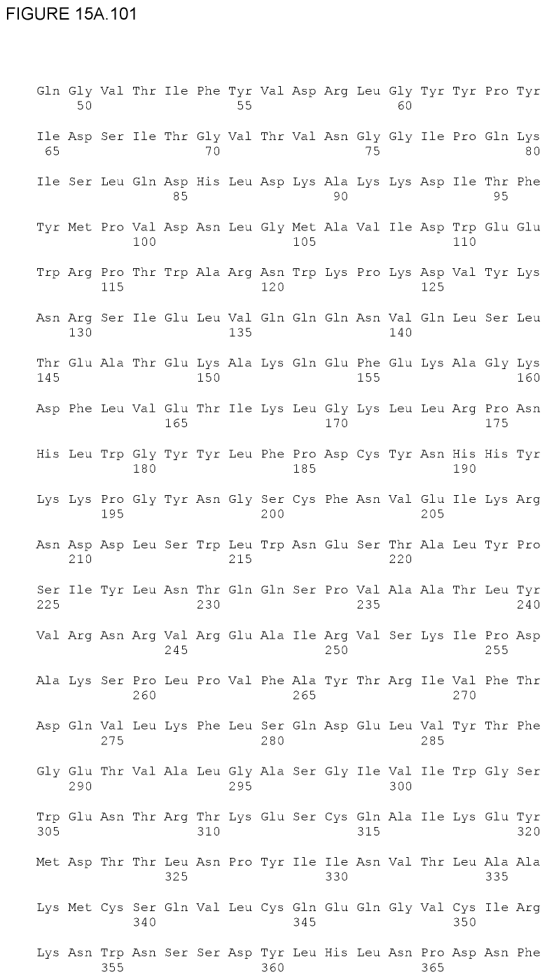

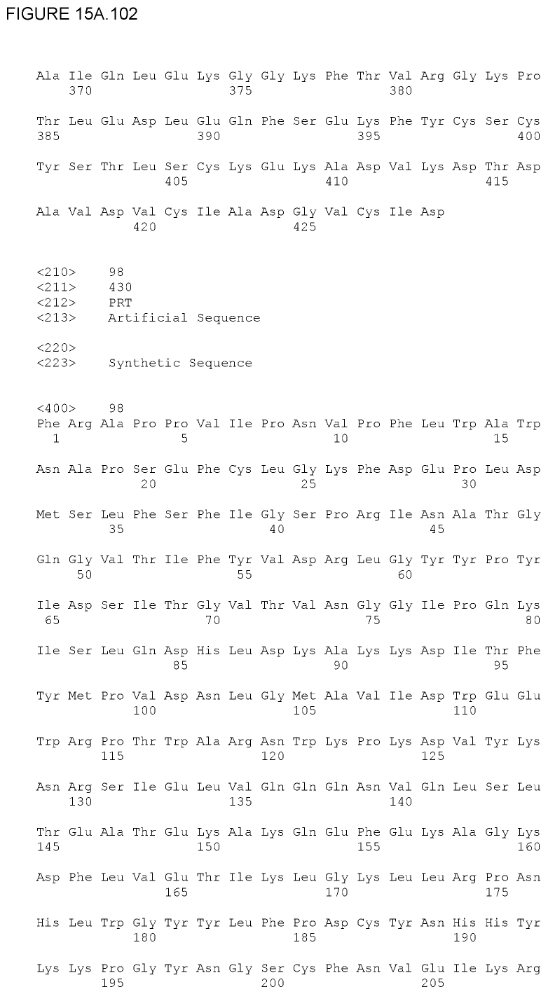

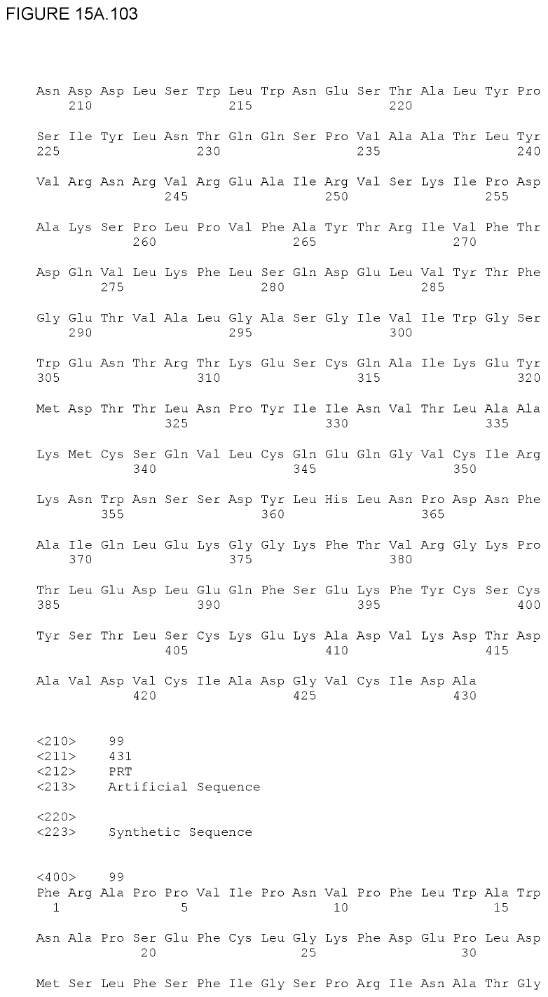

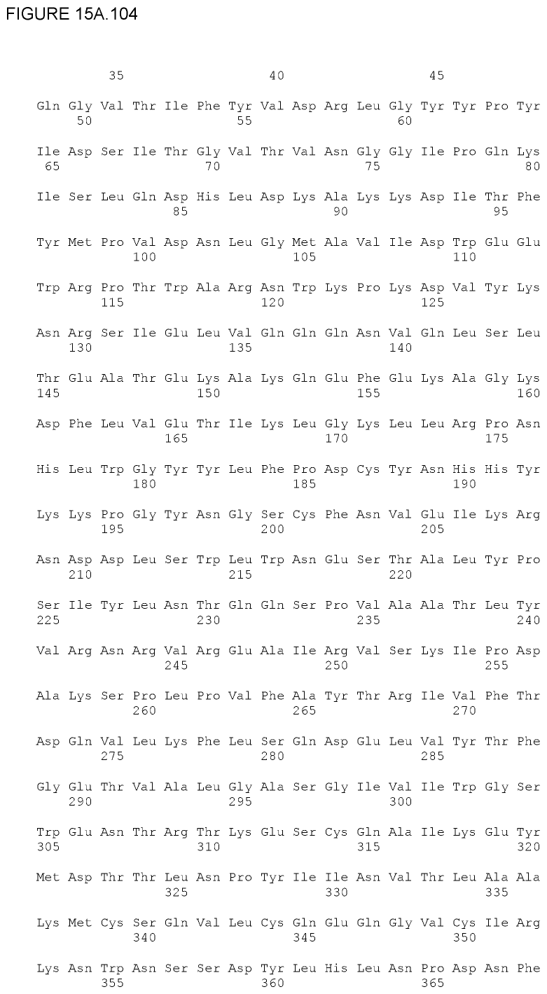

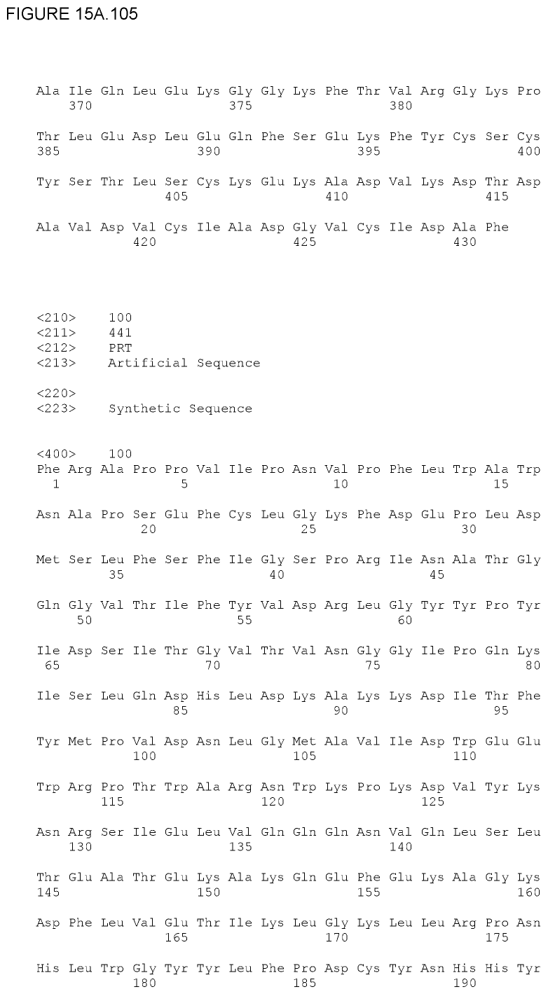

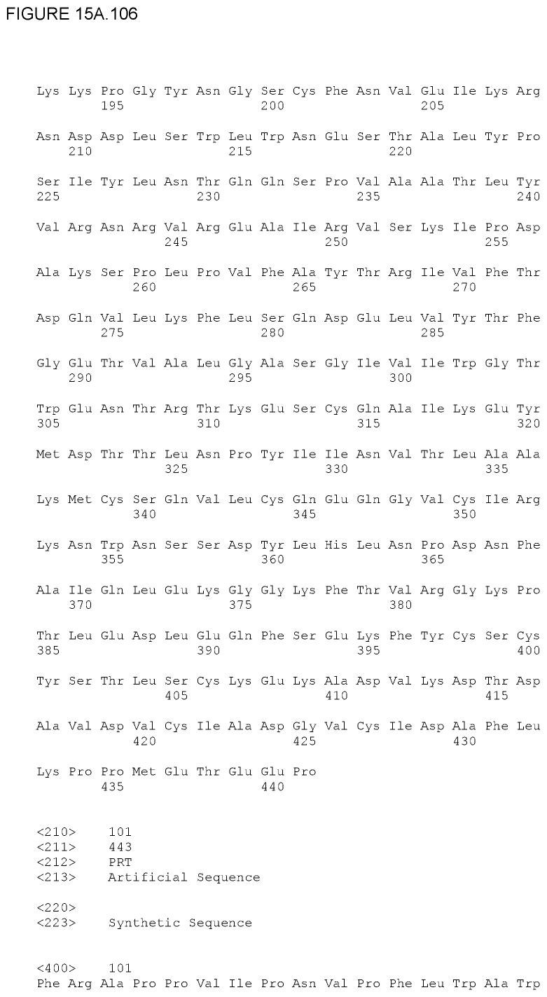

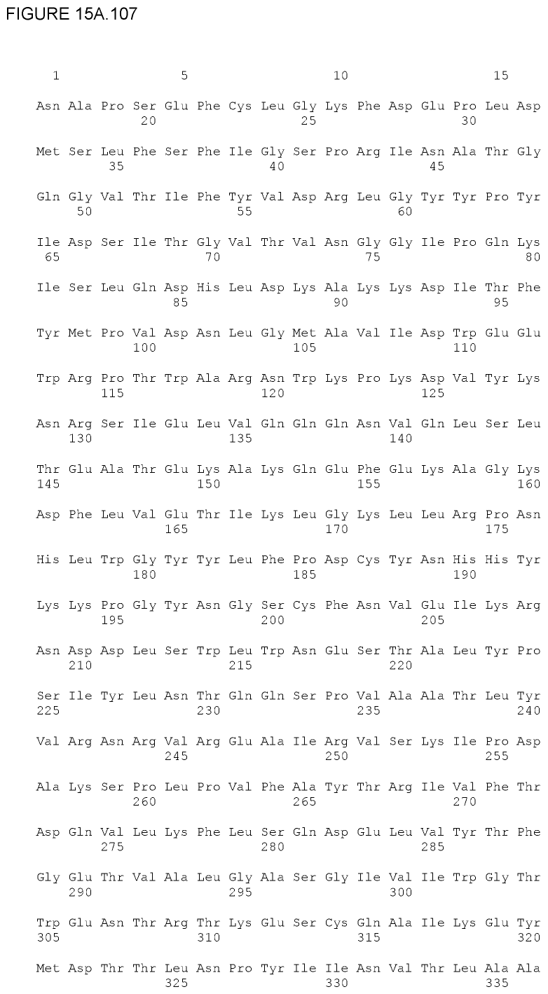

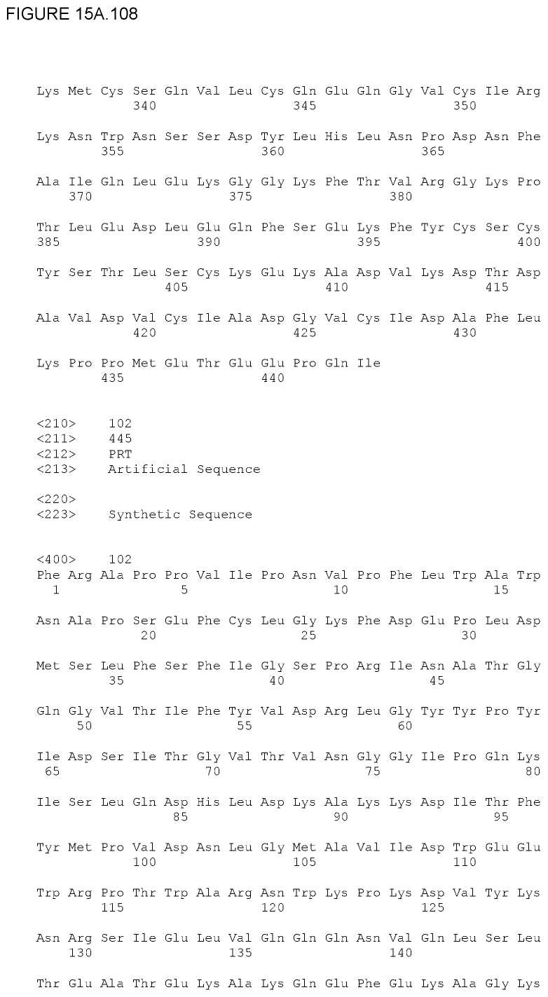

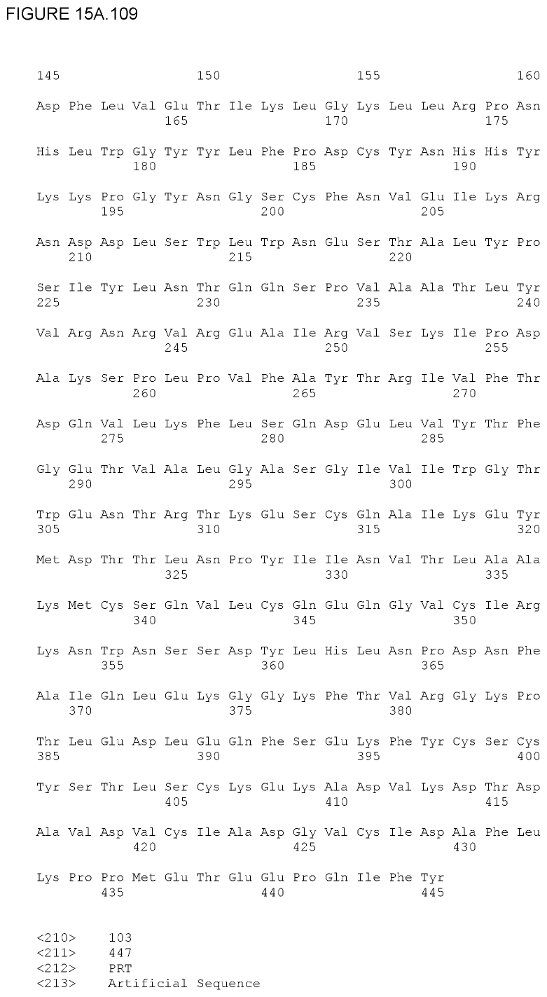

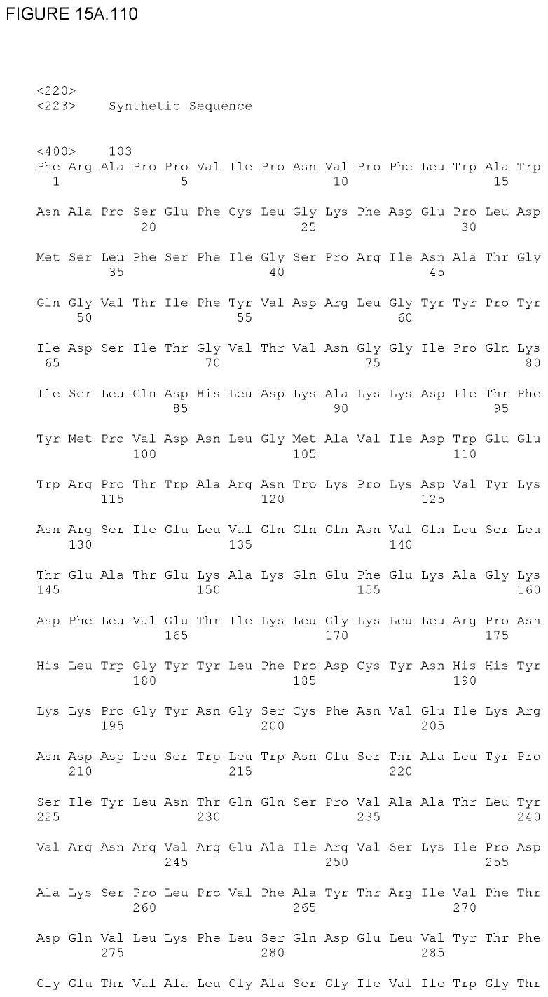

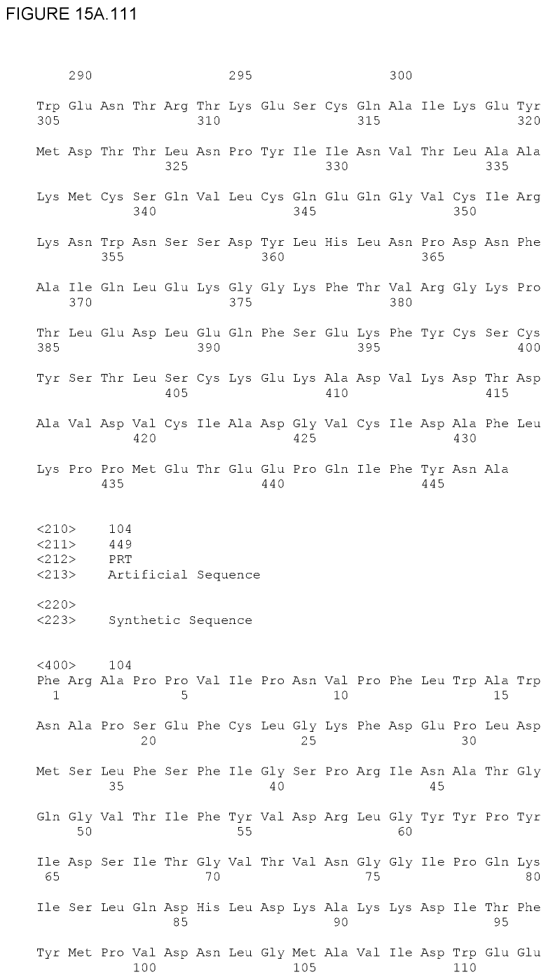

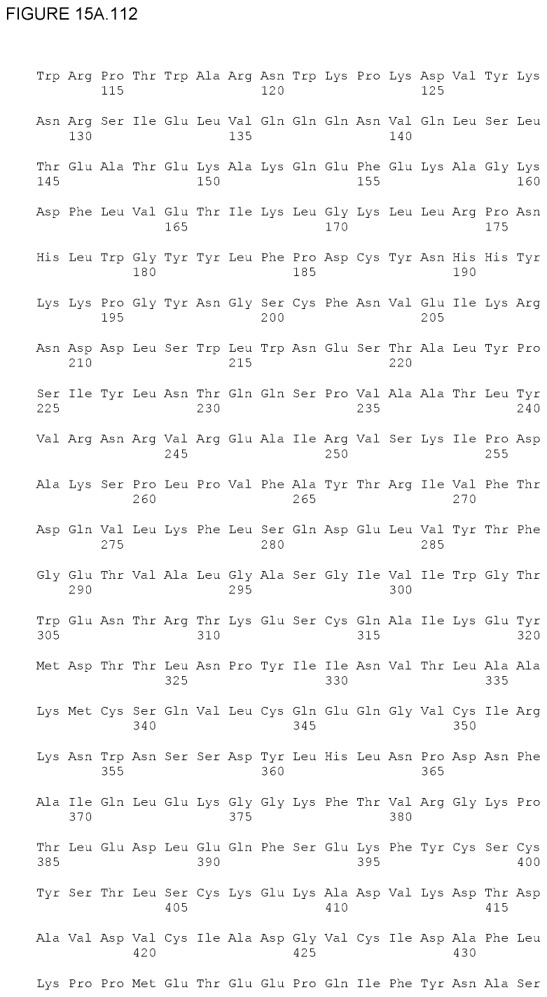

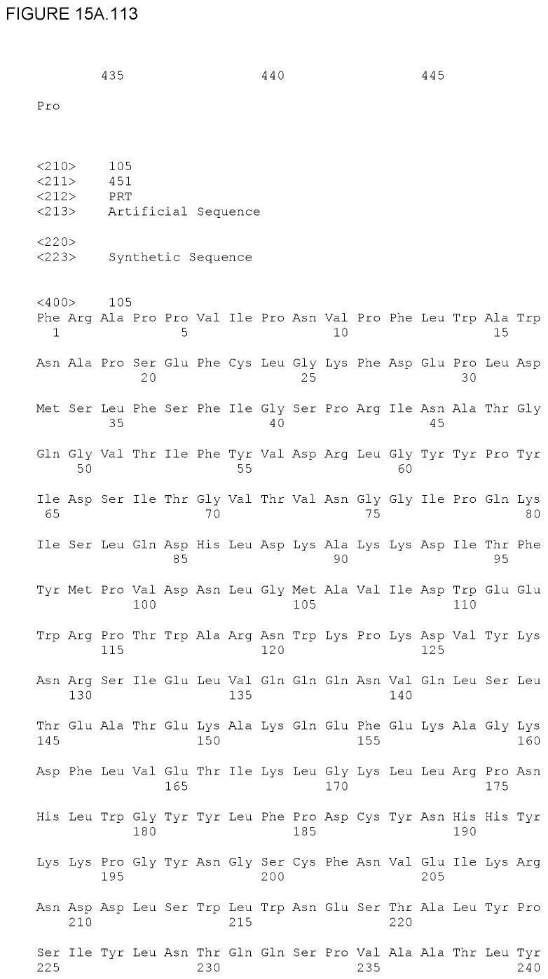

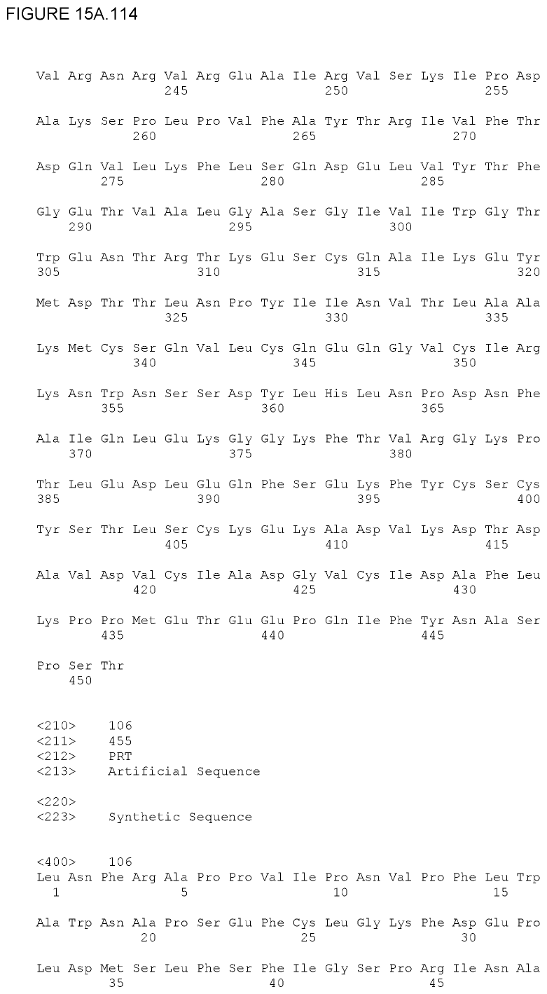

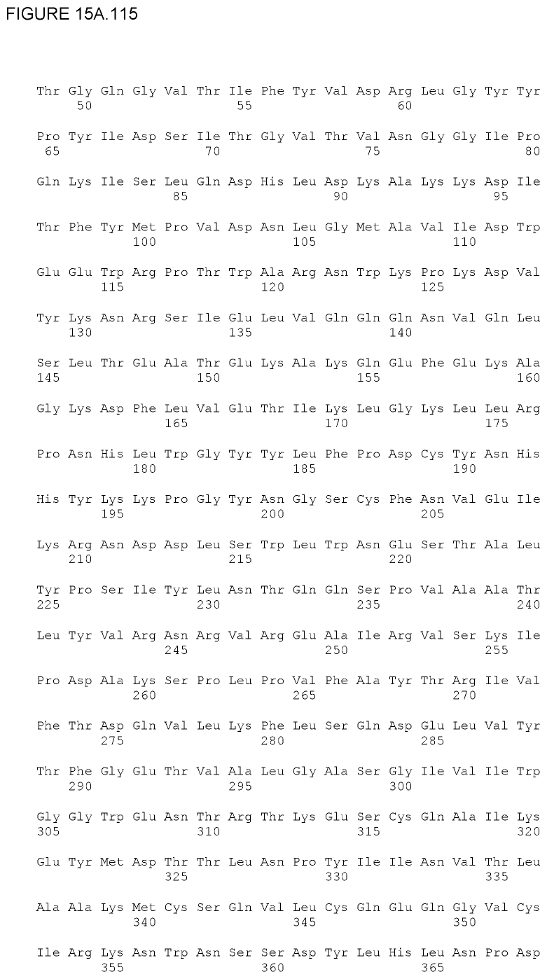

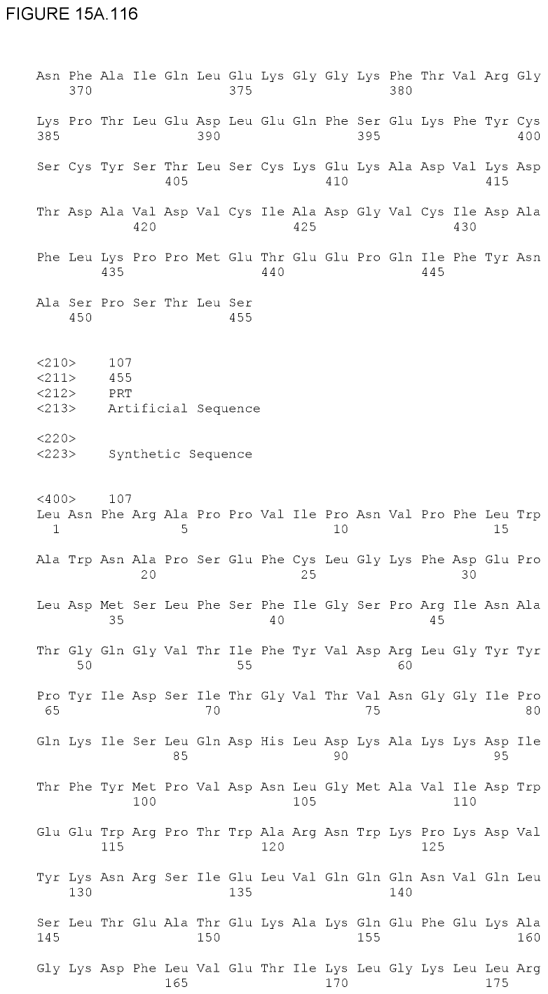

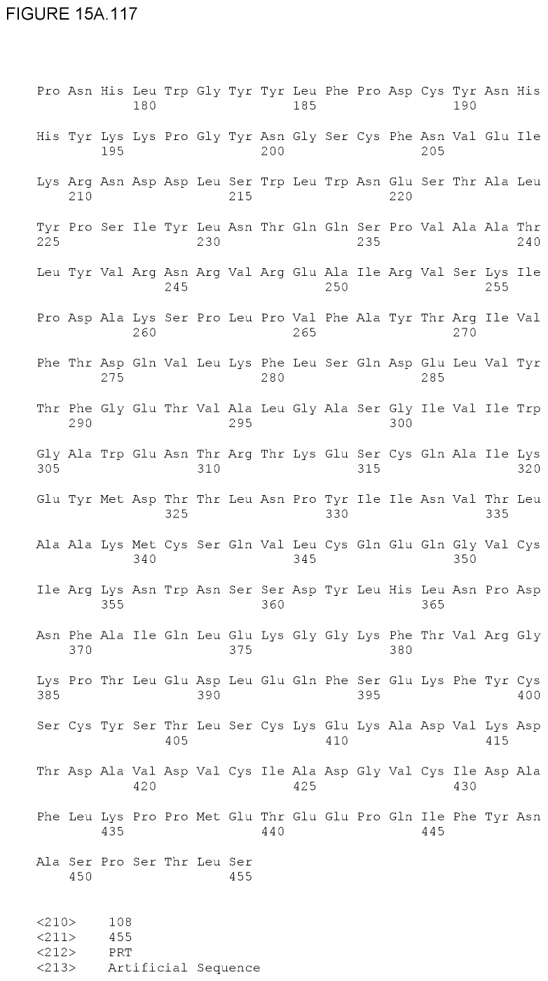

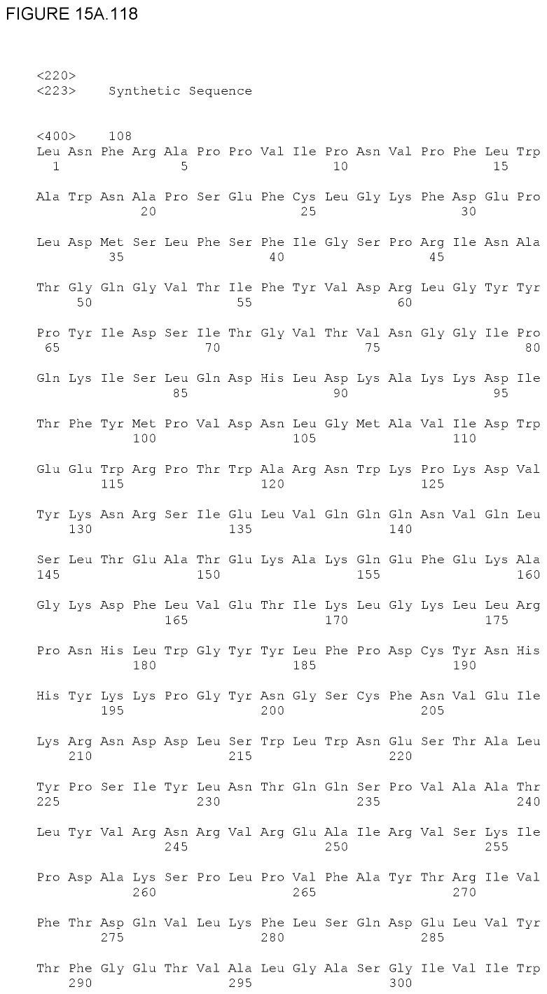

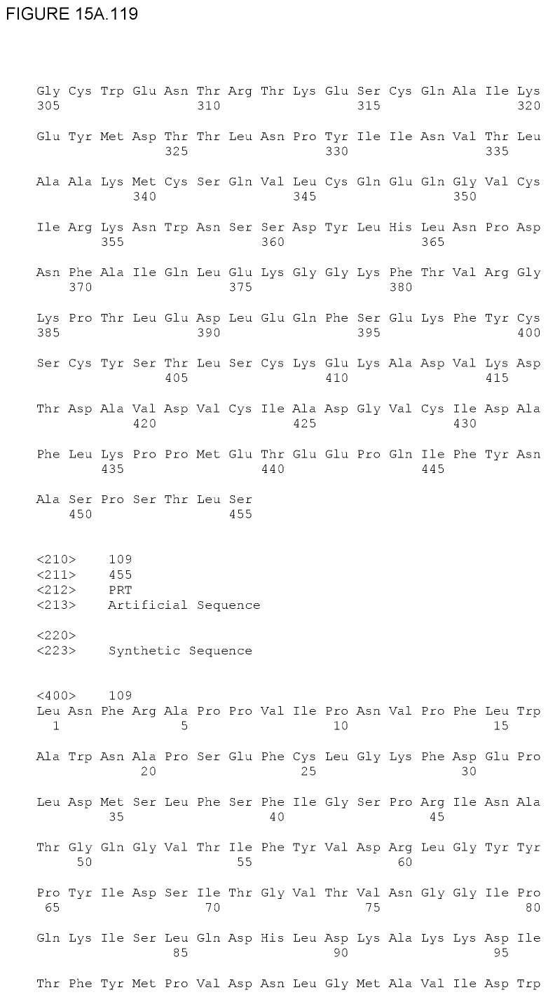

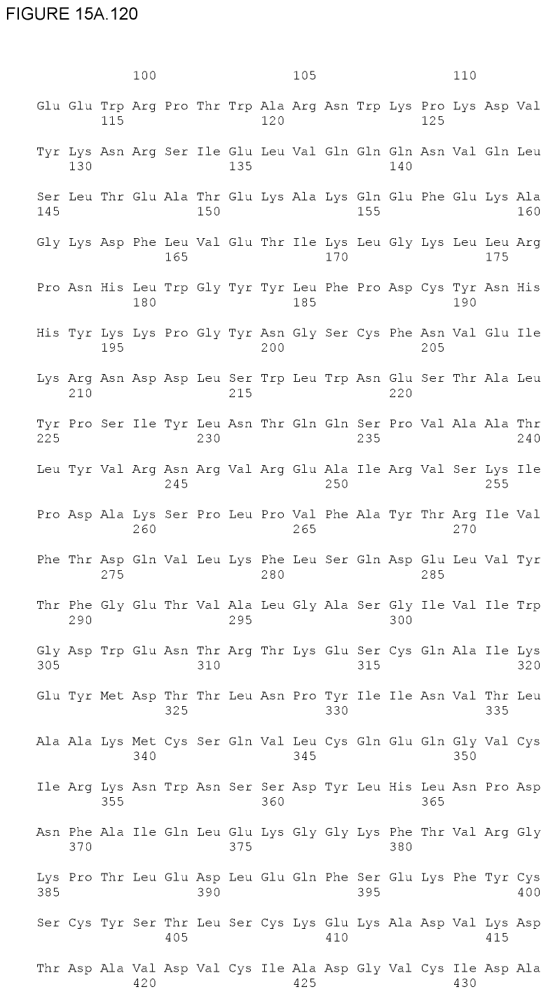

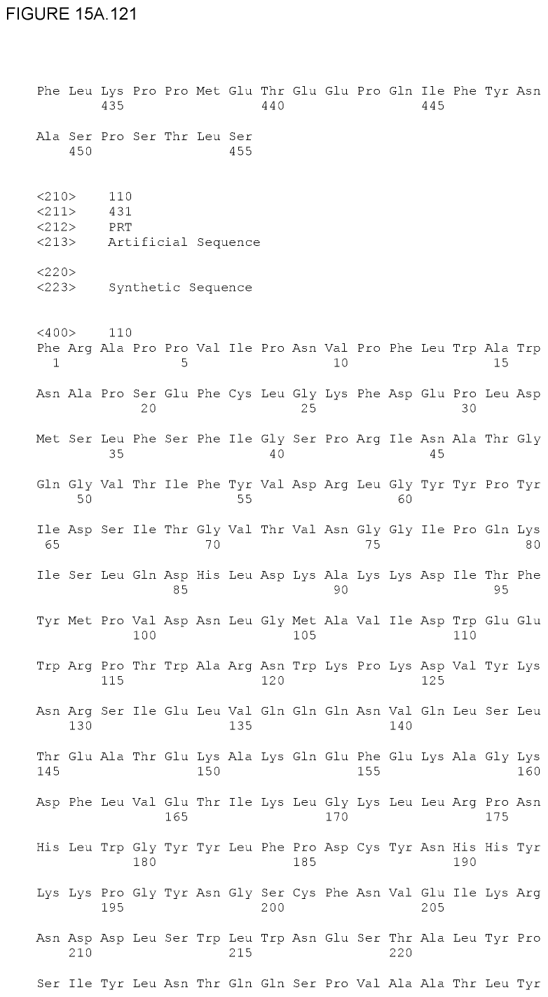

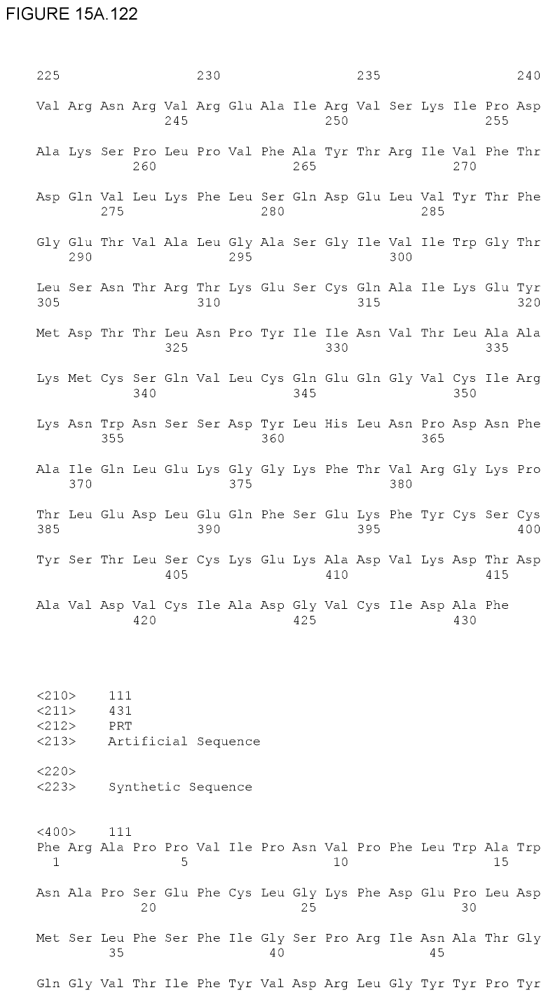

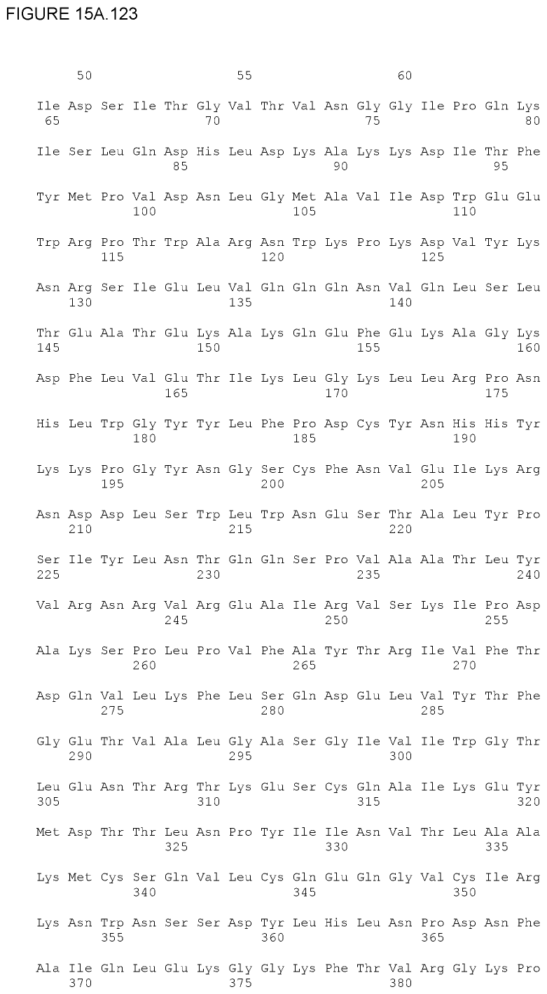

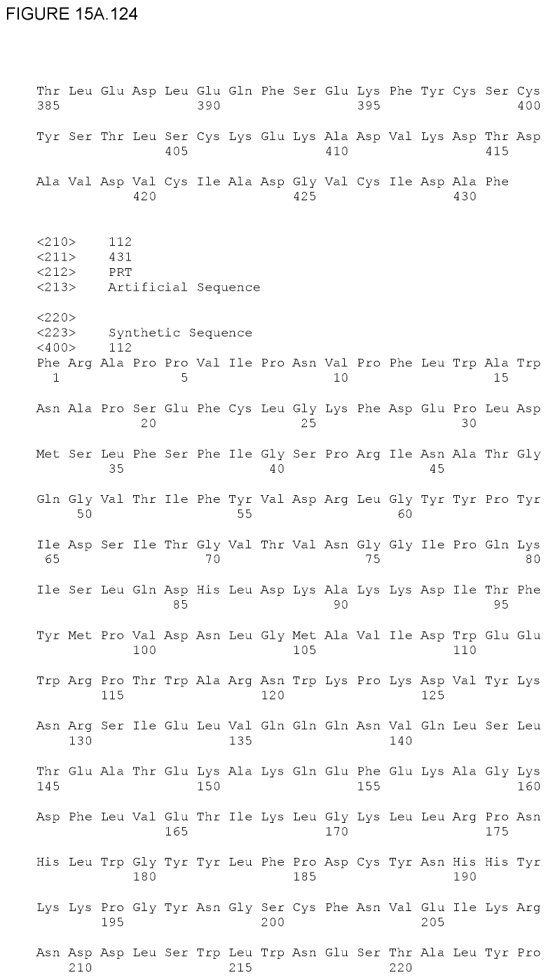

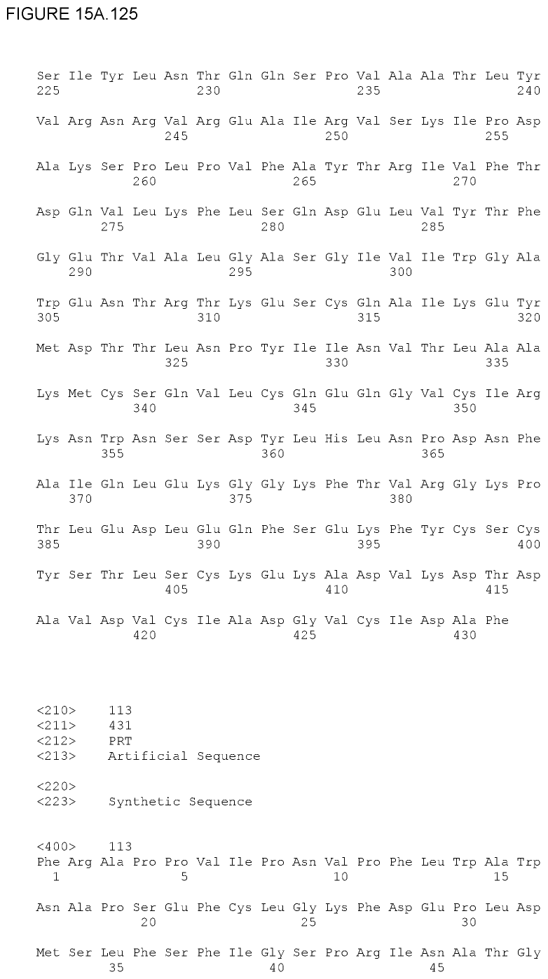

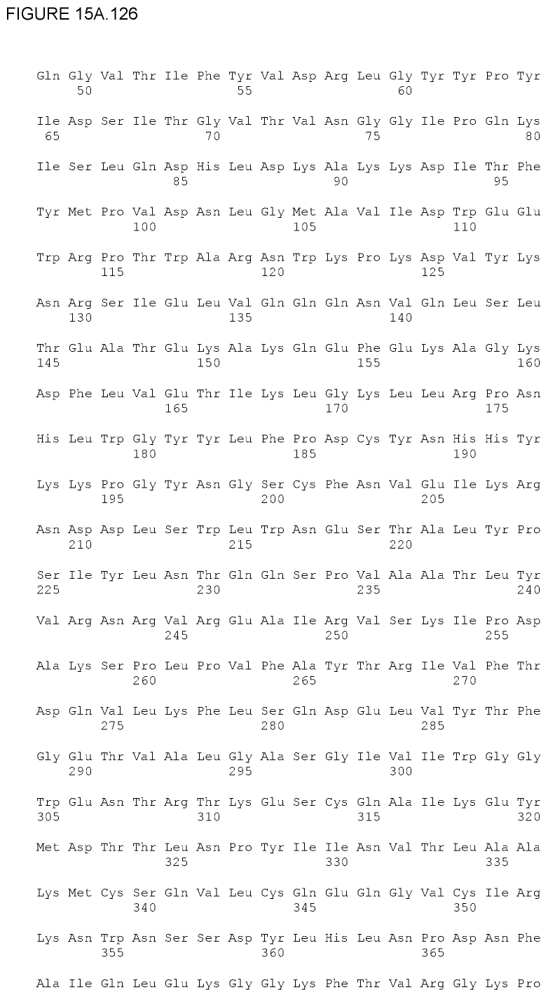

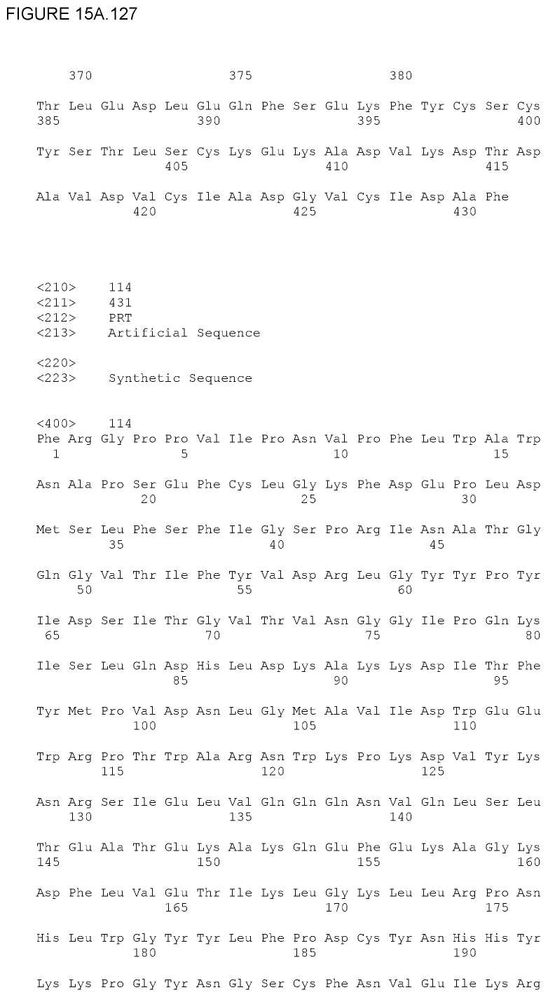

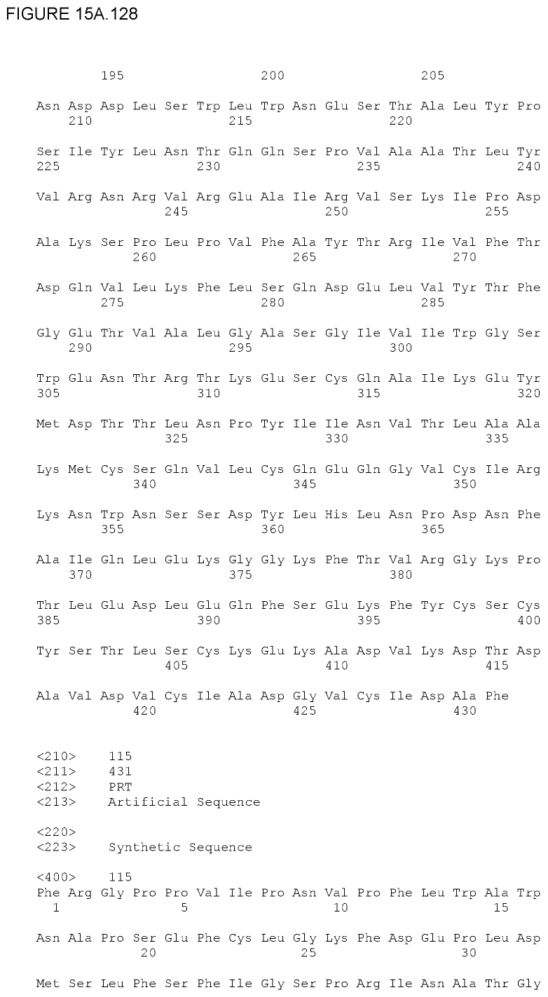

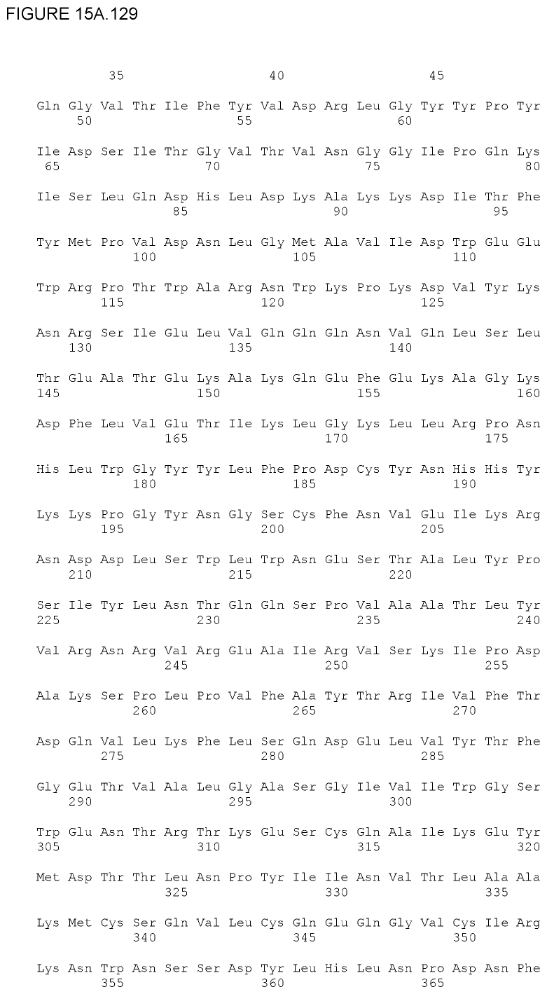

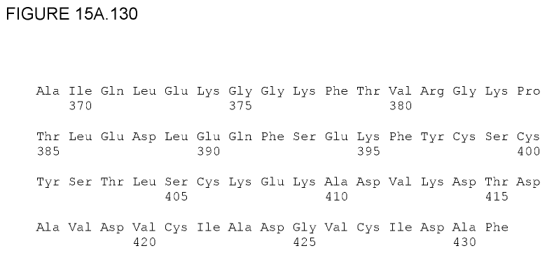

The overall study design is presented in FIG. 14.

This study comprised 3 treatment arms:
1) Treatment Arm 1-Subjects received a single dose of TAK-881 comprising of 0.4 g/kg (in-line warmed) IGSC, 20% at progressively increased infusion rates and rHuPH20 dose of 80 U/g IgG on Day 1 of the study treatment period.
2) Treatment Arm 2-Subjects received a single dose of TAK-881 comprising of 1.0 g/kg (in-line warmed) IGSC, 20% at progressively increased infusion rates and rHuPH20 dose of 80 U/g IgG on Day 1 of the study treatment period.
3) Treatment Arm 3-Subjects received a single dose of TAK-881 comprising 1.0 g/kg (un-warmed) IGSC, 20% at progressively increased infusion rates and rHuPH20 dose of 80 U/g IgG on Day 1 of the study treatment period.

The dosing and infusion rates as presented in Section 5.1.4 were followed for the study.

A total of 24 subjects with 8 subjects enrolled/treated in each of the 3 treatment arms.

### 4.2 Study Periods

The study consisted of 3 periods:
1) Screening period: up to 21 days prior to dosing.
2) Study treatment period: 4 days.
3) Follow-up period: up to 12 (+1) weeks after TAK-881 infusion.

Tolerability and safety including immunogenicity of TAK-881 was assessed during the treatment and follow-up periods for all 3 treatment arms.

All subjects were monitored for the formation of binding anti-rHuPH20 antibodies (binding anti-drug antibody [ADA]) at predose (baseline), postdose (Day 30±3 days), and at the end of study [EOS] (Week 12±1 week). Postdose samples with antibody titers ≥1:160 (ADA positive) were analyzed for the presence of neutralizing antibodies. No subjects had) postdose samples with antibody titers ≥1:160 (ADA positive) in this clinical trial.

After the EOS visit has been completed, no further visits are planned, unless determined necessary by the investigator. Positive binding antibody titers associated with serious or severe AEs required additional follow-up assessments-none occurred in this clinical trial.

### 4.3 Study Schedule

Study Treatment Administration: The dose levels are 0.4 g/kg (in-line warmed), 1.0 g/kg (in-line warmed), and 1.0 g/kg (un-warmed) with rHuPH20 80 U/g IgG for Treatment Arms 1, 2, and 3, respectively. Subjects received a single dose of IP with progressively increased infusion rate per the schedule presented in Section 5.3.2.

Serum Chemistry. Hematology, and Urinalysis:

Hematology included CBC·Serum chemistry included ALT, AST, ALP, K⁺, Na⁺, Cl⁻, Ca²⁺, Mg²⁺, bilirubin (total and direct), LDH, BUN, creatinine, uric acid, glucose, albumin, and lipid profile. A standard urinalysis and hemolytic panel was also tested.

Coagulation Tests included aPTT and INR assessments performed at screening and Day −1 as clinically indicated.

No potential safety signals were seen in any of the above tests, and the test results were mainly within expected ranges.

Immunogenicity Panel: The immunogenicity panel was collected at baseline (Day-1) and any time deemed necessary during the course of the study. Subjects, who had (a) 2 consecutive anti-rHuPH20 antibody titers of ≥1:160 which were elevated from the subject's baseline titers, and (b) a moderate or severe AE (Grade 2 or higher as per CTCAE v5.0) which could have been a result of immune-mediated response to either immunoglobulin, rHuPH20, or other concomitant medications, were asked to return to the CRC as soon as possible to undergo an additional panel of immunogenicity testing. No subject had an anti-rHuPH20 antibody titers of ≥1:160 which were elevated from the subject's baseline titers in this clinical study.

Serum Total IgG Levels: Serum total IgG samples were collected on Day-1, Day 4 (at discharge), Day 30 (+3 days), and at Week 12 (+1 week)/EOS or ET.

### 5. Investigational Product

### 5.1 Identity of Investigational Product(s)

Immune Globulin Subcutaneous (Human), 20% Solution (IGSC, 20%) with Recombinant Human Hyaluronidase (rHuPH20) (also referred to as IGSC, 20% with rHuPH20, or TAK-881). IGSC, 20% (human) was supplied in 8 g/40 mL vials with rHuPH20 160 Units/mL supplied separately in 15 mL vials.

### 5.1.1 Immune Globulin Subcutaneous 20% (Human)-IGSC, 20%

The IGSC, 20% (Human) is a ready-for-use, sterile, liquid preparation of highly purified and concentrated IgG antibodies. The distribution of the IgG subclasses is similar to that of normal plasma. The Fc and the Fab functions are maintained in the primary component. Pre-kallikrein activator activity is not detectable. The IGSC, 20% (Human) contains 200 mg/mL (20%) protein. At least ≥98% of the protein is IgG, contains trace amounts of IgA (average concentration of 80 mcg/mL). The IGSC, 20% (Human) contains a broad spectrum of IgG antibodies against bacterial and viral agents. Glycine (0.25 M) serves as a stabilizing and buffering agent. There is no added sugar, sodium, or preservatives. The pH is 4.6 to 5.1. The osmolality is 280 to 292 milli-osmoles/kg. The IGSC, 20% (Human) is manufactured from large pools of human plasma. IgG preparations are purified from plasma pools using a modified Cohn-Oncley cold ethanol fractionation process, as well as cation and anion exchange chromatography.

To further improve the margin of safety, validated virus inactivation/removal steps have been integrated into the manufacturing and formulation processes, namely solvent/detergent (S/D) treatment, 35 nm nanofiltration, and a low pH incubation at elevated temperature (30° C. to 32° C.). The solvent/detergent process includes treatment with an organic mixture of tri-n-butyl phosphate, octoxynol 9, and polysorbate 80 at 18° C. to 25° C. for a minimum of 60 minutes. Solvent/deterrent treatment inactivates the lipid-enveloped viruses investigated to below detection limits within minutes. The ethanol fractionation process provides an additional virus clearance capacity.

### 5.1.2 Recombinant Human Hyaluronidase-rHuPH20

The rHuPH20 component of TAK-881 is produced from genetically engineered Chinese Hamster Ovary cells containing a DNA plasmid encoding for a soluble fragment of human hyaluronidase PH20. rHuPH20 is used in HyQviaR, Immune Globulin Infusion 10% (Human) with Recombinant Human Hyaluronidase. The purified hyaluronidase glycoprotein contains 447 amino acids with an approximate molecular weight of 61,000 Daltons. This component is supplied as a sterile, clear, colorless, ready-for-use solution and has approximately pH of 7.4 and an osmolality of 290 to 350 milli-osmoles. Each vial contains 160 U/mL of recombinant human hyaluronidase. It does not contain preservatives.

Due to comprehensive virus testing at the master cell bank, working cell bank, and bulk harvest stages, effective virus reduction during the purification process and use of pharmaceutical grade human albumin as an excipient with no other materials of human or animal origin involved in the manufacturing process, rHuPH20 provides for high margins of safety with respect to viruses.

### 5.1.3 Dosing

This study comprised 3 treatment arms:
1) Treatment Arm 1-Subjects received a single dose of TAK-881 comprising of 0.4 g/kg (in-line warmed) IGSC, 20% at progressively increased infusion rates and rHuPH20 dose of 80 U/g IgG on Day 1 of the study treatment period.
2) Treatment Arm 2-Subjects received a single dose of TAK-881 comprising of 1.0 g/kg (in-line warmed) IGSC, 20% at progressively increased infusion rates and rHuPH20 dose of 80 U/g IgG on Day 1 of the study treatment period.
3) Treatment Arm 3-Subjects received a single dose of TAK-881 comprising 1.0 g/kg (un-warmed) IGSC, 20% at progressively increased infusion rates and rHuPH20 dose of 80 U/g IgG on Day 1 of the study treatment period.

The dose for rHuPH20 is 80 U/g IgG. The rHuPH20 units were calculated as per the following:

$$\begin{matrix}The dose of rHuPH 20 is 80 Units \times planned IGSC 20 \% dose in grams = total units to be infused ( e . g . , 80 U \times 40 g = 3200 U ) . & 1 ) \\ Then , to calculate the volume required , divide the prescribed units by 160 as each vial has a concentration of 160 U / mL , ( e . g . , 3200 U \div 160 U / mL = 20 mL ) & 2 )\end{matrix}$$

Dosing was first initiated at the lower dose level (Treatment Arm 1, 0.4 g/kg, in-line warmed) followed by the higher dose level (Treatment Arm 2, 1.0 g/kg, in-line warmed) and then the un-warmed arm (Treatment Arm 3, 1.0 g/kg, un-warmed). Subjects in all 3 treatment arms were dosed according to a sentinel dosing design with ongoing safety monitoring by the investigator to ensure optimal tolerability and safety. Subjects in each treatment arm were grouped into 4 subgroups of 1, 1, 2, and 4 subjects, respectively. The subgroups were dosed sequentially to allow safety and tolerability evaluation prior to initiating dosing of the following subgroup.

Subjects were well hydrated prior to drug administration.

### 5.1.4 Mode of Administration

The TAK-881-1001 investigational product (IP) was administered via a SC route of administration using a 22 to 24-gauge SC needle set. The rHuPH20 solution was administered first followed by IGSC 20% using the same needle set.

The rHuPH20 solution was administered subcutaneously via a peristaltic infusion pump at a rate of 120 mL/hour/site and infusion volumes of up to 30 mL/site.

The infusion site(s) were either the abdomen (middle to upper abdomen) or the thighs (left or right).

The SC infusion of the IGSC 20% solution began within 10 minutes of completion of the SC infusion of the rHuPH20 solution via a peristaltic infusion pump with programmable infusion rates and infusion volumes of up to 300 mL/site and could have required 1 or 2 infusion sites. If 2 infusion sites were required, the doses were administered sequentially; the infusion of up to 300 mL would be administered first. A flushing step of normal saline was required to ensure the total dose is administered due to the large priming volumes of the 2 administration systems. Normal saline was not infused into the subject. For each infusion site, infusion rate ramp-up schedule was followed as shown in Table 14, Table 15, and Table 16.

If the infusion site was reduced or interrupted due to an intolerability event, the infusion rate stayed at the maximally tolerable infusion rate (e.g., if the maximum infusion rate was 300 mL and it is not tolerable, the infusion rate was decreased to the previous infusion rate of 180 mL assuming it was well tolerated). No intolerability events occurred in this clinical study, so the last infusion rate used per infusion site was recorded as the highest tolerated infusion rate for that infusion site (e.g., if a total volume at a second infusion site was 20 ml, the highest tolerated infusion rate recorded for that site would be 120 ml/hr).

A step-wise infusion rate escalation regimen as shown below was followed for the study based on tolerability of each incremental infusion rate increase (Table 14, Table 15, Table 16).

TABLE 14
Infusion Rates for Treatment Arm 1 (0.4 g/kg, in-line warmed)
Using 1 Pump and 1 Single Needle Set.

| | | Rate Per Infusion | | Accumulative |
|---|---|---|---|---|
| | | Site (mL/hour) | Volume | Volume for IG per |
| Administration | | Single Needle Set | Delivered | Site |
| rHuPH20 | Infused first | 120 | TBD | N/A |
| IGSC 20% | First 10 min | 30 | 5 mL | 5 mL |
| (in-line | Next 10 min | 60 | 10 mL | 15 mL |
| warmed) | Next 10 min | 120 | 20 mL | 35 mL |
| | Next 10 min | 180 | 30 mL | 65 mL |
| | Remainder of infusion | 300 | TBD | TBD (up to 300 mL)* |

Abbreviations:
IG = immune globulin;
N/A = not applicable;
TBD = to be determined
*Total volume of up to 300 mL did not include the volume of the rHuPH20 delivered first.

TABLE 15
Infusion Rates for Treatment Arm 2 (1.0 g/kg, in-line warmed) Using 2 Pumps and 1 Single
Needle Set for Each Pump.

| | | Rate Per Infusion | Volume | Accumulative | Accumulative |
|---|---|---|---|---|---|
| | | Site (mL/hour) | Delivered for | Volume Site 1^{#} | Volume Site 2^{#} |
| Administration | | for Each Pump | Each Pump | (Pump A) | (Pump B) |
| rHuPH20 | Infused first | 120 | TBD | TBD | TBD |
| IGSC 20% | First 10 min | 30 | 5 mL | 5 mL | 5 mL |
| (in-line | Next 10 min | 60 | 10 mL | 15 mL | 15 mL |
| warmed) | Next 10 min | 120 | 20 mL | 35 mL | 35 mL |
| | Next 10 min | 180 | 30 mL | 65 mL | 65 mL |
| | Remainder of | 300 | TBD | TBD | TBD (remainder) |
| | infusion | | | (up to 300 mL)* | |

Abbreviations:
TBD = to be determined
^{#}Each site was evaluated separately.
*Total volume of up to 300 mL did not include the volume of the rHuPH20 delivered first.

TABLE 16
Infusion Rates for Treatment Arm 3 (1.0 g/kg, un-warmed)
Using 2 Pumps and 1 Single Needle Set for Each Pump.

| | | Rate Per Infusion | Volume | Accumulative | Accumulative |
|---|---|---|---|---|---|
| | | Site (mL/hour) | Delivered for | Volume Site 1^{#} | Volume Site 2^{#} |
| Administration | | for Each Pump | Each Pump | (Pump A) | (Pump B) |
| rHuPH20 | Infused first | 120 | TBD | TBD | TBD |
| IGSC 20% | First 10 min | 30 | 5 mL | 5 mL | 5 mL |
| (un- | Next 10 min | 60 | 10 mL | 15 mL | 15 mL |
| warmed) | Next 10 min | 120 | 20 mL | 35 mL | 35 mL |
| | Next 10 min | 180 | 30 mL | 65 mL | 65 mL |
| | Remainder | 300 | TBD | TBD | TBD |
| | of infusion | | | (up to 300 mL)* | (remainder) |

Abbreviations:
TBD = to be determined
^{#}Each site was evaluated separately.
*Total volume of up to 300 mL did not include the volume of the rHuPH20 delivered first.

### 6. Clinical Study Results

The results of the Phase I clinical study are presented in Table 17, Table 18. Table 19. Table 20, and Table 21.

TABLE 17
Actual and Baseline Corrected Total IgG Levels by Treatment Arm (Pharmacokinetic Set).

| | TAK-881 0.4 g/kg | | TAK-881 1.0 g/kg | | TAK-881 1.0 g/kg | |
|---|---|---|---|---|---|---|
| | warmed | | warmed | | un-warmed | |
| Parameter | (N = 8) | | (N = 8) | | (N = 8) | |
| Visit | Actual | Baseline | Actual | Baseline | Actual | Baseline |
| Statistic | Value | Corrected | Value | Corrected | Value | Corrected |

Immunoglobulin G (g/L)
Baseline [a]

| | | | |
|---|---|---|---|
| n | 8 | 8 | 8 |
| Mean (SD) | 11.48 | 11.72 | 13.29 |
| | (2.303) | (3.566) | (3.076) |
| Median | 11.97 | 11.01 | 12.82 |
| Min, Max | 7.9, 14.7 | 7.7, 18.9 | 9.1, 17.2 |
| Geo. Mean | 11.26 | 11.29 | 12.97 |
| 95% CI of Geo. Mean | 9.44, 13.43 | 8.87, 14.35 | 10.65, 15.81 |

Day 4

| | | | | | | |
|---|---|---|---|---|---|---|
| n | 8 | 8 | 8 | 8 | 8 | 8 |
| Mean (SD) | 14.67 | 3.19 | 19.84 | 8.12 | 18.49 | 5.20 |
| | (2.141) | (1.662) | (3.089) | (2.130) | (3.808) | (1.981) |
| Median | 14.97 | 3.28 | 18.78 | 8.17 | 18.57 | 5.50 |
| Min, Max | 11.5, 17.3 | 0.3, 6.0 | 16.6, 24.9 | 5.4, 12.1 | 13.8, 23.0 | 2.5, 8.5 |
| Geo. Mean | 14.53 | 2.51 | 19.64 | 7.89 | 18.14 | 4.83 |
| 95% CI of Geo. Mean | 12.81, 16.46 | 1.15, 5.50 | 17.35, 22.24 | 6.35, 9.80 | 15.21, 21.63 | 3.37, 6.92 |

Day 30

| | | | | | | |
|---|---|---|---|---|---|---|
| n | 7 | 7 | 8 | 8 | 8 | 8 |
| Mean (SD) | 11.76 | 0.81 | 14.90 | 3.19 | 15.19 | 1.91 |
| | (3.042) | (0.908) | (3.102) | (0.870) | (2.806) | (0.929) |
| Median | 12.55 | 0.44 | 13.81 | 3.32 | 14.81 | 2.09 |
| Min, Max | 6.4, 15.1 | 0.0, 2.4 | 11.8, 21.2 | 1.8, 4.4 | 11.6, 19.6 | 0.0, 3.1 |
| Geo. Mean | 11.35 | 0.79 | 14.65 | 3.08 | 14.97 | 2.12 |
| 95% CI of Geo. Mean | 8.60, 15.00 | 0.21, 2.93 | 12.45, 17.23 | 2.40, 3.93 | 12.83, 17.45 | 1.65, 2.71 |

Week 12/EOS [b]

| | | | | | | |
|---|---|---|---|---|---|---|
| n | 6 | 6 | 8 | 8 | 7 | 7 |
| Mean (SD) | 11.53 | 0.82 | 12.11 | 0.47 | 12.42 | 0.05 |
| | (2.047) | (0.468) | (3.624) | (0.383) | (2.559) | (0.136) |
| Median | 11.82 | 1.05 | 11.74 | 0.50 | 11.95 | 0.00 |
| Min, Max | 9.0, 13.7 | 0.2, 1.3 | 7.4, 19.3 | 0.0, 1.2 | 9.5, 16.2 | 0.0, 0.4 |
| Geo. Mean | 11.38 | 0.65 | 11.67 | 0.59 | 12.20 | 0.36 |
| 95% CI of Geo. Mean | 9.39, 13.79 | 0.27, 1.57 | 9.15, 14.88 | 0.39, 0.88 | 10.10, 14.72 | NE, NE |

Total IgG levels below the lower limit of quantification were treated as zero for the calculation of summary statistics.
Baseline-corrected total IgG levels were calculated as (total IgG level at post baseline visit)-(total IgG level at baseline visit). Negative baseline-line corrected total IgG levels were treated as zero for calculation of summary statistics.
The table includes only scheduled assessments within the defined visit windows.
[a] Baseline is defined as the last non-missing value before the administration of investigational product.
[b] EOS = End of Study.
NE = Not Estimable.

TABLE 18
Occurrence of Tolerability Events by BMI Group,
Infusion Site and Treatment Arm (Safety Set).

| | TAK-881 | TAK-881 | | |
|---|---|---|---|---|
| | 0.4 g/kg | 1.0 g/kg | TAK-881 1.0 g/kg | |
| BMI Group | warmed | warmed | un-warmed | Total |
| Infusion Site | (N = 8) | (N = 8) | (N = 8) | (N = 24) |
| Tolerability Events | n (%) | n (%) | n (%) | n (%) |
| Overall | | | | |
| Infusion Site 1 | 8 | 8 | 8 | 24 |
| Occurrence of Tolerability Event | 8 (100) | 8 (100) | 8 (100) | 24 (100) |
| Infusion Site 2 | 0 | 4 | 7 | 11 |
| Occurrence of Tolerability Event | 0 | 4 (100) | 7 (100) | 11 (100) |
| BMI 18 to < 25 kg/m² | | | | |
| Infusion Site 1 | 3 | 5 | 4 | 12 |
| Occurrence of Tolerability Event | 3 (100) | 5 (100) | 4 (100) | 12 (100) |
| Infusion Site 2 | 0 | 1 | 3 | 4 |
| Occurrence of Tolerability Event | 0 | 1 (100) | 3 (100) | 4 (100) |
| BMI ≥ 25 to 30 kg/m² | | | | |
| Infusion Site 1 | 5 | 3 | 4 | 12 |
| Occurrence of Tolerability Event | 5 (100) | 3 (100) | 4 (100) | 12 (100) |
| Infusion Site 2 | 0 | 3 | 4 | 7 |
| Occurrence of Tolerability Event | 0 | 3 (100) | 4 (100) | 7 (100) |

N = number of subjects in each treatment arm,
n = number of subjects in each category.
Percentages are based on the number of subjects in each BMI group with non-missing values at the given infusion site.
A tolerability event is considered to have occurred if an infusion was tolerable. An infusion is considered tolerable if the infusion rate was not reduced or the infusion was not interrupted or stopped, due to any TEAE related to TAK 881.

TABLE 19
Treatment-Emergent Adverse Events (TEAEs) by System Organ Class, Preferred Term and Treatment
Arm (Safety Set).

| | TAK-881 0.4 g/kg | | | TAK-881 1.0 g/kg | | | TAK-881 1.0 g/kg | | | | | |
|---|---|---|---|---|---|---|---|---|---|---|---|---|
| | warmed | | | warmed | | | un-warmed | | | Total | | |
| System Organ Class | (N = 8) | | | (N = 8) | | | (N = 8) | | | (N = 24) | | |
| Preferred Term | n | (%) | m | n | (%) | m | n | (%) | m | n | (%) | m |
| Any TEAE | 8 | (100) | 27 | 8 | (100) | 34 | 8 | (100) | 46 | 24 | (100) | 107 |
| Gastrointestinal disorders | 1 | (12.5) | 2 | 0 | | 0 | 0 | | 0 | 1 | (4.2) | 2 |
| Diarrhoea | 1 | (12.5) | 1 | 0 | | 0 | 0 | | 0 | 1 | (4.2) | 1 |
| Nausea | 1 | (12.5) | 1 | 0 | | 0 | 0 | | 0 | 1 | (4.2) | 1 |
| General disorders and | 8 | (100) | 24 | 8 | (100) | 34 | 8 | (100) | 44 | 24 | (100) | 102 |
| administration site conditions | | | | | | | | | | | | |
| Infusion site erythema | 8 | (100) | 9 | 8 | (100) | 11 | 8 | (100) | 17 | 24 | (100) | 37 |
| Infusion site swelling | 7 | (87.5) | 7 | 8 | (100) | 12 | 8 | (100) | 15 | 23 | (95.8) | 34 |
| Infusion site pruritus | 3 | (37.5) | 3 | 5 | (62.5) | 6 | 5 | (62.5) | 8 | 13 | (54.2) | 17 |
| Infusion site pain | 4 | (50.0) | 4 | 5 | (62.5) | 5 | 4 | (50.0) | 4 | 13 | (54.2) | 13 |
| Infusion site | 1 | (12.5) | 1 | 0 | | 0 | 0 | | 0 | 1 | (4.2) | 1 |
| extravasation | | | | | | | | | | | | |
| Nervous system disorders | 1 | (12.5) | 1 | 0 | | 0 | 2 | (25.0) | 2 | 3 | (12.5) | 3 |
| Headache | 1 | (12.5) | 1 | 0 | | 0 | 1 | (12.5) | 1 | 2 | (8.3) | 2 |
| Dizziness | 0 | | 0 | 0 | | 0 | 1 | (12.5) | 1 | 1 | (4.2) | 1 |

N = number of subjects in each treatment arm,
n = number of subjects who experienced the event,
m = number of events.
Percentages are based on the number of subjects in each treatment arm.
Adverse events are classified into system organ class and preferred term using Version 24.1 of MedDRA.
A treatment-emergent adverse event (TEAE) is defined as any adverse event that started at or after the initiation of treatment with TAK-881.
Subjects are counted only once per preferred term.

TABLE 20
Treatment-Emergent Adverse Events (TEAEs) by BMI Group, Category and Treatment Arm (Safety
Set).

| | TAK-881 0.4 g/kg | | | TAK-881 1.0 g/kg | | | TAK-881 1.0 g/kg | | | | | |
|---|---|---|---|---|---|---|---|---|---|---|---|---|
| BMI Group | warmed | | | warmed | | | un-warmed | | | Total | | |
| Category | n | (%) | m | n | (%) | m | n | (%) | m | n | (%) | m |
| Overall | | N = 8 | | | N = 8 | | | N = 8 | | | N = 24 | |
| Any TEAE | 8 | (100) | 27 | 8 | (100) | 34 | 8 | (100) | 46 | 24 | (100) | 107 |
| Serious TEAE | 0 | | 0 | 0 | | 0 | 0 | | 0 | 0 | | 0 |
| TAK-881-related TEAEs | 8 | (100) | 25 | 8 | (100) | 34 | 8 | (100) | 46 | 24 | (100) | 105 |
| within 72 hours | | | | | | | | | | | | |
| Local TEAEs | 8 | (100) | 24 | 8 | (100) | 34 | 8 | (100) | 44 | 24 | (100 | 102 |
| Systemic TEAEs | 2 | (25.0) | 3 | 0 | | 0 | 2 | (25.0) | 2 | 4 | (16.7) | 5 |
| Temporally associated | 8 | (100) | 27 | 8 | (100) | 34 | 8 | (100) | 46 | 24 | (100) | 107 |
| TEAEs within 72 hours | | | | | | | | | | | | |
| TEAEs by maximum | | | | | | | | | | | | |
| severity grade | | | | | | | | | | | | |
| Grade 1 | 8 | (100) | 27 | 8 | (100) | 34 | 8 | (100) | 46 | 24 | (100) | 107 |
| Grade 2 | 0 | | 0 | 0 | | 0 | 0 | | 0 | 0 | | 0 |
| Grade 3 | 0 | | 0 | 0 | | 0 | 0 | | 0 | 0 | | 0 |
| Grade 4 | 0 | | 0 | 0 | | 0 | 0 | | 0 | 0 | | 0 |
| Grade 5 | 0 | | 0 | 0 | | 0 | 0 | | 0 | 0 | | 0 |
| TEAEs leading to | 0 | | 0 | 0 | | 0 | 0 | | 0 | 0 | | 0 |
| discontinuation from the | | | | | | | | | | | | |
| study | | | | | | | | | | | | |
| TEAEs of special interest | 1 | (12.5) | 1 | 0 | | 0 | 0 | | 0 | 1 | (4.2) | 1 |
| Allergy | 0 | | 0 | 0 | | 0 | 0 | | 0 | 0 | | 0 |
| Catheter leakage | 1 | (12.5) | 1 | 0 | | 0 | 0 | | 0 | 1 | (4.2) | 1 |
| Thromboembolic | 0 | | 0 | 0 | | 0 | 0 | | 0 | 0 | | 0 |
| events | | | | | | | | | | | | |

N = number of subjects in each treatment arm,
n = number of subjects who experienced the event,
m = number of events.
Percentages are based on the number of subjects in each treatment arm.
Adverse events are classified into system organ class and preferred term using Version 24.1 of MedDRA.
A treatment-emergent adverse event (TEAE) is defined as any adverse event that started at or after the initiation of treatment with TAK-881.
Subjects are counted only once per preferred term.

TABLE 21
Administration Parameters by BMI group,
Infusion Site and Treatment Arm (Safety Set).

| | | | |
|---|---|---|---|
| BMI Group | TAK-881 | TAK-881 | TAK-881 |
| Infusion Site | 0.4 g/kg | 1.0 g/kg | 1.0 g/kg |
| Parameter | warmed | warmed | un-warmed |
| Statistic/Category | (N = 8) | (N = 8) | (N = 8) |

Overall

| | | | |
|---|---|---|---|
| Infusion Site 1 | | | |
| Maximum tolerable infusion rate | 8 | 8 | 8 |
| for IGSC 20% (mL/hour) [n (%)] [a] | | | |
| 30 | 0 | 0 | 0 |
| 60 | 0 | 0 | 0 |
| 120 | 0 | 0 | 0 |
| 180 | 0 | 0 | 0 |
| 300 | 8 (100) | 8 (100) | 8 (100) |
| Total rHuPH20 volume infused (mL) | | | |
| n | 8 | 8 | 8 |
| Mean (SD) | 14.3 (1.28) | 29.3 (1.39) | 29.4 (1.77) |
| Median | 14.0 | 30.0 | 30.0 |
| Min, Max | 13, 16 | 26, 30 | 25, 30 |
| Total IGSC 20% volume infused (mL) | | | |
| n | 8 | 8 | 8 |
| Mean (SD) | 141.3 (13.30) | 291.3 (14.33) | 293.8 (17.68) |
| Median | 140.0 | 300.0 | 300.0 |
| Min, Max | 125, 160 | 260, 300 | 250, 300 |
| Time to deliver the total volume | | | |
| infused (min) [b] | | | |
| n | 8 | 8 | 8 |
| Mean (SD) | 65.0 (5.24) | 98.8 (3.45) | 102.3 (5.87) |
| Median | 63.5 | 99.5 | 102.0 |
| Min, Max | 59, 74 | 92, 103 | 91, 112 |
| Infusion Site 2 | | | |
| Maximum tolerable infusion rate | 0 | 4 | 7 |
| for IGSC 20% (mL/hour) [n (%)] [a] | | | |
| 30 | 0 | 0 | 0 |
| 60 | 0 | 0 | 2 (28.6) |
| 120 | 0 | 0 | 1 (14.3) |
| 180 | 0 | 1 (25.0) | 1 (14.3) |
| 300 | 0 | 3 (75.0) | 3 (42.9) |
| Total rHuPH20 volume infused (mL) | | | |
| n | 0 | 4 | 7 |
| Mean (SD) | | 10.0 (5.16) | 7.3 (6.16) |
| Median | | 10.0 | 5.0 |
| Min, Max | | 4,16 | 2,16 |
| Total IGSC 20% volume infused (mL) | | | |
| n | 0 | 4 | 4 |
| Mean (SD) | | 97.5 (51.72) | 70.0 (61.64) |
| Median | | 95.0 | 50.0 |
| Min, Max | | 40,160 | 15, 155 |
| Time to deliver the total volume | | | |
| infused (min) [b] | | | |
| n | 0 | 4 | 7 |
| Mean (SD) | | 52.3 (14.36) | 42.3 (20.30) |
| Median | | 53.0 | 41.0 |
| Min, Max | | 35, 68 | 22, 69 |
| Infusion Site 1 and 2 Combined | | | |
| Total rHuPH20 volume infused (mL) | | | |
| n | 8 | 8 | 8 |
| Mean (SD) | 14.3 (1.28) | 34.3 (7.11) | 35.8 (7.17) |
| Median | 14.0 | 32.0 | 33.5 |
| Min, Max | 13, 16 | 26, 46 | 25, 46 |
| Total IGSC 20% volume infused (mL) | | | |
| n | 8 | 8 | 8 |
| Mean (SD) | 141.3 (13.30) | 340.0 (71.01) | 355.0 (71.11) |
| Median | 140.0 | 320.0 | 335.0 |
| Min, Max | 125, 160 | 260, 460 | 250, 455 |
| Time to deliver the total volume | | | |
| infused (min) [c] | | | |
| n | 8 | 8 | 8 |
| Mean (SD) | 65.0 (5.24) | 124.9 (31.99) | 139.3 (26.52) |
| Median | 63.5 | 117.5 | 139.0 |
| Min, Max | 59, 74 | 92, 171 | 91, 175 |

N = number of subjects in each treatment arm,
n = number of subjects with non-missing values for continuous parameters,
n = number of subjects in each category for categorical parameters.
Percentages are based on the number of subjects in each BMI group with non-missing values at the given infusion site.
[a] refers to the administration of IGSC 20%.
[b] Refers to the time from start of rHuPH20 administration to end of IGSC 20% administration.
[c] The time between end of IGSC 20% administration at infusion site 1 and start of rHuPH20 administration at infusion site 2 is not included.
NA = Not available.

The present invention has been illustrated by reference to various exemplary embodiments and examples. As will be apparent to those of skill in the art other embodiments and variations of this invention may be devised by others skilled in the art without departing from the true spirit and scope of the invention. The appended claims are to be construed to include all such embodiments and equivalent variations.

The disclosures of each and every patent, patent application, and publication cited herein are hereby incorporated herein by reference in their entirety.

What is claimed is:

1. A kit comprising:
(a) a first container comprising a pharmaceutical formulation of recombinant human hyaluronidase in a pharmaceutically acceptable carrier;
(b) a second container comprising a pharmaceutical formulation of 20% (w/v) IgG in a pharmaceutically acceptable carrier; and
(c) instructions providing guidance for sequentially subcutaneously infusing into a first infusion site, (i), a first aliquot of a pre-determined dosage of the pharmaceutical formulation of recombinant human hyaluronidase and, (ii), following (i), a first aliquot of a pre-determined dosage of the pharmaceutical formulation of 20% (w/v) IgG.

2. The kit according to claim 1, wherein the pharmaceutical formulation of recombinant human hyaluronidase contains 20% (w/v) recombinant human hyaluronidase.

3. The kit according to any preceding claim, wherein the recombinant human hyaluronidase is rHuPH20.

4. The kit according to any preceding claim, further comprising an infusion apparatus for sequentially or simultaneously subcutaneously infusing (i), the pharmaceutical formulation of recombinant human hyaluronidase and, (ii), following (i), the pharmaceutical formulation of 20% (w/v) IgG.

5. The kit according to any preceding claim, further comprising a subcutaneous needle set.

6. The kit according to any preceding claim, wherein the instructions are a component of a Dosage and Administration section of Complete Prescribing Information.

7. The kit according to any preceding claim, wherein the instructions provide guidance for subcutaneously infusing the pharmaceutical formulation of rHuPH20 to the first infusion site.

8. The kit according to any preceding claim, wherein the instructions provide guidance for subcutaneously infusing from about 50 U/g to about 100 U/g IgG of rHuPH20 to the first infusion site.

9. The kit according to any preceding claim, wherein the instructions provide guidance for subcutaneously infusing at up to at least about 100 mL, at up to at least about 150 mL, up to at least about 200 mL, up to at least about 250 mL, or up to at least about 300 mL of the pharmaceutical formulation of 20% (w/v) IgG to the first infusion site.

10. The kit according to any preceding claim, wherein the instruction provide guidance for subcutaneously infusing the first pre-determined dosage of the pharmaceutical formulation of 20% (w/v) IgG to the first infusion site at a rate of at least about 120 mL/hr, at least about 150 mL/hr, at least about 200 mL/hr, at least about 250 mL/hr, or at least about 300 mL/hr.

11. The kit according to any preceding claim, wherein the instructions provide guidance for subcutaneously infusing at least about 120 mL of the pharmaceutical formulation of 20% (w/v) IgG to the first infusion site at a rate of at least about 120 mL/hr, at least about 150 mL/hr, at least about 200 mL/hr, at least about 250 mL/hr, or at least about 300 mL/hr.

12. The kit according to any preceding claim, wherein the instructions provide, (b) guidance for subcutaneously infusing at least about 300 mL of the pharmaceutical formulation of 20% (w/v) IgG to the first infusion site.

13. The kit according to any preceding claim, wherein the instructions provide (a) guidance for subcutaneously infusing at least about 300 mL of the pharmaceutical formulation of 20% (w/v) IgG to the first infusion site at a rate of at least about 300 mL/hr.

14. The kit according to any preceding claim, wherein the instructions provide guidance for subcutaneously infusing the pharmaceutical formulation of 20% (w/v) IgG warmed to a temperature of from about 30° C. to about 41° C., said pharmaceutical formulation warmed to the temperature prior to the infusing, during the infusing, and a combination thereof.

15. The kit according to any preceding claim, wherein the instructions further provide guidance on simultaneously or sequentially subcutaneously infusing at a second infusion site, (i), a second aliquot of the pre-determined dosage of the pharmaceutical formulation of recombinant human hyaluronidase and, (ii), following (i), subcutaneously infusing a second aliquot of the pre-determined dosage of the pharmaceutical formulation of 20% (w/v) IgG at the second infusion site.

16. The kit according to any preceding claim, wherein the instructions provide guidance on subcutaneously infusing the pharmaceutical formulation of rHuPH20 to the first infusion site, followed by infusing the pharmaceutical formulation of 20% (w/v) IgG to the first infusion site using a member selected from:
(i) a subcutaneous needle set;
(ii) a pooling bag;
(iii) a gravity fill set with vented spike;
(iv) a syringe;
(v) a pump;
(vi) a warming device;
(vii) tubing; and a combination thereof.

17. A method of subcutaneously infusing to a first infusion site a pharmaceutical formulation of 20% (w/v) IgG to a subject in need thereof, the method comprising:
(a) infusing to the first infusion site, a first aliquot of a pre-determined dosage of hyaluronidase by infusing a pre-determined volume of the pharmaceutical formulation of hyaluronidase to the first infusion site; and
(b) following (a), infusing to the first infusion site, a first aliquot of a pre-determined dosage of IgG by infusing a first pre-determined volume of the pharmaceutical formulation of 20% (w/v) IgG to the first infusion site.

18. The method of claim 17, further comprising:
(c) infusing to a second infusion site, a second aliquot of the pre-determined dosage of hyaluronidase by infusing a second pre-determined volume of the pharmaceutical formulation of hyaluronidase to the second infusion site; and
(d) following (c), infusing to the second infusion site, a second aliquot of a pre-determined dosage of IgG by infusing a second pre-determined volume of the pharmaceutical formulation of 20% (w/v) IgG to the second infusion site.

19. The method according to any of claims 17-18, wherein the first pre-determined volume of the pharmaceutical formulation of 20% (w/v) IgG is subcutaneously infused to the first infusion site at a first final pre-determined rate.

20. The method according to any of claims 17-18, wherein the first pre-determined volume of the pharmaceutical formulation of 20% (w/v) IgG is at least about 120 mL, at least about 150 mL, at least about 180 mL, at least about 200 mL, at least about 220 mL, at least about 250 mL, at least about 280 mL, or at least about 300 mL.

21. The method according to any of claims 17-20, wherein the first final pre-determined rate is at least about 120 mL/hr, at least about 150 mL/hr, at least about 180 mL/hr, at least about 200 mL/hr, at least about 220 mL/hr, at least about 250 mL/hr, at least about 280 mL/hr, or at least about 300 mL/hr.

22. The method according to any of claims 17-21, wherein the first predetermined volume of the pharmaceutical formulation of 20% (w/v) IgG is from about 100 mL to about 300 mL, e.g., from about 150 mL to about 200 mL, from about 200 mL to about 250 mL, from about 250 mL to about 300 mL, and is infused at the first infusion site at a first final rate of from about 100 mL/hr to about 300 mL/hr, e.g., from about 150 mL/hr to about 200 mL/hr, from about 200 mL/hr to about 250 mL/hr, or from about 250 mL/hr to about 300 mL/hr.

23. The method according to any of claims 17-22, wherein prior to achieving the first final rate of 300 mL/hr, a first intermediate infusing rate less than 300 mL/hr is maintained for a selected time and increased to the first final pre-determined rate.

24. The method of any of claims 17-23, wherein the first pre-determined volume of the pharmaceutical formulation of 20% (w/v) IgG is infused to the first infusion site at a rate of at least about 300 mL/hr without reduction in rate or cessation of infusion due to subject discomfort, pain or a combination thereof.

25. The method of any of claims 17-24, wherein the first pre-determined volume of the pharmaceutical formulation of 20% (w/v) IgG is infused to the first infusion site at a rate encompassing a ramp up phase followed by a terminal phase, wherein the terminal phase rate is about 200 to about 300 mL/hr, e.g., about 220 mL/hr, about 240 mL/hr, about 260 mL/hr, about 280 mL/hr, the terminal phase ending upon infusion of the last of the first pre-determined volume to the first infusion site, the terminal phase proceeding without reduction in rate or cessation of infusion due to subject discomfort, pain or a combination thereof.

26. The method of claim 25, wherein at least about 60% of the first pre-determined volume of the pharmaceutical formulation of 20% (w/v) IgG is infused to the first infusion site during the terminal phase at the first final rate of at least about 200 mL/hr to about 300 mL/hr, e.g., about 220 mL/hr, about 240 mL/hr, about 260 mL/hr, about 280 mL/hr without reduction in rate or cessation of infusion due to subject discomfort, pain or a combination thereof.

27. The method of claim any of claims 17-26, wherein the first predetermined volume is from about 200 mL to about 300 mL, e.g., about 220 mL, about 240 mL, about 260 mL, about 280 mL and the first final rate is from about 200 mL/hr to about 300 mL/hr, e.g., about 220 mL/hr, about 240 mL/hr, about 260 mL/hr, about 280 mL/hr.

28. The method of claim 18, wherein the second final pre-determined rate is about 300 mL/hr, and prior to achieving the second final pre-determined rate a second intermediate infusing rate is maintained for a selected time and increased to the second final pre-determined rate.

29. The method according to any of claims 17-28, wherein the pre-determined dosage of the pharmaceutical formulation of hyaluronidase is essentially similar between the method of infusing the pharmaceutical formulation of 20% (w/v) IgG, and a method of infusing an otherwise identical pharmaceutical formulation containing 10% (w/v) IgG.

30. The method according to any of claims 17-29, wherein the first predetermined dosage of the pharmaceutical formulation of 20% (w/v) IgG is infused to the first infusion site at a rate of from about 2-times to about 3-times greater than that for infusing a pharmaceutical formulation of 20% (w/v) IgG in the absence of the infusing to the first infusion site of the pre-determined dosage of hyaluronidase prior to infusing the pharmaceutical formulation of 20% (w/v) IgG to the first infusion site.

31. The method according to any of claims 17-30, said method practiced with a system configured to practice the method, the system comprising:
(a) a first container comprising a pharmaceutical formulation of recombinant human hyaluronidase in a pharmaceutically acceptable carrier;
(b) a second container comprising a pharmaceutical formulation of 20% w/v IgG in a pharmaceutically acceptable carrier; and
(c) means for sequentially subcutaneously infusing into a first infusion site, (i), the first aliquot of a pre-determined dosage of the pharmaceutical formulation of recombinant human hyaluronidase and, (ii), following (i), the first aliquot of a pre-determined dosage of the pharmaceutical formulation of 20% IgG.

32. The method according to claim 31, the means for sequentially subcutaneously infusing into a first infusion site, comprising:
(i) a subcutaneous needle set;
(ii) a pooling bag;
(iii) a gravity fill set with vented spike;
(iv) a syringe;
(v) a pump;
(vi) a warming device;
(vii) tubing; and a combination thereof.

33. A system for subcutaneously infusing a pharmaceutical formulation of 20% (w/v) IgG, the system configured for subcutaneously infusing the pharmaceutical formulation to a first infusion site of a subject in need thereof, the pharmaceutical formulation comprising:
at least about 20% (w/v) of IgG and an aqueous pharmaceutically acceptable carrier in which the IgG is dissolved;
the system comprising:
a. a first vessel containing the pharmaceutical formulation of 20% (w/v) IgG;
b. a second vessel containing a pharmaceutical formulation of hyaluronidase:
c. a first hypodermic needle comprising a first terminus configured to penetrate a first infusion site of the subject, and a terminal opening disposed therein through which the pharmaceutical formulation of 20% (w/v) IgG is delivered to the first infusion site;
d. an optional first connecting member configured for fluidic connection with the first vessel and the hypodermic needle; and
e. a first warming device configured for thermal contact with a system component selected from the first vessel, the first connecting member, and a combination thereof, the first warming device configured to heat the pharmaceutical formulation of 20% (w/v) IgG to at least about 30° C., maintain the pharmaceutical formulation of 20% (w/v) IgG at a temperature of at least about 30° C., and a combination thereof.

34. The system of claim 33, wherein at least one component of the system is configured to heat the pharmaceutical formulation of 20% (w/v) IgG to a temperature of from about 30° C. to about 41° C., to maintain the pharmaceutical formulation of 20% (w/v) IgG at a temperature of from about 30° C. to about 41° C., and a combination thereof.

35. The system of any of claims 33-34, wherein the warming device is configured to maintain the pharmaceutical formulation of 20% (w/v) IgG essentially constant through the duration of the infusion to the first infusion site.

36. The system of any of claims 33-35, wherein the system further comprises a means for driving the pharmaceutical formulation of 20% (w/v) IgG from the first vessel through the first connecting member and into the first hypodermic needle, from which the pharmaceutical formulation exits the system via the terminal opening thereof.

37. The system of any of claims 33-36, wherein the system further comprises a pump for driving the pharmaceutical formulation of 20% (w/v) IgG from the first vessel through the first connecting member and into the first hypodermic needle, from which the pharmaceutical formulation exits the system via the terminal opening thereof.

38. The system of any of claims 33-37, wherein the pharmaceutical formulation of 20% (w/v) IgG is infused into the first infusion site at a first final flow rate, which is at least about 2 mL/min, at least about 3 mL/min, or at least about 5 mL/min.

39. The system according to any of claims 33-38, wherein the pharmaceutical formulation of 20% (w/v) IgG is essentially free of a small organic molecule incorporated into the formulation expressly to reduce the viscosity thereof.

40. The system of any of claims 33-39, wherein the first vessel is selected from an infusion bag and a syringe.
